(19) United States
(12) Patent Application Publication
(10) Pub. No.: US 2009/0156578 A1
(43) Pub. Date: Jun. 18, 2009

(54) 3-Heterocyclylacrylamide Compounds as Fab I Inhibitors and Antibacterial Agents

(76) Inventors: Henry Pauls, Oakville (CA); Judd M. Berman, Toronto (CA)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/095,977

(22) PCT Filed: Dec. 1, 2006
(86) PCT No.: PCT/US06/45903

§ 371 Date: Dec. 17, 2008

Related U.S. Application Data
(60) Provisional application No. 60/742,514, filed on Dec. 5, 2005.
(60) Provisional application No. 60/754,024, filed on Dec. 23, 2005.

(51) Int. Cl. A61K 31/553; C07D 405/14; A61K 31/444; A61K 31/44; C07D 471/04; A61K 31/5517; A61K 31/551; A61K 31/55; A61K 31/5383; C07D 487/04; A61K 31/4375; C07D 213/73; C07D 498/04

(52) U.S. Cl. 514/211.06; 546/256; 514/333; 514/349; 546/297; 540/491; 514/300; 546/122; 540/561; 514/220; 514/230.5; 544/105; 540/501; 514/221; 514/212.06; 540/521

(57) ABSTRACT

In part, the present invention is directed towards compounds with FabI inhibiting properties. Such compounds may also inhibit other enzymes, including those similar to FabI either structurally or functionally, for example, Fab K. Kits and compositions that include the disclosed compounds are also provided. Methods of treating a subject with a bacterial illness is also disclosed.

### RELATED APPLICATIONS

This application claims priority to U.S. Ser. No. 60/742,514 filed Dec. 5, 2005 and to U.S. Ser. No. 60/742,024 filed Dec. 23, 2005, both of which are incorporated by reference in their entirety.

### GOVERNMENT SUPPORT

This invention was made with support provided by the National Institute of Health; the government, therefore, has certain rights in the invention.

### INTRODUCTION

Infections caused by or related to bacteria are a major cause of human illness worldwide, and the frequency of resistance to standard antibiotics has risen dramatically over the last decade. Hence, there exists an unmet medical need and demand for new agents acting against bacterial targets.

Examples of potential bacterial targets are those enzymes involved in fatty acid biosynthesis. While the overall pathway of saturated fatty acid biosynthesis is similar in all organisms, the fatty acid synthase (FAS) systems vary considerably with respect to their structural organization. It is believed that vertebrates and yeast possess a FAS in which all the enzymatic activities are encoded on one or two polypeptide chains, respectively, and the acyl carrier protein (ACP) is an integral part of the complex. In contrast, in bacterial FAS, it is known that each of the reactions is catalyzed by a distinct, mono-functional enzyme and the ACP is a discrete protein. Therefore, it may be possible to achieve selective inhibition of the bacterial system by appropriate agents.

One such potential bacterial target is the FabI protein. FabI (previously designated EnvM) is believed to function as an enoyl-ACP reductase in the final step of the four reactions involved in each cycle of bacterial fatty acid biosynthesis. It is believed that in this pathway, the first step is catalyzed by β-ketoacyl-ACP synthase, which condenses malonyl-ACP with acetyl-CoA (FabH, synthase III). It is believed that in subsequent rounds, malonyl-ACP is condensed with the growing-chain acyl-ACP (FabB and FabF, synthases I and II, respectively). The second step in the elongation cycle is thought to be ketoester reduction by NADPH-dependent β-ketoacyl-ACP reductase (FabG). Subsequent dehydration by p-hydroxyacyl-ACP dehydrase (either FabA or FabZ) leads to trans-2-enoyl-ACP. Finally, in step four, trans-2-enoyl-ACP is converted to acyl-ACP by an NADH (or NADPH)-dependent enoyl-ACP reductase (Fab I). Further rounds of this cycle, adding two carbon atoms per cycle, would eventually lead to palmitoyl-ACP (16C), where upon the cycle is stopped largely due to feedback inhibition of Fab I by palmitoyl-ACP. Thus, Fab I is believed to be a major biosynthetic enzyme and is a key regulatory point in the overall synthetic pathway of bacterial fatty acid biosynthesis. Other enzymes such as FabK are also believed to play a role in bacterial fatty acid synthesis

The present invention provides, in part, compounds and compositions with FabI inhibiting properties.

### SUMMARY

In part, the present invention is directed towards compounds with FabI inhibiting properties. Such compounds may also inhibit other enzymes, including those similar to FabI either structurally or functionally, for example, Fab K. Other uses for the subject compounds and compositions will be readily discernable to those of skill in the art.

The subject compounds or compositions may be used to treat bacterial infections. In part, the present invention is directed towards compounds that will affect multiple species, for example, have at least some of the properties of so-called “wide spectrum” anti-bacterials. Alternatively, subject compounds that are selective for one or more bacterial or other non-mammalian species, and not for one or more mammalian species (especially human), are also contemplated.

Non-limiting examples of bacteria that the compounds or compositions of the present invention may be used to either destroy or inhibit the growth of include a member of the genus Streptococcus, Staphylococcus, Bordetella, Corynebacterium, Mycobacterium, Neisseria, Haemophilus, Actinomycetes, Streptomycetes, Nocardia, Enterobacter, Yersinia, Francisella, Pasturella, Moraxella, Acinetobacter, Erysipelothrix, Branhamella, Actinobacillus, Streptobacillus, Listeria, Calymmatobacterium, Brucella, Bacillus, Clostridium, Treponema, Escherichia, Salmonella, Kleibsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia, Propionibacterium acnes, and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, coagulase negative Staphylococci, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomyctes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Kleibsiella pneumoniae, Serratia marcessens, Serratia liquefaciens, Vibrio cholera, Shigella dysenterii, Shigella flexneri, Pseudomonas aeruginosa, Franscisella tularensis, Brucella abortis, Bacillus anthracis, Bacillus cereus, Clostridium perfringens, Clostridium tetani, Clostridium botulinum, Treponema pallidum, Rickettsia rickettsii, Helicobacter pylori or Chlamydia trachomitis.

The compounds of the invention may inhibit FabI with a Kᵢ of about 5 μM or less, about 1 μM or less, about 100 nM or less, about 10 nM or less, or about 1 nM or less. In some embodiments, the compounds of the invention may inhibit FabI with an IC₅₀ of about 30 μM or less, about 1 μM or less, about 100 nM or less, or about 10 nM or less. In other embodiments, the compounds may inhibit FabI with an MIC of about 32 μg/mL or less, about 16 μg/mL or less, or about 8 μg/mL or less, about 4 μg/mL or less, about 2 μg/mL or less, about 1 μg/mL or less, about 0.5 μg/mL or less, about 0.25 μg/mL or less, or about 0.125 μg/mL or less.

Pharmaceutical compositions comprising compounds disclosed herein are also contemplated. Such compositions may include a pharmaceutically acceptable carrier or excipient. Methods for formulating compounds of the present invention in a pharmaceutically acceptable carrier or excipient are also provided.

The subject compositions may be administered by one of a variety of means known to those of skill in the art. The subject compounds may be prepared as described herein and as known to those of skill in the art.

In certain embodiments, the present invention provides antibacterial compositions including compounds of the present invention, and methods of using the same, for the reduction and abatement of at least one of the bacteria caused disorders or conditions based on a therapeutic regimen. In certain aspects, the present invention contemplates monitoring such disorders or conditions as part of any therapeutic regimen, which may be administered over the short-term and/or long-term. These aspects of the invention may be particularly helpful in preventive care regimens.

The compounds or compositions of the present invention may be used, for example, in the manufacture of a medicament to treat any of the foregoing bacteria related conditions or diseases.

In another aspect of the present invention, the disclosed compounds may be used to disinfect an inanimate surface by contacting the antibacterial compound to the inanimate surface.

Monitoring or diagnostic regimens or kits that include subject compounds or methods, and instructions for use of these compositions or methods are also comtemplated. For example, the present invention provides for kits containing at least one dose of a subject composition and other materials for a treatment regimen. For example, in one embodiment, a kit of the present invention contains sufficient subject composition for from five to thirty days and optionally equipment and supplies necessary to measure one or more indices relevant to the treatment regiment. In another embodiment, kits of the present invention contain all the materials and supplies, including subject compositions, for carrying out any methods of the present invention. In still another embodiment, kits of the present invention, as described above, additionally include instructions for the use and administration of the subject compositions.

A dosage of the disclosed compounds may be selected to modulate metabolism of the bacteria in such a way as to inhibit or stop growth of said bacteria or by killing said bacteria. The skilled artisan may identify this amount as provided herein as well as by using other methods known in the art.

As explained herein in greater detail, the invention will readily enable the design and implementation of trials in warm-blooded animals, including humans and mammals, necessary for easily determining or tailoring the form and dose for any composition of the present invention.

These embodiments of the present invention, other embodiments, and their features and characteristics, will be apparent from the description, drawings and claims that follow.

### BRIEF DESCRIPTION OF DRAWINGS

FIG. 1 depicts the bacterial fatty acid biosynthesis cycle via a Type II or dissociated fatty acid synthase system.

FIG. 2 depicts a simplified view of ene-amide core flanked by LHS (left-hand side) and RHS (right-hand side) moieties.

### DETAILED DESCRIPTION

### Introduction

The present invention is directed in part towards novel compositions that inhibit bacterial enzymes, and methods of making and using the same. In certain aspects, inhibitors and other compounds of the invention may be found by a structure-guided medicinal chemistry effort.

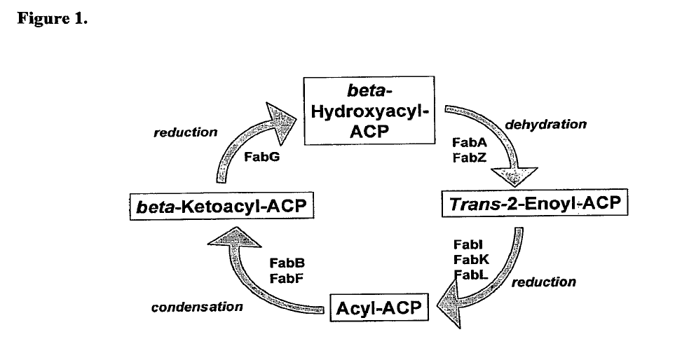

Bacterial fatty acid biosynthesis is believed to proceed via a Type II or dissociated fatty acid synthase system, in contrast to the mammalian Type I system. The overall process is believed to proceed in two stages—initiation and cyclical elongation. Enoyl-ACP reductase is part of the elongation cycle, in which malonyl-ACP is condensed with a growing acyl chain by b-ketoacyl-ACP synthase (FabB, FabF, FabH). The β-ketoester is reduced by β-ketoacyl-ACP reductase, which is then dehydrated to the trans-unsaturated acyl-ACP. The trans-unsaturated acyl-ACP is then reduced by enoyl-ACP reductase. (See FIG. 1).

The enoyl-ACP reductase step is believed to be accomplished by FabI in E. coli and other gram negative organisms and Staphylococci. In certain gram-positive organisms, FabI paralogs exist. In Streptococcus pneumoniae, the enzymatic step is believed to be accomplished by the FabK protein, which has limited homology with the S. aureus FabI protein. In B. subtilis and E. faecalis, genes encoding both FabI and FabK exist. In Mycobacterium tuberculosis a FabI paralog termed InhA exists.

Enoyl-ACP reductase is believed to be the enzymatic target of the antimicrobial product triclosan.

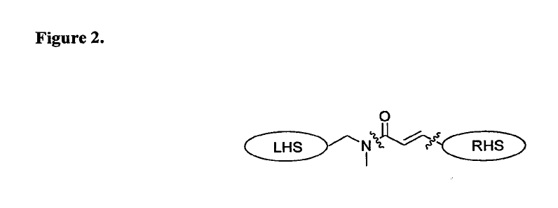

In certain embodiments, the design of new analogs having FabI inhibiting properties is based on viewing the analogs as consisting of a central acrylamide flanked by two relatively hydrophobic groups, conveniently denoted as left-hand side (LHS) and right-hand side (RHS) as put forth in PCT Patent Application WO04/05289. Schematically this is depicted in FIG. 2, where a dumbbell like structure provides one way of viewing certain of the subject compositions (the central bond disconnections that is envisioned in a retrosynthetic sense are shown with dashed lines).

### DEFINITIONS

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used to mean “including but not limited to”. “Including” and “including but not limited to” are used interchangeably.

The term “FabI” is art-recognized and refers to the bacterial enzyme believed to function as an enoyl-acyl carrier protein (ACP) reductase in the final step of the four reactions involved in each cycle of bacterial fatty acid biosynthesis. This enzyme is believed to be widely distributed in bacteria and plants.

The term “enzyme inhibitor” refers to any compound that prevents an enzyme from effectively carrying out its respective biochemical roles. Therefore a “FabI inhibitor” is any compound that inhibits FabI from carrying out its biochemical role. The amount of inhibition of the enzyme by any such compound will vary and is described herein and elsewhere.

The term “antibiotic agent” shall mean any drug that is useful in treating, preventing, or otherwise reducing the severity of any bacterial disorder, or any complications thereof, including any of the conditions, disease, or complications arising therefrom and/or described herein. Antibiotic agents include, for example, cephalosporins, quinolones and fluoroquinolones, penicillins, penicillins and beta lactamase inhibitors, carbepenems, monobactams, macrolides and lincosamines, glycopeptides, rifampin, oxazolidonones, tetracyclines, aminoglycosides, streptogramins, sulfonamides, and the like. Other general categories of antibiotic agents which may be part of a subject composition include those agents known to those of skill in the art as antibiotics and that qualify as (with defined terms being in quotation marks): “drug articles” recognized in the official United States Pharmacopoeia or official National Formulary (or any supplement thereto); “new drug” and “new animal drug” approved by the FDA of the U.S. as those terms are used in Title 21 of the United States Code; any drug that requires approval of a government entity, in the U.S. or abroad (“approved drug”); any drug that it is necessary to obtain regulatory approval so as to comply with 21 U.S.C. §355(a) (“regulatory approved drug”); any agent that is or was subject to a human drug application under 21 U.S.C. §379(g) (“human drug”). (All references to statutory code for this definition refer to such code as of the original filing date of this provisional application.) Other antibiotic agents are disclosed herein, and are known to those of skill in the art. In certain embodiments, the term “antibiotic agent” does not include an agent that is a FabI inhibitor, so that the combinations of the present invention in certain instances will include one agent that is a FabI inhibitor and another agent that is not.

The term “synergistic” is art recognized and refers to two or more components working together so that the total effect is greater than the sum of the effect of the components.

The term “illness” as used herein refers to any illness caused by or related to infection by an organism.

The term “bacterial illness” as used herein refers to any illness caused by or related to infection by bacteria.

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “covalent bond” is art-recognized and refers to a bond between two atoms where electrons are attracted electrostatically to both nuclei of the two atoms, and the net effect of increased electron density between the nuclei counterbalances the internuclear repulsion. The term covalent bond includes coordinate bonds when the bond is with a metal ion.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Antibiotic agents and Fab I/Fab K inhibitors are examples of therapeutic agents.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and/or conditions in an animal or human. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions of the present invention may be administered in a sufficient amount to produce a at a reasonable benefit/risk ratio applicable to such treatment.

The term “synthetic” is art-recognized and refers to production by in vitro chemical or enzymatic synthesis.

The term “meso compound” is art-recognized and refers to a chemical compound which has at least two chiral centers but is achiral due to a plane or point of symmetry.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “Kᵢ” is art-recognized and refers to the dissociation constant of the enzyme-inhibitor complex.

The term “antimicrobial” is art-recognized and refers to the ability of the compounds of the present invention to prevent, inhibit or destroy the growth of microbes such as bacteria, fungi, protozoa and viruses.

The term “antibacterial” is art-recognized and refers to the ability of the compounds of the present invention to prevent, inhibit or destroy the growth of microbes of bacteria.

The term “microbe” is art-recognized and refers to a microscopic organism. In certain embodiments the term microbe is applied to bacteria. In other embodiments the term refers to pathogenic forms of a microscopic organism.

The term “prodrug” is art-recognized and is intended to encompass compounds which, under physiological conditions, are converted into the antibacterial agents of the present invention. A common method for making a prodrug is to select moieties which are hydrolyzed under physiological conditions to provide the desired compound. In other embodiments, the prodrug is converted by an enzymatic activity of the host animal or the target bacteria.

The term “structure-activity relationship” or “(SAR)” is art-recognized and refers to the way in which altering the molecular structure of a drug or other compound alters its interaction with a receptor, enzyme, nucleic acid or other target and the like.

The term “aliphatic” is art-recognized and refers to a linear, branched, cyclic alkane, alkene, or alkyne. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” is also defined to include halosubstituted alkyls.

Moreover, the term “alkyl” (or “lower alkyl”) includes “substituted alkyls”, which refers to alkyl moieties having substituents replacing a hydrogen on one or more carbons of the hydrocarbon backbone. Such substituents may include, for example, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CN and the like. Exemplary substituted alkyls are described below. Cycloalkyls may be further substituted with alkyls, alkenyls, alkoxys, alkylthios, aminoalkyls, carbonyl-substituted alkyls, —CN, and the like.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas:

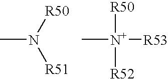

wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula:

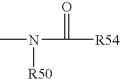

wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula:

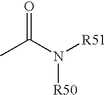

wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas:

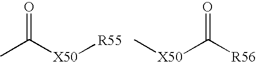

wherein X50 is a bond or represents an oxygen or a sulfur, and R55 and R56 represents a hydrogen, an alkyl, an alkenyl, —(CH₂)ₘ—R61, or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula:

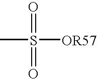

in which R57 is an electron pair, hydrogen, alkyl, cycloalkyl, or aryl.

The term “sulfate” is art recognized and includes a moiety that may be represented by the general formula:

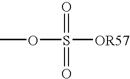

in which R57 is as defined above.

The term “sulfonamido” is art recognized and includes a moiety that may be represented by the general formula:

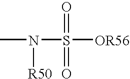

in which R50 and R56 are as defined above.

The term “sulfamoyl” is art-recognized and refers to a moiety that may be represented by the general formula:

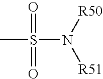

in which R50 and R51 are as defined above.

The term “sulfonyl” is art-recognized and refers to a moiety that may be represented by the general formula:

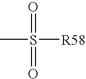

in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula:

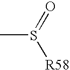

in which R58 is defined above.

The term “phosphoryl” is art-recognized and may in general be represented by the formula:

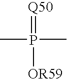

wherein Q50 represents S or O, and R59 represents hydrogen, a lower alkyl or an aryl. When used to substitute, e.g., an alkyl, the phosphoryl group of the phosphorylalkyl may be represented by the general formulas:

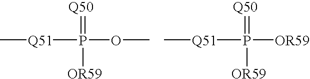

wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas:

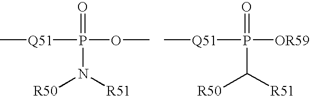

wherein Q51, R50, R51 and R59 are as defined above.

The term “phosphonamidite” is art-recognized and may be represented in the general formulas:

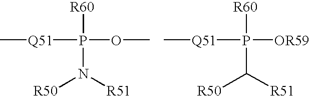

wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67^{th} Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds that may be substituted or unsubstituted.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

The term “hydroxyl-protecting group” is art-recognized and refers to those groups intended to protect a hydrozyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides or polypeptides.

The term “modulation” is art-recognized and refers to up regulation (i.e., activation or stimulation), down regulation (i.e., inhibition or suppression) of a response, or the two in combination or apart.

The term “treating” is art-recognized and refers to curing as well as ameliorating at least one symptom of any condition or disease.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

A “patient,” “subject” or “host” to be treated by the subject method may mean either a human or non-human animal.

The term “mammal” is known in the art, and exemplary mammals include humans, primates, bovines, porcines, canines, felines, and rodents (e.g., mice and rats).

The term “bioavailable” is art-recognized and refers to a form of the subject invention that allows for it, or a portion of the amount administered, to be absorbed by, incorporated to, or otherwise physiologically available to a subject or patient to whom it is administered.

The term “pharmaceutically-acceptable salts” is art-recognized and refers to the relatively non-toxic, inorganic and organic acid addition salts of compounds, including, for example, those contained in compositions of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof from one organ, or portion of the body, to another organ, or portion of the body. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes, for example, subcutaneous administration.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

Contemplated equivalents of the compositions described herein include compositions which otherwise correspond thereto, and which have the same general properties thereof (such as other compositions comprising FabI/Fab K inhibitors), wherein one or more simple variations of substituents or components are made which do not adversely affect the characteristics of the compositions of interest. In general, the components of the compositions of the present invention may be prepared by the methods illustrated in the general reaction schema as, for example, described below, or by modifications thereof, using readily available starting materials, reagents and conventional synthesis procedures. In these reactions, it is also possible to make use of variants which are in themselves known, but are not mentioned here.

### FabI Inhibitors

The FabI inhibitor compounds of the present invention include those depicted by formula I:

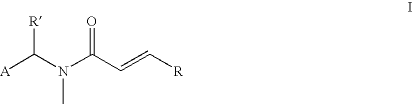

wherein, independently for each occurrence,

A is a monocyclic ring of 4-7 atoms containing 0-2 heteroatoms, a bicyclic ring of 8-12 atoms containing 0-4 heteroatoms or a tricyclic ring of 8-12 atoms containing 0-6 heteroatoms wherein the rings are independently aliphatic, aromatic, heteroaryl or heterocyclic in nature, the heteroatoms are selected from N, S or O and the rings are optionally substituted with one or more groups selected from C₁₋₄ alkyl, OR″, CN, OCF₃, F, Cl, Br, I; wherein R″ is H, alkyl, aralkyl, or heteroaralkyl;

R is

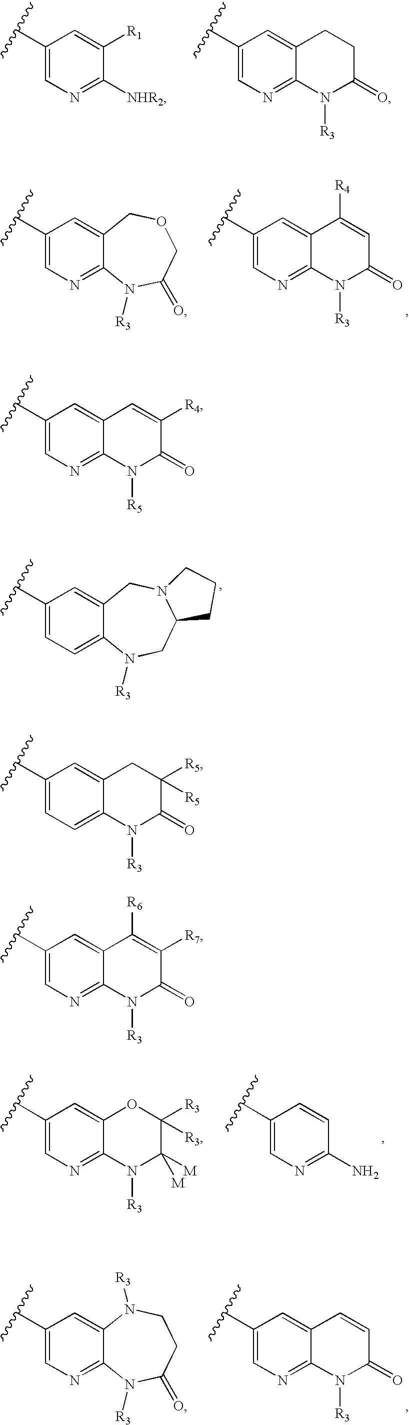

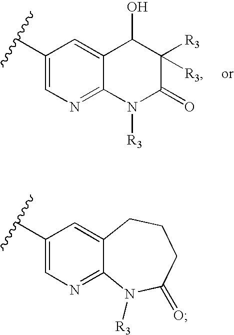

wherein, independently for each occurrence,
R₁ is OH or —O(CH₂)ₙ—Ar;
   wherein,
   n is an integer from 1 to 6 inclusive, and
   Ar is aryl or heteroaryl;
R₂ is H or —C(O)R₃;
R₃ is H, alkyl, or aryl;
R₄ is OH or N(R₃)₂ wherein the two R₃ may form a ring comprising 1 or more heteroatoms;
the two R₅ taken together form a spirocyloalkane, a spiroaryl, or a spiroheterocycloalkane;
R₆ is H, OH, alkyl, or aryl;
R₇ is alkyl, aryl, cycloalkane, or heterocycloalkane; and
M is H or OH, or two M taken together form O or N(R₃); provided that when R is

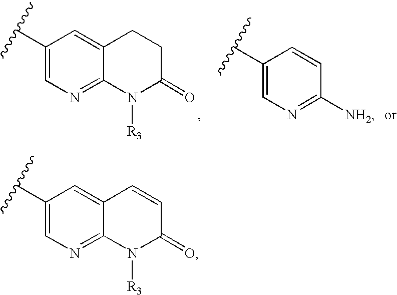

### R′ is (R)-Me.

In a further embodiment, the present invention includes compounds of formula I and the attendant definitions, wherein A is selected from the following:

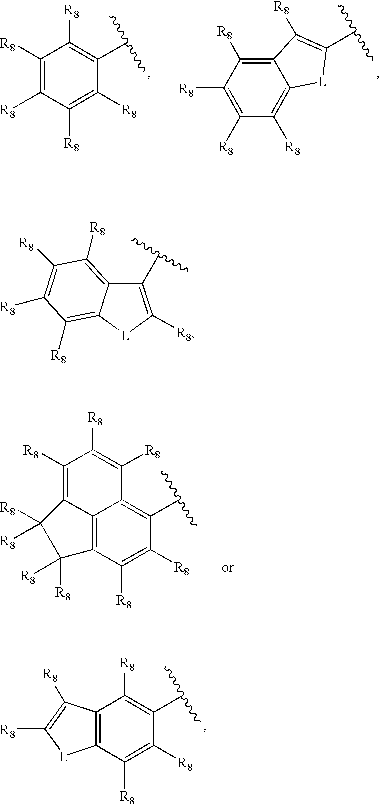

wherein, independently for each occurrence,
R₈ is H, C₁₋₄ alkyl, C₁₋₄ haloalkyl, C₁₋₄ alkenyl, OR″, CN, OCF₃, F, Cl, Br, I; wherein R″ is H, alkyl, aralkyl, or heteroaralkyl; and
L is O, S, or NR₃.

In a further embodiment, the present invention includes compounds of formula I and the attendant definitions, wherein A is selected from the following:

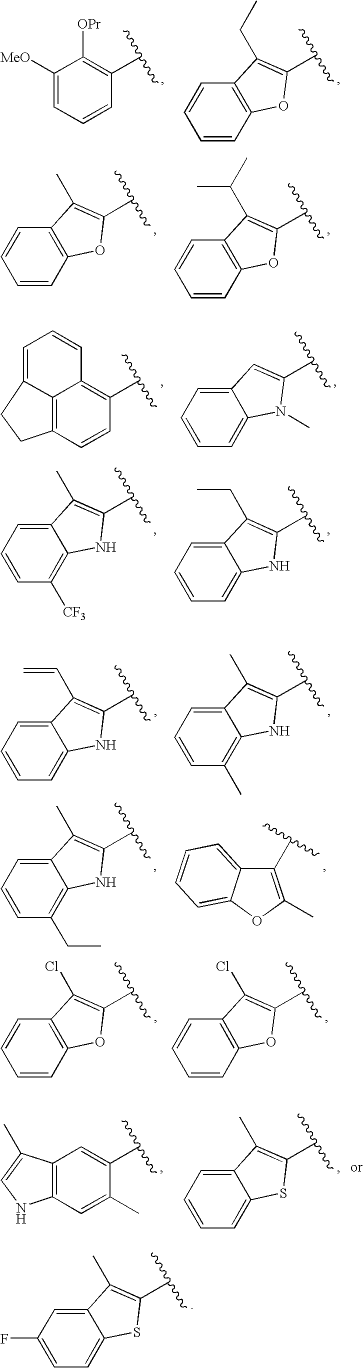

In a further embodiment, the present invention relates to compounds of formula I, wherein the compound has formula Ia:

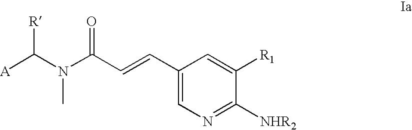

wherein,
R′, R₁ and R₂ are as previously defined, and
A is selected from the following:

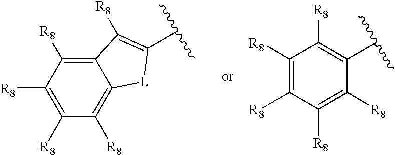

wherein L and R₈ are as previously defined.

In a further embodiment, the present invention relates to compounds of formula Ia and the attendant definitions, wherein R′ is H.

In a further embodiment, the present invention relates to compounds of formula Ia and the attendant definitions, wherein R₁ is —O(CH₂)ₙ—Ar.

In a further embodiment, the present invention relates to compounds of formula Ia and the attendant definitions, wherein R₁ is —OCH₂(C₅H₄N).

In a further embodiment, the present invention relates to compounds of formula Ia and the attendant definitions, wherein R₁ is OH.

In a further embodiment, the present invention relates to compounds of formula Ia and the attendant definitions, wherein R₂ is H.

In a further embodiment, the present invention relates to compounds of formula Ia and the attendant definitions, wherein R₂ is —C(O)CH₃.

In a further embodiment, the present invention relates to compounds of formula Ia and the attendant definitions, wherein A is

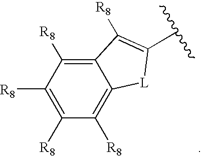

In a further embodiment, the present invention relates to compounds of formula Ia and the attendant definitions, wherein A is

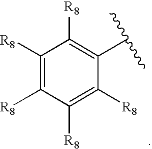

In a further embodiment, the present invention relates to compounds of formula Ia and the attendant definitions, wherein A is

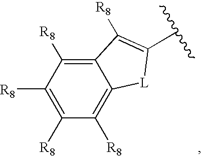

### and L is O.

In a further embodiment, the present invention relates to compounds of formula Ia and the attendant definitions, wherein A is

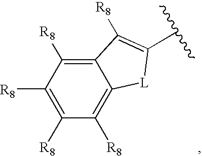

L is O, and R₈ is H or alkyl.

In a further embodiment, the present invention relates to compounds of formula Ia and the attendant definitions, wherein A is

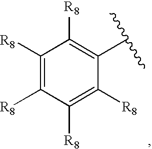

wherein R₈ is OR″ or H.

In a further embodiment, the present invention relates to compounds of formula Ia and the attendant definitions, wherein A is

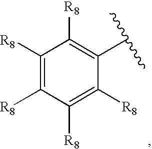

wherein R₈ is OR″ or H, and R″ is alkyl.

In a further embodiment, the present invention relates to compounds of formula I, wherein the compound has formula Ib:

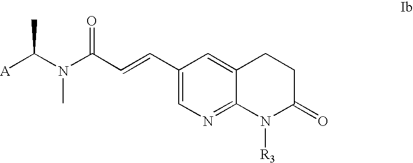

wherein,
R₃ is as defined previously, and
A is selected from the following:

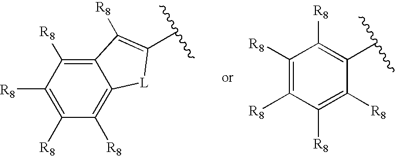

wherein L and R₈ are as previously defined.

In a further embodiment, the present invention relates to compounds of formula Ib and the attendant definitions, wherein R₃ is H.

In a further embodiment, the present invention relates to compounds of formula Ib and the attendant definitions, wherein A is

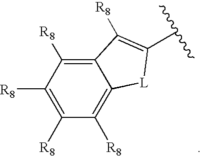

In a further embodiment, the present invention relates to compounds of formula Ib and the attendant definitions, wherein A is

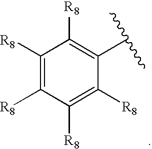

In a further embodiment, the present invention relates to compounds of formula Ib and the attendant definitions, wherein A is

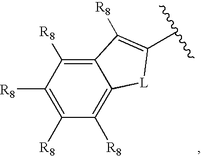

### and L is O.

In a further embodiment, the present invention relates to compounds of formula Ib and the attendant definitions, wherein A is

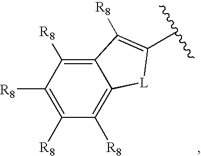

### and L is S.

In a further embodiment, the present invention relates to compounds of formula Ib and the attendant definitions, wherein A is

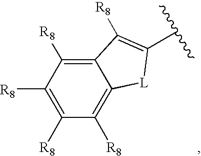

and R₈ is H or alkyl.

In a further embodiment, the present invention relates to compounds of formula Ib and the attendant definitions, wherein A is

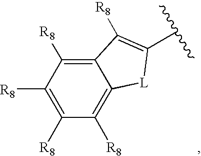

L is O or S, and R₈ is H or alkyl.

In a further embodiment, the present invention relates to compounds of formula Ib and the attendant definitions, wherein A is

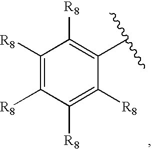

### and R₈ is H or OR″.

In a further embodiment, the present invention relates to compounds of formula Ib and the attendant definitions, wherein A is

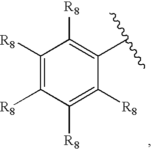

R₈ is H or OR″, and R″ is alkyl.

In a further embodiment, the present invention relates to compounds of formula I, wherein the compound has formula Ic:

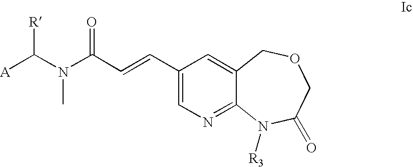

wherein,
R₁ and R₃ are as defined previously, and
A is selected from the following:

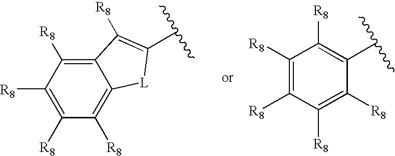

wherein L and R₈ are as previously defined.

In a further embodiment, the present invention relates to compounds of formula Ic and the attendant definitions, wherein R′ is H.

In a further embodiment, the present invention relates to compounds of formula Ic and the attendant definitions, wherein R′ is Me.

In a further embodiment, the present invention relates to compounds of formula Ic and the attendant definitions, wherein R′ is (R)-Me.

In a further embodiment, the present invention relates to compounds of formula Ic and the attendant definitions, wherein R₃ is H.

In a further embodiment, the present invention relates to compounds of formula Ic and the attendant definitions, wherein A is

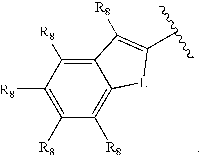

In a further embodiment, the present invention relates to compounds of formula Ic and the attendant definitions, wherein A is

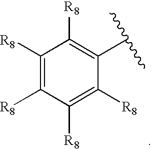

In a further embodiment, the present invention relates to compounds of formula Ic and the attendant definitions, wherein A is

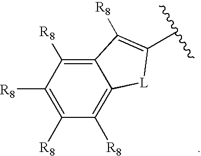

### and L is O.

In a further embodiment, the present invention relates to compounds of formula Ic and the attendant definitions, wherein A is

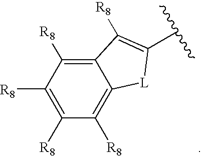

### and L is NH.

In a further embodiment, the present invention relates to compounds of formula Ic and the attendant definitions, wherein A is

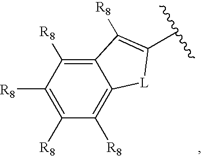

### and L is NMe.

In a further embodiment, the present invention relates to compounds of formula Ic and the attendant definitions, wherein A is

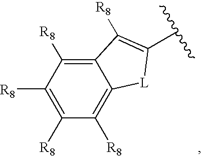

### and L is S.

In a further embodiment, the present invention relates to compounds of formula Ic and the attendant definitions, wherein A is

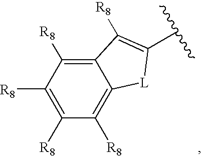

and R₈ is H, alkyl, or Cl.

In a further embodiment, the present invention relates to compounds of formula Ic and the attendant definitions, wherein A is

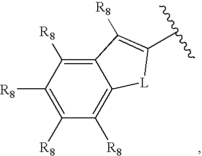

L is O, NH, or S, and R₈ is H, alkyl, or Cl.

In a further embodiment, the present invention relates to compounds of formula Ic and the attendant definitions, wherein A is

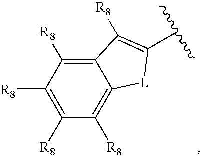

L is O, NMe, or S, and R₈ is H, alkyl, or Cl.

In a further embodiment, the present invention relates to compounds of formula Ic and the attendant definitions, wherein A is

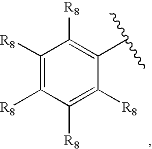

### and R₈ is H or OR″.

In a further embodiment, the present invention relates to compounds of formula Ic and the attendant definitions, wherein A is

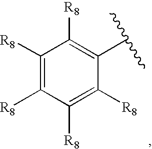

R₈ is H or OR″, and R″ is alkyl.

In a further embodiment, the present invention relates to compounds of formula I, wherein the compound has formula Id:

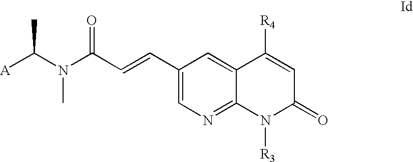

wherein,
R′, R₃ and R₄ are as defined previously, and
A is selected from the following:

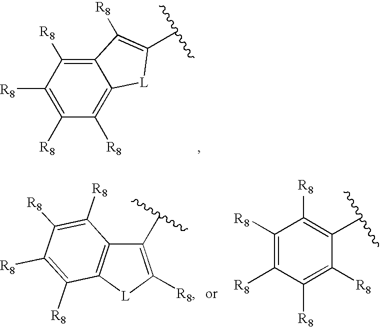

wherein L and R₈ are as previously defined.

In a further embodiment, the present invention relates to compounds of formula Id and the attendant definitions, wherein R′ is H.

In a further embodiment, the present invention relates to compounds of formula Id and the attendant definitions, wherein R₃ is H.

In a further embodiment, the present invention relates to compounds of formula Id and the attendant definitions, wherein R₄ is NH₂.

In a further embodiment, the present invention relates to compounds of formula Id and the attendant definitions, wherein R₄ is OH.

In a further embodiment, the present invention relates to compounds of formula Id and the attendant definitions, wherein A is

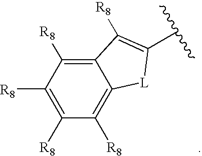

In a further embodiment, the present invention relates to compounds of formula Id and the attendant definitions, wherein A is

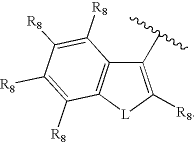

In a further embodiment, the present invention relates to compounds of formula Id and the attendant definitions, wherein A is

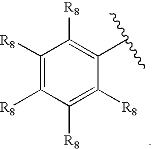

In a further embodiment, the present invention relates to compounds of formula Id and the attendant definitions, wherein A is

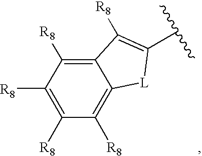

### and L is O.

In a further embodiment, the present invention relates to compounds of formula Id and the attendant definitions, wherein A is

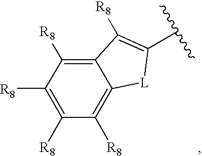

### and L is NMe.

In a further embodiment, the present invention relates to compounds of formula Id and the attendant definitions, wherein A is

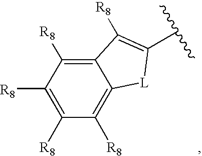

and R₈ is H or alkyl.

In a further embodiment, the present invention relates to compounds of formula Id and the attendant definitions, wherein A is

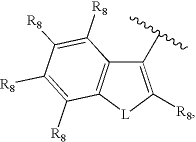

### and L is O.

In a further embodiment, the present invention relates to compounds of formula Id and the attendant definitions, wherein A is

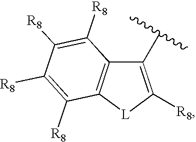

### and R₈ is H or Me.

In a further embodiment, the present invention relates to compounds of formula Id and the attendant definitions, wherein A is

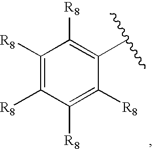

### and R₈ is H or OR″.

In a further embodiment, the present invention relates to compounds of formula Id and the attendant definitions, wherein A is

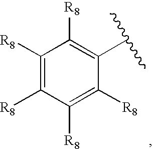

R₈ is H or OR″, and R″ is alkyl.

In a further embodiment, the present invention relates to compounds of formula I, wherein the compound has formula Ie:

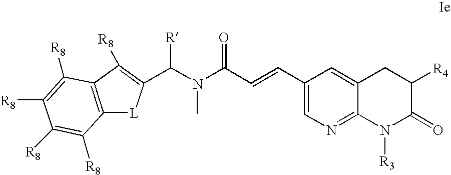

wherein R₁, R₄, R₅, R₈ and L are as defined previously.

In a further embodiment, the present invention relates to compounds of formula Ie and the attendant definitions, wherein R′ is H.

In a further embodiment, the present invention relates to compounds of formula Ie and the attendant definitions, wherein R₄ is N-morpholine.

In a further embodiment, the present invention relates to compounds of formula Ie and the attendant definitions, wherein R₄ is N-piperazine.

In a further embodiment, the present invention relates to compounds of formula Ie and the attendant definitions, wherein R₅ is H.

In a further embodiment, the present invention relates to compounds of formula Ie and the attendant definitions, wherein L is O.

In a further embodiment, the present invention relates to compounds of formula Ie and the attendant definitions, wherein R₈ is H or Me.

In a further embodiment, the present invention relates to compounds of formula Ie and the attendant definitions, wherein R₄ is N-morpholine, and L is O.

In a further embodiment, the present invention relates to compounds of formula Ie and the attendant definitions, wherein R₄ is N-piperazine, and L is O.

In a further embodiment, the present invention relates to compounds of formula I, wherein the compound has formula If:

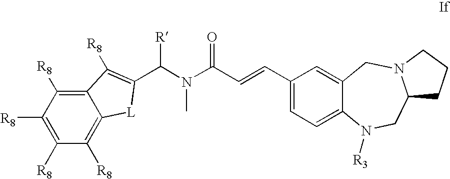

wherein R′, R₃, R₈, and L are as defined previously.

In a further embodiment, the present invention relates to compounds of formula If and the attendant definitions, wherein R′ is H.

In a further embodiment, the present invention relates to compounds of formula If and the attendant definitions, wherein R₃ is H.

In a further embodiment, the present invention relates to compounds of formula If and the attendant definitions, wherein L is O.

In a further embodiment, the present invention relates to compounds of formula If and the attendant definitions, wherein R₈ is H or alkyl.

In a further embodiment, the present invention relates to compounds of formula I, wherein the compound has formula Ig:

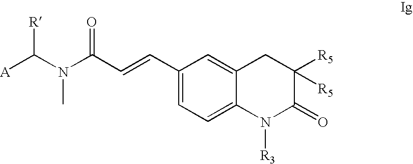

wherein,

R′, R₃, and R₅ are as defined previously, and

A is selected from the following:

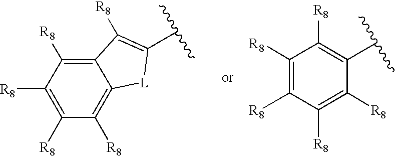

wherein L and R₈ are as previously defined.

In a further embodiment, the present invention relates to compounds of formula Ig and the attendant definitions, wherein R′ is H.

In a further embodiment, the present invention relates to compounds of formula Ig and the attendant definitions, wherein R₃ is H.

In a further embodiment, the present invention relates to compounds of formula Ig and the attendant definitions, wherein the two R₅ taken together form a piperidine ring.

In a further embodiment, the present invention relates to compounds of formula Ig and the attendant definitions, wherein the two R₅ taken together form an N-methyl piperidine ring.

In a further embodiment, the present invention relates to compounds of formula Ig and the attendant definitions, wherein A is

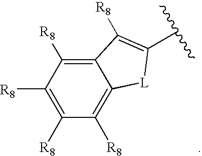

In a further embodiment, the present invention relates to compounds of formula Ig and the attendant definitions, wherein A is

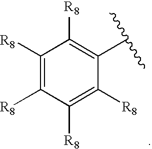

In a further embodiment, the present invention relates to compounds of formula Ig and the attendant definitions, wherein A is

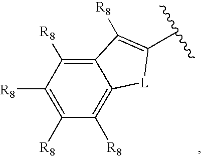

### and L is O.

In a further embodiment, the present invention relates to compounds of formula Ig and the attendant definitions, wherein A is

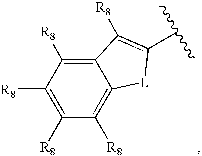

### and L is NMe.

In a further embodiment, the present invention relates to compounds of formula Ig and the attendant definitions, wherein A is

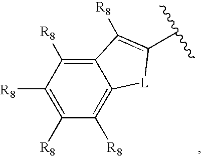

### and L is S.

In a further embodiment, the present invention relates to compounds of formula Ig and the attendant definitions, wherein A is

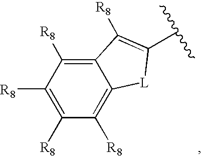

and R₈ is H, alkyl, or F.

In a further embodiment, the present invention relates to compounds of formula Ig and the attendant definitions, wherein A is

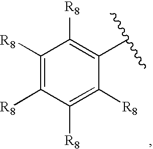

### and R₈ is H or OR″.

In a further embodiment, the present invention relates to compounds of formula Ig and the attendant definitions, wherein A is

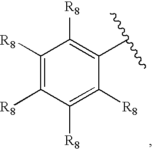

R₈ is H or OR″, and R″ is alkyl.

In a further embodiment, the present invention relates to compounds of formula I, wherein the compound has formula Ih:

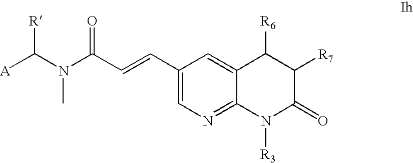

wherein,

R₁, R₃, R₆, R₇, R₈, and L are as previously defined; and

A is selected from the following:

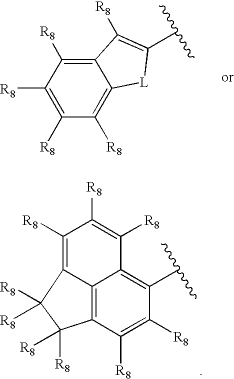

In a further embodiment, the present invention relates to compounds of formula Ih and the attendant definitions, wherein R′ is H.

In a further embodiment, the present invention relates to compounds of formula Ih and the attendant definitions, wherein R′ is Me.

In a further embodiment, the present invention relates to compounds of formula Ih and the attendant definitions, wherein R′ is (R)-Me.

In a further embodiment, the present invention relates to compounds of formula Ih and the attendant definitions, wherein R₃ is H.

In a further embodiment, the present invention relates to compounds of formula Ih and the attendant definitions, wherein R₆ is OH.

In a further embodiment, the present invention relates to compounds of formula Ih and the attendant definitions, wherein R₇ is isopropyl.

In a further embodiment, the present invention relates to compounds of formula Ih and the attendant definitions, wherein R₇ is ethyl.

In a further embodiment, the present invention relates to compounds of formula Ih and the attendant definitions, wherein R₆ is OH and R₇ is isopropyl.

In a further embodiment, the present invention relates to compounds of formula Ih and the attendant definitions, wherein R₆ is OH and R₇ is ethyl.

In a further embodiment, the present invention relates to compounds of formula Ih and the attendant definitions, wherein A is

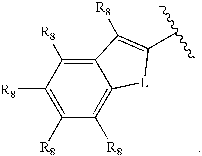

In a further embodiment, the present invention relates to compounds of formula Ih and the attendant definitions, wherein A is

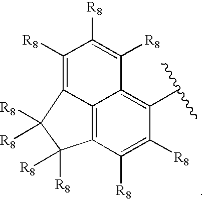

In a further embodiment, the present invention relates to compounds of formula Ih and the attendant definitions, wherein A is

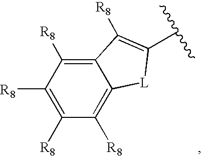

### and L is O.

In a further embodiment, the present invention relates to compounds of formula Ih and the attendant definitions, wherein A is

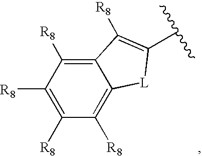

and R₈ is H or alkyl.

In a further embodiment, the present invention relates to compounds of formula Ih and the attendant definitions, wherein A is

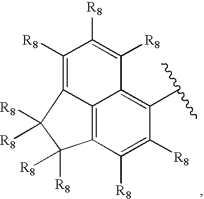

### and R₈ is H.

In a further embodiment, the present invention relates to compounds of formula I, wherein the compound has formula Ii:

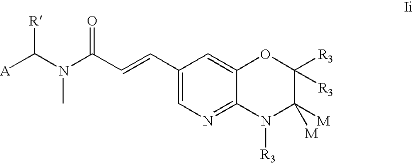

wherein,

R′, R₃, and M are as previously defined, and

A is selected from the following:

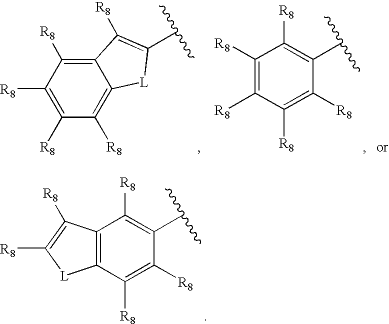

In a further embodiment, the present invention relates to compounds of formula Ii and the attendant definitions, wherein R′ is Me.

In a further embodiment, the present invention relates to compounds of formula Ii and the attendant definitions, wherein the nitrogen bonded R₃ is H.

In a further embodiment, the present invention relates to compounds of formula Ii and the attendant definitions, wherein each geminal R₃ is Me.

In a further embodiment, the present invention relates to compounds of formula Ii and the attendant definitions, wherein each geminal R₃ is H.

In a further embodiment, the present invention relates to compounds of formula Ii and the attendant definitions, wherein each L is H.

In a further embodiment, the present invention relates to compounds of formula Ii and the attendant definitions, wherein one L is H and the other L is OH.

In a further embodiment, the present invention relates to compounds of formula Ii and the attendant definitions, wherein the two L taken together form ═O.

In a further embodiment, the present invention relates to compounds of formula Ii and the attendant definitions, wherein the two L taken together form ═NH.

In a further embodiment, the present invention relates to compounds of formula Ii and the attendant definitions, wherein the two L taken together form ═NMe.

In a further embodiment, the present invention relates to compounds of formula Ii and the attendant definitions, wherein A is

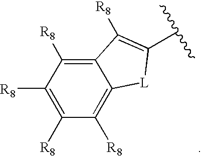

In a further embodiment, the present invention relates to compounds of formula Ii and the attendant definitions, wherein A is

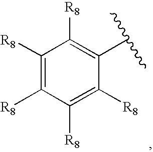

In a further embodiment, the present invention relates to compounds of formula Ii and the attendant definitions, wherein A is

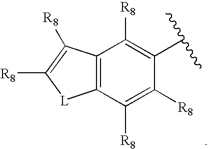

In a further embodiment, the present invention relates to compounds of formula Ii and the attendant definitions, wherein A is

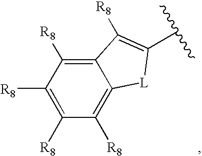

### and L is O.

In a further embodiment, the present invention relates to compounds of formula Ii and the attendant definitions, wherein A is

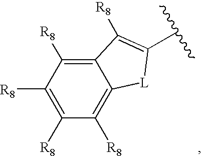

### and L is NH.

In a further embodiment, the present invention relates to compounds of formula Ii and the attendant definitions, wherein A is

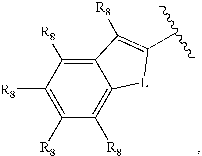

### and L is S.

In a further embodiment, the present invention relates to compounds of formula Ii and the attendant definitions, wherein A is

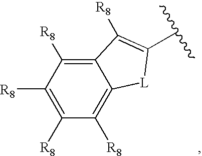

and R₈ is H, alkyl, alkenyl, Cl, F, or CF₃.

In a further embodiment, the present invention relates to compounds of formula Ii and the attendant definitions, wherein A is

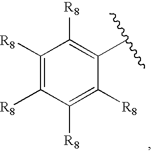

### and R₈ is H or OR″.

In a further embodiment, the present invention relates to compounds of formula Ii and the attendant definitions, wherein A is

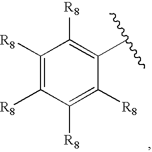

R₈ is H or OR″, and R″ is alkyl.

In a further embodiment, the present invention relates to compounds of formula Ii and the attendant definitions, wherein A is

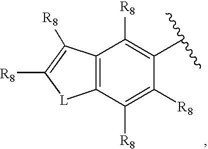

### and L is NH.

In a further embodiment, the present invention relates to compounds of formula Ii and the attendant definitions, wherein A is

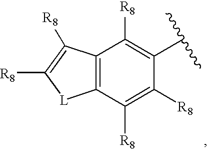

and R₈ is H or alkyl.

In a further embodiment, the present invention relates to compounds of formula I, wherein the compound has formula Ij:

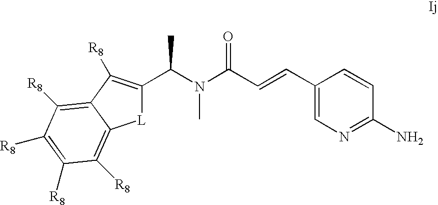

wherein,

R₈ and L are as previously defined.

In a further embodiment, the present invention relates to compounds of formula Ij and the attendant definitions, wherein L is O.

In a further embodiment, the present invention relates to compounds of formula Ij and the attendant definitions, wherein R₈ is H or alkyl.

In a further embodiment, the present invention relates to compounds of formula Ij and the attendant definitions, wherein L is O, and R₈ is H or alkyl.

In a further embodiment, the present invention relates to compounds of formula I, wherein the compound has formula Ik:

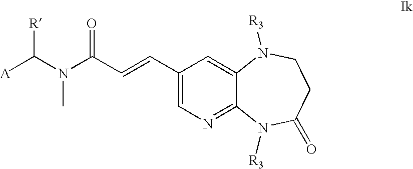

wherein,

R′ and R₃ are as defined previously, and

A is selected from the following:

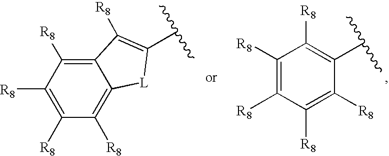

wherein R₈ and L are as defined previously.

In a further embodiment, the present invention relates to compounds of formula Ik and the attendant definitions, wherein R′ is H.

In a further embodiment, the present invention relates to compounds of formula Ik and the attendant definitions, wherein R′ is (R)-Me.

In a further embodiment, the present invention relates to compounds of formula Ik and the attendant definitions, wherein R₃ is H.

In a further embodiment, the present invention relates to compounds of formula Ik and the attendant definitions, wherein A is

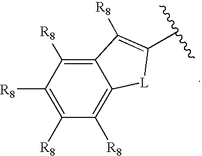

In a further embodiment, the present invention relates to compounds of formula Ik and the attendant definitions, wherein A is

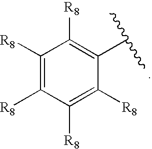

In a further embodiment, the present invention relates to compounds of formula Ik and the attendant definitions, wherein A is

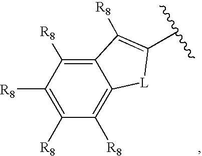

### and L is O.

In a further embodiment, the present invention relates to compounds of formula Ik and the attendant definitions, wherein A is

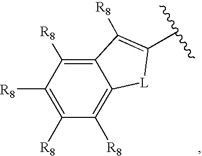

### and L is NMe.

In a further embodiment, the present invention relates to compounds of formula Ik and the attendant definitions, wherein A is

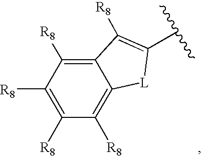

### and L is S.

In a further embodiment, the present invention relates to compounds of formula Ik and the attendant definitions, wherein A is

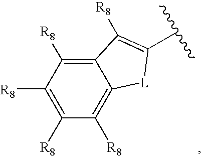

and R₈ is H, F, or alkyl.

In a further embodiment, the present invention relates to compounds of formula Ik and the attendant definitions, wherein A is

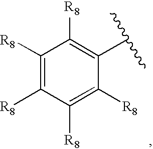

### and R₈ is H or OR″.

In a further embodiment, the present invention relates to compounds of formula Ik and the attendant definitions, wherein A is

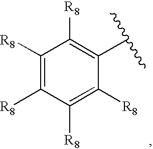

R₈ is H or OR″, and R″ is alkyl.

In a further embodiment, the present invention relates to compounds of formula I, wherein the compound has formula Il:

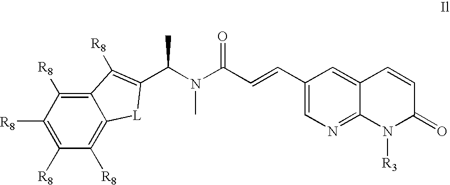

wherein,

R₃, R₈, and L are as previously defined.

In a further embodiment, the present invention relates to compounds of formula Il and the attendant definitions, wherein R₃ is H.

In a further embodiment, the present invention relates to compounds of formula Il and the attendant definitions, wherein L is O.

In a further embodiment, the present invention relates to compounds of formula Il and the attendant definitions, wherein R₈ is H or alkyl.

In a further embodiment, the present invention relates to compounds of formula Il and the attendant definitions, wherein R₃ is H, L is O, and R₈ is H or alkyl.

In a further embodiment, the present invention relates to compounds of formula I, wherein the compound has formula Im:

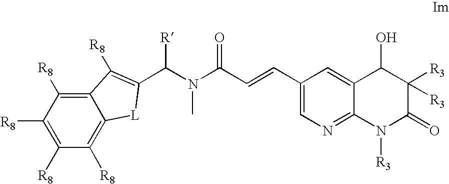

wherein,

R′, R₃, and R₈ are as previously defined.

In a further embodiment, the present invention relates to compounds of formula Im and the attendant definitions, wherein R′ is H.

In a further embodiment, the present invention relates to compounds of formula Im and the attendant definitions, wherein the nitrogen bound R₃ is H.

In a further embodiment, the present invention relates to compounds of formula Im and the attendant definitions, wherein the geminal R₃ are Me.

In a further embodiment, the present invention relates to compounds of formula Im and the attendant definitions, wherein L is O.

In a further embodiment, the present invention relates to compounds of formula Im and the attendant definitions, wherein L is NMe.

In a further embodiment, the present invention relates to compounds of formula Im and the attendant definitions, wherein R₈ is H or alkyl.

In a further embodiment, the present invention relates to compounds of formula Im and the attendant definitions, wherein R′ is H, the nitrogen bound R₃ is H, the geminal R₃ are Me, L is O, and R₈ is H or alkyl.

In a further embodiment, the present invention relates to compounds of formula Im and the attendant definitions, wherein R′ is H, the nitrogen bound R₃ is H, the geminal R₃ are Me, L is NMe, and R₈ is H or alkyl.

In a further embodiment, the present invention relates to compounds of formula I, wherein the compound has formula In:

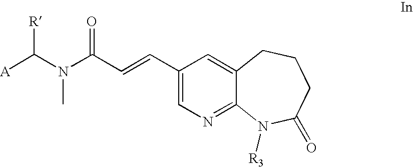

wherein,

R′ and R₃ are as defined previously, and

A is selected from the following:

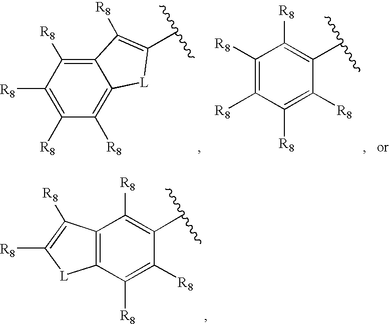

wherein R₈ and L are as defined previously.

In a further embodiment, the present invention relates to compounds of formula In and the attendant definitions, wherein R′ is H.

In a further embodiment, the present invention relates to compounds of formula In and the attendant definitions, wherein R₃ is H.

In a further embodiment, the present invention relates to compounds of formula Im and the attendant definitions, wherein A is

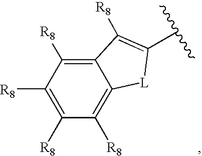

In a further embodiment, the present invention relates to compounds of formula Im and the attendant definitions, wherein A is

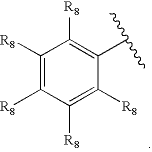

In a further embodiment, the present invention relates to compounds of formula Im and the attendant definitions, wherein A is

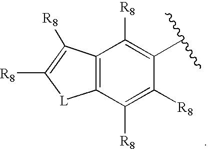

In a further embodiment, the present invention relates to compounds of formula Im and the attendant definitions, wherein A is

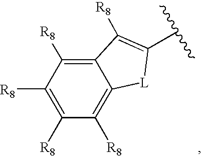

### and L is O.

In a further embodiment, the present invention relates to compounds of formula Im and the attendant definitions, wherein A is

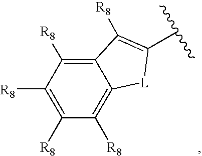

### and L is S.

In a further embodiment, the present invention relates to compounds of formula Im and the attendant definitions, wherein A is

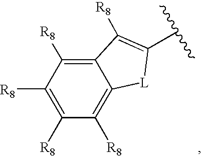

and R₈ is H or alkyl.

In a further embodiment, the present invention relates to compounds of formula Im and the attendant definitions, wherein A is

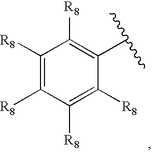

### and R₈ is H or OR″.

In a further embodiment, the present invention relates to compounds of formula Im and the attendant definitions, wherein A is

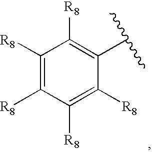

R₈ is H or OR″, and R″ is alkyl.

In a further embodiment, the present invention relates to compounds of formula Im and the attendant definitions, wherein A is

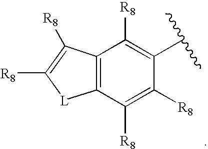

In a further embodiment, the present invention relates to compounds of formula Im and the attendant definitions, wherein A is

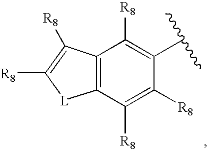

### and L is NH.

In a further embodiment, the present invention relates to compounds of formula Im and the attendant definitions, wherein A is

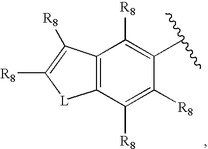

and R₈ is H or alkyl.

The present invention relates to, but is not limited to, the compounds of formula I wherein the compound is selected from the following list:
(E)-3-[6-Amino-5-(pyridin-2-ylmethoxy)-pyridin-3-yl]-N-methyl-N-(3-methyl-benzofuran-2-ylmethyl)acrylamide;
(E)-3-[6-Amino-5-(pyridin-3-ylmethoxy)-pyridin-3-yl]-N-methyl-N-(3-methyl-benzofuran-2-ylmethyl)acrylamide;
(E)-3-(6-Acetylamino-5-hydroxy-pyridin-3-yl)-N-(3-methoxy-2-propoxy-benzyl)-N-methylacrylamide hydrochloride;
(E)-3-(6-Acetylamino-5-hydroxy-pyridin-3-yl)-N-methyl-N-(3-methyl-benzofuran-2-ylmethyl)acrylamide hydrochloride;
(E)-3-(6-Amino-5-benzyloxy-pyridin-3-yl)-N-methyl-N-(3-methyl-benzofuran-2-ylmethyl)acrylamide;
(E)-N-methyl-N-[1-(R)-(3-methyl-benzofuran-2-yl)-ethyl]-3-(7-oxo-5,6,7,8-tetrahydro-[1,8]naphthyridin-3-yl)acrylamide;
(E)-N-Methyl-N-(1-methyl-1H-indol-2-ylmethyl)-3-(8-oxo-5,7,8,9-tetrahydro-6-oxa-1,9-diazabenzocyclohepten-3-yl)-acrylamide hydrochloride;
(E)-N-Methyl-N-(3-methyl-benzofuran-2-ylmethyl)-3-(8-oxo-5,7,8,9-tetrahydro-6-oxa-1,9-diazabenzocyclohepten-3-yl)-acrylamide;
(E)-N-(3-Methoxy-2-propoxy-benzyl)-N-3-(8-oxo-5,7,8,9-tetrahydro-6-oxa-1,9-diazabenzocyclohepten-3-yl)-acrylamide;
(E)-N-Methyl-N-[1-(R)-(3-methyl-benzo[b]thiophen-2-yl)-ethyl]-3-(7-oxo-5,6,7,8-tetrahydro-[1,8]naphthyridin-3-yl)-acrylamide;
(E)-N-Methyl-N-[1-(3-methyl-benzo[b]thiophen-2-yl)-ethyl]-3-(8-oxo-5,7,8,9-tetrahydro-6-oxa-1,9-diaza-benzocyclohepten-3-yl)-acrylamide;
(E)-3-(5-Amino-7-oxo-7,8-dihydro-[1,8]naphthyridin-3-yl)-N-methyl-N-(1-methyl-1H-indol-2-ylmethyl)-acrylamide hydrochloride;
(E)-3-(5-hydroxy-7-oxo-7,8-dihydro-[1,8]naphthyridin-3-yl)-N-methyl-N-(1-methyl-1H-indol-2-ylmethyl)-acrylamide;
(E)-3-(5-hydroxy-7-oxo-7,8-dihydro-[1,8]naphthyridin-3-yl)-N-methyl-N-(3-methyl-benzofuran-2-ylmethyl)-acrylamide;
(E)-N-(2-ethoxy-3-methoxy-benzyl)-3-(5-hydroxy-7-oxo-7,8-dihydro-[1,8]naphthyridin-3-yl)-N-methyl-acrylamide;
(E)-3-(5-hydroxy-7-oxo-7,8-dihydro-[1,8]naphthyridin-3-yl)-N-(3-methoxy-2-propoxy-benzyl)-N-methyl-acrylamide;
(E)-N-(3-Chloro-benzofuran-2-ylmethyl)-N-methyl-3-(2-oxo-1,2,3,5-tetrahydro-benzo[e][1,4]oxazepin-7-yl)-acrylamide;
(S,E)-3-(3,4-cyclopentyl-2,3,4,5-tetrahydro-1H-pyrido[2,3-e][1,4]diazepin-7-yl)-N-methyl-(E)-N-((3-methylbenzofuran-2-yl)methyl)acrylamide trifluoroacetic acid salt;
(E)-N-methyl-N-((3-methylbenzofuran-2-yl)methyl)-3-[7-oxo-(4′-N-Boc-6-spiropiperidinyl)-1,8-naphthyridin-3-yl]acrylamide;
(E)-N-methyl-N-((3-methylbenzofuran-2-yl)methyl)-3-[7-oxo-(6-spiropiperidinyl)-1,8-naphthyridin-3-yl]acrylamide hydrochloride;
(E)-N-methyl-N-((3-methylbenzothiophene-2-yl)methyl)-3-[7-oxo-(4′-N-Boc-6-spiropiperidinyl)-1,8-naphthyridin-3-yl]acrylamide;
(E)-N-methyl-N-((3-methylbenzothiophen-2-yl)methyl)-3-[7-oxo-(6-spiropiperidinyl)-1,8-naphthyridin-3-yl]acrylamide trifluoroacetate salt;
(E)-N-methyl-N-((1-methyl-1H-indol-2-yl)methyl)-3-[7-oxo-(4′-N-Boc-6-spiropiperidinyl)-1,8-naphthyridin-3-yl]acrylamide;
(E)-N-methyl-N-((1-methyl-1H-indol-2-yl)methyl)-3-[7-oxo-(4′-6-spiropiperidinyl)-1,8-naphthyridin-3-yl]acrylamide trifluoroacetate salt;
(E)-N-(3-methoxy-2-propoxybenzyl)-N-methyl-3-[7-oxo-(4′-N-Boc-6-spiropiperidinyl)-1,8-naphthyridin-3-yl]acrylamide;
(E)-N-(3-methoxy-2-propoxybenzyl)-N-methyl-3-[7-oxo-(6-spiropiperidinyl)-1,8-naphthyridin-3-yl]acrylamide hydrochloride;
(E)-N-methyl-N-((3-methylbenzofuran-2-yl)methyl)-3-[7-oxo-(6-4′-N-methyl-spiropiperidinyl)-1,8-naphthyridin-3-yl]acrylamide hydrochloride;
(E)-N-(3-methoxy-2-propoxybenzyl)-N-methyl-3-[7-oxo-(4′-N-methyl-6-spiropiperidinyl)-1,8-naphthyridin-3-yl]acrylamide hydrochloride;
(E)-N-methyl-N-((3-methylbenzothiophen-2-yl)methyl)-3-[7-oxo-(4′-N-methyl-6-spiropiperidinyl)-1,8-naphthyridin-3-yl]acrylamide trifluoroacetate salt;
(E)-3-(6,6-(4-N-methylpiperidine)-7-oxo-5,6,7,8-tetrahydro-1,8-naphthyridin-3-yl)-N-((5-fluoro-3-methylbenzo[b]thiophen-2-yl)methyl)-N-methylacrylamide;
(E)-N-methyl-N-((3-methylbenzofuran-2-yl)methyl)-3-(6-morpholino-7-oxo-7,8-dihydro-1,8-naphthyridin-3-yl)acrylamide;
(E)-N-methyl-N-((3-methylbenzofuran-2-yl)methyl)-3-(6-morpholino-7-oxo-7,8-dihydro-1,8-naphthyridin-3-yl)acrylamide;
(E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride;
(E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride;
(R,E)-N-(1-(3-ethylbenzofuran-2-yl)ethyl)-N-methyl-3-(7-oxo-5,6,7,8-tetrahydro-1,8-naphthyridin-3-yl)acrylamide;
(R,E)-3-(2,2-dimethyl-3-oxo-3,4-dihydro-2H-pyrido[3,2-b][1,4]oxazin-7-yl)-N-(1-(3-ethylbenzofuran-2-yl)ethyl)-N-methylacrylamide;
(R,E)-3-(6-aminopyridin-3-yl)-N-(1-(3-ethylbenzofuran-2-yl)ethyl)-N-methylacrylamide;
(E)-3-(3-hydroxy-2,2-dimethyl-3,4-dihydro-2H-pyrido[3,2-b][1,4]oxazin-7-yl)-N-methyl-N-((3-methylbenzofuran-2-yl)methyl)acrylamide trifluoroacetate salt;
(E)-3-(5-hydroxy-6-isopropyl-7-oxo-7,8-dihydro-1,8-naphthyridin-3-yl)-N-methyl-N-((3-methylbenzofuran-2-yl)methyl)acrylamide,
(E)-N-((1,2-dihydroacenaphthylen-5-yl)methyl)-3-(5-hydroxy-6-isopropyl-7-oxo-7,8-dihydro-1,8-naphthyridin-3-yl)-N-methylacrylamide;
(E)-3-(5-hydroxy-6-ethyl-7-oxo-7,8-dihydro-1,8-naphthyridin-3-yl)-N-methyl-N-((3-methylbenzofuran-2-yl)methyl)acrylamide;
(E)-N-((1,2-dihydroacenaphthylen-5-yl)methyl)-3-(6-ethyl-5-hydroxy-7-oxo-7,8-dihydro-1,8-naphthyridin-3-yl)-N-methylacrylamide;
(E)-3-((E)-2,2-dimethyl-3-(methylimino)-3,4-dihydro-2H-pyrido[3,2-b][1,4]oxazin-7-yl)-N-methyl-N-((3-methylbenzofuran-2-yl)methyl)acrylamide hydrochloride;
(E)-3-((E)-2,2-dimethyl-3-(methylimino)-3,4-dihydro-2H-pyrido[3,2-b][1,4]oxazin-7-yl)-N-methyl-N-((3-methylbenzo[b]thiophen-2-yl)methyl)acrylamide;
(E)-3-(3-imino-2,2-dimethyl-3,4-dihydro-2H-pyrido[3,2-b][1,4]oxazin-7-yl)-N-methyl-N-((3-methylbenzofuran-2-yl)methyl)acrylamide;
(E)-N-methyl-N-((3-methyl-1H-indol-2-yl)methyl)-3-(2-oxo-1,2,3,5-tetrahydropyrido[2,3-e][1,4]oxazepin-7-yl)acrylamide;
(E)-N-((1,3-dimethyl-1H-indol-2-yl)methyl)-N-methyl-3-(2-oxo-2,3,4,5-tetrahydro-1H-pyrido[2,3-b][1,4]diazepin-8-yl)acrylamide;
(E)-N-methyl-N-((3-methylbenzofuran-2-yl)methyl)-3-(4-oxo-2,3,4,5-tetrahydro-1H-pyrido[2,3-b][1,4]diazepin-8-yl)acrylamide;
(E)-N-methyl-N-((3-methylbenzofuran-2-yl)methyl)-3-(4-oxo-2,3,4,5-tetrahydro-1H-pyrido[2,3-b][1,4]diazepin-8-yl)acrylamide;
(E)-N-methyl-N-((3-methyl-1H-indol-2-yl)methyl)-3-(4-oxo-2,3,4,5-tetrahydro-1H-pyrido[2,3-b][1,4]diazepin-8-yl)acrylamide;
(R,E)-N-methyl-N-(1-(3-methylbenzofuran-2-yl)ethyl)-3-(2-oxo-1,2,3,5-tetrahydropyrido[2,3-e][1,4]oxazepin-7-yl)acrylamide;
(R,E)-N-methyl-N-(1-(3-methylbenzofuran-2-yl)ethyl)-3-(7-oxo-7,8-dihydro-1,8-naphthyridin-3-yl)acrylamide;
(R,E)-N-methyl-N-(1-(3-methylbenzofuran-2-yl)ethyl)-3-(3-oxo-3,4-dihydro-2H-pyrido[3,2-b][1,4]oxazin-7-yl)acrylamide;
(R,E)-N-(1-(3-methoxy-2-propoxyphenyl)ethyl)-N-methyl-3-(7-oxo-5,6,7,8-tetrahydro-1,8-naphthyridin-3-yl)acrylamide;
(E)-N-(3-methoxy-2-propoxybenzyl)-N-methyl-3-(4-oxo-2,3,4,5-tetrahydro-1H-pyrido[2,3-b][1,4]diazepin-8-yl)acrylamide;
(R,E)-N-methyl-N-(1-(3-methylbenzofuran-2-yl)ethyl)-3-(4-oxo-2,3,4,5-tetrahydro-1H-pyrido[2,3-b]diazepin-8-yl)acrylamide;
(E)-N-methyl-N-((1-methyl-1H-indol-2-yl)methyl)-3-(4-oxo-2,3,4,5-tetrahydro-1H-pyrido[2,3-b][1,4]diazepin-8-yl)acrylamide;
(E)-N-((5-fluoro-3-methylbenzo[b]thiophen-2-yl)methyl)-N-methyl-3-(4-oxo-2,3,4,5-tetrahydro-1H-pyrido[2,3-b][1,4]diazepin-8-yl)acrylamide;
(R,E)-N-(1-(3-ethylbenzofuran-2-yl)ethyl)-N-methyl-3-(4-oxo-2,3,4,5-tetrahydro-1H-pyrido[2,3-b][1,4]diazepin-8-yl)acrylamide;
(E)-3-(5-hydroxy-6,6-dimethyl-7-oxo-5,6,7,8-tetrahydro-1,8-naphthyridin-3-yl)-N-methyl-N-((3-methylbenzofuran-2-yl)methyl)acrylamide;
(E)-3-(5-hydroxy-6,6-dimethyl-7-oxo-5,6,7,8-tetrahydro-1,8-naphthyridin-3-yl)-N-methyl-N-((1-methyl-1H-indol-2-yl)methyl)acrylamide;
(R,E)-3-(5-hydroxy-6,6-dimethyl-7-oxo-5,6,7,8-tetrahydro-1,8-naphthyridin-3-yl)-N-methyl-N-(1-(3-methylbenzofuran-2-yl)ethyl)acrylamide;
(R,E)-N-(1-(3-ethylbenzofuran-2-yl)ethyl)-N-methyl-3-(7-oxo-5,6,7,8-tetrahydro-1,8-naphthyridin-3-yl)acrylamide;
N-methyl-N-[1-(R)-(3-ethyl-benzofuran-2-yl)-ethyl]-3-(7-oxo-5,6,7,8-tetrahydro-[1,8]naphthyridin-3-yl)acrylamide;
(R,E)-3-(2,2-dimethyl-3-oxo-3,4-dihydro-2H-pyrido[3,2-b][1,4]oxazin-7-yl)-N-(1-(3-ethylbenzofuran-2-yl)ethyl)-N-methylacrylamide;
(R,E)-3-(6-aminopyridin-3-yl)-N-(1-(3-ethylbenzofuran-2-yl)ethyl)-N-methylacrylamide;
(E)-3-(3-hydroxy-2,2-dimethyl-3,4-dihydro-2H-pyrido[3,2-b][1,4]oxazin-7-yl)-N-methyl-N-((3-methylbenzofuran-2-yl)methyl)acrylamide trifluoroacetate salt;
(E)-3-(5-hydroxy-6-isopropyl-7-oxo-7,8-dihydro-1,8-naphthyridin-3-yl)-N-methyl-N-((3-methylbenzofuran-2-yl)methyl)acrylamide;
(E)-N-((1,2-dihydroacenaphthylen-5-yl)methyl)-3-(5-hydroxy-6-isopropyl-7-oxo-7,8-dihydro-1,8-naphthyridin-3-yl)-N-methylacrylamide;
(E)-3-(5-hydroxy-6-ethyl-7-oxo-7,8-dihydro-1,8-naphthyridin-3-yl)-N-methyl-N-((3-methylbenzofuran-2-yl)methyl)acrylamide;
(E)-N-((1,2-dihydroacenaphthylen-5-yl)methyl)-3-(6-ethyl-5-hydroxy-7-oxo-7,8-dihydro-1,8-naphthyridin-3-yl)-N-methylacrylamide;
(E)-3-((E)-2,2-dimethyl-3-(methylimino)-3,4-dihydro-2H-pyrido[3,2-b][1,4]oxazin-7-yl)-N-methyl-N-((3-methylbenzofuran-2-yl)methyl)acrylamide hydrochloride;
(E)-3-((E)-2,2-dimethyl-3-(methylimino)-3,4-dihydro-2H-pyrido[3,2-b][1,4]oxazin-7-yl)-N-methyl-N-((3-methylbenzo[b]thiophen-2-yl)methyl)acrylamide;
(E)-3-(3-imino-2,2-dimethyl-3,4-dihydro-2H-pyrido[3,2-b][1,4]oxazin-7-yl)-N-methyl-N-((3-methylbenzofuran-2-yl)methyl)acrylamide;
(E)-N-methyl-N-((3-methyl-1H-indol-2-yl)methyl)-3-(2-oxo-1,2,3,5-tetrahydropyrido[2,3-e][1,4]oxazepin-7-yl)acrylamide;
(E)-N-methyl-N-((3-methyl-1H-indol-2-yl)methyl)-3-(3-oxo-3,4-dihydro-2H-pyrido[3,2-b][1,4]oxazin-7-yl)acrylamide;
(E)-N-methyl-N-((3-methyl-1H-indol-2-yl)methyl)-3-(1,2,3,5-tetrahydropyrido[2,3-e][1,4]oxazepin-7-yl)acrylamide;
(E)-N-((3-ethyl-1H-indol-2-yl)methyl)-N-methyl-3-(3-oxo-3,4-dihydro-2H-pyrido[3,2-b][1,4]oxazin-7-yl)acrylamide;
(E)-N-methyl-3-(3-oxo-3,4-dihydro-2H-pyrido[3,2-b][1,4]oxazin-7-yl)-N-((3-vinyl-1H-indol-2-yl)methyl)acrylamide;
(E)-N-((1,3-dimethyl-1H-indol-2-yl)methyl)-N-methyl-3-(3-oxo-3,4-dihydro-2H-pyrido[3,2-b][1,4]oxazin-7-yl)acrylamide;
(E)-N-((1,3-dimethyl-1H-indol-2-yl)methyl)-N-methyl-3-3-(7-oxo-5,6,7,8-tetrahydro-1,8-naphthyridin-3-yl)acrylamide;
(E)-N-((1,3-dimethyl-1H-indol-2-yl)methyl)-N-methyl-3-(2-oxo-2,3,4,5-tetrahydro-1H-pyrido[2,3-b][1,4]diazepin-8-yl)acrylamide;
(E)-N-((3,7-dimethyl-1H-indol-2-yl)methyl)-N-methyl-3-(3-oxo-3,4-dihydro-2H-pyrido[3,2-b][1,4]oxazin-7-yl)acrylamide;
(E)-N-methyl-N-((3,7-methyl-1H-indol-2-yl)methyl-3-(7-oxo-5,6,7,8-tetrahydro-1,8-naphthyridin-3-yl)acrylamide;
(E)-N-((3,7-dimethyl-1H-indol-2-yl)methyl)-N-methyl-3-(8-oxo-6,7,8,9-tetrahydro-5H-pyrido[2,3-b]azepin-3-yl)acrylamide;
(E)-N-methyl-N-((3-methyl-7-(trifluoromethyl)-1H-indol-2-yl)methyl)-3-(3-oxo-3,4-dihydro-2H-pyrido[3,2-b][1,4]oxazin-7-yl)acrylamide;
(E)-N-((7-ethyl-3-methyl-1H-indol-2-yl)methyl)-N-methyl-3-(3-oxo-3,4-dihydro-2H-pyrido[3,2-b][1,4]oxazin-7-yl)acrylamide;
(E)-N-((3,6-dimethyl-1H-indol-5-yl)methyl)-N-methyl-3-(3-oxo-3,4-dihydro-2H-pyrido[3,2-b][1,4]oxazin-yl)acrylamide);
(E)-3-(2,2-dimethyl-3,4-dihydro-2H-pyrido[3,2-b][1,4]oxazin-7-yl)-N-methyl-N-((3-methylbenzofuran-2-yl)methyl)acrylamide hydrochloride;
(E)-N-((3-chlorobenzofuran-2-yl)methyl)-N-methyl-3-(3-oxo-3,4-dihydro-2H-pyrido[3,2-b][1,4]oxazin-7-yl)acrylamide;
(E)-N-(3-methoxy-2-propoxybenzyl)-N-methyl-3-(3-oxo-3,4-dihydro-2H-pyrido[3,2-b][1,4]oxazin-7-yl)acrylamide;
(E)-N-((3-isopropylbenzofuran-2-yl)methyl)-N-methyl-3-(3-oxo-3,4-dihydro-2H-pyrido[3,2-b][1,4]oxazin-7-yl)acrylamide;
(E)-N-((3-ethylbenzofuran-2-yl)methyl)-N-methyl-3-(3-oxo-3,4-dihydro-2H-pyrido[3,2-b][1,4]oxazin-7-yl)acrylamide;
(E)-N-((5-fluoro-3-methylbenzo[b]thiophen-2-yl)methyl)-N-methyl-3-(3-oxo-3,4-dihydro-2H-pyrido[3,2-b][1,4]oxazin-7-yl)acrylamide;
(E)-N-methyl-N-((3-methylbenzofuran-2-yl)methyl)-3-(8-oxo-6,7,8,9-tetrahydro-5H-pyrido[2,3-b]azepin-3-yl)acrylamide;
(E)-N-methyl-N-((3-methylbenzofuran-2-yl)methyl)-3-(8-oxo-6,7,8,9-tetrahydro-5H-pyrido[2,3-b]azepin-3-yl)acrylamide;
(E)-N-methyl-N-((3-methylbenzo[b]thiophen-2-yl)methyl)-3-(8-oxo-6,7,8,9-tetrahydro-5H-pyrido[2,3-b]azepin-3-yl)acrylamide;
(E)-N-(3-methoxy-2-propoxybenzyl)-N-methyl-3-(8-oxo-6,7,8,9-tetrahydro-5H-pyrido[2,3-b]azepin-3-yl)acrylamide;
(E)-N-((3,6-dimethyl-1H-indol-5-yl)methyl)-N-methyl-3-(8-oxo-6,7,8,9-tetrahydro-5H-pyrido[2,3-b]azepin-3-yl)acrylamide;
(E)-N-((3,6-dimethyl-1H-indol-5-yl)methyl)-N-methyl-3-(8-oxo-6,7,8,9-tetrahydro-5H-pyrido[2,3-b]azepin-3-yl)acrylamide;
(S,E)-3-(3,4-cyclopentyl-2,3,4,5-tetrahydro-1H-pyrido[2,3-e][1,4]diazepin-7-yl)-N-methyl-N-((3-methylbenzofuran-2-yl)methyl)acrylamide trifluoroacetic acid salt;
(E)-N-methyl-N-((3-methylbenzofuran-2-yl)methyl)-3-[7-oxo-(4′-N-Boc-6-spiropiperidinyl)-1,8-naphthyridin-3-yl]acrylamide;
(E)-N-methyl-N-((3-methylbenzofuran-2-yl)methyl)-3-[7-oxo-(6-spiropiperidinyl)-1,8-naphthyridin-3-yl]acrylamide hydrochloride;
(E)-N-methyl-N-((3-methylbenzothiophen-2-yl)methyl)-3-[7-oxo-(6-spiropiperidinyl)-1,8-naphthyridin-3-yl]acrylamide trifluoroacetate salt;
(E)-N-methyl-N-((3-methylbenzothiophene-2-yl)methyl)-3-[7-oxo-(4′-N-Boc-6-spiropiperidinyl)-1,8-naphthyridin-3-yl]acrylamide;
(E)-N-methyl-N-((1-methyl-1H-indol-2-yl)methyl)-3-[7-oxo-(4′-6-spiropiperidinyl)-1,8-naphthyridin-3-yl]acrylamide trifluoroacetate salt;
(E)-N-methyl-N-((1-methyl-1H-indol-2-yl)methyl)-3-[7-oxo-(4′-N-Boc-6-spiropiperidinyl)-1,8-naphthyridin-3-yl]acrylamide;
(E)-N-(3-methoxy-2-propoxybenzyl)-N-methyl-3-[7-oxo-(4′-N-Boc-6-spiropiperidinyl)-1,8-naphthyridin-3-yl]acrylamide;
(E)-N-(3-methoxy-2-propoxybenzyl)-N-methyl-3-[7-oxo-(6-spiropiperidinyl)-1,8-naphthyridin-3-yl]acrylamide hydrochloride;
(E)-N-methyl-N-((3-methylbenzofuran-2-yl)methyl)-3-[7-oxo-(6-4′-N-methyl-spiropiperidinyl)-1,8-naphthyridin-3-yl]acrylamide hydrochloride;
(E)-N-(3-methoxy-2-propoxybenzyl)-N-methyl-3-[7-oxo-(4′-N-methyl-6-spiropiperidinyl)-1,8-naphthyridin-3-yl]acrylamide hydrochloride;
(E)-N-methyl-N-((3-methylbenzothiophen-2-yl)methyl)-3-[7-oxo-(4′-N-methyl-6-spiropiperidinyl)-1,8-naphthyridin-3-yl]acrylamide trifluoroacetate salt;
(E)-3-(6,6-(4-N-methylpiperidine)-7-oxo-5,6,7,8-tetrahydro-1,8-naphthyridin-3-yl)-N-((5-fluoro-3-methylbenzo[b]thiophen-2-yl)methyl)-N-methylacrylamide;
(E)-N-methyl-N-((3-methylbenzofuran-2-yl)methyl)-3-(6-morpholino-7-oxo-7,8-dihydro-1,8-naphthyridin-3-yl)acrylamide;
(E)-N-methyl-N-((3-methylbenzofuran-2-yl)methyl)-3-(6-morpholino-7-oxo-7,8-dihydro-1,8-naphthyridin-3-yl)acrylamide;
(E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride;
(E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride;
(E)-N-methyl-N-((3-methylbenzofuran-2-yl)methyl)-3-(4-oxo-2,3,4,5-tetrahydro-1H-pyrido[2,3-b][1,4]diazepin-8-yl)acrylamide;
(E)-3-(4-oxo-2,3,4,5-tetrahydro-1H-pyrido[2,3-b][1,4]diazepin-8-yl)acrylic acid hydrochloride;
(E)-N-methyl-N-((3-methyl-1H-indol-2-yl)methyl)-3-(4-oxo-2,3,4,5-tetrahydro-1H-pyrido[2,3-b][1,4]diazepin-8-yl)acrylamide;
(R,E)-3-(2,2-dimethyl-3-oxo-3,4-dihydro-2H-pyrido[3,2-b]oxazin-7-yl)-N-methyl-N-(1-(3-methylbenzofuran-2-yl)ethyl)acrylamide;
(R,E)-N-methyl-N-(1-(3-methylbenzofuran-2-yl)ethyl)-3-(2-oxo-1,2,3,5-tetrahydropyrido[2,3-e][1,4]oxazepin-7-yl)acrylamide;
(R,E)-N-methyl-N-(1-(3-methylbenzofuran-2-yl)ethyl)-3-(7-oxo-7,8-dihydro-1,8-naphthyridin-3-yl)acrylamide;
(R,E)-N-methyl-N-(1-(3-methylbenzofuran-2-yl)ethyl)-3-(3-oxo-3,4-dihydro-2H-pyrido[3,2-b][1,4]oxazin-7-yl)acrylamide;
(R,E)-N-(1-(3-methoxy-2-propoxyphenyl)ethyl)-N-methyl-3-(7-oxo-5,6,7,8-tetrahydro-1,8-naphthyridin-3-yl)acrylamide;
(E)-N-(3-methoxy-2-propoxybenzyl)-N-methyl-3-(4-oxo-2,3,4,5-tetrahydro-1H-pyrido[2,3-b][1,4]diazepin-8-yl)acrylamide;
(R,E)-N-methyl-N-(1-(3-methylbenzofuran-2-yl)ethyl)-3-(4-oxo-2,3,4,5-tetrahydro-1H-pyrido[2,3-b][1,4]diazepin-8-yl)acrylamide;
(E)-N-methyl-N-((1-methyl-1H)-indol-2-yl)methyl)-3-(4-oxo-2,3,4,5-tetrahydro-1H-pyrido[2,3-b][1,4]diazepin-8-yl)acrylamide;
(E)-N-((5-fluoro-3-methylbenzo[b]thiophen-2-yl)methyl)-N-methyl-3-(4-oxo-2,3,4,5-tetrahydro-1H-pyrido[2,3-b][1,4]diazepin-8-yl)acrylamide;
(R,E)-N-(1-(3-ethylbenzofuran-2-yl)ethyl)-N-methyl-3-(4-oxo-2,3,4,5-tetrahydro-1H-pyrido[2,3-b][1,4]diazepin-8-yl)acrylamide;
(E)-3-(5-hydroxy-6,6-dimethyl-7-oxo-5,6,7,8-tetrahydro-1,8-naphthyridin-3-yl)-N-methyl-N-((3-methylbenzofuran-2-yl)methyl)acrylamide;
(E)-3-(5-hydroxy-6,6-dimethyl-7-oxo-5,6,7,8-tetrahydro-1,8-naphthyridin-3-yl)-N-methyl-N-((1-methyl-1H-indol-2-yl)methyl)acrylamide; or
(R,E)-3-(5-hydroxy-6,6-dimethyl-7-oxo-5,6,7,8-tetrahydro-1,8-naphthyridin-3-yl)-N-methyl-N-(1-(3-methylbenzofuran-2-yl)ethyl)acrylamide.

Also included in the antibacterial compositions of the present invention are pharmaceutically acceptable addition salts and complexes of the FabI inhibitors. In cases wherein the inhibitors may have one or more chiral centers, unless specified, the present invention comprises each unique racemic compound, as well as each unique nonracemic compound.

In cases in which the inhibitors have unsaturated carbon-carbon double bonds, both the cis (Z) and trans (E) isomers are within the scope of this invention. In cases wherein inhibitors may exist in tautomeric forms, such as keto-enol tautomers, such as

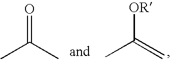

each tautomeric form is contemplated as being included within this invention, whether existing in equilibrium or locked in one form by appropriate substitution with R′. The meaning of any substituent at any one occurrence is independent of its meaning, or any other substituent's meaning, at any other occurrence.

Also included in the antibiotic compounds of the present invention are prodrugs of the FabI inhibitors.

A variety of subject compounds and intermediates of them may be made by a person of ordinary skill in the art using conventional reaction techniques. Non-limiting examples of compounds and methods of making them may be found in U.S. patent application Ser. Nos. 08/790,043, 10/009,219, 10/089,019, 09/968,129, 09/968,123, 09/968,236, 09/959,172, 09/979,560, 09/980,369, 10/089,755, 10/089,739, 10/089,740, and PCT Application Nos. PCT/US03/38706, WO 0027628 and WO 0210332.

Synthetic Routes to Compounds of Formula I

A generalized chemical approach to assembling compounds of formula I is based on viewing the analogs as consisting of a central ene-amide flanked by left-hand side (LHS) and right-hand side (RHS) moieties. Schematically, this is depicted in FIG. 2. Two possible bond disconnections envisioned in a retrosynthetic sense are shown with dashed lines. Schemes 1 to 48 illustrate some of the general methods that can be used in the synthesis of compounds of formula I wherein the final covalent bond formed is via a fleck coupling between an alkene and a suitably halogenated right hand side moiety, or via a dehydrative coupling between a left hand side alkyl amine and an ene-carboxylic acid. It will be recognized by one skilled in the art that other disconnections are possible resulting in alternative modes of assembly of the compounds of the invention.

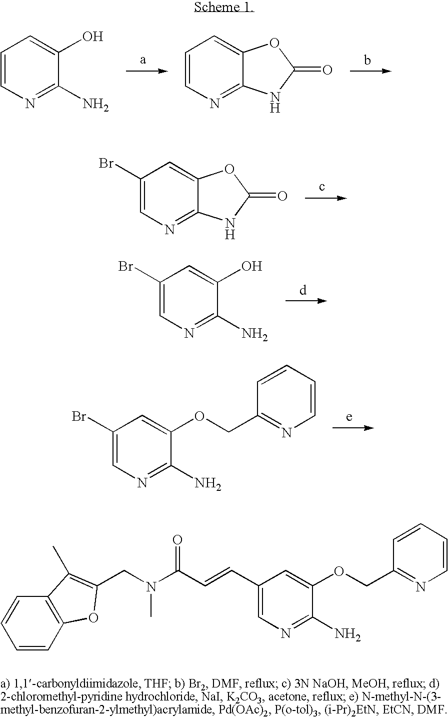

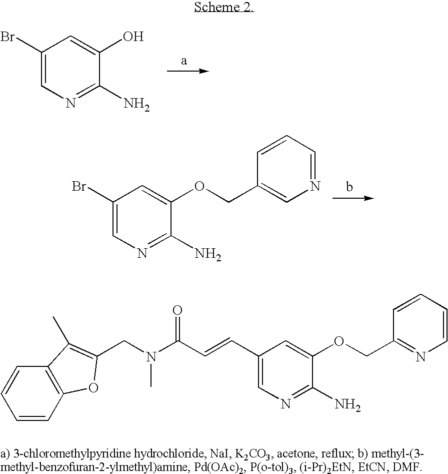

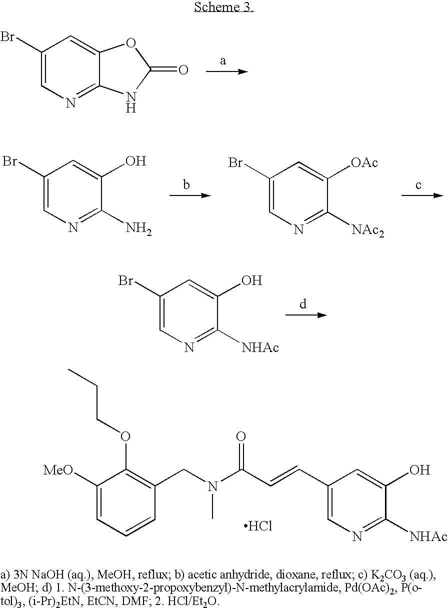

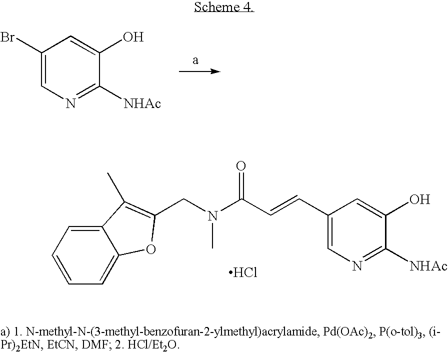

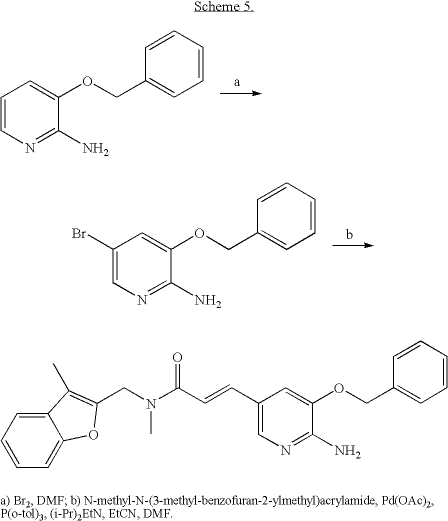

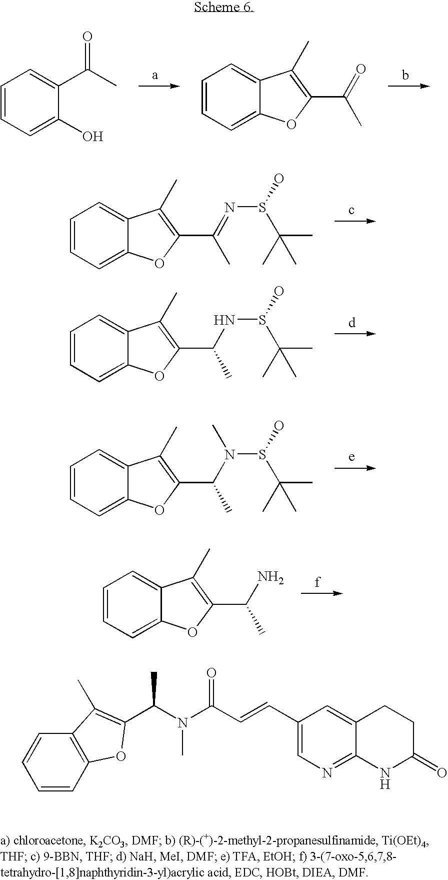

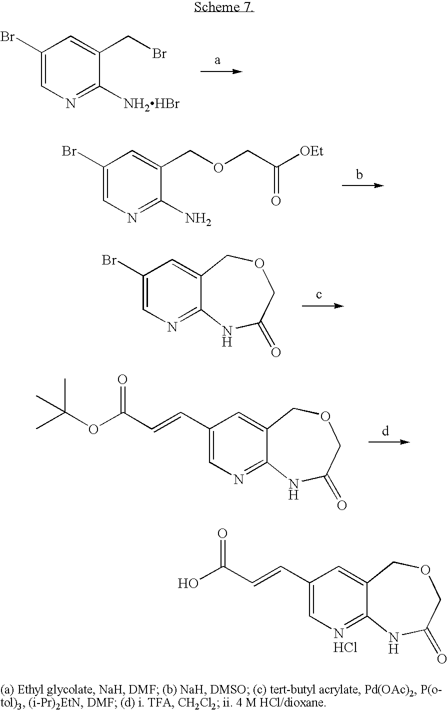

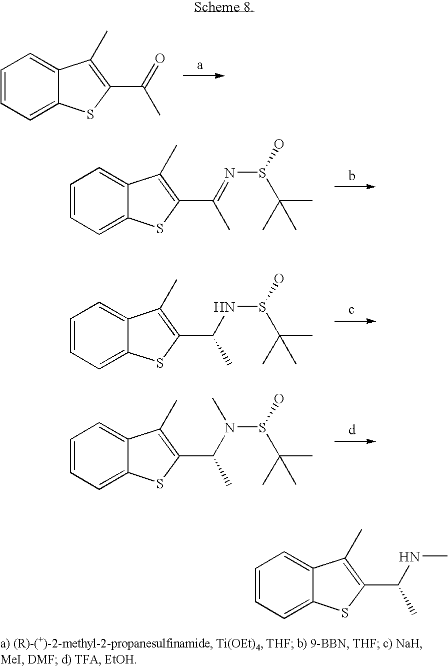

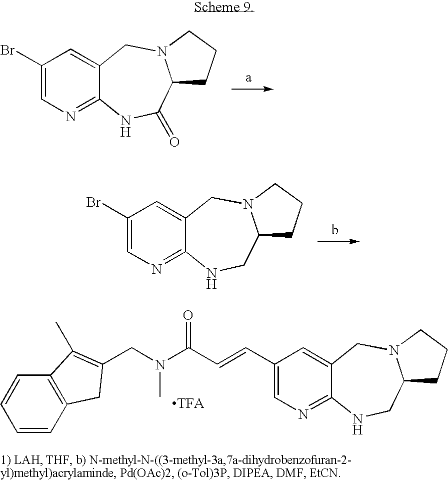

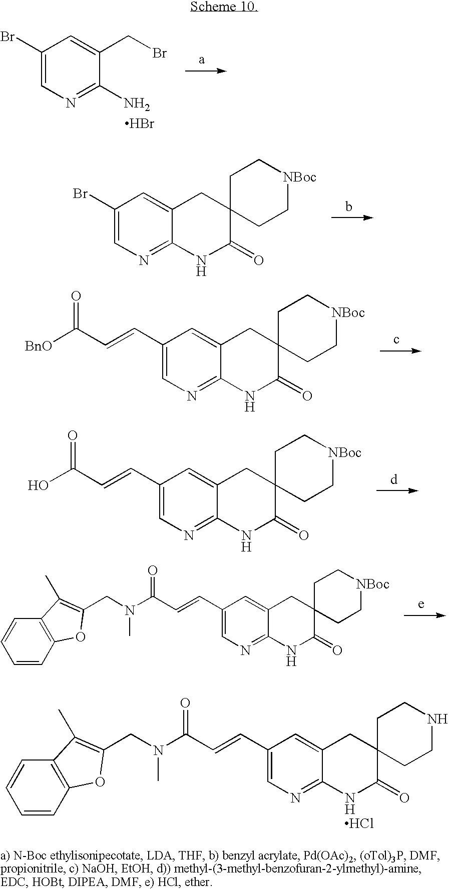

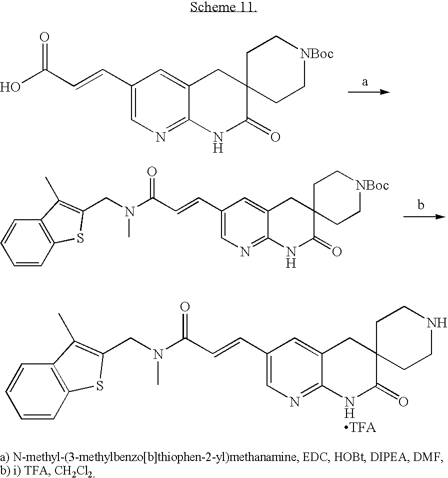

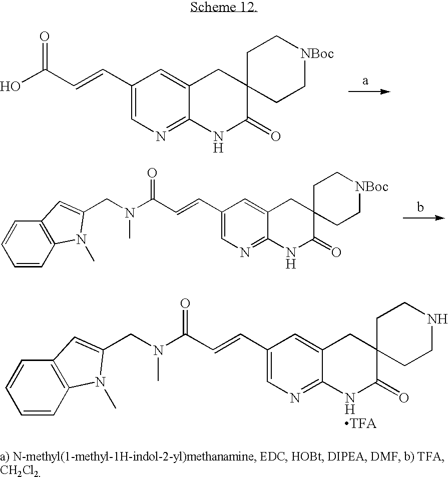

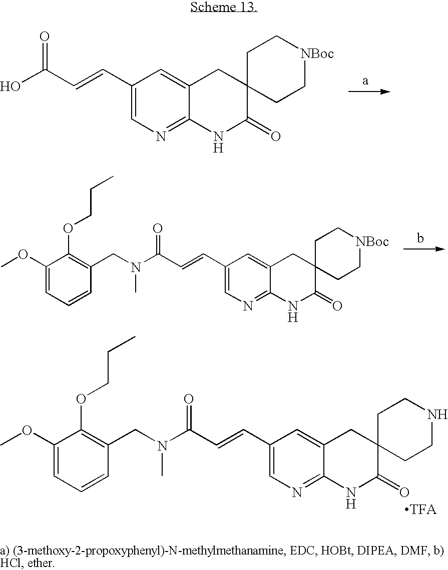

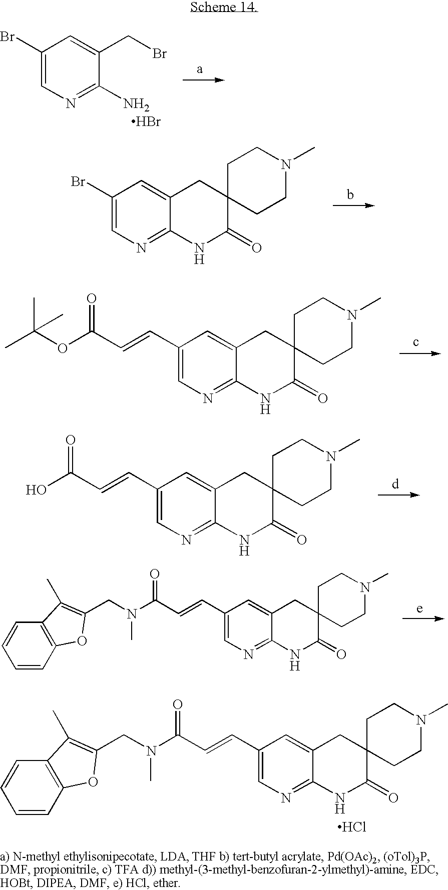

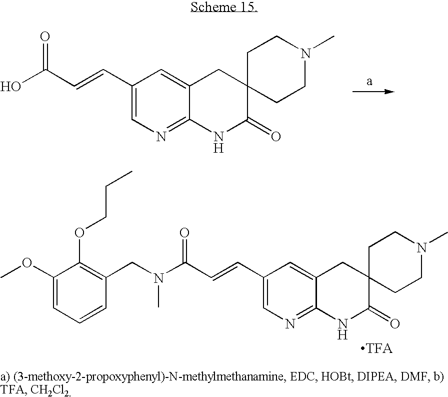

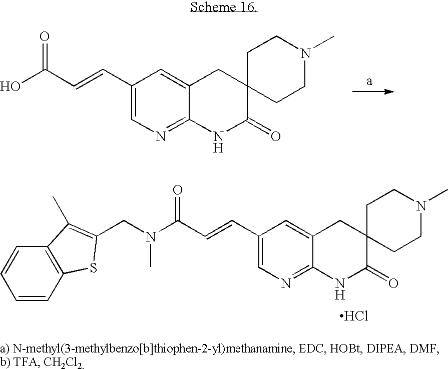

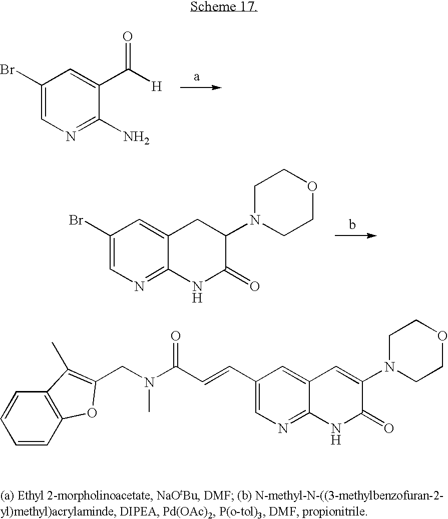

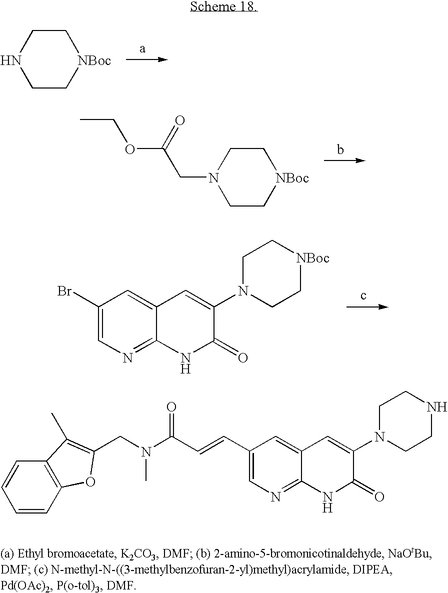

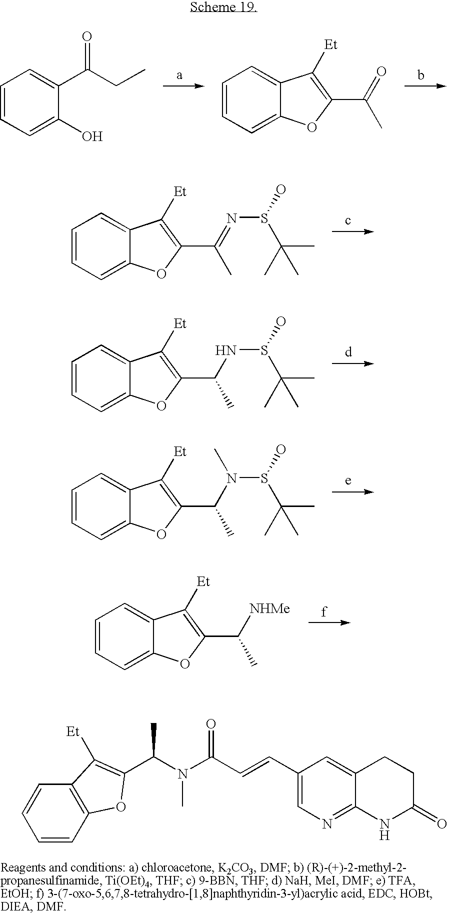

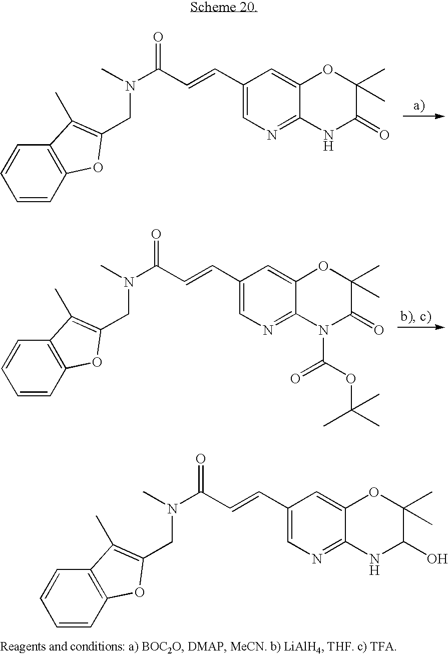

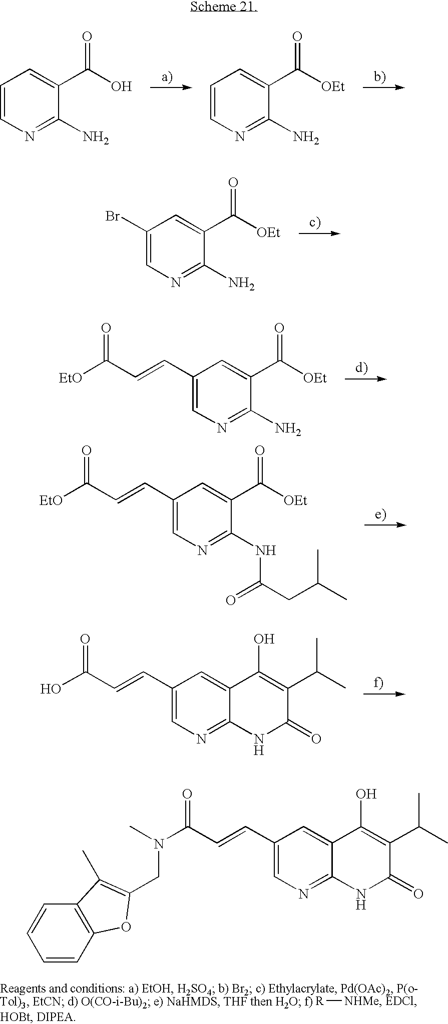

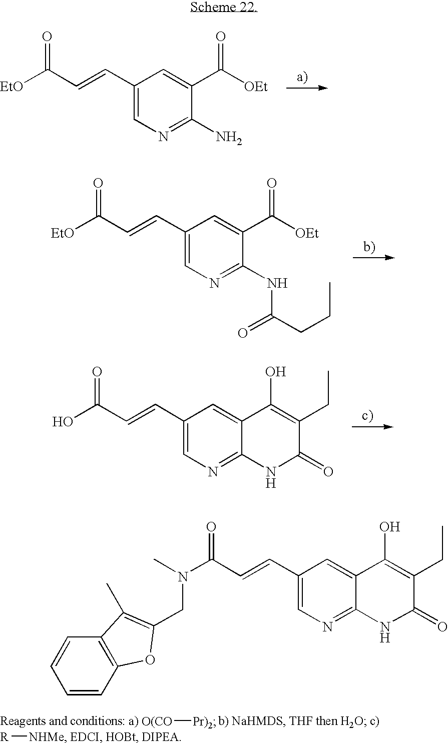

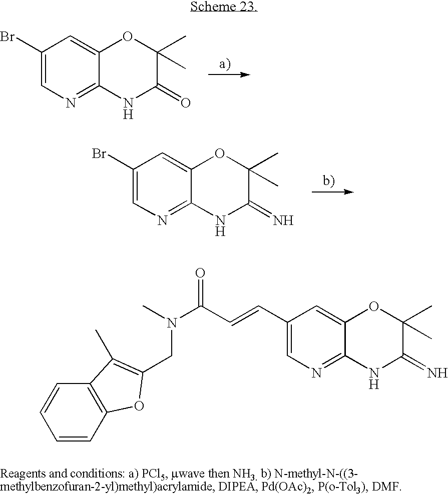

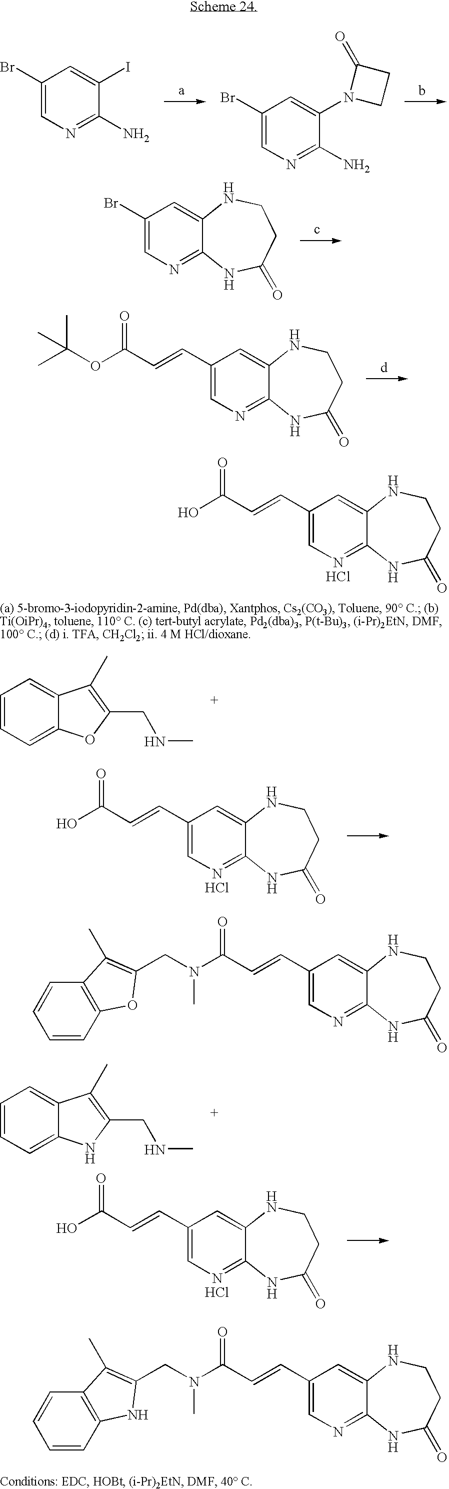

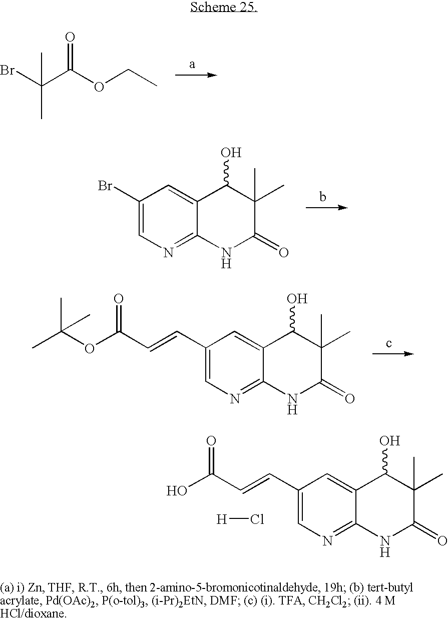

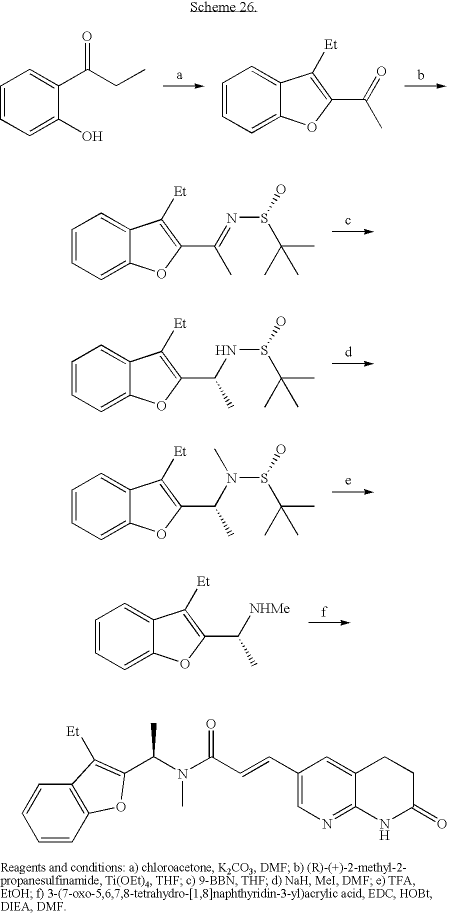

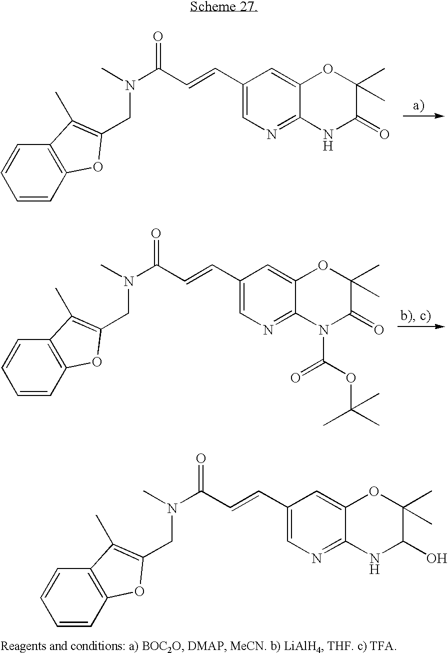

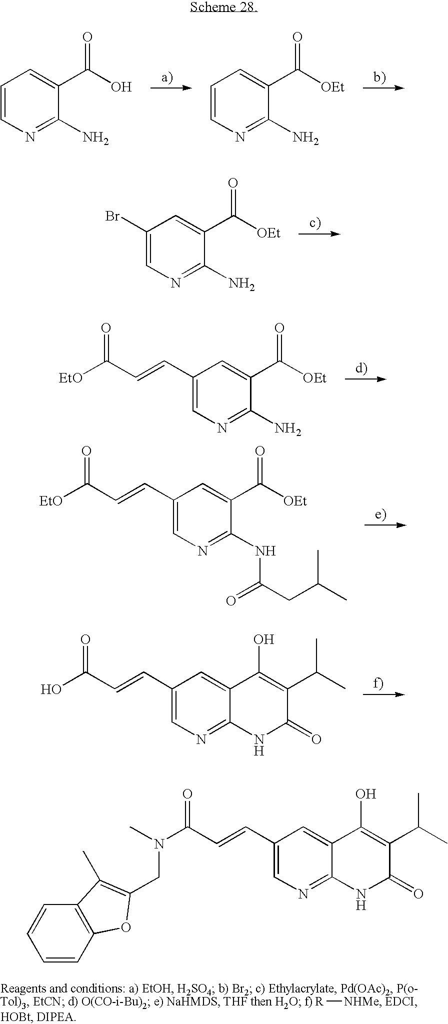

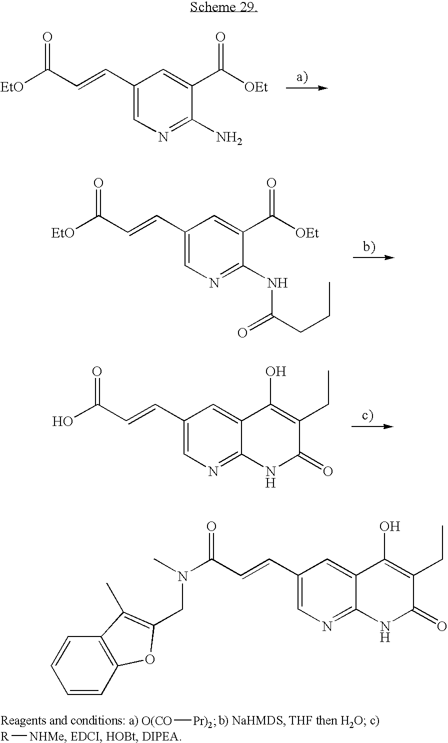

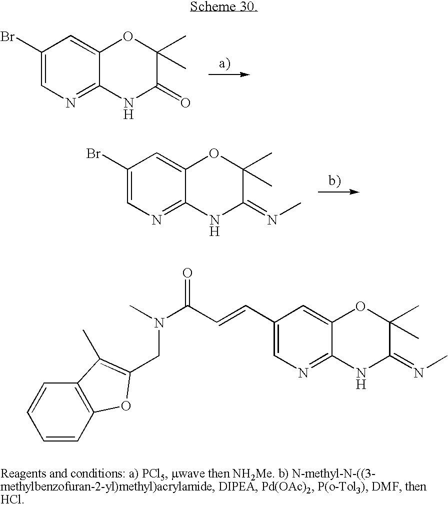

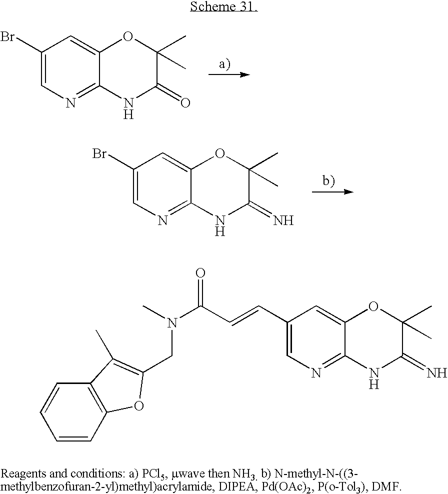

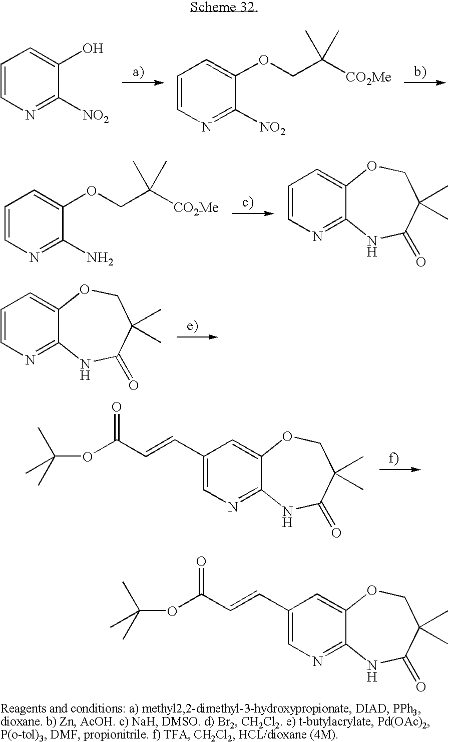

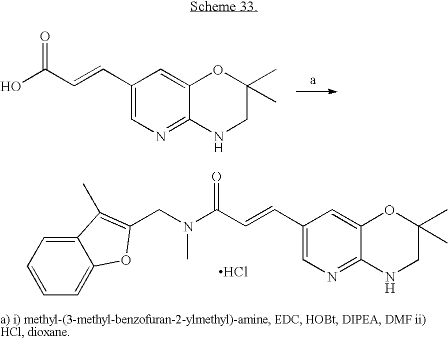

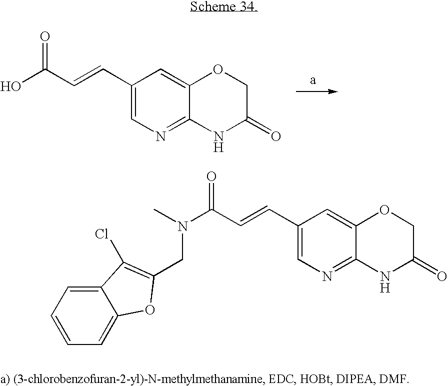

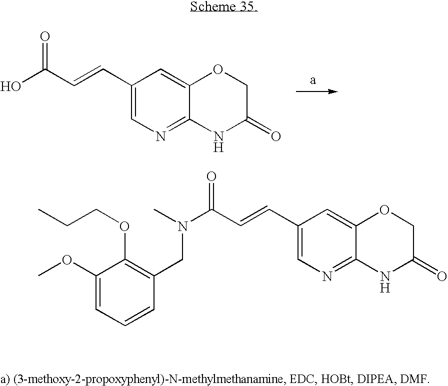

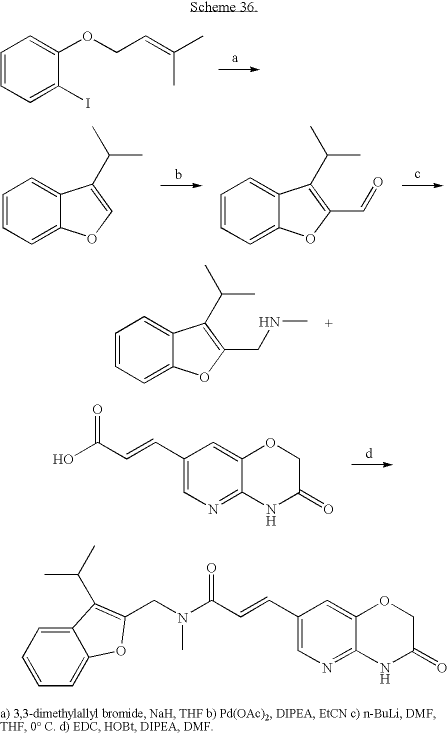

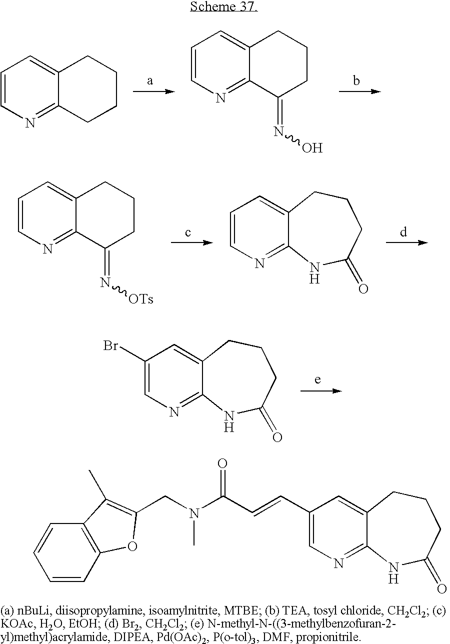

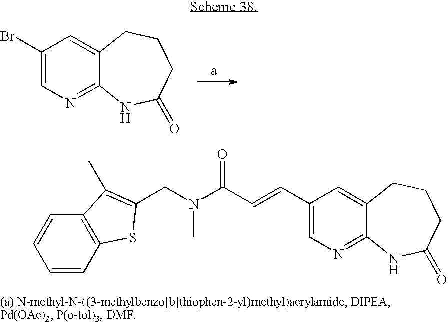

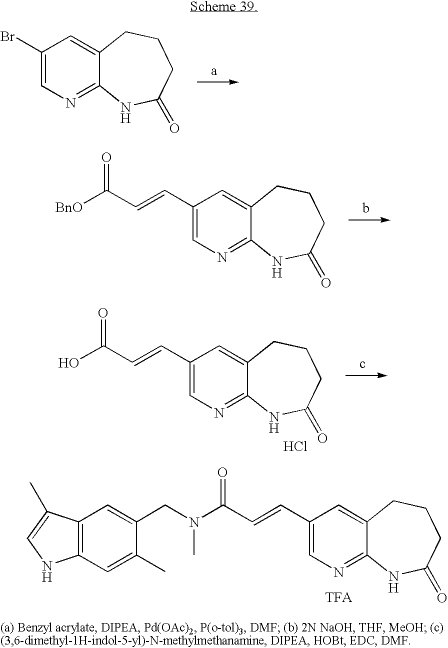

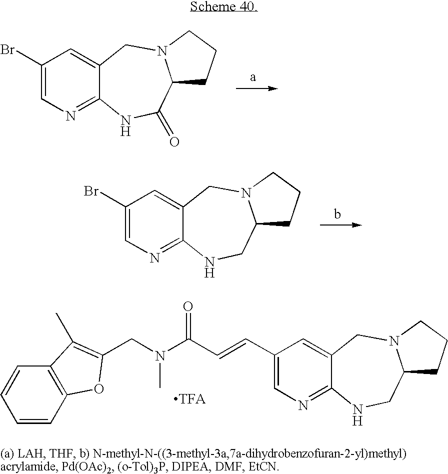

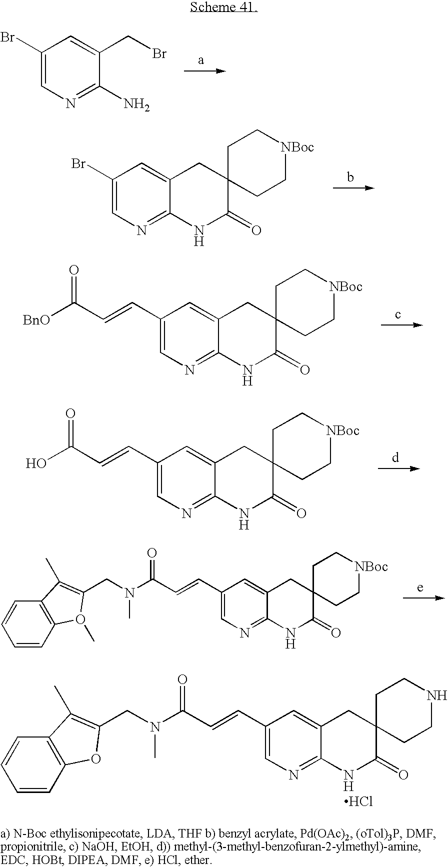

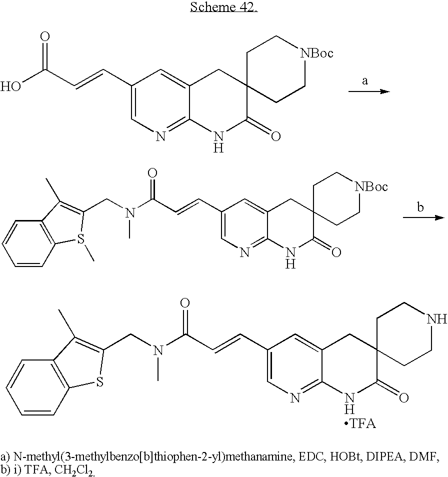

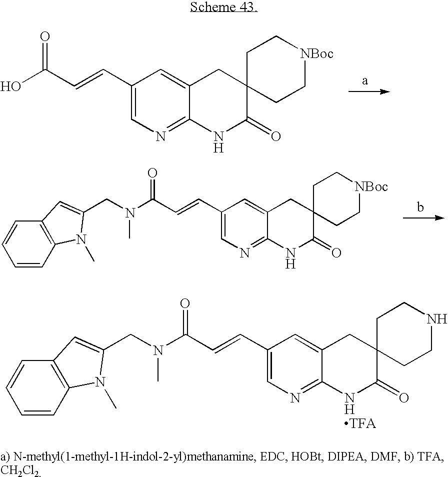

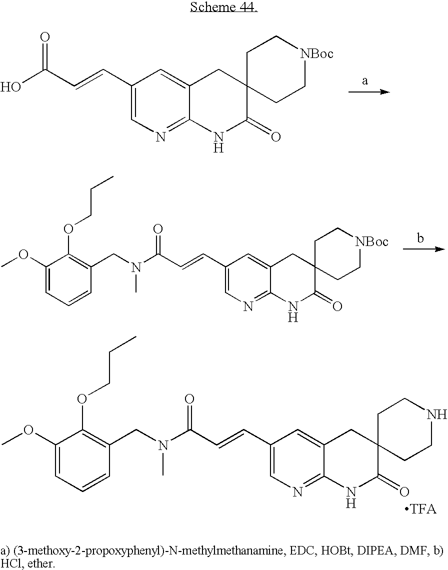

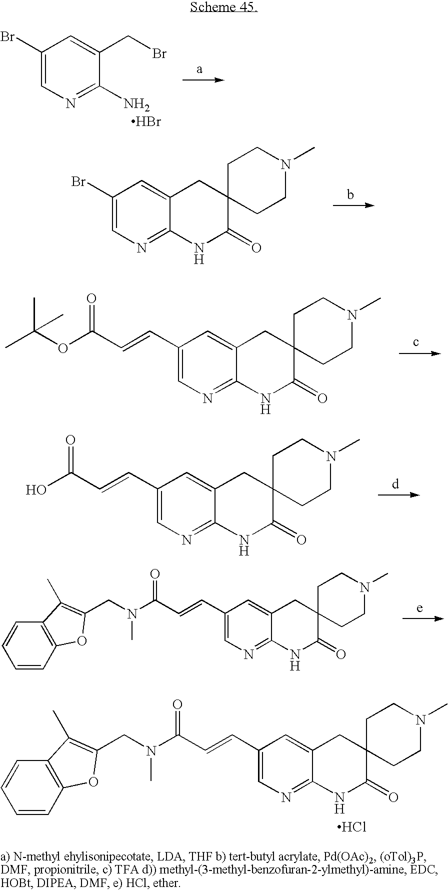

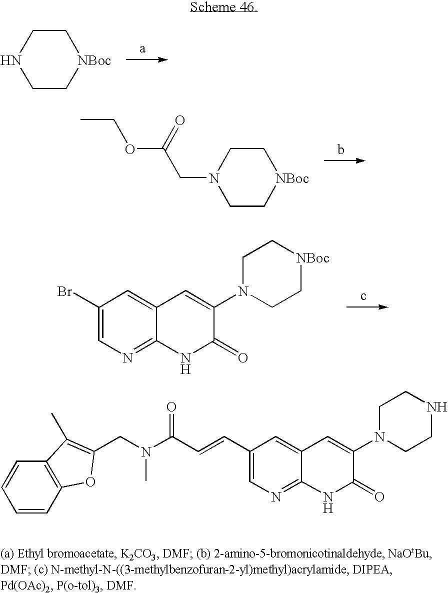

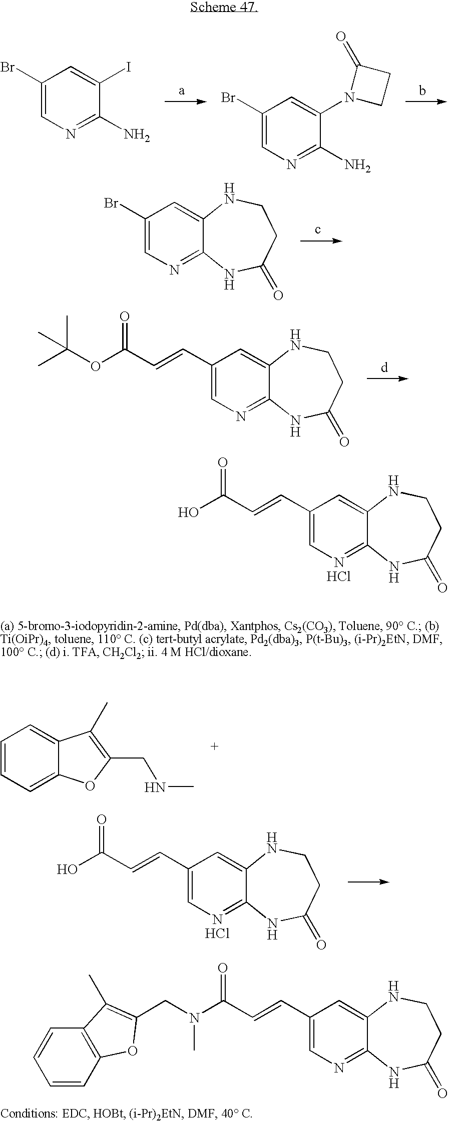

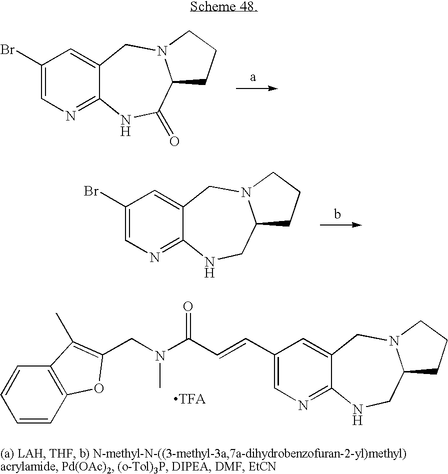

It will be recognized by one skilled in the art that other methods of LHS and RHS synthesis can be employed in the preparation of said intermediates. Likewise other methods of amide and/or carbon-carbon bond formation may be used to assemble the compounds of the invention. It is also apparent that combinations of LHS and RHS other than those described above can be envisioned to prepare compounds falling within the scope of the invention as represented by formula I. These possibilities are further detailed in the preparations and examples section to follow.

Acid addition salts of the compounds of formula I can be prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. This is illustrated by the preparation of hydrochloric acid salts as a final step in several of the general schemes shown above. Certain of the compounds form inner salts or zwitterions which may be acceptable. Cationic salts may be prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are some non-limiting examples of cations present in pharmaceutically acceptable salts.

### Toxicology of Compounds

Acute toxicity can be assessed using increasing doses in mice and rodents. Exploratory acute toxicity in mice and/or rats after single dose may be undertaken to begin estimation of the therapeutic window of inhibitors and to identify the potential target organs of toxicity. As candidate selection nears, these studies may provide guidance for the selection of proper doses in multi-dose studies, as well as establish any species specific differences in toxicities. These studies may be combined with routine PK measurements to assure proper dosages were achieved. Generally 3-4 doses will be chosen that are estimated to span a range having no effect through to higher doses that cause major toxic, but non-lethal, effects. Animals will be observed for effects on body weight, behavior and food consumption, and after euthanasia, hematology, blood chemistry, urinalysis, organ weight, gross pathology and histopathology will be undertaken.

Resistance Frequencies and Mechanisms of Compounds

In vitro resistance frequencies in bacteria of interest can be estimated for compounds of formula I. Experiments can determine whether resistant isolates arise when challenged to grow on solid media at 1×, 2× and 4×MIC concentrations. For example with respect to S. aureus or E. coli, the experiments may use several recent clinical isolates of methicillin-sensitive and methicillin-resistant S. aureus and a laboratory strain of E. coli with acrA efflux pump defect. In addition, experiments may use several characterized triclosan-resistant S. aureus strains. The MICs of resistant strains isolated in this manner can then be determined. Subsequent experiments can determine whether resistant strains arise after serial passage of the strains in 0.5×MIC concentrations of each lead compound.

Mechanism of resistance may be determined in S. aureus laboratory strain, RN450 and in an E. coli laboratory strain carrying an acrA efflux pump mutation. Both high dose challenge (4×MIC) and sub-MIC serial passage may be used to obtain spontaneously arising resistant isolates. If no isolates are obtained with reasonable frequencies, chemical and physical mutagenesis methods can be used to obtain resistant isolates. The fabI gene from the chromosome of resistant isolates may be PCR amplified, then may be sequenced to determine whether changes in the FabI protein caused resistance. Triplicate PCR amplifications and sequences may be performed to assure that the observed sequence changes are correct, and did not arise from PCR errors during amplification. Strains carrying resistance mutations outside of the gene of interest may be documented and saved, characterized for their effects on susceptibilities of other antibiotics as evidence of possible efflux-mediated resistance mechanisms, characterized for their ability to alter compounds characterized for their effects on the expression of the specific mRNA and FabI protein.

### Assays

Many different assay methods can be used to determine the activity of the compounds of the present invention. These assay methods include, for example, the following but also include other methods known to one of ordinary skill in the art.

S. aureus FabI Enzyme Inhibition Assay (NADH)

Assays are carried out in half-area, 96-well microtitre plates. Compounds are evaluated in 50-uL assay mixtures containing 100 mM NaADA, pH 6.5 (ADA=N-[2-acetamido]-2-iminodiacetic acid), 4% glycerol, 0.25 mM crotonoyl CoA, 1 mM NADH, and an appropriate dilution of S. aureus FabI. Inhibitors are typically varied over the range of 0.01-10 uM. The consumption of NADH is monitored for 20 minutes at 30° C. by following the change in absorbance at 340 nm. Initial velocities are estimated from an exponential fit of the non-linear progress curves represented by the slope of the tangent at t=0 min. IC₅₀'s are estimated from a fit of the initial velocities to a standard, 4-parameter model and are typically reported as the mean±S.D. of duplicate determinations. Triclosan, a commercial antibacterial agent and inhibitor of FabI, may be included in an assay as a positive control. Compounds of this invention may have IC₅₀'s from about 5.0 micromolar to about 0.05 micromolar.

S. aureus FabI Enzyme Inhibition Assay (NADPH) (Modified)

Assays are carried out in half-area, 96-well microtitre plates. Compounds are evaluated in 150-uL assay mixtures containing 100 mM NaADA, pH 6.5 (ADA=N-[2-acetamido]-2-iminodiacetic acid), 4% glycerol, 0.25 mM crotonoyl CoA, 50 uM NADPH, and an appropriate dilution of S. aureus FabI. Inhibitors are typically varied over the range of 0.01-10 uM. The consumption of NADPH is monitored for 20 minutes at 30° C. by following the change in absorbance at 340 nm. Initial velocities are estimated from an exponential fit of the non-linear progress curves represented by the slope of the tangent at t=0 min. IC₅₀'s are estimated from a fit of the initial velocities to a standard, 4-parameter model and are typically reported as the mean±S.D. of duplicate determinations. Triclosan, a commercial antibacterial agent and inhibitor of FabI, is currently included in all assays as a positive control.

H. influenzae FabI Enzyme Inhibition Assay

Assays are carried out in half-area, 96-well microtiter plates. Compounds are evaluated in 150-uL assay mixtures containing 100 mM MES, 51 mM diethanolamine, 51 mM triethanolamine, pH 6.5 (MES=2-(N-morpholino)ethanesulfonic acid), 4% glycerol, 25 uM crotonoyl-ACP, 50 uM NADH, and an appropriate dilution of H. influenzae FabI (approximately 20 nM). Inhibitors are typically varied over the range of 0.01-10 uM. The consumption of NADH is monitored for 20 minutes at 30° C. by following the change in absorbance at 340 nm. Initial velocities are estimated from an exponential fit of the non-linear progress curves. IC₅₀'s are estimated from a fit of the initial velocities to a standard, 4-parameter model, and are typically reported as the mean±S.D. of duplicate determinations. The apparent Ki is calculated assuming the inhibition is competitive with crotonoyl-ACP. A proprietary lead compound is currently included in all assays as a positive control.

E. coli FabI Enzyme Inhibition Assay

Assays are carried out in half-area, 96-well microtitre plates. Compounds are evaluated in 150-uL assay mixtures containing 100 mM NaADA, pH 6.5 (ADA=N-[2-acetamido]-2-iminodiacetic acid), 4% glycerol, 0.25 mM crotonoyl CoA, 50 uM NADH, and an appropriate dilution of E. coli FabI. Inhibitors are typically varied over the range of 0.01-10 uM. The consumption of NADH is monitored for 20 minutes at 30° C. by following the change in absorbance at 340 nm. Initial velocities are estimated from an exponential fit of the non-linear progress curves represented by the slope of the tangent at t=0 min. IC₅₀'s are estimated from a fit of the initial velocities to a standard, 4-parameter model and are typically reported as the mean±S.D. of duplicate determinations. Triclosan, a commercial antibacterial agent and inhibitor of FabI, is currently included in all assays as a positive control. Compounds of this invention have IC₅₀'s from about 100.0 micromolar to about 0.05 micromolar.

### Preparation and Purification of Crotonoyl-ACP

Reactions contain 5 mg/mL E. coli apo-ACP, 0.8 mM crotonoyl-CoA (Fluka), 10 mM MgCl₂, and 30 uM S. pneumoniae ACP synthase in 50 mM NaHEPES, pH 7.5. The mixture is gently mixed on a magnetic stirrer at 23° C. for 2 hr, and the reaction is terminated by the addition of 15 mM EDTA and cooling on ice. The reaction mixture is filtered through a 0.2 micron filter (Millipore) and applied to a MonoQ column (Pharmacia) equilibrated with 20 mM Tris-Cl, pH 7.5. The column is washed with buffer until all non-adherent material is removed (as observed by UV detection), and the crotonoyl-ACP is eluted with a linear gradient of 0 to 400 mM NaCl.

S. aureus FabI Enzyme Inhibition Assay Using Crotonoyl-ACP

Assays are carried out in half-area, 96-well microtitre plates. Compounds are evaluated in 100 uL assay mixtures containing 100 mM NaADA, pH 6.5 (ADA=N-(2-acetamido)-2-iminodiacetic acid), 4% glycerol, 25 uM crotonoyl-ACP, 50 uM NADPH, and an appropriate dilution of S. aureus Fab I (approximately 20 nM). Inhibitors are typically varied over the range of 0.01-30 uM. The consumption of NADPH is monitored for 30 minutes at 30° C. by following the change in absorbance at 340 nm. Initial velocities are estimated from a linear fit of the progress curves. IC₅₀'s are estimated from a fit of the initial velocities to a standard, 4-parameter model (Equation 1) and are typically reported as the mean±S.D. of duplicate determinations. Compounds of this invention in this assay have IC₅₀'s from about 60.0 micromolar to about 0.01 micromolar. The apparent Ki is calculated from Equation 2 assuming the inhibition is competitive with crotonoyl-ACP. More specifically, measured IC₅₀ values for 24 compounds of the present invention, as provided in the representative list above, ranged from less than about 0.02 μM to about 25 μM with 11 of these compounds having an IC₅₀ of less than 1.

H. pylori FabI Enzyme Inhibition Assay Using Crotonoyl-ACP

Assays are carried out in half-area, 96-well microtitre plates. Compounds are evaluated in 100 uL assay mixtures containing 100 mM NaADA, pH 6.5 (ADA=N-(2-acetamido)-2-iminodiacetic acid), 4% glycerol, 10 uM crotonoyl-ACP, 50 uM NADH, 100 mM ammonium acetate, and an appropriate dilution of H. pylori Fab I (approximately 15 nM). Inhibitors are typically varied over the range of 0.025-30 uM. The consumption of NADH is monitored for 30 minutes at 25° C. by following the change in absorbance at 340 nm. Initial velocities are estimated from a linear fit of the progress curves. IC50's are estimated from a fit of the initial velocities to a standard, 4-parameter model (Equation 1) and are typically reported as the mean±S.D. of duplicate determinations. Compounds of this invention in this assay have IC₅₀'s from about 60.0 micromolar to about 0.01 micromolar. The apparent Kᵢ is calculated from Equation 2 assuming the inhibition is competitive with crotonoyl-ACP.

v=Range/(1+[I]/IC50)s+Background Equation 1

Ki(app)=IC50/(1+[S]/Ks) Equation 2

S. pneumoniae FabK Enzyme Inhibition Assay Using Crotonoyl-ACP

Assays are carried out in half-area, 96-well microtitre plates. Compounds are evaluated in 100 uL assay mixtures containing 100 mM MES, 51 mM diethanolamine, 51 mM triethanolamine, pH 6.5 [MES=2-(N-morpholino)ethanesulfonic acid], 4% glycerol buffer, 100 mM NH₄Cl, 25 μM crotonoyl-ACP, 50 μM NADH, and 15 nM S. pneumoniae FabK. Inhibitors are typically varied over the range of 0.025-30 uM. The consumption of NADH is monitored for 30 minutes at 30° C. by following the change in absorbance at 340 nm. Initial velocities are estimated from a linear fit of the progress curves. IC₅₀'s are estimated from a fit of the initial velocities to a standard, 4-parameter model (Equation 1) and are typically reported as the mean±S.D. of duplicate determinations. Compounds of this invention in this assay have IC₅₀'s from about 60.0 micromolar to about 0.01 micromolar. The apparent Kᵢ is calculated from Equation 2 assuming the inhibition is competitive with crotonoyl-ACP.

### Antimicrobial Activity Assay

Whole-cell antimicrobial activity is determined by broth microdilution using the National Committee for Clinical Laboratory Standards (NCCLS) recommended procedure, Document M7-A5, “Methods for Dilution Susceptibility Tests for Bacteria that Grow Aerobically”. The compound is tested in serial two-fold dilutions ranging from 0.06 to 64 mcg/mL. A panel of 12 strains are evaluated in the assay. This panel consists of the following laboratory strains: Enterococcus faecalis 29212, Staphylococcus aureus 29213, Staphylococcus aureus 43300, Moraxella catarrhalis 49143, Haemophilus influenzae 49247, Streptococcus pneumoniae 49619, Staphylococcus epidermidis 1024939, Staphylococcus epidermidis 1024961, Escherichia coli AG100 (AcrAB⁺), Escherichia coli AG100A (AcrAB⁻), Pseudomonas aeruginosa K767 (MexAB⁺, OprM⁺), Pseudomonas aeruginosa K1119 (MexAB⁻, OprM⁻). The minimum inhibitory concentration (MIC) is determined as the lowest concentration of compound that inhibited visible growth. A spectrophotometer is used to assist in determining the MIC endpoint.

MIC assays may be performed using the microdilution method in a 96 well format. The assays may be performed in 96 well plates with a final volume of 100 μl cation-adjusted Mueller Hinton broth containing 2 fold serial dilutions of compounds ranging from 32 to 0.06 μg/ml. Bacterial growth may be measured at 600 nm using a Molecular Devices SpectraMax 340PC spectrophotometer. MICs can then be determined by an absorbance threshold algorithm and confirmed in some cases by inspecting the plates over a light box.

Minimum Bactericidal Concentration (MBC) may be determined by plating aliquots of MIC dilution series that did not show bacterial growth onto Petri plates containing appropriate semi-solid growth media. The lowest compound concentration that resulted in >99% killing of bacterial cells (relative to initial bacterial inocula in MIC test) is defined as the MBC.

Several strain panels may be used at various points in the compound progression scheme. The primary panel may include single prototype strains of both community- and hospital-acquired pathogens for determining initial activities and spectra of activity. Secondary panel compositions will depend on the results of the primary panels, and will include 10-20 strains of relevant species that will include community acquired and antibiotic-resistant hospital acquired strains of Staphylococcus aureus and coagulase negative Staphylococci together with other strains that are sensitive to the new compounds, and negative control strains. The secondary panels will be used during optimization of lead chemical series. Tertiary panels will include 100-200 clinical strains of S. aureus and coagulase negative Staphylococci together with other relevant strains as for the secondary panels. The tertiary panels will be utilized during the compound candidate selection stage and preclinical studies to generate bacterial population efficacy parameters such as MIC₅₀ and MIC₉₀.

Using the assay described above, measured MIC values against Staphylococcus aureus 29213 for 24 compounds of the present invention, as provided in the representative list above, ranged from less than about 0.06 μg/ml to greater than about 30 μg/ml with 9 of these compounds having an MIC of less than 1.

Franciscella tularensis In Vitro Efficacy Studies

Routine MIC testing of F. tularensis may be undertaken on compounds that have demonstrated enzymatic activity inhibition against the F. tularensis FabI protein. The MIC testing of F. tularensis may be outsourced to a facility with BL3 capabilities, and with experience in handling F. tularensis cultures in the laboratory. The studies may be undertaken with the recommended methods for antimicrobial susceptibility testing of F. tularensis.

Helicobacter pylori In Vitro Efficacy Studies

Routine MIC testing of H. pylori may be undertaken on compounds that have demonstrated enzymatic activity inhibition against the H. pylori FabI protein. The studies may be undertaken with the recommended methods for antimicrobial susceptibility testing of H. pylori.

### Cytotoxicity Assays

Cytotoxicity of the new compounds may be evaluated by the Alamar Blue assay according the manufacturers instructions. Human cell lines (e.g. Jurkat) grown in 96 well plates may be exposed to serial dilutions of the tested compounds. After adding Alamar Blue, cell viability may be determined by measuring the absorbance of the reduced and oxidized forms of Alamar Blue at 570 nm and 600 nm. Cytotoxicity may be reported as LD₅₀, the concentration that causes a 50% reduction in cell viability.

### Dosages

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions (e.g., the FabI inhibitor) because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

### Formulation

The antibacterial compositions of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In certain embodiments, the subject compounds may be formulated as a tablet, pill capsule or other appropriate ingestible formulation (collectively hereinafter “tablet”), to provide a therapeutic dose in 10 tablets or fewer. In another example, a therapeutic dose is provided in 50, 40, 30, 20, 15, 10, 5 or 3 tablets.

In a certain embodiment, the antibacterial agent is formulated for oral administration as a tablet or an aqueous solution or suspension. In another embodiment of the tablet form of the antibacterial agent, the tablets are formulated such that the amount of antibacterial agent (or antibacterial agents) provided in 20 tablets, if taken together, would provide a dose of at least the median effective dose (ED₅₀), e.g., the dose at which at least 50% of individuals exhibited the quantal effect of inhibition of bacterial cell growth or protection (e.g., a statistically significant reduction in infection). In a further embodiment, the tablets are formulated such that the total amount of antibacterial agent (or antibacterial agents) provided in 10, 5, 2 or 1 tablets would provide at least an ED₅₀ dose to a patient (human or non-human mammal). In other embodiments, the amount of antibacterial agent (or antibacterial agents) provided in 20, 10, 5 or 2 tablets taken in a 24 hour time period would provide a dosage regimen providing, on average, a mean plasma level of the antibacterial agent(s) of at least the ED₅₀ concentration (the concentration for 50% of maximal effect of, e.g., inhibiting bacterial cell growth). In other embodiments less than 100 times, 10 times, or 5 times the ED50 is provided. In other embodiments, a single dose of tablets (1-20 tablets) provides about 0.25 mg to 1250 mg of an antibacterial agent(s).

Likewise, the antibacterial agents can be formulated for parenteral administration, as for example, for subcutaneous, intramuscular or intravenous injection, e.g., the antibacterial agent can be provided in a sterile solution or suspension (collectively hereinafter “injectable solution”). The injectable solution is formulated such that the amount of antibacterial agent (or antibacterial agents) provided in a 200 cc or 20 cc bolus injection would provide a dose of at least the median effective dose, or less than 100 times the ED₅₀, or less than 10 or 5 times the ED₅₀. The injectable solution may be formulated such that the total amount of antibacterial agent (or antibacterial agents) provided in 100, 50, 25, 10, 5, 2.5, or 1 cc injections would provide an ED₅₀ dose to a patient, or less than 100 times the ED₅₀, or less than 10 or 5 times the ED₅₀. In other embodiments, the amount of antibacterial agent (or antibacterial agents) provided in a total volume of 100 cc, 50, 25, 5 or 2 cc to be injected at least twice in a 24 hour time period would provide a dosage regimen providing, on average, a mean plasma level of the antibacterial agent(s) of at least the ED₅₀ concentration, or less than 100 times the ED₅₀, or less than 10 or 5 times the ED₅₀. In other embodiments, a single dose injection provides about 0.25 mg to 1250 mg of antibacterial agent.

### Efficacy of Treatment

The efficacy of treatment with the subject compositions may be determined in a number of fashions known to those of skill in the art.

In one exemplary method, the median survival rate of the bacteria or bacteria median survival time or life span for treatment with a subject composition may be compared to other forms of treatment with the particular FabI inhibitor, or with other antibiotic agents. The decrease in median bacteria survival rate or time or life span for treatment with a subject composition as compared to treatment with another method may be 10, 25, 50, 75, 100, 150, 200, 300, 400% even more. The period of time for observing any such decrease may be about 3, 5, 10, 15, 30, 60 or 90 or more days. The comparison may be made against treatment with the particular FabI inhibitor contained in the subject composition, or with other antibiotic agents, or administration of the same or different agents by a different method, or administration as part of a different drug delivery device than a subject composition. The comparison may be made against the same or a different effective dosage of the various agents. The different regiments compared may use measurements of bacterial levels to assess efficacy.

Alternatively, a comparison of the different treatment regimens described above may be based on the effectiveness of the treatment, using standard indicies for bacterial infections known to those of skill in the art. One method of treatment may be 10%, 20%, 30%, 50%, 75%, 100%, 150%, 200%, 300% more effective, than another method.

Alternatively, the different treatment regimens may be analyzed by comparing the therapeutic index for each of them, with treatment with a subject composition as compared to another regimen having a therapeutic index two, three, five or seven times that of, or even one, two, three or more orders of magnitude greater than, treatment with another method using the same or different FabI inhibitor.

As a non-limiting example, to determine if compounds are bactericidal or bacteriostatic at relevant concentrations, and to examine the kinetics of bacterial killing the following experiment may be performed with S. aureus, S. epidermidis and appropriate control strains and antibiotics. To fresh logarithmic cultures at 107 viable cells/ml, compound may be added to reach concentrations of ×1, ×2 or ×4 the MIC. Control cultures will receive no compound. At 1 hour intervals, aliquots will be diluted and plated for determining viable counts. Plots of viable cells vs. time for up to 24 hours will reveal bactericidal/bacteriostatic properties of the compounds, and also show the kill kinetics. These experiments are important to determine whether these inhibitors have time-dependent or concentration-dependent effects, and will be used to help set appropriate dosages in vivo in combination with pharmacokinetic and pharmacodynamic measurements.

In the practice of the instant methods, the antibacterial compositions of the present invention inhibit bacterial FabI with a Kᵢ of 5 μM or less, 1 μM or less, 100 nM or less, 10 nM or less or even 1 nM or less. In treatment of humans or other animals, the subject method may employ FabI inhibitors which are selective for the bacterial enzyme relative to the host animals' enoyl CoA hydratase, e.g., the Kᵢ for inhibition of the bacterial enzyme is at least one order, two orders, three orders, or even four or more orders of magnitude less than the Kᵢ for inhibition of enoyl CoA hydratase from the human (or other animal). That is, the practice of the subject method in vivo in animals utilizes FabI inhibitors with therapeutic indexes of at least 10, 100 or 1000.

Similarly, in the practice of the instant method, the antibacterial compounds of the present invention inhibit FabI with an IC₅₀ of 30 μM or less, 10 μM or less, 100 nM or less, or even 10 nM or less. In treatment of humans or other animals, the subject method may employ FabI inhibitors which are selective for the bacterial enzyme relative to the host animals' enoyl CoA hydratase, e.g., the IC₅₀ for inhibition of the bacterial enzyme is at least one order, two orders, three orders, or even four orders of magnitude less than the IC₅₀ for inhibition of enoyl CoA hydratase from the human (or other animal). That is, in preferred embodiments, the practice of the subject method in vivo in animals utilizes FabI inhibitors with therapeutic indexes of at least 10, 100 or 1000.

Alternatively, bacterial inhibition by an antibacterial compound of the present invention may also be characterized in terms of the minimum inhibitory concentration (MIC), which is the highest concentration of compound required to achieve complete inhibition of bacterial cell growth. Such values are well known to those in the art as representative of the effectiveness of a particular antibacterial agent against a particular organism or group of organisms. In the practice of the instant methods, the antibacterial compositions of the present invention inhibit bacterial growth with MIC values of about 32 μg/mL, less than about 16 μg/mL, less than about 8 μg/mL, less than about 4 μg/mL, less than about 2 μg/mL, less than about 1 μg/mL, less than about 0.5 μg/mL, less than about 0.25 μg/mL, or even less than about 0.125 μg/mL. The value of MIC90, defined as the concentration of a compound required to inhibit the growth of 90% of bacterial strains within a given bacterial strain population, can also be used. In certain embodiments, the compounds of the present invention are selected for use based, inter alia, on having MIC90 values of less than about 32 μg/mL, less than about 16 μg/mL, less than about 8 μg/mL, less than about 4 μg/mL, less than about 2 μg/mL, less than about 1 μg/mL, less than about 0.5 μg/mL, less than about 0.25 μg/mL, or even less than about 0.125 μg/mL.

In other embodiments, the subject compounds are selected for use in animals, or animal cell/tissue culture based at least in part on having LD₅₀'s at least one order, or two orders, or three orders, or even four orders or more of magnitude greater than the ED₅₀. That is, in certain embodiments where the subject compounds are to be administered to an animal, a suitable therapeutic index is preferably greater than 10, 100, 1000 or even 10,000.

### Kits

This invention also provides kits for conveniently and effectively implementing the methods of this invention. Such kits comprise any subject composition, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use.

The examples which follow are intended in no way to limit the scope of this invention but are provided to illustrate how to prepare and use compounds of the present invention. Many other embodiments of this invention will be apparent to one skilled in the art.

### EXEMPLIFICATION

### General

Proton nuclear magnetic resonance (¹H NMR) spectra were recorded at either 300 or 500 MHz, and chemical shifts are reported in parts per million (δ) downfield from the internal standard tetramethylsilane (TMS) or from deuterated solvent. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuteriochloroform, DMSO-d₆ is hexadeuteriodimethylsulfoxide, CD₃OD is tetradeuteriomethanol and D₂O is deuterated oxide. Mass spectra were obtained using electrospray (ESI) ionization techniques. Flash chromatography was carried out on E. Merck Kieselgel 60 (230-400 mesh) silica gel. Analytical HPLC was performed on Varian chromatography systems. Celite® is a filter aid composed of acid-washed diatomaceous silica, and is a registered trademark of Manville Corp., Denver, Colo. General abbreviations are as follows: EDC=1-(3-dimethylaminopropyl)-3-ethylcarbodiimide hydrochloride, HOBt=1-hydroxybenzotriazole hydrate, (i-Pr)₂EtN=N,N-diisopropylethylamine, DMF=N,N-dimethylformamide, MeOH=methanol, EtOH=ethanol, THF=tetrahydrofuran, DMSO=dimethylsulfoxide, Et₂O=diethyl ether, Ar=argon, Pd(OAc)₂=palladium(II) acetate, P(o-tol)₃=tri-ortho-tolylphoshine, EtOAc=ethyl acetate, ACE-Cl=1-chloroethyl chloroformate, satd=saturated, Et₃N=triethylamine, TFA=trifluoroacetic acid, NaBH(OAc)₃=sodium triacetoxyborohydride, HOAc=acetic acid, EtCN=proprionitrile, CBzCl=benzyl chloroformate, MeCN=acetonitrile.

### Example 1

### Preparation of (E)-3-[6-Amino-5-(pyridin-2-ylmethoxy)-pyridin-3-yl]-N-methyl-N-(3-methyl-benzofuran-2-ylmethyl)acrylamide

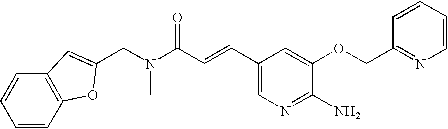

### a) 3H-oxazolo[4,5-b]pyridin-2-one

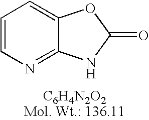

A suspension of 2-amino-pyridin-3-ol (20.0 g, 181 mmol) in THF (200 mL) was added to 1,1′-carbonyldiimidazole (44.1 g, 272 mmol). The mixture was heated to reflux overnight. After cooling, the reaction was concentrated and the residue was taken up with CH₂Cl₂ (400 mL) and extracted with 1M NaOH (3×). The combined liquids were cooled in an ice-bath and adjusted to pH 5-6 by adding 3M HCl. The resulting precipitate was isolated by filtration, washed with THF, and dried under vacuum at 50° C. overnight to give the title compound (20.0 g, 81%) as a tan solid: ¹H NMR (300 MHz, DMSO-d₆) δ 8.04 (dd, J=5.4, 1.2 Hz, 1H), 7.64 (dd, J=8.1, 1.2 Hz, 1H), 7.14-7.05 (m, 1H); ESI MS m/z 137 (M+H)⁺.

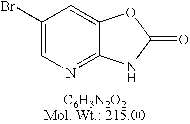

### b) 6-Bromo-3H-oxazolo[4,5-b]pyridin-2-one

A solution of 3H-oxazolo[4,5-b]pyridin-2-one (20.0 g, 147 mmol) in DMF was added dropwise to a solution of Br₂ (8.30 mL, 162 mmol) in DMF at 0° C. The mixture was heated to reflux under a nitrogen atmosphere for 2 h. After 2 h, ice water (250 mL) was added dropwise and the mixture was stirred for 15 minutes. The resulting precipitate was isolated by filtration, washed with water and dried under vacuum at 50° C. overnight to give the title compound (28.0 g, 89%) as a yellow solid: ¹H NMR (500 MHz, DMSO-d₆) δ 12.6 (br s, 1H), 8.10 (s, 1H), 8.01 (s, 1H); ESI MS m/z 216 (M+H)⁺.

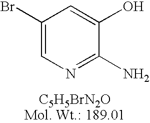

### c) 2-Amino-5-bromo-pyridin-3-ol

To a solution of 6-bromo-3H-oxazolo[4,5-b]pyridin-2-one (28.0 g, 130 mmol) in MeOH (280 mL) was added a solution of NaOH (28.1 g, 703 mmol) in water (280 mL). The mixture was heated to reflux overnight. The organic solvent was removed in vacuo and the aqueous mixture was adjusted to pH 5-6 with 12 M HCl. The resulting precipitate was isolated by filtration, washed with Et₂O and dried under vacuum at 50° C. overnight to give the title compound (19.6 g, 80%) as a gray solid: ¹H NMR (500 MHz, DMSO-d₆) δ 10.1 (s, 1H), 7.46 (d, J=1.5 Hz, 1H), 6.88 (s, 1H), 5.70 (s, 2H); ESI MS m/z 190 (M+H)⁺.

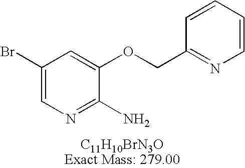

### C₁₁H₁₀BrN₃O

### Exact Mass: 279.00

### d) 5-Bromo-3-(pyridine-2-ylmethoxy)-pyridin-2-ylamine

To a suspension of 2-amino-5-bromo-pyridin-3-ol (2.00 g, 10 mmol) in acetone (80 mL) was added to 2-chloromethyl-pyridine hydrochloride (2.24 g, 13.7 mmol), K₂CO₃ (4.38 g, 31.7 mmol) and NaI (4.74 g, 31.7 mmol). The mixture was heated to reflux overnight. After cooling, the mixture was diluted with water and then extracted with EtOAc (3×). The combined organics were washed with brine, dried (Na₂SO₄) and concentrated. Purification by column chromatography (silica gel, hexanes/EtOAc, 1:4) gave the title compound (830 mg, 27%) as a brown oil: ¹H NMR (300 MHz, DMSO-d₆) δ 8.61-8.57 (m, 1H), 7.88-7.84 (m, 1H), 7.66 (d, J=8.1 Hz, 1H), 7.60 (d, J=1.8 Hz, 1H), 7.35 (dd, J=7.5, 3.0 Hz, 1H), 7.28 (d, J=2.1 Hz, 1H) 6.06 (s, 2H), 5.21 (s, 2H); ESI MS m/z 280 (M+H)⁺.

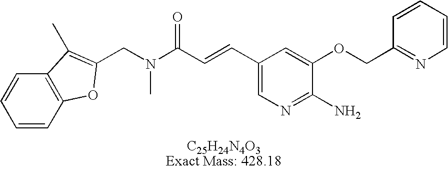

### e) (E)-3-[6-Amino-5-(pyridin-2-ylmethoxy)-pyridin-3-yl]-N-methyl-N-(3-methyl-benzofuran-2-ylmethyl)acrylamide

To a solution of 5-bromo-3-(pyridine-2-ylmethoxy-pyridin-2-ylamine (500 mg, 1.78 mmol) in propionitrile (40 mL) and DMF (10 mL) were added N-methyl-N-(3-methyl-benzofuran-2-ylmethyl)acrylamide (530 mg, 2.32 mmol), (i-Pr)₂EtN (0.621 mL, 4.64 mmol), Pd(OAc)₂ (0.399 g, 0.178 mmol) and P(o-tol)₃ (108 mg, 0.356 mmol), and the mixture was de-oxygenated with argon for 15 min. The mixture was heated to reflux overnight, allowed to cool and then filtered. The filtrate was concentrated and the residue was dissolved in CH₂Cl₂ (150 mL). The organic solution was washed with water and brine, dried and the solvent was removed in vacuo. Purification by column chromatography (silica gel, CH₂Cl₂/MeOH, 20:1) gave the title compound (146 mg, 20%) as a yellow solid and as a mixture of amide rotamers: ¹H NMR (500 MHz, DMSO-d₆) δ 8.56 (br s, 1H), 7.85 (t, J=7.5 Hz, 1H), 7.80-7.77 (m, 2H), 7.63-7.61 (m, 1H), 7.75 (d, J=0.5 Hz, 1H), 7.50-7.40 (m, 2H), 7.38-7.32 (m, 1H), 7.30-7.20 (m, 2H), 7.05-6.95 (m, 1H), 6.41 (br s, 2H), 5.24 (s, 2H), 4.98-4.78 (m, 2H), 3.18-2.92 (m, 3H), 2.62 (s, 3H); ESI MS m/z 429 (M+H).

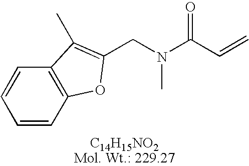

### N-Methyl-N-(3-methyl-benzofuran-2-ylmethyl)acrylamide

A solution of methyl-(−3-methyl-benzofuran-2-ylmethyl)amine (3.00 g, 171 mmol) in CH₂Cl₂ (160 mL) was treated with acryloyl chloride (1.46 mL, 17.9 mmol) and triethylamine (3.46 mL, 342 mmol). The mixture was stirred at room temperature for 2 h. The solution was washed with water and brine, dried and concentrated under reduced pressure. Purification by column chromatography (silica gel, hexanes/EtOAc, 5:1) gave the title compound (2.98 g, 76%) as a yellow solid: ¹H NMR (500 MHz, DMSO-d₆); δ 7.50-7.46 (m, 1H), 7.39-7.33 (m, 1H), 7.31-7.16 (m, 2H), 7.10-7.00 (m, 1H), 6.40 (s, 1H), 5.30-5.22 (m, 1H), 4.77-4.63 (m, 2H), 3.13-3.02 (m, 3H), 2.29-2.25 (m, 3H); ESI MS m/z 230 (M+H)⁺.

### Example 2

### Preparation of (E)-3-[6-Amino-5-(pyridin-3-ylmethoxy)-pyridin-3-yl]-N-methyl-N-(3-methyl-benzofuran-2-ylmethyl)acrylamide

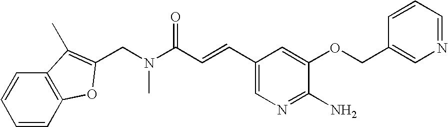

### a) 5-Bromo-3-(pyridine-3-ylmethoxy)-pyridin-2-ylamine

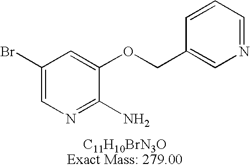

A suspension of 2-amino-5-bromo-pyridin-3-ol (1.00 g, 5.29 mmol) in acetone (40 mL) was added to a solution of 3-chloromethylpyridine hydrochloride (1.12 g, 6.87 mmol), K₂CO₃ (2.19 g, 15.8 mmol) and NaI (2.37 g, 15.8 mmol). The mixture was heated to reflux overnight. After cooling, the mixture was dissolved in water and extracted with EtOAc (3×). The combined organics were washed with brine, dried and concentrated. Purification by column chromatography (silica gel, hexanes/EtOAc, 1:4) gave the title compound (430 mg, 29%) as a brown oil: ¹H NMR (300 MHz, DMSO-d₆) δ 8.71 (d, J=1.8 Hz, 1H), 8.55 (d, J=3.0 Hz 1H), 7.96-7.92 (m, 1H), 7.60 (d, J=3.0 Hz, 1H), 7.45-7.35 (m, 1H), 7.34 (d, J=3.0 Hz, 1H), 6.04 (br s, 2H), 5.20 (s, 2H); ESI MS m/z 280 (M+H)⁺.

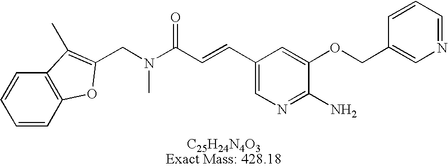

### b) (E)-3-[6-Amino-5-(pyridin-3-ylmethoxy)-pyridin-3-yl]-N-methyl-N-(3-methyl-benzofuran-2-ylmethyl)acrylamide

To a solution of 5-bromo-3-(pyridine-3-ylmethoxy-pyridin-2-ylamine (390 mg, 1.39 mmol) in propionitrile (40 mL) and DMF (10 mL) were added N-methyl-N-(3-methyl-benzofuran-2-ylmethyl)acrylamide (414 mg, 1.80 mmol), (i-Pr)₂EtN (0.48 mL, 2.78 mmol), Pd(OAc)₂ (31.2 mg, 0.139 mmol) and P(o-tol)₃ (84.6 mg, 0.278 mmol), and the mixture was de-oxygenated with argon for 15 min. The mixture was heated to reflux overnight, allowed to cool and filtered. The filtrate was concentrated, and the residue was dissolved in CH₂Cl₂ (150 mL). The organic solution was washed with water and brine, dried and the solvent was removed in vacuo. Purification by column chromatography (silica gel, CH₂Cl₂/MeOH, 20:1) gave the title compound (72.0 mg, 17%) as a pale solid and as a mixture of amide rotamers: ¹H NMR (500 MHz, DMSO-d₆) δ 8.75 (d, J=1.5 Hz, 1H), 8.55 (dd, J=1.5, 4.5 Hz, 1H), 7.97 (dd, J=2.0, 8.0 Hz, 1H), 7.85 (s, 1H), 7.68-7.60 (m, 1H), 7.67-7.55 (m, 1H), 7.52-7.41 (m, 3H), 7.31-7.22 (m, 2H), 7.25-6.95 (m, 1H), 6.31 (s, 2H), 5.23 (s, 2H), 4.97-4.78 (m, 2H), 3.18-2.92 (m, 3H), 2.26 (s, 3H); ESI MS m/z 429 (M+H)⁺.

### Example 3

### Preparation of (E)-3-(6-Acetylamino-5-hydroxy-pyridin-3-yl)-N-(3-methoxy-2-propoxy-benzyl)-N-methylacrylamide hydrochloride

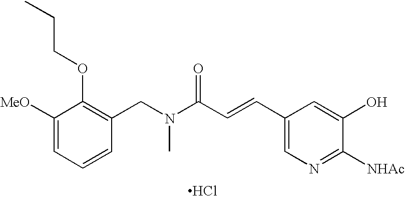

### a) 2-Amino-5-bromopyridin-3-ol

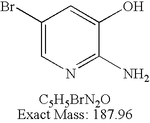

To a suspension of 6-bromo-3H-oxazolo[4,5-b]pyridin-2-one (20.0 g, 93.0 mmol) in methanol (200 mL) was added NaOH (20.0 g, 500 mmol) in H₂O (200 mL) and the mixture was heated to reflux overnight. After cooling, methanol was removed in vacuo. The aqueous residue was acidified with 3N HCl to pH 5-6. The resulting precipitate was collected by filtration, and dried overnight under vacuum at 45° C. to give the title compound as a tan solid (16.4 g, 93%): ¹H NMR (300 MHz, DMSO-d₆) δ 10.03 (s, 1H), 7.47 (d, J=2.0 Hz, 1H), 6.93 (d, J=2.0 Hz, 1H), 5.72 (s, 2H); ESI MS m/e 188 (M+H)⁺.

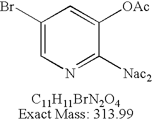

### b) Acetic acid 5-bromo-2-diacetylaminopyridin-3-yl ester

To a solution of 2-amino-5-bromopyridin-3-ol (1.89 g, 10.0 mmol) in 1,4-dioxane (20 mL) was added acetic anhydride (4.7 mL, 50 mmol) and the mixture was heated to reflux for 20 h. After cooling, the solvent was removed in vacuo. Purification by column chromatography (silica gel, hexanes/EtOAc, 70:30 to 50:50) gave the title compound (2.30 g, 73%) as a white solid: ¹H NMR (300 MHz, CDCl₃) δ 8.52 (d, J=2.1 Hz, 1H), 7.87 (d, J=2.1 Hz, 1H), 2.30 (s, 6H), 2.29 (s, 3H); ESI MS m/e 315 (M+H)⁺.

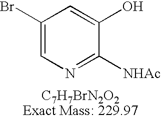

### c) N-(5-Bromo-3-hydroxy-pyridin-2-yl)acetamide

To a solution of acetic acid 5-bromo-2-diacetylaminopyridin-3-yl ester (100 mg, 0.317 mmol) in methanol (10 mL) was added K₂CO₃ (219 mg, 1.59 mmol) and water (2 mL), and the mixture was stirred overnight at room temperature. Methanol was removed in vacuo. The residue was diluted with water (8 mL) and the mixture was acidified with 3N HCl to pH 5-6. The resulting precipitate was collected by filtration to give the title compound (70 mg, 96%) as a pale yellow solid: ¹H NMR (500 MHz, DMSO-d₆) δ 10.83 (s, 1H), 10.55 (br s, 1H), 7.99 (d, J=2.0 Hz, 1H), 7.50 (d, J=2.0 Hz, 1H), 2.14 (s, 3H); ESI MS m/z 231 (M+H)⁺.

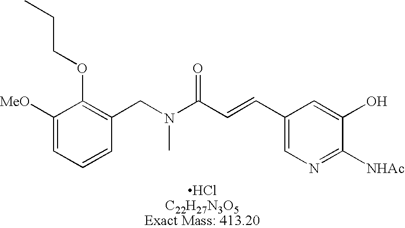

### d) (E)-3-(6-Acetylamino-5-hydroxy-pyridin-3-yl)-N-(3-methoxy-2-propoxy-benzyl)-N-methylacrylamide hydrochloride

To a solution of N-(5-bromo-3-hydroxy-pyridin-2-yl)acetamide (462 mg, 2.00 mmol) in propionitrile (40 mL) and DMF (10 mL) were added N-(3-methoxy-2-propoxy-benzyl)-N-methylacrylamide (648 mg, 2.60 mmol), (i-Pr)₂EtN (0.70 mL, 4.0 mmol), Pd(OAc)₂ (45 mg, 0.20 mmol) and P(o-tol)₃ (122 mg, 0.400 mmol), and the mixture was de-oxygenated with argon for 15 min. The mixture was heated to reflux overnight, allowed to cool and then filtered through a pad of diatomaceous earth. The filtrate was concentrated and the residue was dissolved in CH₂Cl₂ (150 mL). The solution was washed with water and brine, dried (Na₂SO₄) and the solvent was removed in vacuo. Purification by column chromatography (silica gel, CH₂Cl₂/MeOH, 96:4) gave the free base (500 mg, 60%) of the title compound. A solution of the free base (195 mg, 0.472 mmol) in CH₂Cl₂ (8 mL) was treated with anhydrous HCl (0.47 mL of 11.0M solution in Et₂O, 0.47 mmol). After stirring for 1 h, the mixture was diluted with Et₂O (15 mL) and allowed to stir for 15 min. The solid was isolated by filtration, washed with Et₂O and dried under vacuum at 45° C. overnight to give the title compound (190 mg, 90%) as a pale yellow solid and as a mixture of amide rotamers: ¹H NMR (300 MHz, DMSO-d₆) δ 11.38 (br s, 1H), 11.13-11.11 (m, 1H), 8.22-8.18 (m, 1H), 7.90-7.83 (m, 1H), 7.57-7.50 (m, 1H), 7.34-7.27 (m, 1H), 7.07-6.94 (m, 3H), 6.69-6.62 (m, 1H), 4.80-4.64 (m, 2H), 3.92-3.85 (m, 2H), 3.80 (s, 3H), 3.11-2.88 (m, 3H), 2.26-2.24 (m, 3H), 1.75-1.67 (m, 2H), 1.01-0.93 (m, 3H); MS (ESI) m/e 414 (M+H)⁺.

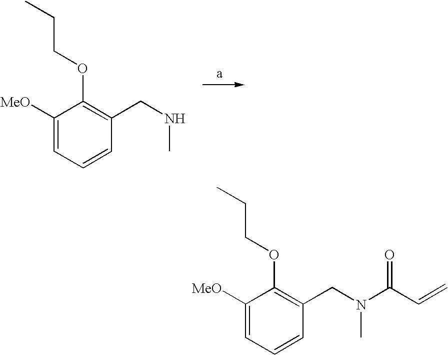

a) acryloyl chloride, Et₃N, CH₂Cl₂

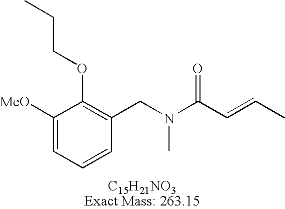

### a) N-(3-Methoxy-2-propoxy-benzyl)-N-methylacrylamide

A solution of (3-methoxy-2-propoxy-benzyl)methylamine (3.50 g, 16.7 mmol) in CH₂Cl₂ (80 mL) was treated with acryloyl chloride (1.3 mL, 17 mmol) and triethylamine (4.6 mL, 33 mmol). The mixture was stirred at room temperature for 2 h. The solution was washed with water and brine, dried (Na₂SO₄), filtered and concentrated under reduced pressure to give the title compound (4.10 g, 93%) as a tan oil and as a mixture of amide rotamers: ¹H NMR (300 MHz, DMSO-d₆) δ 7.10-6.90 (m, 2H), 6.87-6.56 (m, 2H), 6.18-6.12 (m, 1H), 5.74-5.62 (m, 1H), 4.64-4.58 (m, 2H), 3.89-3.83 (m, 3H), 3.79-3.78 (m, 2H), 2.99-2.86 (m, 3H), 1.73-1.63 (m, 2H), 0.97 (t, J=7.5 Hz, 3H).

### Example 4

### Preparation of (E)-3-(6-Acetylamino-5-hydroxy-pyridin-3-yl)-N-methyl-N-(3-methyl-benzofuran-2-ylmethyl)acrylamide hydrochloride

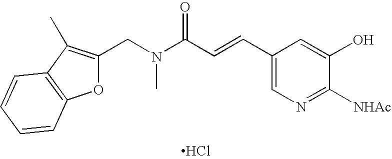

### a) (E)-3-(6-Acetylamino-5-hydroxy-pyridin-3-yl)-N-methyl-N-(3-methyl-benzofuran-2-ylmethyl)acrylamide

To a solution of N-(5-bromo-3-hydroxy-pyridin-2-yl)acetamide (360 mg, 1.54 mmol) in propionitrile (40 mL) and DMF (10 mL) were added N-methyl-N-(3-methyl-benzofuran-2-ylmethyl)acrylamide (390 mg, 1.79 mmol), (i-Pr)₂EtN (0.50 mL, 3.1 mmol), Pd(OAc)₂ (35 mg, 0.15 mmol) and P(o-tol)₃ (94 mg, 0.31 mmol), and the mixture was de-oxygenated with argon for 15 min. The mixture was heated to reflux overnight, allowed to cool and then filtered through a pad of diatomaceous earth. The filtrate was concentrated and the residue was dissolved in CH₂Cl₂ (100 mL). The solution was washed with water and brine, dried (Na₂SO₄) and the solvent was removed in vacuo. Purification by slow precipitation from CH₂Cl₂ gave the free base (245 mg, 42%) of the title compound. A solution of the free base (201 mg, 0.532 mmol) in CH₂Cl₂ (80 mL) was treated with anhydrous HCl (0.55 mL of 1.0M solution in Et₂O, 0.55 mmol). After stirring for 1 h, the volume of the solution was reduced to about 20 mL by evaporation. The mixture was then diluted with Et₂O (15 mL) and allowed to stir for 15 min. The solid was isolated by filtration, washed with Et₂O and dried under vacuum at 45° C. overnight to give the title compound (200 mg, 90%) as a pale-yellow solid and as a mixture of amide rotamers: ¹H NMR (300 MHz, DMSO-d₆) δ 11.44 (br s, 1H), 11.15 (br s, 1H), 8.23-8.21 (m, 1H), 7.95-7.90 (m, 1H), 7.58-7.48 (m, 4H), 7.30-7.21 (m, 3H), 5.00-4.80 (m, 2H), 3.19-2.95 (m, 3H), 2.28-2.20 (m, 6H); MS (ESI) m/e 380 (M+H)⁺.

### Example 5

### Preparation of (E)-3-(6-Amino-5-benzyloxy-pyridin-3-yl)-N-methyl-N-(3-methyl-benzofuran-2-ylmethyl)acrylamide

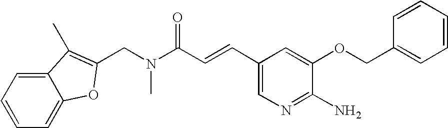

### a) 3-Benzyloxy-5-bromo-pyridin-2-ylamine

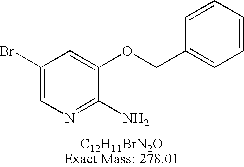

To a solution of 3-benzyloxy-pyridin-2-ylamine (2.00 g, 10.0 mmol) in DMF (15 mL) was slowly added bromine (0.56 mL, 11 mmol) at 0° C., and the mixture was stirred at room temperature for 2 h. The mixture was diluted with ice-water (25 mL) and neutralized with 3 N NaOH to pH 5-6. The resulting precipitate was collected by filtration to give the title compound (1.6 g, 57%) as a brown solid: ¹H NMR (300 MHz, CDCl₃) δ 7.73 (d, J=1.8 Hz, 1H), 7.42-7.37 (m, 5H), 7.08 (d, J=2.1 Hz, 1H), 5.05 (s, 2H), 4.74 (br s, 2H); ESI MS m/e 279 (M+H)⁺.

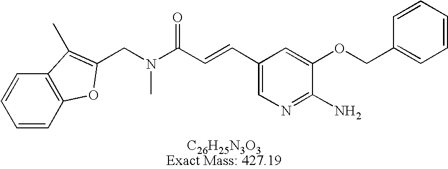

### b) (E)-3-(6-Amino-5-benzyloxy-pyridin-3-yl)-N-methyl-N-(3-methyl-benzofuran-2-ylmethyl)acrylamide

To a solution of 3-benzyloxy-5-bromo-pyridin-2-ylamine (278 mg, 1.00 mmol) in propionitrile (24 mL) and DMF (6 mL) were added N-methyl-N-(3-methyl-benzofuran-2-ylmethyl)acrylamide (252 mg, 1.10 mmol), (i-Pr)₂EtN (0.35 mL, 2.00 mmol), Pd(OAc)₂ (22 mg, 0.10 mmol) and P(o-tol)₃ (61 mg, 0.20 mmol), and the mixture was de-oxygenated with argon for 15 min. The mixture was heated to reflux overnight, allowed to cool, filtered through a pad of diatomaceous earth, and the filtrate was concentrated. Purification by column chromatography (silica gel, CH₂Cl₂/MeOH, 96:4) and then by slow precipitation from CH₂Cl₂/hexanes gave the title compound (165 mg, 39%) as a pale yellow solid and as a mixture of amide rotamers: ¹H NMR (300 MHz, DMSO-d₆) δ 7.77 (s, 1H), 7.58-6.94 (m, 12H), 6.27 (s, 2H), 5.19 (s, 2H), 5.00-4.78 (m, 2H), 3.18-2.92 (m, 3H), 2.27 (s, 3H); MS (ESI) m/e 428 (M+H)⁺.

### Example 6

### Preparation of N-methyl-N-[1-(R)-(3-methyl-benzofuran-2-yl)-ethyl]-3-(7-oxo-5,6,7,8-tetrahydro-[1,8]naphthyridin-3-yl)acrylamide

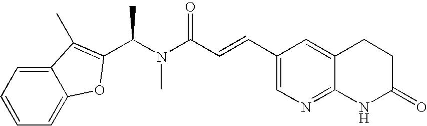

### a) 1-(3-methyl-benzofuran-2-yl)ethanone

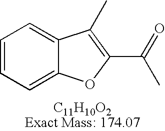

To a solution of 1-(2-hydroxy-phenyl)ethanone (10.0 mL, 83.0 mmol) and chloroacetone (6.60 mL, 83.0 mmol) was added K₂CO₃ (23.0 g, 166 mmol). The resulting dark orange suspension was stirred at room temperature overnight. The mixture was diluted with H₂O and extracted with Et₂O (3×). The combined organics were washed with NaOH (0.5 N in H₂O, 3×) and satd NaCl (1×), dried (Na₂SO₄) and concentrated. Purification by column chromatography (silica gel, hexanes to 95:5 hexanes/EtOAc) gave the title compound (9.19 g, 64%) as a yellow oil: ¹H NMR (300 MHz, CDCl₃) δ 7.67-7.64 (m, 1H), 7.53-7.45 (m, 2H), 7.33-7.28 (m, 1H), 2.62 (s, 3H), 2.60 (s, 3H).

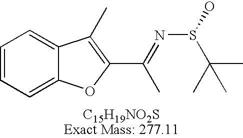

### b) (R)-2-methyl-propane-2-sulfinic acid [1-(3-methyl-benzofuran-2-yl)ethylidene]amide

To a solution of 1-(3-methyl-benzofuran-2-yl)ethanone (4.35 g, 25.0 mmol) and (R)-(+)-2-methyl-2-propanesulfinamide (3.03 g, 25.0 mmol) in THF (100 mL) was added Ti(OEt)₄ (10.5 mL, 50.0 mmol). The mixture was heated to reflux overnight. The solution was cooled to room temperature and poured into rapidly stirring satd NaCl (100 mL). The solution was filtered through diatamaceous earth, washing with EtOAc. The organic layer was dried (Na₂SO₄) and concentrated. Purification by flash column chromatography (silica gel, hexanes/EtOAc, 8:2) gave the title compound (3.56 g, 52%) as an orange solid: ¹H NMR (300 MHz, CDCl₃) δ 7.64-7.61 (m, 1H), 7.50-7.39 (m, 2H), 7.32-7.26 (m, 1H), 2.82 (s, 3H), 2.60 (s, 3H), 1.34 (s, 9H).

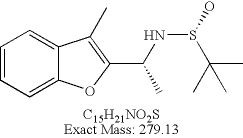

### c) (R)-2-methyl-propane-2-sulfinic acid [1-(3-methyl-benzofuran-2-yl)ethyl]amide

To an ice-cold solution of (R)-2-methyl-propane-2-sulfinic acid [1-(3-methyl-benzofuran-2-yl)ethylidene]amide (3.56 g, 12.8 mmol) in THF (60 mL) was added 9-BBN (26.9 mL of a 0.5 M solution in THF, 13.4 mmol) dropwise. The mixture was slowly warmed to room temperature and stirred overnight. The mixture was quenched with H₂O (10 mL) and concentrated. The residue was partitioned between Et₂O and water. The organic layer was washed with satd NaCl, dried (Na₂SO₄) and concentrated. Purification by column chromatography (silica gel, 1:1 hexanes/EtOAc) gave the title compound (3.44 g, 96%) as a brown oil: ¹H NMR (300 MHz, CDCl₃) δ 7.48-7.46 (m, 1H), 7.42-7.40 (m, 1H), 7.28-7.25 (m, 1H), 7.23-7.20 (m, 1H), 4.79-4.74 (m, 1H), 3.55 (d, J=3.0 Hz, 1H), 2.26 (s, 3H), 1.61 (d, J=4.1 Hz, 3H), 1.22 (s, 9H).

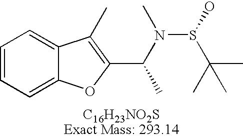

### d) (R,R)-2-methyl-propane-2-sulfinic acid methyl-[1-(3-methyl-benzofuran-2-yl)-ethyl]amide

To an ice-cold suspension of NaH (517 mg of a 60% dispersion in mineral oil, 12.9 mmol) in DMF (40 mL) was added a solution of (R)-2-methyl-propane-2-sulfinic acid [1-(3-methyl-benzofuran-2-yl)ethyl]amide (3.44 g, 12.3 mmol) in DMF (20 mL) drop-wise. The mixture was stirred 1 h then MeI (0.84 mL, 13.5 mmol) was added drop-wise. The mixture was slowly warmed to room temperature and stirred overnight. The mixture was quenched with H₂O and extracted with Et₂O (3×). The combined organics were washed with H₂O (2×) and satd NaCl, dried (Na₂SO₄) and concentrated. Purification by column chromatography (silica gel, 7:3 hexanes/EtOAc to 6:4 hexanes/EtOAc) gave the title compound (3.09 g, 85%) as a yellow oil: ¹H NMR (300 MHz, CDCl₃) δ 7.49-7.47 (m, 1H), 7.41-7.40 (m, 1H), 7.28-7.20 (m, 2H), 4.74-4.70 (m, 1H), 2.62 (s, 3H), 2.29 (s, 3H), 1.65 (d, J=4.3 Hz, 3H), 1.18 (s, 9H).

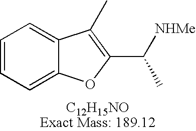

### e) (R)-methyl-[1-(3-methyl-benzofuran-2-yl)-ethyl]amine

To a solution of (R,R)-2-methyl-propane-2-sulfinic acid methyl-[1-(3-methyl-benzofuran-2-yl)-ethyl]amide (3.09 g, 10.5 mmol) in EtOH (35 mL) was added TFA (1.6 mL, 21.0 mmol) slowly. The resulting solution was heated to 40° C. overnight. The mixture was cooled to room temperature and concentrated. The residue was diluted with H₂O and partitioned between Et₂O and satd NaHCO₃. The aqueous layer was extracted with Et₂O (2×). The combined organics were washed with satd NaCl, dried (Na₂SO₄) and concentrated. Purification by column chromatography (silica gel, 95:5 CH₂Cl₂/MeOH) gave the title compound (1.73 mg, 87%) as a pale yellow oil: ¹H NMR (300 MHz, CDCl₃) δ 7.48-7.38 (m, 2H), 7.28-7.18 (m, 2H), 3.97-3.90 (m, 1H), 2.31 (s, 3H), 2.23 (s, 3H), 1.53-1.48 (m, 4H); HPLC (Chiralcel OD, 4.6×250 mm, UV=254 nm, 1.0 mL/min, hexanes/2-propanol 99.5:0.5)>99% ee, t_{R}=12.1 min (major isomer), t_{R}=16.5 min (minor isomer).

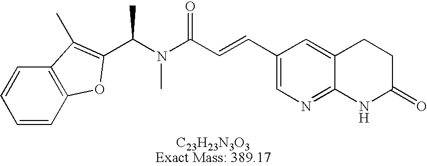

### f) N-methyl-N-[1-(R)-(3-methyl-benzofuran-2-yl)-ethyl]-3-(7-oxo-5,6,7,8-tetrahydro-[1,8]naphthyridin-3-yl)acrylamide

To a solution of 3-(7-oxo-5,6,7,8-tetrahydro-[1,8]naphthyridin-3-yl)acrylic acid hydrochloride (695 mg, 2.73 mmol) in DMF (15 mL) was added (R)-methyl-[1-(3-methyl-benzofuran-2-yl)-ethyl]amine (567 mg, 3.00 mmol), EDC (574 mg, 3.00 mmol), HOBt (405 mg, 3.00 mmol) and DIEA (1.4 mL, 8.2 mmol). The mixture was stirred at room temperature overnight. The mixture was diluted with H₂O and the solid collected by filtration. Purification by column chromatography (silica gel, 95:5 CH₂Cl₂/MeOH) gave an off-white solid. The solid was suspended in MeCN and sonicated for 20 min. The solid was collected by filtration to give the title compound (137 mg, 13%) as a white solid: ¹H NMR (300 MHz, DMSO-d₆) δ 10.67 (s, 1H), 8.37 (s, 1H), 8.15-8.07 (m, 1H), 7.58-7.7.51 (m, 3H), 7.36-7.7.13 (m, 3H), 6.20-5.95 (m, 1H), 3.33-3.04 (m, 4H), 2.93-2.79 (m, 3H), 2.18 (s, 3H), 1.66-1.54 (m, 3H); MS (ESI) m/e 390 (M+H)⁺.

### Example 7

### Preparation of N-Methyl-N-(1-methyl-1H-indol-2-ylmethyl)-3-(8-oxo-5,7,8,9-tetrahydro-6-oxa-1,9-diazabenzocyclohepten-3-yl)-acrylamide hydrochloride

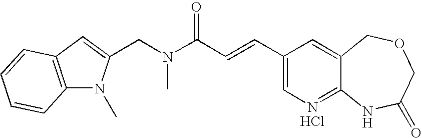

### a) Preparation of (2-Amino-5-bromo-pyridin-3-ylmethoxy)-acetic acid ethyl ester

A solution of ethyl glycolate (2.73 mL, 28.8 mmol) in anhydrous DMF (100 mL) was cooled to 0° C. then treated with NaH (60% in mineral oil, 1.15 g, 28.8 mmol). The resulting suspension was stirred at 0° C. for 30 min then 5-bromo-3-bromomethyl-pyridin-2-ylamine hydrobromide (5.00 g, 14.4 mmol) was added in portions. The mixture was allowed to warm to room temperature over 1.5 h then quenched with H₂O (200 mL). This was extracted with 2×150 mL ethyl acetate (2×150 mL). The combined organic fractions were washed with H₂O (100 mL), brine (100 mL), dried over MgSO₄ then concentrated to give a yellow residue. This residue was subjected to flash chromatography on silica gel using 40% ethyl acetate:hexanes to give the title compound as an off white solid. Yield: 2.9 g (70%); ¹H NMR (300 MHz, DMSO-d₆) δ 7.99 (d, J=2.5 Hz, 1H), 7.57 (d, J=2.5 Hz, 1H), 6.25 (br s, 2H), 4.38 (s, 2H), 4.22 (s, 2H), 4.17 (q, J=7.2 Hz, 2H), 1.24 (t, J=7.2 Hz, 3H); ESI MS nm/z 289 (100%); 291 (1000%) [C₁₀H₁₃BrN₂O₃]⁺

### b) 3-Bromo-5,9-dihydro-6-oxa-1,9-diazabenzocyclohepten-8-one

A solution of (2-Amino-5-bromo-pyridin-3-ylmethoxy)-acetic acid ethyl ester (2.8 g, 9.68 mmol) in anhydrous DMSO (120 mL) was treated with NaH (60% in mineral oil, 0.39 g, 9.68 mmol). After stirring for 5 h, the mixture was poured slowly onto ice-cooled H₂O (300 mL) with rapid stirring. A white precipitate formed and it was filtered, washed with cold H₂O (100 mL) then a 1:1 mixture of ethyl acetate:hexanes (100 mL). The solid was collected and dried under high vacuum overnight. Yield: 2.0 g (85%); ¹H NMR (300 MHz, DMSO-d₆) δ 10.56 (s, 1H), 8.35 (d, J=2.3 Hz, 1H), 7.91 (d, J=2.3 Hz, 1H), 4.76 (s, 2H), 4.52 (s, 2H); ESI MS m/z 242 (100%); 244 (100%) [C₈H₇BrN₂O₂]⁺

### c) 3-(8-Oxo-5,7,8,9-tetrahydro-6-oxa-1,9-diaza-benzocyclohepten-3-yl)-acrylic acid tert-butyl ester

A suspension of 3-Bromo-5,9-dihydro-6-oxa-1,9-diazabenzocyclohepten-8-one (1.0 g, 4.1 mmol), tert-butyl acrylate (3.0 mL, 20.7 mmol) and (i-Pr)₂EtN (2.2 mL, 12.4 mmol) in DMF (50 mL) was de-oxygenated with Ar for 30 min. The mixture was treated with Pd(OAc)₂ (93 mg, 0.41 mmol) and P(o-tol)₃ (250 mg, 0.82 mmol) then heated to 110° C. for 22 h. The hot mixture was filtered through a pad of celite. The filtrate was diluted with H₂O (100 mL) then extracted with ethyl acetate (2×150 mL). The combined organic fractions were dried over MgSO₄, pre-adsorbed onto silica gel and subjected to flash chromatography on silica gel using 5% methanol:dichloromethane. The appropriate fractions were collected, concentrated and triturated with Et₂O (20 mL) to give a cream solid. Yield: 0.85 g (71%); ¹H NMR (300 MHz, DMSO-d₆) δ 10.61 (s, 1H), 8.49 (d, J=2.1 Hz, 1H), 8.04 (d, J=2.1 Hz, 1H), 7.50 (d, J=16.1 Hz, 1H), 6.53 (d, J=16.1 Hz, 1H), 4.75 (s, 2H), 4.52 (s, 2H), 1.47 (s, 9H); ESI MS m/z 291 [C₁₅H₁₈N₂O₄+H]⁺

### d) 3-(8-Oxo-5,7,8,9-tetrahydro-6-oxa-1,9-diaza-benzocyclohepten-3-yl)-acrylic acid hydrochloride

A suspension of 3-(8-Oxo-5,7,8,9-tetrahydro-6-oxa-1,9-diaza-benzocyclohepten-3-yl)-acrylic acid tert-butyl ester (0.80 g, 2.7 mmol) in CH₂Cl₂ (10 mL) was treated with TFA (10 mL). After stirring at room temperature for 2 h 15 min, the clear tan solution was concentrated in vacuo. The resulting oil was treated with anhydrous HCl in dioxane (10 mL, 4.0 M) and sonicated until the oil was converted to a fine off-white solid. After stirring for 20 min, the suspension was concentrated. The solid was washed with Et₂O, isolated by filtration and dried under vacuum. Yield: 0.7 g (94%); ¹H NMR (300 MHz, DMSO-d₆) δ 10.62 (s, 1H), 8.51 (d, J=2.1 Hz, 1H), 8.06 (d, J=2.1 Hz, 1H), 7.58 (d, J=16.1 Hz, 1H), 6.55 (d, J=16.1 Hz, 1H), 4.79 (s, 2H), 4.55 (s, 2H); ESI MS m/z 235 [C₁₁H₁₀N₂O₄+H]⁺

### e) N-Methyl-N-(1-methyl-1H-indol-2-ylmethyl)-3-(8-oxo-5,7,8,9-tetrahydro-6-oxa-1,9-diazabenzocyclohepten-3-yl)-acrylamide hydrochloride

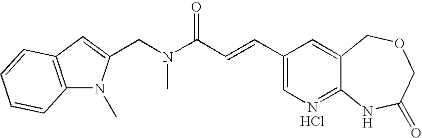

EDC (0.21 g, 1.1 mmol) was added to a suspension of 3-(8-Oxo-5,7,8,9-tetrahydro-6-oxa-1,9-diaza-benzocyclohepten-3-yl)-acrylic acid hydrochloride (0.25 g, 0.9 mmol), HOBT (0.14 g, 1.0 mmol), Methyl-(1-methyl-1H-indol-2-ylmethyl)-amine (0.18 g, 1.0 mmol) and (i-Pr)₂EtN (0.9 mL, 5.5 mmol) in DMF (20 mL). The mixture was allowed to stir overnight at 40° C. The mixture was cooled to 0° C. and diluted with H₂O (60 mL) with rapid stirring. The resulting precipitate was filtered, washed with H₂O (20 mL) then dried under high vacuum. The solid was then subjected to flash chromatography on silica gel using 5% methanol:dichloromethane. The fractions were collected and treated with 5 mL of 2.0M HCl in Et₂O. The suspension was concentrated, triturated with Et₂O (50 mL) then filtered to give a beige solid as a mixture of amide rotamers. Yield: 0.2 g (68%); ¹H NMR (300 MHz, DMSO-d₆) δ 10.59 and 10.56 (2×s, 1H), 8.55 and 8.52 (2×s, 1H), 8.11 and 8.04 (2×s, 1H), 7.61-7.00 (m, 6H), 6.44 and 6.22 (2×s, 1H), 6.43 (br s, 1H), 5.07 and 4.87 (2×s, 2H), 4.80 and 4.75 (2×s, 2H), 4.55 and 4.52 (2×s, 2H), 3.73 and 3.70 (2×s, 3H), 3.13 and 3.00 (2×s, 3H); ESI MS m/z 391 [C₂₂H₂₃N₄O₃+H]⁺

### Example 8

### Preparation of N-Methyl-N-(3-methyl-benzofuran-2-ylmethyl)-3-(8-oxo-5,7,8,9-tetrahydro-6-oxa-1,9-diazabenzocyclohepten-3-yl)-acrylamide

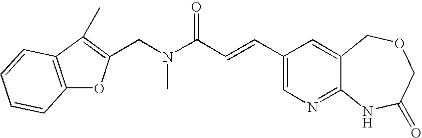

EDC (0.13 g, 0.66 mmol) was added to a suspension of 3-(8-Oxo-5,7,8,9-tetrahydro-6-oxa-1,9-diaza-benzocyclohepten-3-yl)-acrylic acid hydrochloride (0.15 g, 0.55 mmol), HOBt (82 mg, 0.61 mmol), Methyl-(3-methyl-benzofuran-2-ylmethyl)-amine (0.11 g, 0.61 mmol) and (i-Pr)₂EtN (0.46 mL, 2.75 mmol) in DMF (10 mL). The mixture was stirred overnight at room temperature then cooled to 0° C. and diluted with H₂O (20 mL) with rapid stirring. The resulting precipitate was filtered, washed with H₂O (20 mL) and dried under high vacuum. The residue was triturated with Et₂O (50 mL) then filtered to give a beige solid as a mixture of amide rotamers. Yield: 90 mg (42%); ¹H NMR (300 MHz, DMSO-d₆) δ 10.57 (s, 1H), 8.54 and 8.52 (2×s, 1H), 8.07 (br s, 1H), 7.57-7.21 (m, 6H), 4.97 and 4.79 (2×s, 2H), 4.80 (br s, 2H), 4.81 and 4.57 (2×s, 2H), 3.19 and 2.58 (2×s, 3H), 2.95 and 2.33 (2×s, 3H); ESI MS m/z 392 [C₂₂H₂₁N₃O₄+H]⁺

### Example 9

### Preparation of N-(3-Methoxy-2-propoxy-benzyl)-N-3-(8-oxo-5,7,8,9-tetrahydro-6-oxa-1,9-diazabenzocyclohepten-3-yl)-acrylamide

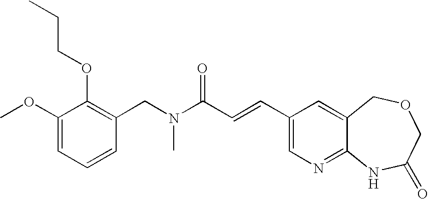

EDC (0.13 g, 0.66 mmol) was added to a suspension of 3-(8-Oxo-5,7,8,9-tetrahydro-6-oxa-1,9-diaza-benzocyclohepten-3-yl)-acrylic acid hydrochloride (0.15 g, 0.55 mmol), HOBt (82 mg, 0.61 mmol), (3-Methoxy-2-propoxy-benzyl)-methyl-amine (0.13 g, 0.61 mmol) and (i-Pr)₂EtN (0.46 mL, 2.75 mmol) in DMF (10 mL). The mixture was allowed to stir overnight at room temperature. The mixture was cooled to 0° C. and diluted with H₂O (20 mL) with rapid stirring. The resulting precipitate was filtered, washed with H₂O (20 mL) then dried under high vacuum. The solid was then subjected to flash chromatography on silica gel using 5% methanol:dichloromethane. The fractions were concentrated, triturated with Et₂O (50 mL) then filtered to give a beige solid as a mixture of amide rotamers. Yield: 50 mg (21%); ¹H NMR (300 MHz, DMSO-d₆) δ 10.58 and 10.56 (2×s, 1H), 8.54-6.65 (m, 7H), 4.80-4.53 (5×s, 6H), 3.90 (q, J=6.3 Hz, 2H), 3.81 (s, 3H), 3.12 and 2.88 (2×s, 3H), 1.72 (m, 2H), 0.99 (m, 3H); ESI MS m/z 426 [C₂₃H₂₇N₃O₅+H]⁺

### Example 10

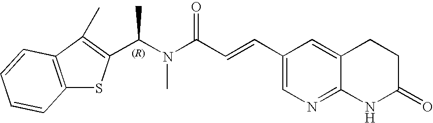

### N-Methyl-N-[1-(R)-(3-methyl-benzo[b]thiophen-2-yl)-ethyl]-3-(7-oxo-5,6,7,8-tetrahydro-[1,8]naphthyridin-3-yl)-acrylamide

### a) (R)-2-methyl-propane-2-sulfinic acid [1-(3-methyl-benzo[b]thiophen-2-yl)-ethylidene]amide

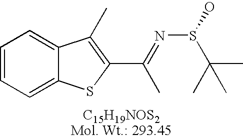

To a solution of 1-(3-methyl-benzo[b]thiophen-2-yl)ethanone (2.62 g, 13.8 mmol) and (R)-(+)-2-methyl-2-propanesulfinamide (1.67 g, 13.8 mmol) in THF (70 mL) was added Ti(OEt)₄ (6.4 mL, 27.6 mmol). The mixture was heated to reflux overnight. The solution was cooled to room temperature and poured into rapidly stirring satd NaCl (70 mL). The solution was filtered through diatomaceous earth and then washed with EtOAc. The organic layer was dried (Na₂SO₄) and concentrated. Purification by flash column chromatography (silica gel, hexanes/EtOAc, 7:3) gave the title compound (3.11 g, 77%) as an yellow solid: ¹H NMR (300 MHz, CDCl₃) δ 7.82-7.79 (m, 2H), 7.46-7.40 (m, 2H), 2.88 (s, 3H), 2.75 (s, 3H), 1.38 (s, 9H).

### b) (R)-2-methyl-propane-2-sulfinic acid [1-(3-methyl-benzo[b]thiophen-2-yl)-ethyl]amide

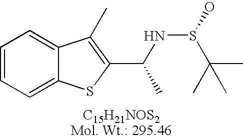

To an ice-cold solution of (R)-2-methyl-propane-2-sulfinic acid [1-(3-methyl-benzo[b]thiophen-2-yl)-ethylidene]amide (3.10 g, 10.6 mmol) in THF (50 mL) was added 9-BBN (22.0 mL of a 0.5 M solution in THF, 11.2 mmol) dropwise. The mixture was stirred at 0° C. for 2 h then warmed to room temperature. After 4 h, the mixture was quenched with H₂O (10 mL) and concentrated. The residue was partitioned between Et₂O and H₂O. The organic layer was washed with satd NaCl, dried (Na₂SO₄) and concentrated. Purification by column chromatography (silica gel, 7:3 hexanes/EtOAc to 3:7 hexanes/EtOAc) gave the title compound (2.76 g, 88%) as a light yellow oil: ¹H NMR (300 MHz, CDCl₃) δ 7.78 (d, J=7.1 Hz, 1H), 7.66 (d, J=7.3 Hz, 1H), 7.38-7.32 (m, 2H), 5.12-5.05 (m, 1H), 3.57 (s, 1H), 2.36 (s, 3H), 1.61 (d, J=6.5 Hz, 3H), 1.28 (s, 9H).

### c) (R,R)-2-methyl-propane-2-sulfinic acid methyl-[1-(3-methyl-benzo[b]thiophen-2-yl)-ethyl]amide

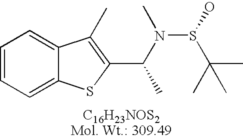

To an ice-cold suspension of NaH (394 mg of a 60% dispersion in mineral oil, 9.83 mmol) in DMF (30 mL) was added a solution of (R)-2-methyl-propane-2-sulfinic acid [1-(3-methyl-benzo[b]thiophen-2-yl)-ethyl]amide (2.76 g, 9.36 mmol) in DMF (15 mL) dropwise. The mixture was stirred 1 h then MeI (0.64 mL, 10.3 mmol) was added dropwise. The mixture was slowly warmed to room temperature. After 3 h, the mixture was quenched with H₂O and extracted with Et₂O (3×). The combined organics were washed with H₂O (2×) and satd NaCl, dried (Na₂SO₄) and concentrated. Purification by column chromatography (silica gel, 7:3 hexanes/EtOAc to 1:1 hexanes/EtOAc) gave the title compound (2.31 g, 80%) as a white solid: ¹H NMR (500 MHz, CDCl₃) δ 7.78 (d, J=7.8 Hz, 1H), 7.66 (dd, J=7.2, 0.4 Hz, 1H), 7.39-7.35 (m, 1H), 7.33-7.30 (m, 1H), 4.85 (q, J=6.7 Hz, 1H), 2.61 (s, 3H), 2.40 (s, 3H), 1.65 (d, J=6.7 Hz, 3H), 1.22 (s, 9H).

### d) (R)-methyl-[1-(3-methyl-benzo[b]thiophen-2-yl)-ethyl]amine

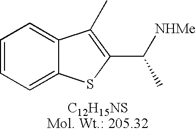

To a solution of (R,R)-2-methyl-propane-2-sulfinic acid methyl-[1-(3-methyl-benzo[b]thiophen-2-yl)-ethyl]amide (2.31 g, 7.48 mmol) in EtOH (40 mL) was added TFA (1.1 mL, 15.0 mmol) slowly. The resulting solution was heated to 40° C. overnight. The mixture was cooled to room temperature and concentrated. The residue was diluted with H₂O and partitioned between Et₂O and satd NaHCO₃. The aqueous layer was extracted with Et₂O (2×). The combined organics were washed with satd NaCl, dried (Na₂SO₄) and concentrated. Purification by column chromatography (silica gel, 95:5 CH₂Cl₂/MeOH) gave the title compound (1.46 g, 95%) as a pale yellow oil: ¹H NMR (500 MHz, CDCl₃) δ 7.79 (d, J=7.9 Hz, 1H), 7.63 (d, J=7.9 Hz, 1H), 7.37-7.33 (m, 1H), 7.30-7.27 (m, 1H), 4.19 (q, J=6.5 Hz, 1H), 2.40 (s, 3H), 2.37 (s, 3H), 1.47-1.44 (m, 4H).

### e) N-Methyl-N-[1-(R)-(3-methyl-benzo[b]thiophen-2-yl)-ethyl]-3-(7-oxo-5,6,7,8-tetrahydro-[1,8]naphthyridin-3-yl)-acrylamide

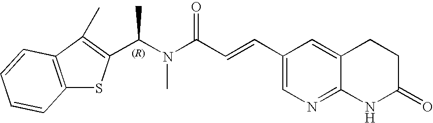

EDC (0.18 g, 0.95 mmol) was added to a suspension of 3-(7-Oxo-5,6,7,8-tetrahydro-[1,8]naphthyridin-3-yl)-acrylic acid hydrochloride (0.20 g, 0.79 mmol), HOBt (116 mg, 0.86 mmol), (R)-methyl-[1-(3-methyl-benzo[b]thiophen-2-yl)-ethyl]-amine (0.18 g, 0.86 mmol) and (i-Pr)₂EtN (0.80 mL, 4.74 mmol) in DMF (10 mL). The mixture was allowed to stir overnight at 40° C. The mixture was cooled to 0° C. and diluted with H₂O (30 mL) with rapid stirring. The resulting precipitate was filtered, washed with H₂O (20 mL) then dried under high vacuum. The solid was triturated and sonicated with Et₂O (30 mL), stirred for 1 hour then filtered to give a beige solid as a mixture of amide rotamers. Yield: 215 mg (67%); ¹H NMR (400 MHz, DMSO-d₆) δ 10.65 (s, 1H), 8.37 (s, 1H), 8.07 (s, 1H), 7.94 (d, J=8.0 Hz, 1H), 7.74 (d, J=8.0 Hz, 1H), 7.56-7.17 (m, 4H), 6.30 and 6.15 (2×m, 1H), 2.97 and 2.73 (2×s, 3H), 2.93-2.89 (m, 2H), 2.55-2.53 (m, 2H), 2.27 (s, 3H), 1.70-1.55 (2×m, 3H); ESI MS m/z 406 [C₂₃H₂₃N₃O₂S+H]⁺

### Example 11

### N-Methyl-N-[1-(3-methyl-benzo[b]thiophen-2-yl)-ethyl]-3-(8-oxo-5,7,8,9-tetrahydro-6-oxa-1,9-diaza-benzocyclohepten-3-yl)-acrylamide

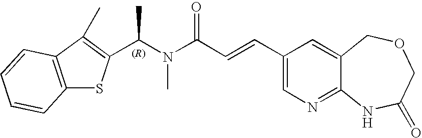

EDC (0.18 g, 0.95 mmol) was added to a suspension of 3-(8-Oxo-5,7,8,9-tetrahydro-6-oxa-1,9-diaza-benzocyclohepten-3-yl)-acrylic acid hydrochloride (0.21 g, 0.79 mmol), HOBt (116 mg, 0.86 mmol), (R)-methyl-[1-(3-methyl-benzo[b]thiophen-2-yl)-ethyl]-amine (0.18 g, 0.86 mmol) and (i-Pr)₂EtN (0.80 mL, 4.74 mmol) in DMF (10 mL). The mixture was allowed to stir overnight at 40° C. The mixture was cooled to 0° C. and diluted with H₂O (30 mL) with rapid stirring. The resulting precipitate was filtered, washed with H₂O (20 mL) then dried under high vacuum. The solid was triturated and sonicated with Et₂O (30 mL), stir for 30 minutes then filtered to give a beige solid as a mixture of amide rotamers. Yield: 249 mg (75%); ¹H NMR (400 MHz, DMSO-d₆) δ 10.58 (s, 1H), 8.53 (s, 1H), 8.07 (s, 1H), 7.94 (d, J=7.7 Hz, 1H), 7.74 (d, J=7.7 Hz, 1H), 7.58-7.20 (m, 4H), 6.30 and 6.05 (2×m, 1H), 4.77 (s, 2H), 4.53 (s, 2H), 2.97 and 2.74 (2×s, 3H), 2.27 (s, 3H), 1.67 and 1.56 (2×m, 3H); ESI MS m/z 422 [C₂₃H₂₃N₃O₃S+H]⁺

### Example 12

### Preparation of 3-(5-Amino-7-oxo-7,8-dihydro-[1,8]naphthyridin-3-yl)-N-methyl-N-(1-methyl-1H-indol-2-ylmethyl)-acrylamide hydrochloride

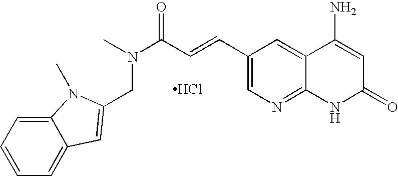

### a) Preparation of N-(5-Bromo-3-cyano-pyridin-2-yl)-acetamide

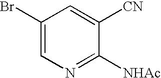

Potassium bis(trimethylsilyl)amide (25 mL, 12.5 mmol, 0.5M in toluene) was added to a solution of 2-amino-5-bromo-nicotinonitrile (496 mg, 2.5 mmol) in THF (10 mL) at −78° C. which was followed by a dropwise addition of acetyl chloride (533 μL, 7.5 mmol). The reaction was stirred at ambient temperature for 4 h then quenched with ammonium chloride (10 mL, sat). The resulting mixture was extracted with methylene chloride; the organic layer was washed with water and dried over MgSO₄. The volatiles were removed under vacuum to give the title compound (420 mg, 70%). ¹H NMR (300 MHz, DMSO-d₆, 8): 10.92 (s, 1H), 8.82 (d, J=2.3 Hz, 1H), 8.66 (d, J=2.3 Hz, 1H), 2.12 (s, 3H). MS (ESI) m/e: 240 and 242 (M+H)⁺.

### b) Preparation of 4-Amino-6-bromo-1H-[1,8]naphthyridin-2-one

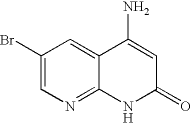

Potassium bis(trimethylsilyl)amide (10.2 mL, 10.1 mmol, 0.5M in toluene) was added to a solution of N-(5-Bromo-3-cyano-pyridin-2-yl)-acetamide (408 mg, 1.7 mmol) in THF (10 mL) at −78° C. The reaction was stirred at ambient temperature for 3 h then quenched with water; the mixture was extracted with ethyl acetate. The organic extract was washed with water, dried over MgSO₄ and evaporated. The remaining solid was stirred with CH₂Cl₂ (20 mL) which upon filtration and drying gave the title compound (220 mg, 54%). ¹H NMR (300 MHz, DMSO-d₆, δ): 11.27 (s, 1H), 8.58 (d, J=2.4 Hz, 1H), 8.53 (d, J=2.4 Hz, 1H), 6.75 (s, 2H), 5.46 (s, 1H). MS (ESI) m/e: 240 and 242 (M+H)⁺.

### c) Preparation of 3-(5-Amino-7-oxo-7,8-dihydro-[1,8]naphthyridin-3-yl)-N-methyl-N-(1-methyl-1H-indol-2-ylmethyl)-acrylamide hydrochloride

A DMF (10 mL) solution of 4-Amino-6-bromo-1H-[1,8]naphthyridin-2-one (220 mg, 0.92 mmol), N-methyl-N-(1-methyl-1H-indol-2-ylmethyl)-acrylamide (630 mg, 2.75 mmol) and diisopropylethylamine (480 μL, 2.75 mmol) was purged with Argon for 10 min. Pd(OAc)₂ (41 mg, 0.18 mmol) and P(o-Tol)₃ (112 mg, 0.37 mmol) were added and the Argon purge was repeated. The mixture was heated to 120° C. and stirred for 5 h under Argon. Upon cooling, water was added and the mixture was extracted with ethyl acetate. The extract was washed with water and brine, dried over MgSO₄ and evaporated. The crude product was purified by Flash chromatography (silica, 3-5% MeOH in CH₂Cl₂). The purified free base was converted to its HCl salt by addition of HCl (1 mL, 1 mmol, 1M in ether). The salt was washed with ether and dried to afford 50 mg (13%) of the title compound. ¹H NMR (300 MHz, DMSO-d₆, δ): 9.95 and 9.75 (rotamers, 2s, 1H), 9.08 and 9.02 (rotamers, 2s, 1H), 8.00-7.58 (m, 4H), 7.18-6.92 (m, 2H), 6.41, 6.38, 6.33 and 6.12 (rotamers, 4s, 2H), 5.28 and 4.87 (rotamers, 2s, 2H), 3.78 and 3.69 (rotamers, 2s, 3H), 3.21 and 3.01 (rotamers, 2s, 3H). MS (ESI) m/e: 388.1780 (M+H)⁺.

### Example 13

### Preparation of 3-(5-hydroxy-7-oxo-7,8-dihydro-[1,8]naphthyridin-3-yl)-N-methyl-N-(1-methyl-1H-indol-2-ylmethyl)-acrylamide

### Preparation of 3-(5-hydroxy-7-oxo-7,8-dihydro-[1,8]naphthyridin-3-yl)-acrylic acid

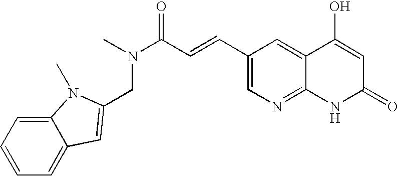

### a) Preparation of 2-Amino-5-bromonicotinic acid

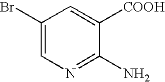

A solution of bromine (7 mL, 136 mmol) in acetic acid (20 mL) was added dropwise to an acetic acid (230 mL) suspension of 2-aminonicotinic acid (9.37 g, 68 mmol) at 10° C. After stirring the mixture for 6 hours, ether (600 mL) was added and the stirring was continued for 14 hours. The mixture was cooled to 0° C., the precipitate was filtered, washed with ether and dried to give 18.2 g (90%) of a hydrobromide salt. The salt was dissolved in water (200 mL) and sodium hydroxide solution (60 mL, 2N) then neutralized with 1N HCl. The resulting precipitate was filtered and dried to afford of the title compound (12.01 g, 82%). ¹H NMR (300 MHz, DMSO-d₆, δ): 8.25 (d, J=2.6 Hz, 1H), 8.09 (d, J=2.6 Hz, 1H).

### b) Preparation of Methyl 2-Amino-5-bromonicotinate

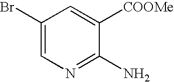

Trimethylsilyldiazomethane (15 mL, 30 mmol, 2M in ether) was added dropwise to a stirred suspension of 2-amino-5-bromonicotinic acid (2.51 g, 11.6 mmol) in methylene chloride (50 mL) and methanol (50 mL) at 10° C. After stirring one hour, the excess reagent was destroyed with acetic acid and the mixture was evaporated to a paste. The crude product was recrystallized from methanol to give the title compound (2.41 g, 90%). ¹H NMR (300 MHz, DMSO-d₆, δ): 8.29 (d, J=2.5 Hz, 1H), 8.12 (d, J=2.5 Hz, 1H), 7.33 (s, br, 2H), 3.82 (s, 3H).

### c) Preparation of 2-acetylamino-5-(2-ethoxycarbonyl-vinyl)-nicotinic acid methyl ester

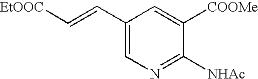

Acetic anhydride (9.5 mL, 100 mmol) was added to a dioxane (30 mL) solution of 2-acetylamino-5-bromo-nicotinic acid (4.63 g, 20 mmol). The mixture was heated at 135° C. in a sealed tube for 32 h. Upon cooling, the solvent was removed under vacuum. The residue was evaporated from methanol (3×20 mL) then once from hexanes to afford 5.89 g (100%) of a 1:1 mixture of mono- and bis-acetylated products. This mixture was submitted to Heck-coupling without further purification. The mixture was dissolved in propionitrile (100 mL), ethyl acrylate (6.2 mL, 60 mmol) and diisopropylethylamine (10.5 mL, 60 mmol) were added and the reaction vessel was purged with Argon for 10 min. Pd(OAc)₂ (449 mg, 2 mmol) and P(o-Tol)₃ (1.22 g, 4 mmol) were added and the Argon purge was repeated. The mixture was stirred at 100° C., for 20 h, under Argon. Upon cooling, the volatiles were evaporated, water was added then the mixture was extracted with methylene chloride. The extract was washed with water and dilute HCl, dried over MgSO₄ and evaporated to dryness. The crude product was purified by chromatography to afford 2.01 g (34%) of the title compound. ¹H NMR (300 MHz, DMSO-d₆, δ): 10.81 (s, 1H), 8.85 (d, J=2.2 Hz, 1H), 8.39 (d, J=2.2 Hz, 1H), 7.69 (d, J=16.2 Hz, 1H), 6.78 (d, J=16.2 Hz, 1H), 4.19 (q, J=7.1 Hz, 2H), 3.75 (s, 3H), 2.10 (s, 3H), 1.26 (t, J=7.1 Hz, 3H).

### d) Preparation of 3-(5-hydroxy-7-oxo-7,8-dihydro-[1,8]naphthyridin-3-yl)-acrylic acid

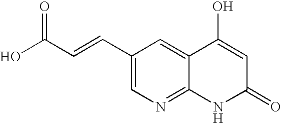

Sodium bis(trimethylsilyl)amide (28.7 mL, 28.7 mmol, 1M in THF) was added to a solution of 2-acetylamino-5-(2-ethoxycarbonyl-vinyl)-nicotinic acid methyl ester (2.00 g, 7.18 mmol) in THF (10 mL) at −78° C. The mixture was stirred at ambient temperature for 3 h then methanol (10 mL) and water (10 mL) were added at 0° C. The mixture was stirred at ambient temperature for 25 h then concentrated to 30 mL. This crude aqueous solution was washed with methylene chloride (2×20 mL) and acidified with concentrated HCl. The precipitate was filtered and dried to afford the title compound (1.05 g, 63%). ¹H NMR (300 MHz, DMSO-d₆, δ): 11.8 (s, br, 2H), 8.83 (d, J=2.2 Hz, 1H), 8.69 (d, J=2.2 Hz, 1H), 7.68 (d, J=16.1 Hz, 1H), 6.64 (d, J=16.1 Hz, 1H), 5.86 (s, 1H). MS (EI) m/e: 232 (M)⁺.

### e) Preparation of 3-(5-hydroxy-7-oxo-7,8-dihydro-[1,8]naphthyridin-3-yl)-N-methyl-N-(1-methyl-1H-indol-2-ylmethyl)-acrylamide

EDC (231 mg, 1.2 mmol) was added to a solution of 3-(5-hydroxy-7-oxo-7,8-dihydro-[1,8]naphthyridin-3-yl)-acrylic acid (233 mg, 1.0 mmol), methyl-(1-methyl-1H-indol-2-ylmethyl)-amine (192 mg, 1.1 mmol), HOBt H₂O (150 mg, 1.1 mmol) and DIPEA (700 μL, 4.0 mmol) in dry DMF (10 mL). After 17 hr of stirring, the mixture was diluted with water (50 mL), washed with EtOAc (2×10 mL) and acidified. The precipitate was filtered, washed with water and dried to afford the title compound (184 mg, 47%). ¹H NMR (300 MHz, DMSO-d₆, δ): 11.7 (m, 2H), 8.85 and 8.81 (rotamers, 2s, 1H), 8.51 and 8.46 (rotamers, 2s, 1H), 7.67 (d, J=15.5 Hz, 1H), 7.55-7.35 (m, 3H) 7.12 (m, 1H), 7.01 (m, 1H), 6.43 and 6.20 (rotamers, 2s, 1H), 5.78 and 5.75 (rotamers, 2s, 1H), 5.10 and 4.86 (rotamers, 2s, 2H), 3.73 and 3.69 (rotamers, 2s, 3H), 3.14 and 2.99 (rotamers, 2s, 3H). MS (ESI) m/e 389 (M+H)⁺.

### Example 14

### Preparation of 3-(5-hydroxy-7-oxo-7,8-dihydro-[1,8]naphthyridin-3-yl)-N-methyl-N-(1-methyl-1H-indol-2-ylmethyl)-acrylamide

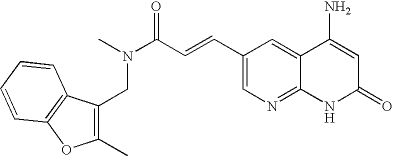

EDC (250 mg, 1.3 mmol) was added to a solution of 3-(5-hydroxy-7-oxo-7,8-dihydro-[1,8]naphthyridin-3-yl)-acrylic acid (233 mg, 1.0 mmol), methyl-(2-methyl-benzofuran-3-ylmethyl)-amine (220 μL, 1.2 mmol), HOBt.H₂O (150 mg, 1.1 mmol) and DIPEA (700 μL, 4.0 mmol) in dry DMF (10 mL). After 17 hr of stirring, the mixture was diluted with water (50 mL) at 10° C., washed with EtOAc (2×10 mL) and acidified. The precipitate was filtered, washed with water and dried to afford the title compound (206 mg, 53%). ¹H NMR (300 MHz, DMSO-d₆, δ): 11.73 (m, 2H), 8.85 and 8.84 (rotamers, 2s, 1H), 8.60 and 8.45 (rotamers, 2s, 1H), 7.68 (d, J=15.4 Hz, 1H), 7.6-7.1 (m, 5H), 5.77 (s, 1H), 4.97 and 4.74 (rotamers, 2s, 2H), 3.07 and 2.83 (rotamers, 2s, 3H) 2.57 (s, 3H). MS (ESI): m/e 390 (M+H)⁺.

### Example 15

### Preparation of 3-(5-hydroxy-7-oxo-7,8-dihydro-[1,8]naphthyridin-3-yl)-N-methyl-N-(3-methyl-benzofuran-2-ylmethyl)-acrylamide

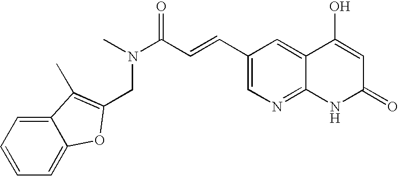

EDC (250 mg, 1.3 mmol) was added to a solution of 3-(5-hydroxy-7-oxo-7,8-dihydro-[1,8]naphthyridin-3-yl)-acrylic acid (233 mg, 1.0 mmol), methyl-(3-methyl-benzofuran-2-ylmethyl)-amine (220 μL, 1.2 mmol), HOBt.H₂O (150 mg, 1.1 mmol) and DIPEA (700 mL, 4.0 mmol) in dry DMF (10 mL). After 17 hr of stirring, the mixture was diluted with water (50 mL) at 10° C., washed with EtOAc (2×10 mL) and acidified. The precipitate was filtered, washed with water and dried to afford the title compound (193 mg, 50%). ¹H NMR (300 MHz, DMSO-d₆, δ): 11.76 (m, 2H), 8.84 (s, 1H), 8.57 and 8.50 (rotamers, 2s, 1H), 7.2-7.7 (m, 6H), 5.78 (s, 1H), 5.04 and 4.80 (rotamers, 2s, 2H), 3.21 and 2.95 (rotamers, 2s, 3H), 2.29 and 2.27 (rotamers, 2s, 3H). MS (ESI): m/e 390 (M+H)⁺.

### Example 16

### Preparation of N-(2-ethoxy-3-methoxy-benzyl)-3-(5-hydroxy-7-oxo-7,8-dihydro-[1,8]naphthyridin-3-yl)-N-methyl-acrylamide

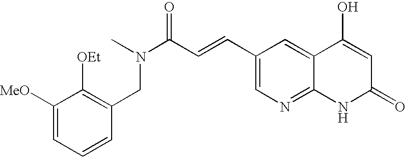

EDC (250 mg, 1.3 mmol) was added to a solution of 3-(5-hydroxy-7-oxo-7,8-dihydro-[1,8]naphthyridin-3-yl)-acrylic acid (233 mg, 1.0 mmol), (2-ethoxy-3-methoxy-benzyl)-methyl-amine (240 μL, 1.2 mmol), HOBt H₂O (150 mg, 1.1 mmol) and DIPEA (700 μL, 4.0 mmol) in dry DMF (10 mL). After 17 hr of stirring, the mixture was diluted with water (50 mL) at 10° C., washed with EtOAc (2×10 mL) and acidified. The precipitate was filtered, washed with water and dried to afford the title compound (200 mg, 49%). ¹H NMR (300 MHz, DMSO-d₆, δ): 11.74 (m, 2H), 8.85 and 8.78 (rotamers, 2s, 1H), 8.50 and 8.44 (rotamers, 2s, 1H), 6.99 (m, 2H), 6.67 (m, 1H), 5.78 and 5.76 (rotamers, 2s, 1H), 4.83 and 4.65 (rotamers, 2s, 2H), 4.00 (m, 2H), 3.79 (s, 3H), 3.13 and 2.88 (rotamers, 2s, 3H), 1.29 (m, 3H). MS (ESI): m/e 410 (M+H)⁺.

### Example 17

### Preparation of 3-(5-hydroxy-7-oxo-7,8-dihydro-[1,8]naphthyridin-3-yl)-N-(3-methoxy-2-propoxy-benzyl)-N-methyl-acrylamide

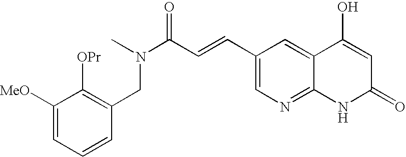

EDC (250 mg, 1.3 mmol) was added to a solution of 3-(5-hydroxy-7-oxo-7,8-dihydro-[1,8]naphthyridin-3-yl)-acrylic acid (233 mg, 1.0 mmol), (2-propoxy-3-methoxy-benzyl)-methyl-amine (260 μL, 1.2 mmol), HOBt 1H₂O (150 mg, 1.1 mmol) and DIPEA (700 μL, 4.0 mmol) in dry DMF (10 mL). After 17 hr of stirring, the mixture was diluted with water (50 mL) at 10° C., washed with EtOAc (2×10 mL) and acidified. The precipitate was filtered, washed with water and dried to afford title compound (190 mg, 42%). ¹H NMR (300 MHz, DMSO-d₆, δ): 11.75 (m, 2H), 8.85 and 8.79 (rotamers, 2s, 1H), 8.51 and 8.45 (rotamers, 2s, 1H), 7.62 (d, J=15 Hz, 1H), 7.40 (d, J=15 Hz, 1H), 6.99 (m, 2H), 6.68 (m, 1H), 5.78 and 5.76 (rotamers, 2s, 1H), 4.82 and 4.65 (rotamers, 2s, 2H), 3.89 (m, 2H), 3.79 (s, 3H), 3.13 and 2.88 (rotamers, 2s, 3H), 1.70 (m, 2H), 0.97 (m, 3H). MS (ESI): m/e 424 (M+H)⁺.

### Example 18

(E)-N-(3-Chloro-benzofuran-2-ylmethyl)-N-methyl-3-(2-oxo-1,2,3,5-tetrahydro-benzo[e][1,4]oxazepin-7-yl)-acrylamide

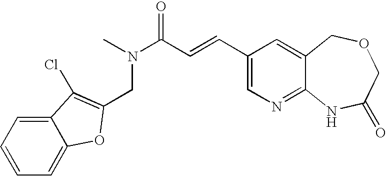

According to the procedure of Example 48 (3-chloro-benzofuran-2-ylmethyl)-methyl-amine (157 mg, 0.8 mmol) and 3-(2-oxo-1,2,3,5-tetrahydro-benzo[e][1,4]oxazepin-7-yl)-acrylic acid (198 mg, 0.73 mmol) were coupled to yield the title compound (166 mg, 55%) as a white solid and as a mixture of amide rotomers. ¹H NMR (400 MHz, DMSO-d₆) δ 10.61 (1s, 1H), 8.54 (1s, 1H), 8.07-8.09 (m, 1H), 7.36-7.67 (m, 5H), 8.25-7.29 (m, 1H), 4.89-5.09 (m, 2H), 4.78 (s, 2H), 4.54 (s, 2H), 3.23-3.34 (m, 3H); MS (ESI) m/e 411 (C₂₂H₁₉ClN₂O₄+H)⁺.

### Example 19

### (S,E)-3-(3,4-cyclopentyl-2,3,4,5-tetrahydro-1H-pyrido[2,3-e][1,4]diazepin-7-yl)-N-methyl-N-((3-methylbenzofuran-2-yl)methyl)acrylamide trifluoroacetic acid salt

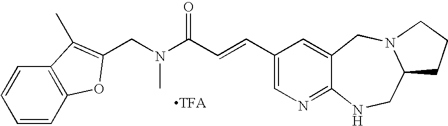

### (S)-7-bromo-3,4-cyclopentyl-2,3,4,5-tetrahydro-1H-pyrido[2,3-e][1,4]diazepine

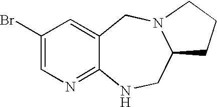

To a cooled solution (0° C.) of (S)-7-bromo-3,4-cyclopentyl-4,5-dihydro-1H-pyrido[2,3-e][1,4]diazepin-2(3H)-one (542 mg, 2 mmol) in THF (25 mL) is added a 1M solution of LAH in THF (3 mL, 3 mmol). The solution was slowly warmed to room temperature and stirred for 6 h. The reaction was cooled again and carefully quenched with water (10 mL), extracted with ethyl acetate (3×15 mL), dried over sodium sulfate and concentrated. The crude product was purified on a silica gel column (10% MeOH in CH₂Cl₂) to yield the title compound (115 mg, 21%): ¹H NMR (400 MHz, CD₃OD) δ 8.59 (s, 1H), 7.47 (s, 1H), 3.67-3.55 (m, 2H), 3.01-3.23 (m, 2H), 2.66 (m, 1H), 2.25 (m, 2H), 1.67 (m, 2H), 1.33 (m, 2H).

### (S,E)-3-(3,4-cyclopentyl-2,3,4,5-tetrahydro-1H-pyrido[2,3-e][1,4]diazepin-7-yl)-N-methyl-N-((3-methylbenzofuran-2-yl)methyl)acrylamide trifluoroacetic acid salt

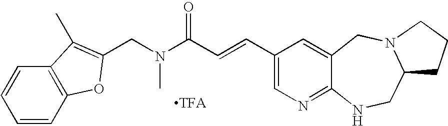

To a solution of (S)-7-bromo-3,4-cyclopentyl-2,3,4,5-tetrahydro-1H-pyrido[2,3-e][1,4]diazepine (115 mg, 0.43 mmol), tri(o-tolyl)phosphine (27 mg, 0.086 mmol), diisopropylethylamine (242 uL, 1.29 mmol), N-methyl-N-((3-methyl-3a,7a-dihydrobenzofuran-2-yl)methyl)acrylamide (148 mg, 0.65 mmol) in DMF (5 mL) was added palladium acetate (10 mg, 0.043 mmol) and the reaction heated to 90° C. overnight. The reaction was cooled to room temperature and passed through a pad of celite and washed with ethyl acetate (10 mL). The reaction was washed with water (10 mL) and extracted with ethyl acetate (3×15 mL), dried over sodium sulfate and concentrated. The residue was then re-dissolved in methylene chloride (5 mL) and cooled to 0° C. Trifluoroacetic acid (1 mL) was added and reaction stirred at room temperature for 1 h. The solution was concentrated and purified using preparative HPLC to yield a white solid (19 mg, 5%) as the TFA salt: ¹H NMR (400 MHz, CD₃OD) δ 8.31 (m, 1H), 8.03 (s, 1H), 7.54-7.49 (m, 2H), 7.39-7.37 (m, 1H), 7.29-7.23 (m, 2H), 4.93 (2s, 2H, rotamers), 3.67-3.55 (m, 2H), 3.32 (s, 3H), 3.25-3.23 (m, 2H), 2.67-2.66 (m, 1H), 2.25-2.24 (m, 2H), 1.67-1.66 (m, 2H), 1.33-1.32 (m, 2H). MS (ESI) m/e 417 (C₂₅H₂₈N₄O₂+H)⁺.

### Example 20

### (E)-N-methyl-N-((3-methylbenzofuran-2-yl)methyl)-3-[7-oxo-(6-spiropiperidinyl)-1,8-naphthyridin-3-yl]acrylamide hydrochloride

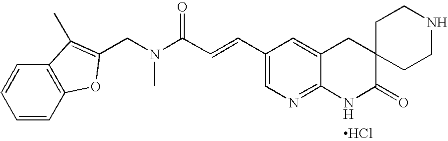

### 6-bromo-(4′-N-Boc-3-spiropiperidinyl)-1,8-naphthyridin-2(1H)-one

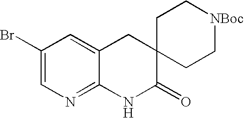

5-Bromo-3-(bromomethyl)pyridin-2-amine hydrobromide (4.8 g, 14 mmol) was added to anhydrous THF (56 mL) under argon. The slurry was cooled to −78° C. and freshly prepared LDA (14 mL, 1 M in THF, 14 mmol) was added dropwise and stirred 15 min. In a separate flask, N-Boc ethylisonipecotate (10.9 g, 42 mmol) was dissolved into anhydrous THF (100 mL) and cooled to −78° C. under argon. Freshly prepared LDA (42 mL of a 1M solution in THF) was added dropwise over 30 min and stir an additional 30 min. Via canula, the lithium salt of N-boc ethylisopecotate was added dropwise over 30 min. The mixture was stirred at −78° C. for 2 h and allowed to warm to room temperature. The reaction was quenched with sat. NH₄Cl (20 mL) and ethyl acetate (200 mL) was added. The solution was washed with water (2×50 mL), brine (50 mL), dried over MgSO₄ and concentrated. The solid was triturated with hexanes to afford the title compound as a beige solid (3.0 g, 55%): ¹H NMR (400 MHz, DMSO-d₆) δ 8.22 (s, 1H), 7.85 (s, 1H), 3.59-3.50 (m, 2H), 3.38-3.21 (m, 2H), 2.95 (s, 2H), 1.74-1.67 (m, 2H), 1.39 (s, 9H), 1.33-1.27 (m, 2H).

### (E)-benzyl 3-[7-oxo-(4′-N-Boc-6-spiropiperidinyl)-1,8-naphthyridin-3-yl]acrylate

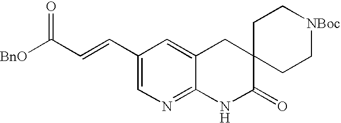

6-Bromo-(4′-N-Boc-3-spiropiperidinyl)-1,8-naphthyridin-2(1H)-one (2.8 g, 7 mmol) was dissolved into 4:1 DMF/propionitrile (37 mL) and degassed with argon. Diisopropylethylamine (4.9 mL, 28 mmol) and benzyl acrylate (4.8 g, 28 mmol) were added and the solution was degassed with argon. Palladium acetate (150 mg, 0.7 mmol) and tri-o-tolylphosphine (425 mg, 1.4 mmol) were added the solution was degassed. The mixture was heated in a sealed tube at 100° C. for 16 h. The solution was filtered through celite. The filter cake was washed with methylene chloride and the filtrate was concentrated to dryness to afford the title compound as a grey solid (1.7 g, 50%): ¹H NMR (400 MHz, DMSO-d₆) δ 9.88 (s, 1H), 8.41 (s, 1H), 7.68 (s, 1H) 7.67 (d, J=15.6 Hz, 1H), 7.45-7.32 (m, 5H), 6.47 (d, J=15.6 Hz, 1H), 5.27 (s, 2H), 3.68-3.62 (m, 2H), 3.52-3.46 (m, 2H), 2.90 (s, 2H), 2.05-1.92 (m, 2H), 1.47 (s, 9H), 1.48-1.45 (m, 2H); ESI MS m/z 478 [C₂₇H₃₁N₃O₅+H]⁺.

### (E)-3-[7-oxo-(4′-N-Boc-6-spiropiperidinyl)-1,8-naphthyridin-3-yl]acrylic acid

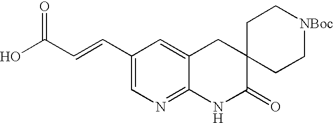

(E)-Benzyl 3-[7-oxo-(4′-N-Boc-6-spiropiperidinyl)-1,8-naphthyridin-3-yl]acrylate (1.65 g, 3.45 mL) was dissolved in a 1:1 solution of CH₂Cl₂/EtOH (60 mL). Sodium hydroxide (10.4 mL of 1M solution, 10.4 mmol) was added and the solution was stirred overnight. The solvents were removed in vacuo and the mixture was acidified to pH 2 with 2N HCl. The solution was extracted with ethyl acetate (3×50 mL), dried over MgSO₄ and concentrated to give the title compound as a white solid (1.01 g, 76%): ¹H NMR (400 MHz, CD₃OD) δ 8.29 (s, 1H), 7.91 (s, 1H) 7.62 (d, J=15.6 Hz, 1H), 6.47 (d, J=15.6 Hz, 1H), 3.68-3.62 (m, 2H), 3.52-3.41 (m, 2H), 2.98 (s, 2H), 1.95-1.86 (m, 2H), 1.49-1.42 (m, 11H).

### (E)-N-methyl-N-((3-methylbenzofuran-2-yl)methyl)-3-[7-oxo-(4′-N-Boc-6-spiropiperidinyl)-1,8-naphthyridin-3-yl]acrylamide

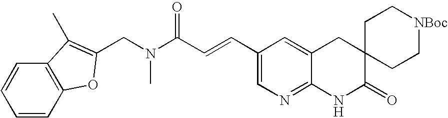

The title compound was prepared according to the standard procedure. The compound was purified on silica gel (3% MeOH in CH₂Cl₂) to give a white solid (299 mg, 70%): ESI MS m/z 545 [C₃₁H₃₆N₄O₅+H]⁺.

### (E)-N-methyl-N-((3-methylbenzofuran-2-yl)methyl)-3-[7-oxo-(6-spiropiperidinyl)-1,8-naphthyridin-3-yl]acrylamide hydrochloride

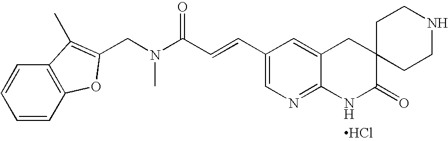

(E)-N-methyl-N-((3-methylbenzofuran-2-yl)methyl)-3-[7-oxo-(4′-N-Boc-6-spiropiperidinyl)-1,8-naphthyridin-3-yl]acrylamide (299 mg, 0.55 mmol) was dissolved into CH₂Cl₂ (25 mL). HCl (5.5 mL of 1M solution in ether) was added and the reaction was stirred overnight. The solid was collected by filtration and triturated with ether. The title compound was isolated as a white solid (240 mg, 90%): ¹H NMR (400 MHz, DMSO-d₆) δ 10.85 (s, 1H), 8.95 (bs, 1H), 8.75 (bs, 1H), 8.40 (s, 1H), 8.08 (s, 1H), 7.55-7.46 (m, 3H), 7.27-7.22 (m, 2H), 5.02-4.78 (2s, rotomers, 2H), 3.17-3.11 (m, 6H), 2.95-2.92 (m, 331), 2.25 (s, 3H), 2.05-1.92 (m, 2H), 1.70-1.60 (m, 2H); ESI MS m/z 445 [C₂₆H₂₈N₄O₃+H]⁺.

### Example 21

### (E)-N-methyl-N-((3-methylbenzothiophen-2-yl)methyl)-3-[7-oxo-(6-spiropiperidinyl)-1,8-naphthyridin-3-yl]acrylamide trifluoroacetate salt

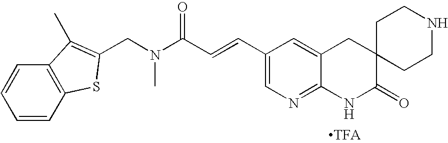

### (E)-N-methyl-N-((3-methylbenzothiophene-2-yl)methyl)-3-[7-oxo-(4′-N-Boc-6-spiropiperidinyl)-1,8-naphthyridin-3-yl]acrylamide

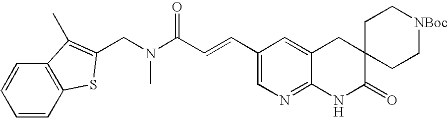

The title compound was prepared according to the standard procedure. The compound was isolated by filtration and triturated from ether to give the product as a yellow solid: ESI MS m/z 561 [C₃₁H₃₆N₄O₄S+H]⁺.

### (E)-N-methyl-N-((3-methylbenzothiophen-2-yl)methyl)-3′-[7-oxo-(6-spiropiperidinyl)-1,8-naphthyridin-3-yl]acrylamide trifluoroacetate salt

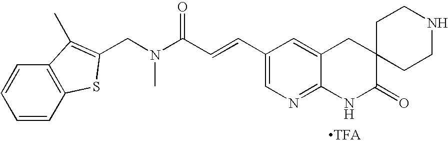

(E)-N-methyl-N-((3-methylbenzothiophene-2-yl)methyl)-3-[7-oxo-(4′-N-Boc-6-spiropiperidinyl)-1,8-naphthyridin-3-yl]acrylamide was dissolved into methylene chloride (10 mL) and treated with TFA (2 mL) for 1 h. The solution was concentrated and the title compound was isolated by preparative HPLC as the trifluoroacetate salt (121 mg, 54%): ¹H NMR (400 MHz, DMSO-d₆) δ 10.86 (s, 1H), 8.46-8.44 (m, 3H), 8.09-8.03 (m, 1H), 7.88 (d, J=7.6 Hz, 1H), 7.72 (d, J=7.4 Hz, 1H), 7.56 (2s, rotomers, 1H) 7.42-7.18 (m, 3H), 5.11-4.88 (2s, rotomers, 2H), 3.17-3.11 (m, 6H), 2.98-2.92 (m, 3H), 2.42 (s, 3H), 2.02-1.92 (m, 21, 1.60-1.50 (m, 2H); ESI MS m/z 461 [C₂₆H₂₈N₄O₂S+H]⁺.

### Example 22

### (E)-N-methyl-N-((1-methyl-1H-indol-2-yl)methyl)-3-[7-oxo-(4′-6-spiropiperidinyl)-1,8-naphthyridin-3-yl]acrylamide trifluoroacetate salt

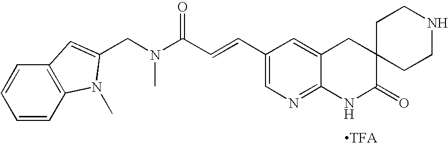

### (E)-N-methyl-N-((1-methyl-1H-indol-2-yl)methyl)-3-[7-oxo-(4′-N-Boc-6-spiropiperidinyl)-1,8-naphthyridin-3-yl]-acrylamide

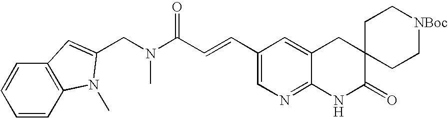

The title compound was prepared according to the standard procedure. The product was isolated by silica gel chromatography (3% MeOH/CH₂Cl₂) as a yellow solid (105 mg, 49%): ESI MS m/z 544 [C₃₁H₃₇N₅O₄+H]⁺.

### (E)-N-methyl-N-((1-methyl-1H-indol-2-yl)methyl)-3-[7-oxo-(4′-6-spiropiperidinyl)-1,8-naphthyridin-3-yl]acrylamide trifluoroacetate salt

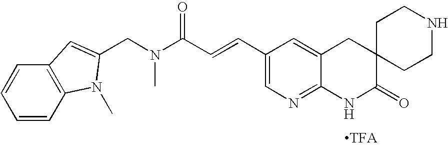

The title compound was prepared following the standard procedure and purified by prep HPLC to give the product as a purple powder (6 mg, 6%): ¹H NMR (400 MHz, CD₃OD) δ 8.42-8.36 (m, 1H), 8.05-7.90 (m, 1H), 7.62 (2s, rotomers, 1H), 7.52-7.48 (m, 1H), 7.36-7.30 (m, 1H) 7.21-7.14 (m, 2H), 7.04-6.92 (m, 1H) 6.48-6.36 (2s, rotomers, 1H), 5.05-4.93 (2s, rotomers, 2H), 3.71-3.65 (m, 3H), 3.49-3.30 (m, 7H), 3.18-3.12 (m, 2H), 3.04-2.92 (m, 2H), 2.15-2.06 (m, 2H), 1.75-1.65 (m, 2H); ESI MS m/z 444 [C₂₆H₂₉N₅O₂+H]⁺.

### Example 23

### (E)-N-(3-methoxy-2-propoxybenzyl)-N-methyl-3-[7-oxo-(6-spiropiperidinyl)-1,8-naphthyridin-3-yl]acrylamide hydrochloride

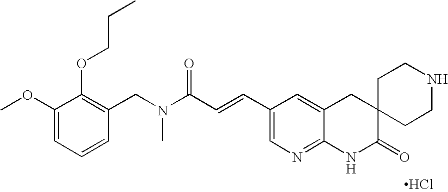

### (E)-N-(3-methoxy-2-propoxybenzyl)-N-methyl-3-[7-oxo-(4′-N-Boc-6-spiropiperidinyl)-1,8-naphthyridin-3-yl]acrylamide

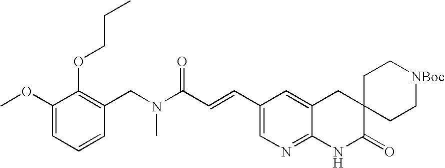

The title compound was prepared according to the standard procedure. The compound was purified by silica gel chromatography (3% MeOH/CH₂Cl₂) as a white solid (160 mg, 71%): ESI MS m/z 579 [C₃₂H₄₂N₄O₆+H]⁺.

### (E)-N-(3-methoxy-2-propoxybenzyl)-N-methyl-3-[7-oxo-(6-spiropiperidinyl)-1,8-naphthyridin-3-yl]acrylamide hydrochloride

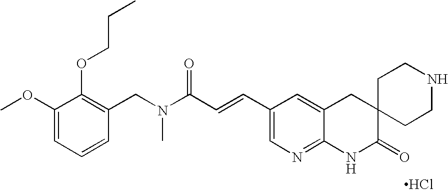

(E)-N-(3-methoxy-2-propoxybenzyl)-N-methyl-3-[7-oxo-(4′-N-Boc-6-spiropiperidinyl)-1,8-naphthyridin-3-yl]acrylamide (150 mg, 0.26 mmol) was dissolved into CH₂Cl₂ (25 mL) and treated with HCl (5 mL of 1 M solution in ether). The solution was stirred for 2 h and concentrated to dryness. The solid was triturated with ether to give the title compound (105 mg, 79%): ¹H NMR (400 MHz, DMSO-d₆) δ 10.88-10.86 (2s, rotomers, 1H), 8.95 (bs, 1H), 8.75 (bs, 1H), 8.42-8.38 (2s, rotomers, 1H), 8.05 (2s, rotomers, 1H), 7.52-7.48 (m, 1H), 7.24-7.20 (m, 1H) 7.08-6.94 (m, 2H), 6.65 (t, J=7.9 Hz, 1H), 4.78-4.64 (2s, rotomers, 2H), 3.92-3.82 (m, 2H), 3.79 (s, 3H) 3.16-3.10 (m, 5H), 2.98-2.88 (m, 4H), 2.06-1.91 (m, 2H), 1.75-1.65 (m, 2H), 1.65-1.55 (m, 2H), 1.01-0.96 (m, 3H); ESI MS m/z 479 [C₂₇H₃₄N₄O₄+H]⁺.

### Example 24

### (E)-N-methyl-N-((3-methylbenzofuran-2-yl)methyl)-3-[7-oxo-(6-4′-N-methyl-spiropiperidinyl)-1,8-naphthyridin-3-yl]acrylamide hydrochloride

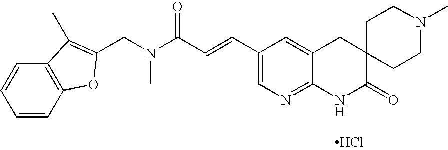

### 6-bromo-(4′-N-methyl-3-spiropiperidinyl)-1,8-naphthyridin-2(1H)-one

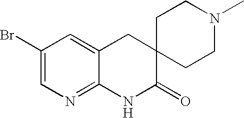

5-Bromo-3-(bromomethyl)pyridin-2-amine hydrobromide (6.8 g, 20 mmol) was added to anhydrous THF (80 mL) under argon. The slurry was cooled to −78° C. and freshly prepared LDA (20 mL, 1 M in THF, 20 mmol) was added dropwise and stirred 15 min. In a separate flask, N-methyl ethylisonipecotate (10.2 g, 60 mmol) was dissolved into anhydrous THF (150 mL) and cooled to −78° C. under argon. Freshly prepared LDA (60 mL of a 1M solution in THF) was added dropwise over 30 min and stirred an additional 30 min. Via canula, the lithium salt of N-methyl ethylisopecotate was added dropwise over 30 min. The mixture was stirred at −78° C. for 2 h and allowed to warm to room temperature. The reaction was quenched with sat. NH₄Cl (20 mL) and ethyl acetate (200 mL) was added. The solution was washed with water (2×50 mL), brine (50 mL), dried over MgSO₄ and concentrated. The solid was triturated with hexanes to afford the title compound as a yellow solid (3.65 g, 59%): ¹H NMR (400 MHz, CDCl₃) δ 8.88 (bs, 1H), 8.27 (s, 1H), 7.63 (s, 1H), 2.86 (s, 2H), 2.71-2.69 (m, 2H), 2.50-2.42 (m, 2H), 2.35 (s, 3H), 2.10-2.05 (m, 2H), 1.60-1.54 (m, 2H).

### (E)-tert-butyl-3-[7-oxo-(4′-N-methyl-6-spiropiperidinyl)-1,8-naphthyridin-3-yl]acrylate

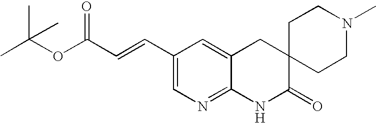

6-Bromo-(4′-N-methyl-3-spiropiperidinyl)-1,8-naphthyridin-2(1H)-one (3.6 g, 11.6 mmol) was dissolved into 4:1 DMF/propionitrile (62 mL) and degassed with argon. Diisopropylethylamine (4.9 mL, 28 mmol) and tert-butyl acrylate (6.8 mL, 46 mmol) were added and the solution was degassed with argon. Palladium acetate (260 mg, 1.16 mmol) and tri-o-tolylphosphine (705 mg, 2.32 mmol) were added the solution was degassed. The mixture was heated in a sealed tube at 100° C. for 16 h. The solution was filtered through celite. The filter cake was washed with methylene chloride and 10% MeOH/methylene chloride. The filtrates were concentrated and residue preaborbed onto silica gel. The title compound was eluted (20% MeOH/CH₂Cl₂) as a beige solid (1.2 g, 28%): ESI MS m/z 358 [C₂₀H₂₇N₃O₃+H]⁺.

### (E)-3-[7-oxo-(4′-methyl-6-spiropiperidinyl)-1,8-naphthyridin-3-yl]acrylic acid

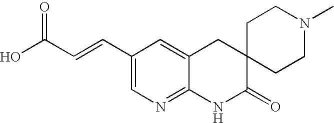

(E)-tert-butyl-3-[7-oxo-(4′-methyl-6-spiropiperidinyl)-1,8-naphthyridin-3-yl]acrylate (1.2 g, 3.3 mmol) was dissolved in CH₂Cl₂ (5 mL) and treated with TFA (5 mL) for 1 h. The solvents were removed in vacuo and redissolved in CH₂Cl₂ (5 mL). A solution of HCl in dioxane (1 mL of 4M solution) was added until the product precipitated from solution. The solid was collected, triturated with ether and dried to give the title compound as a white solid (1.1 g, 100%): ¹H NMR (400 MHz, DMSO-d₆) δ 10.91 (s, 1H), 8.42-8.39 (m, 1H), 8.09-7.92 (m, 1H), 7.56-7.53 (m, 1H), 6.55-6.43 (m, 1H), 3.35-3.30 (m, 4H), 3.26-3.17 (m, 2H), 2.89-2.73 (m, 4H), 2.18-2.12 (m, 1H), 1.96-1.93 (m, 1H), 1.83-1.77 (m, 1H), 1.60-1.56 (m, 1H).

### (E)-N-methyl-N-((3-methylbenzofuran-2-yl)methyl)-3-[7-oxo-(6-4′-N-methyl-spiropiperidinyl)-1,8-naphthyridin-3-yl]acrylamide hydrochloride

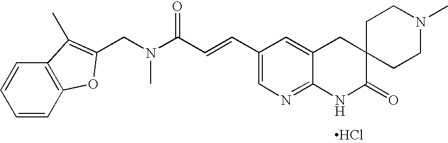

The compound was prepared according to the standard procedure. The compound was purified by preparative HPLC. The hydrochloride salt was prepared by dissolving the free base into CH₂Cl₂ and adding HCl in dioxane. The precipitate was collected and triturated with ether to give the title compound (65 mg, 15%): ¹H NMR (400 MHz, CDCl₃) δ 8.45-8.31 (m, 2H), 8.34-8.29 (m, 1H), 7.78-7.76 (m, 1H), 7.73-7.71 (m, 1H), 7.43-7.27 (m, 3H), 5.13-4.98 (2s, rotomers, 2H), 3.47-3.86 (m, 4H), 3.36-3.30 9m, 2H), 3.28-3.27 (m, 2H), 2.95-2.90 (m, 4H), 2.46 (s, 3H), 2.29-2.17 (m, 2H) 1.92-1.86 (m, 2H); ESI MS m/z 459 [C₂₇H₃₀N₄O₃+H]⁺.

### Example 25

### (E)-N-(3-methoxy-2-propoxybenzyl)-N-methyl-3-[7-oxo-(4′-N-methyl-6-spiropiperidinyl)-1,8-naphthyridin-3-yl]acrylamide trifluoroacetate

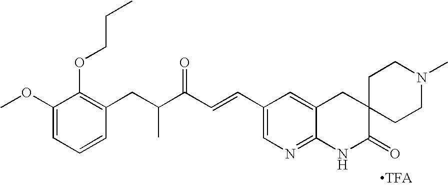

The title compound was prepared according to the standard procedure. The compound was extracted with ethyl acetate, dried over MgSO₄ and concentrated. The residue was purified by preparative HPLC to give the title compound as the trifluoroacetate salt (100 mg, 28%): ¹H NMR (400 MHz, DMSO-d₆) δ 10.94-10.92 (2s, rotomers, 1H), 9.45-9.30 (m, 1H), 8.47-8.38 (m, 1H), 8.13-8.05 (2s, rotomers, 1H), 7.94-7.85 (2s, rotomers, 1H), 7.58-7.50 (m, 1H), 7.27-7.15 (m, 1H), 7.08-6.92 (m, 2H), 6.65-6.58 (m, 1H), 4.78-4.64 (2s, rotomers, 2H), 3.92-3.82 (m, 2H), 3.79 (s, 3H) 3.40-3.30 (m, 2H), 3.25-3.08 (m, 4H), 2.87-2.80 (m, 4H) 2.06-1.91 (m, 2H), 1.75-1.65 (m, 2H), 1.65-1.55 (m, 2H), 1.01-0.96 (m, 3H); ESI MS m/z 493 [C₂₈H₃₆N₄O₄+H]⁺.

### Example 26

(E)-N-methyl-N-((3-methylbenzothiophen-2-yl)methyl)-3-[7-oxo-(4′-N-methyl-6-spiropiperidinyl)-1,8-naphthyridin-3-yl]acrylamide hydrochloride

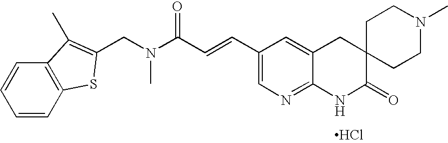

The compound was prepared according to the standard procedure. The compound was purified by preparative HPLC. The hydrochloride salt was prepared by dissolving the free base into CH₂Cl₂ (2 mL) and addition of HCl in dioxane (1 mL of a 4 M solution). The precipitate was collected and triturated with ether to give the title compound (70 mg, 15%): ¹H NMR (400 MHz, DMSO-d₆) δ 10.68 (s, 1H) 8.42-8.37 (m, 1H), 8.16 (s, 1H), 7.57-7.47 (m, 3H), 7.27-7.18 (m, 3H), 5.00-4.79 (2s, rotomers, 2H), 3.18-3.16 (m, 5H), 2.91-2.90 (m, 3H) 2.25 (s, 3H), 2.17-2.15 (m, 6H), 1.34-1.31 (m, 2H); ESI MS t/z 475 [C₂₇H₃₀N₄O₂S+H]⁺.

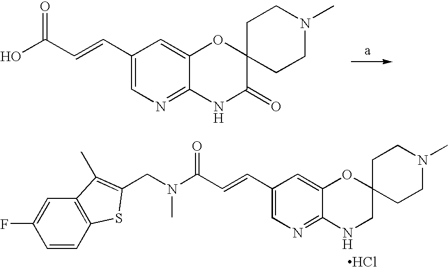

a) (5-fluoro-3-methylbenzo[b]thiophen-2-yl)-N-methyl methanamine, EDC, HOBt, DIPEA, DMF, b) TFA, CH₂Cl₂.

### Example 27

### (E)-3-(6,6-(4-N-methylpiperidine)-7-oxo-5,6,7,8-tetrahydro-1,8-naphthyridin-3-yl)-N-((5-fluoro-3-methylbenzo[b]thiophen-2-yl)methyl)-N-methylacrylamide

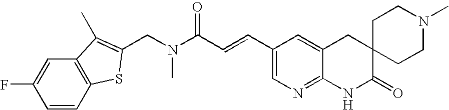

To a solution of (5-fluoro-3-methylbenzo[b]thiophen-2-yl)-N-methyl methanamine (157 mg, 0.75 mmol) in DMF (5 mL) were added in sequential order (E)-3-(6,6-(4-N-methylpiperidine)-7-oxo-5,6,7,8-tetrahydro-1,8-naphthyridin-3-yl)acrylic acid (204 mg, 0.68 mmol), 1-hydroxybenzotriazole (105 mg, 0.75 mmol), diisopropylethylamine (355 uL, 2.04 mmol), and N-(3-dimethylaminopropyl)-N′-ethylcarbodiimide (150 mg, 0.75 mmol). The mixture was stirred at room temperature overnight, cooled in an ice bath and water was added with rapid stirring. The product was extracted with ethyl acetate (3×10 mL), dried with sodium sulfate, filtered and concentrated. The crude product was purified using preparative HPLC to yield the title compound as a white solid (99 mg, 30%). ¹H NMR (400 MHz, DMSO-d₆) δ 10.90 (bs, 1H), 9.29 (bs, 1H), 8.46 (d, 1H, J=17.2 Hz), 7.90 (bs, 2H), 7.59-7.54 (m, 2H), 7.24-7.19 (m, 2H), 5.11-4.88 (2s, 2H, rotamers), 3.36 (s, 2H), 3.21-3.16 (bs, 4H), 2.76-2.82 (m, 4H), 2.39 (s, 3H), 2.06 (s, 3H), 1.64 (bs, 3H); MS (ESI) m/e 493 (C₂₇H₂₉FN₄O₂S+H)⁺.

### Example 28

### (E)-N-methyl-N-((3-methylbenzofuran-2-yl)methyl)-3-(6-morpholino-7-oxo-7,8-dihydro-1,8-naphthyridin-3-yl)acrylamide

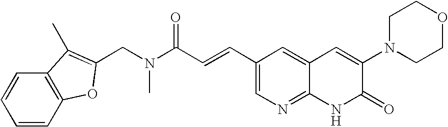

### 6-bromo-3-morpholino-1,8-naphthyridin-2(1H)-one

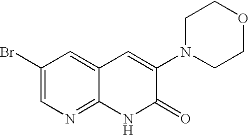

To a solution of ethyl 2-morpholinoacetate (1.00 g, 5.77 mmol) in anhydrous DMF (25 mL) at 0° C. was added sodium tert-butoxide (526 mg, 5.47 mmol). After stirring for 5 min, 2-amino-5-bromonicotinaldehyde (639 mg, 3.15 mmol) was added and the reaction was allow to warm to room temperature and continue stirring for 12 h. The mixture was diluted with water (20 mL) and the aqueous layer was extracted with EtOAc (3×20 mL). The combined organic layers were washed with brine (30 mL), dried over Na₂SO₄, filtered and concentrated. The resulting solid was stirred in a DMSO:Water (1:3) system, filtered and washed with water to give the title compound (6-bromo-3-morpholino-1,8-naphthyridin-2(1H)-one) (164 mg, 8.0%) as a yellow solid: ¹H NMR (400 MHz, DMSO-d₆) δ 12.31 (s, 1H), 8.37 (s, 1H), 8.22 (s, 1H), 7.05 (s, 1H), 3.76-3.74 (m, 4H), 3.23, 3.21 (m, 4H).

### (E)-N-methyl-N-((3-methylbenzofuran-2-yl)methyl)-3-(6-morpholino-7-oxo-7,8-dihydro-1,8-naphthyridin-3-yl)acrylamide

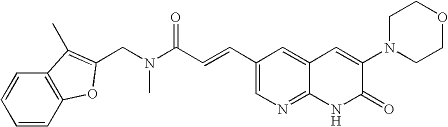

Prepared according to the standard procedure. Purification by preparative HPLC (water/acetonitrile/0.05% TFA mixture) gave the title compound ((E)-N-methyl-N-((3-methylbenzofuran-2-yl)methyl)-3-(6-morpholino-7-oxo-7,8-dihydro-1,8-naphthyridin-3-yl)acrylamide) (18.9 mg, 14%) as a yellow solid and a mixture of amide rotomers: ¹H NMR (400 MHz, DMSO-d₆) δ 12.31 (s, 1H), 8.67-8.65 (m, 1H), 8.37 (bs, 1H), 7.63-7.56 (m, 2H), 7.50-7.48 (m, 1H), 7.31-7.22 (3H), 7.03 (s, 1H), 5.01-4.81 (m, 2H), 3.76-3.74 (m, 4H), 3.22-2.94 (m, 7H), 2.27 (s, 3H); ESI MS m/z 459 [C₂₆H₂₆N₄O₄+H]⁺.

### Example 29

### Preparation of (E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride

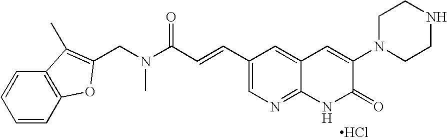

### tert-butyl 4-(2-ethoxy-2-oxoethyl)piperazine-1-carboxylate

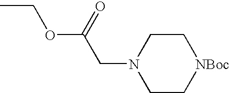

To a solution of tert-butyl piperazine-1-carboxylate (2.00 g, 10.7 mmol) and K₂CO₃ (3.71 g, 26.8 mmol) in anhydrous DMF (14 mL) was added ethyl bromoacetate (1.2 mL, 10.8 mmol). After stirring for 12 h at room temperature water (50 mL) was added and the aqueous layer was extracted with EtOAc (3×50 mL). The combined organic layers were washed with brine (2×75 mL), dried over Na₂SO₄, filtered and concentrated to give the title compound (tert-butyl 4-(2-ethoxy-2-oxoethyl)piperazine-1-carboxylate) (3.04 g, 100%) as a yellow oil: ¹H NMR (400 MHz, DMSO-d₆) δ 4.08 (q, J=6.8 Hz, 2H), 3.31-3.29 (m, 4H), 3.24 (s, 2H), 2.47-2.44 (m, 4H), 1.39 (s, 9H), 1.18 (t, J=7.2 Hz, 3H).

### tert-butyl 4-(6-bromo-2-oxo-1,2-dihydro-1,8-naphthyridin-3-yl)piperazine-1-carboxylate

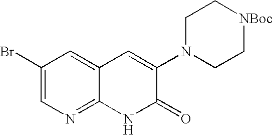

To a solution of tert-butyl 4-(2-ethoxy-2-oxoethyl)piperazine-1-carboxylate (2.00 g, 7.34 mmol) in anhydrous DMF (40 mL) at 0° C. was added sodium tert-butoxide (559 mg, 5.82 mmol) followed by 2-amino-5-bromonicotinaldehyde (974 mg, 4.85 mmol). After allowing the reaction mixture to warm to room temperature and stir for 12 h the reaction was diluted with water (100 mL) and the aqueous layer was extracted with CH₂Cl₂ (4×50 mL). The combined organic layers were washed with brine (3×100 mL), dried over Na₂SO₄, filtered and concentrated to give an orange oil. Purification by column chromatography (gradient elution of CH₂Cl₂ to 10% MeOH/CH₂Cl₂) gave the title compound (tert-butyl 4-(6-bromo-2-oxo-1,2-dihydro-1,8-naphthyridin-3-yl)piperazine-1-carboxylate) (114 mg, 4.0%) as a yellow solid: ¹H NMR (400 MHz, DMSO-d₆) δ 12.33 (s, 1H), 8.38 (s, 1H), 8.21 (s, 1H), 7.08 (s, 1H), 3.47 (m, 4H), 3.18 (m, 4H), 1.43 (s, 9H).

### (E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride

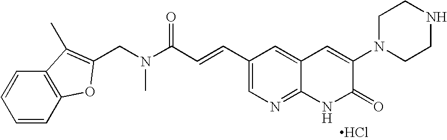

A solution of tert-butyl 4-(6-bromo-2-oxo-1,2-dihydro-1,8-naphthyridin-3-yl)piperazine-1-carboxylate (48.9 mg, 0.120 mmol), N-methyl-N-((3-methylbenzofuran-2-yl)methyl)acrylamide (50.0 mg, 0.218 mmol) and DIPEA (0.05 mL, 0.287 mmol) in anhydrous DMF (1.6 mL) was prepared. Argon was bubbled into the mixture with stirring for 30 min. Next P(o-tol)₃ (8.0 mg, 0.0262 mmol) and Pd(OAc)₂ (3.0 mg, 0.0134 mmol) were added to the mixture and argon was bubbled into the reaction for an additional 5 min. The reaction was then sealed and put in a microwave for 5 min at 160° C. The reaction was then diluted with water (10 mL) and the aqueous layer was extracted with CH₂Cl₂ (3×10 mL). The combined organic layers were washed with brine (20 mL), dried over Na₂SO₄, filtered and concentrated to give a yellow oil. Purification by preparative HPLC (water/acetonitrile/0.05% TFA mixture) gave the desired product as an orange solid which was dissolved in THF (2.0 mL). To the mixture was added HCl (2.0 mL of 4M solution in dioxane) and the mixture was stirred for 12 h at room temperature. After concentrating under high vacuum and triturating with diethyl ether the title compound ((E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride) (20.4 mg, 35%) was obtained as an orange solid and a mixture of amide rotomers: ¹H NMR (400 MHz, DMSO-d₆) δ 12.44 (s, 1H), 9.09 (bs, 1H), 8.74-8.71 (m, 1H), 8.38-8.37 (m, 1H), 7.64-7.48 (m, 3H), 7.31-7.17 (m, 4H), 5.01-4.81 (m, 2H), 3.44-3.40 (m, 4H), 3.26-2.94 (m, 7H), 2.27 (s, 3H); ESI MS m/z 458 [C₂₆H₂₇N₅O₃+H]⁺.

### Example 30

### Preparation of (R,E)-N-(1-(3-ethylbenzofuran-2-yl)ethyl)-N-methyl-3-(7-oxo-5,6,7,8-tetrahydro-1,8-naphthyridin-3-yl)acrylamide

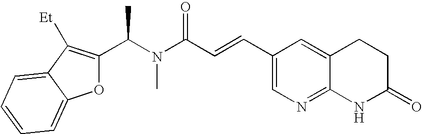

### a) 1-(3-ethyl-benzofuran-2-yl)ethanone

To a solution of 1-(2-hydroxy-phenyl)propanone (15.02 g, 100.0 mmol) and chloroacetone (9.26 g, 100.0 mmol) in DMF (100 mL) was added K₂CO₃ (27.64 g, 200 mmol). The resulting suspension was stirred at room temperature for 24 hours. The mixture was diluted with H₂O and extracted with hexane (500 mL). The combined organics were washed with NaOH (0.5 N, 5×150 mL) and brine, dried over MgSO₄ and concentrated to give the title compound (11.31 g, 60%) as a yellow oil: ¹H NMR (300 MHz, DMSO, δ) 7.85 (d, J=7.9 Hz, 1H), 7.67 (d, J=7.8 Hz, 1H), 7.54 (t, J=7.8 Hz, 1H), 7.36 (t, J=7.8 Hz, 1H), 3.05 (q, J=7.5 Hz, 2H), 2.56 (s, 3H), 1.20 (t, J=7.5 Hz, 3H). MS (ESI) m/e: 189 (M+H)⁺.

### b) (R)-2-methyl-propane-2-sulfinic acid [1-(3-ethyl-benzofuran-2-yl)ethylidene]amide

To a solution of 1-(3-ethyl-benzofuran-2-yl)ethanone (3.77 g, 20.0 mmol) and (R)-(+)-2-methyl-2-propanesulfinamide (2.55 g, 21.0 mmol) in anhydrous THF (100 mL) was added Ti(OEt)₄ (9.3 mL, 44.0 mmol). The mixture was heated at reflux overnight. The solution was cooled to room temperature and poured into rapidly stirring satd NaCl (100 mL). The solution was filtered through celite, which was washed with EtOAc. The organic layer was dried (Na₂SO₄) and concentrated. Purification by flash column chromatography (silica gel, 0-10% EtOAc in hexanes) gave the title compound (3.48 g, 60%) as an orange solid: ¹H NMR (300 MHz, DMSO, δ) 7.81 (d, J=7.9 Hz, 1H), 7.65 (d, J=8.4 Hz, 1H), 7.54 (t, J=7.7 Hz, 1H), 7.36 (t, J=7.3 Hz, 1H), 3.11 (q, J=7.5 Hz, 2H), 2.75 (s, 3H), 1.25 (s, 9H), 1.20 (t, J=7.5 Hz, 3H). MS (ESI) m/e: 292 (M+H)⁺.

### c) (R)-2-methyl-propane-2-sulfinic acid [1-(3-ethyl-benzofuran-2-yl)ethyl]amide

To an ice-cold solution of (R)-2-methyl-propane-2-sulfinic acid [1-(3-ethyl-benzofuran-2-yl)ethylidene]amide (3.47 g, 11.92 mmol) in THF (60 mL) was added 9-BBN (26.3 mL, 0.5 M solution in THF, 13.11 mmol) dropwise. The mixture was slowly warmed to room temperature and stirred overnight. The mixture was quenched with H₂O (10 mL) and concentrated. The residue was partitioned between Et₂O and water. The organic layer was washed with satd NaCl, dried (Na₂SO₄) and concentrated. Purification by column chromatography (silica gel, 10-30% EtOAc in hexanes) gave the title compound (3.48 g, 99%) as an oil: ¹H NMR (300 MHz, DMSO, δ) 7.59 (d, J=7.5 Hz, 1H), 7.49 (d, J=7.5 Hz, 1H), 7.24 (m, 2H), 5.71 (d, J=7.4 Hz, 1H), 4.63 (m, 1H), 2.70 (q, J=7.6 Hz), 1.51 (d, J=6.9 Hz, 3H), 1.22 (t, J=7.5 Hz, 3H), 1.09 (s, 9H). MS (ESI) m/e: 294 (M+H)⁺.

### d) (R,R)-2-methyl-propane-2-sulfinic acid methyl-[1-(3-ethyl-benzofuran-2-yl)-ethyl]amide

To an ice-cold suspension of NaH (530 mg, 60% dispersion in mineral oil, 13.3 mmol) in DMF (40 mL) was added a solution of (R)-2-methyl-propane-2-sulfinic acid [1-(3-ethyl-benzofuran-2-yl)ethyl]amide (3.48 g, 11.9 mmol) in DMF (20 mL) drop-wise. The mixture was stirred 1 h then MeI (0.83 mL, 13.3 mmol) was added drop-wise. The mixture was slowly warmed to room temperature and stirred overnight. The mixture was quenched with H₂O and extracted with Et₂O. The combined organics were washed with H₂O and brine, dried (Na₂SO₄) and concentrated to give the title compound (3.65 g, 99%) as an oil: ¹H NMR (300 MHz, DMSO, δ) 7.62 (d, J=7.8 Hz, 1H), 7.51 (d, J=7.5 Hz, 1H), 7.24 (m, 2H), 4.76 (q, J=7.0 Hz, 1H), 2.73 (q, J=7.6 Hz), 1.57 (d, J=7.2 Hz, 3H), 1.23 (t, J=7.5 Hz, 3H), 1.08 (s, 9H). MS (ESI) m/e: 308 (M+H)⁺.

### e) (R)-methyl-[1-(3-ethyl-benzofuran-2-yl)-ethyl]amine

To a solution of (R,R)-2-methyl-propane-2-sulfinic acid methyl-[1-(3-ethyl-benzofuran-2-yl)-ethyl]amide (3.65 g, 11.9 mmol) in MeOH (40 mL) was added TFA (1.85 mL, 23.8 mmol) slowly. The resulting solution was heated to 40° C. overnight. The mixture was cooled to room temperature and concentrated. The residue was diluted with H₂O and partitioned between CH₂Cl₂ and satd. NaHCO₃. The aqueous layer was extracted with CH₂Cl₂. The combined organics were washed with brine, dried (Na₂SO₄) and concentrated. Purification by column chromatography (silica gel, 3% MeOH in CH₂Cl₂) give the title compound (2.42 g, 99%) as a wax: ¹H NMR (300 MHz, CDCl₃, δ) 7.51 (d, J=7.5 Hz, 1H), 7.42 (d, J=7.5 Hz, 1H), 7.22 (m, 2H), 3.92 (q, J=7.0 Hz, 1H), 2.70 (q, J=7.6 Hz), 2.31 (s, 3H), 1.48 (d, J=7.0 Hz, 3H), 1.27 (t, J=7.5 Hz, 3H), 1.19 (s, 9H). MS (ESI) m/e: 204 (M+H)⁺.

### f) N-methyl-N-[1-(R)-(3-ethyl-benzofuran-2-yl)-ethyl]-3-(7-oxo-5,6,7,8-tetrahydro-[1,8]naphthyridin-3-yl)acrylamide

To a solution of 3-(7-oxo-5,6,7,8-tetrahydro-[1,8]naphthyridin-3-yl)acrylic acid hydrochloride (244 mg, 1.2 mmol) in DMF (5 mL) was added (R)-methyl-[1-(3-ethyl-benzofuran-2-yl)-ethyl]amine (255 mg, 1.0 mmol), EDCI (250 mg, 1.3 mmol), HOBt (150 mg, 1.1 mmol) and DIPEA (0.54 mL, 3.1 mmol). The mixture was stirred at 40° C. for 60 hours. The mixture was diluted with H₂O (30 mL) and the solid collected by filtration. The solid was washed with 100 mL H₂O and then dried under reduced pressure overnight. The solid was the suspended in Et₂O, sonicated and stirred for 2 hr then filtered and dried under reduced pressure overnight. Yield: 324 mg (80%) as a mixture of amide rotamers. ¹H NMR (300 MHz, DMSO-d₆, δ) 10.64 (s, 1H), 8.36 (s, 1H), 8.17-8.02 (m, 1H), 7.64-7.48 (m, 3H), 7.36-7.11 (m, 3H), 6.30-5.92 (2m, 1H), 3.1-2.5 (m, 9H), 1.7-1.5 (m, 3H), 1.2-0.9 (m, 3H); MS (ESI) m/e 404 (M+H)⁺.

### Example 31

### Preparation of (R,E)-3-(2,2-dimethyl-3-oxo-3,4-dihydro-2H-pyrido[3,2-b][1,4]oxazin-7-yl)-N-(1-(3-ethylbenzofuran-2-yl)ethyl)-N-methylacrylamide

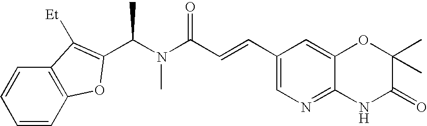

To a solution of a TFA salt of (E)-3-(2,2-dimethyl-3-oxo-3,4-dihydro-2H-pyrido[3,2-b][1,4]oxazin-7-yl)acrylic acid (183 mg, 0.9 mmol) in DMF (4 mL) was added (R)-methyl-[1-(3-ethyl-benzofuran-2-yl)-ethyl]amine (272 mg, 0.75 mmol), EDCI (187 mg, 0.98 mmol), HOBt (112 mg, 0.83 mmol) and DIPEA (0.45 mL, 2.33 mmol). The mixture was stirred at 40° C. for 60 hours. The mixture was diluted with H₂O (30 mL) and the solid collected by filtration. The solid was washed with 100 mL H₂O and then dried under reduced pressure overnight. The solid was purified by chromatography (silica, 1% MeOH in CH₂Cl₂) to give 78 mg (20%) of title compound as a mixture of amide rotamers. ¹H NMR (300 MHz, CDCl₃, 8) 10.56 (s, 1H), 8.19 (s, 1H), 7.8-6.9 (m, 7H), 6.82 (d, J=15.5 Hz, 1H), 6.33 (m, 1H), 3.2-2.6 (m, 5H), 2.0-0.8 (m, 12H); MS (ESI) m/e 434 (M+H)⁺.

### Example 32

### Preparation of (R,E)-3-(6-aminopyridin-3-yl)-N-(1-(3-ethylbenzofuran-2-yl)ethyl)-N-methylacrylamide

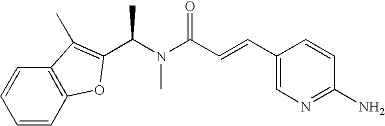

To a solution of (E)-3-(6-aminopyridin-3-yl)acrylic acid (123 mg, 0.75 mmol) in DMF (4 mL) was added (R)-methyl-[1-(3-ethyl-benzofuran-2-yl)-ethyl]amine (183 mg, 0.90 mmol), EDCI (187 mg, 0.98 mmol), HOBt (112 mg, 0.83 mmol) and DIPEA (0.45 mL, 2.33 mmol). The mixture was stirred at 40° C. for 60 hours. The mixture was diluted with H₂O (30 mL) and the solid collected by filtration. The solid was washed with 100 mL H₂O and then dried under reduced pressure overnight. It was purified by chromatography (silica, 1.5% MeOH in CH₂Cl₂) to give 79 mg (25%) of title compound as a mixture of amide rotamers. ¹H NMR (300 MHz, CDCl₃, 8) 8.22 (s, 1H), 7.7-7.1 (m, 6H), 6.8-6.2 (m, 3H), 4.72 (s, 2H) 3.06 (s, 3H), 2.74 (s, 2H), 1.9-1.1 (m, 6H); MS (ESI) m/e 350 (M+H)⁺.

### Example 33

### Preparation of (E)-3-(3-hydroxy-2,2-dimethyl-3,4-dihydro-2H-pyrido[3,2-b][1,4]oxazin-7-yl)-N-methyl-N-((3-methylbenzofuran-2-yl)methyl)acrylamide trifluoroacetate salt

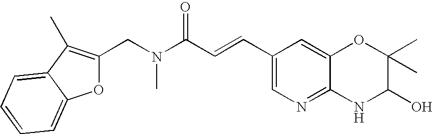

### a) (E)-tert-butyl 2,2-dimethyl-7-(3-(methyl((3-methylbenzofuran-2-yl)methyl)amino)-3-oxoprop-1-enyl)-3-oxo-2,3-dihydropyrido[3,2-b][1,4]oxazine-4-carboxylate

Di-tert-butyl dicarbonate (1.76 g, 8.0 mmol) and 4-dimethylaminopyridine (74 mg, 0.6 mmol) was added to an acetonitrile (100 mL) suspension of (E)-3-(2,2-dimethyl-3-oxo-3,4-dihydro-2H-pyrido[3,2-b][1,4]oxazin-7-yl)-N-methyl-N-((3-methylbenzofuran-2-yl)methyl)acrylamide (811 mg, 2 mmol). After stirring it for 14 hours at 40° C., the solution was evaporated to dryness. The crude product was purified by chromatography (silica, 0-1.2% MeOH in CH₂Cl₂) to give 824 mg (81%) title compound. MS (ESI) m/e 406 (M-C₅H₇O₂)⁺, 506 (M+H)⁺.

### b) (E)-3-(3-hydroxy-2,2-dimethyl-3,4-dihydro-2H-pyrido[3,2-b][1,4]oxazin-7-yl)-N-methyl-N-((3-methylbenzofuran-2-yl)methyl)acrylamide trifluoroacetate salt

LiAlH₄ (2.2 mL, 1M in THF) was added slowly to a THF (10 mL) solution of (E)-tert-butyl 2,2-dimethyl-7-(3-(methyl((3-methylbenzofuran-2-yl)methyl)amino)-3-oxoprop-1-enyl)-3-oxo-2,3-dihydropyrido[3,2-b][1,4]oxazine-4-carboxylate (547 mg, 1.08 mmol) at −78° C. After an hour stirring at this temperature, HCl (0.4 mL, 37%) and CH₂Cl₂ (50 mL) was added. After another 30 min stirring, the mixture was filtered through celite and MgSO₄ and evaporated to 20 mL. Trifluoroacetic acid (4 mL) was added and the resulting mixture was further stirred for 2 hours. Upon evaporation, the crude mixture was purified by preparative HPLC to give 120 mg (21%) title compound as rotamers. ¹H NMR (300 MHz, DMSO-d₆, δ) 8.69 (s, 1H), 7.96-7.75 (m, 2H), 7.6-7.1 (m, 6H), 4.98 (s, 1H), 4.77 and 4.73 (2s, 2H), 3.16 and 2.91 (2s, 3H), 2.26, 1.33 and 1.18 (3s, 3×3H); MS (ESI) m/e 408 (M+H)⁺.

### Example 34

### Preparation of (E)-3-(5-hydroxy-6-isopropyl-7-oxo-7,8-dihydro-1,8-naphthyridin-3-yl)-N-methyl-N-((3-methylbenzofuran-2-yl)methyl)acrylamide

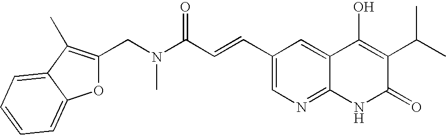

### a) ethyl 2-aminonicotinate

An ethanol (250 mL) solution of aminonicotinic acid (20.80 g, 150 mmol) and sulfuric acid (25 mL) was refluxed for three days. Upon cooling, the mixture was evaporated to 100 mL, poured to ice (800 mL) and neutralized with Na₂CO₃. It was extracted with CH₂Cl₂, dried over MgSO₄ and evaporated to afford 20.75 g (83%) of the title compound. ¹H NMR (300 MHz, CDCl₃, δ) 8.20 (dd, J=4.7 and 1.9 Hz, 1H), 8.13 (dd, J=7.8 and 1.9 Hz, 1H), 6.61 (dd, J=4.7 and 7.8 Hz, 1H), 4.34 (q, J=7.2 Hz, 2H), 1.38 (t, J=7.2 Hz, 3H).

### b) ethyl 2-amino-5-bromonicotinate

A CH₂Cl₂ (30 mL) solution of bromine (16.80 g, 105 mmol) was added to a CH₂Cl₂ (150 mL) suspension of ethyl 2-aminonicotinate (11.64 g, 70 mmol) and NaHCO₃ (17.7 g, 210 mmol) at 0° C. After an hour stirring at this temperature, water (50 mL) was added. The solution was decolorized with a saturated solution of NaHSO₃, extracted with CH₂Cl₂, dried over MgSO₄ and evaporated. Recrystallization from a CH₂Cl₂/hexane mixture afforded 16.02 g (91%) of the title compound. ¹H NMR (300 MHz, CDCl₃, δ) 8.22 (m, 2H), 4.35 (q, J=7.2 Hz, 2H), 1.39 (t, J=7.2 Hz, 3H). MS (ESI) n/e 245 (M+H>⁺.

### c) (E)-ethyl 2-amino-5-(3-ethoxy-3-oxoprop-1-enyl)nicotinate

An acetonitrile (100 mL) solution of ethyl 2-amino-5-bromonicotinate (2.46 g, 10 mmol), ethyl acrylate (3.1 mL, 30 mmol) and diisopropylethylamine (5.3 mL, 30 mmol) was purged with Argon for 10 min. Pd(OAc)₂ (225 mg, 1 mmol) and P(o-Tol)₃ (609 mg, 2 mmol) was added and then the Argon purge was repeated. The mixture was heated to 100° C. and stirred for 4 hours under Argon. Upon cooling, the mixture was evaporated and purified by chromatography (silica, 1-2% MeOH in CH₂Cl₂). Crystallization from a CH₂Cl₂/hexane mixture afforded 1.73 g (66%) of the title compound. ¹H NMR (300 MHz, CDCl₃, δ) 8.36 and 8.33 (2 AA′ d, J=2.3 Hz, 2H), 7.58 (d, J=16.0 Hz, 1H), 6.32 (d, J=16.0 Hz, 1H), 4.37 and 4.26 (2q, J=7.2 Hz, 2×2H), 1.42 and 1.34 (2t, J=7.2 Hz, 2×3H).

### d) (E)-ethyl 5-(3-ethoxy-3-oxoprop-1-enyl)-2-(3-methylbutanamido)nicotinate

A mixture of (E)-ethyl 2-amino-5-(3-ethoxy-3-oxoprop-1-enyl)nicotinate (798 mg, 3 mmol) in 3-methylbutanoic anhydride (3 mL, 15 mmol) was heated in a closed tube at 140° C. for 16 hours. Upon cooling, it was diluted with CH₂Cl₂ (5 mL) and concentrated NH₄OH solution was added slowly while cooling with ice (CAUTION!, very exothermic). After an hour stirring, it was further diluted with CH₂Cl₂ and water. Work-up of the organic layer and chromatography (silica, 0-1% MeOH in CH₂Cl₂). afforded 762 mg (73%) of the title compound. ¹H NMR (300 MHz, CDCl₃, δ) 10.89 (s, 1H), 8.73 (d, J=2.4 Hz, 1H), 8.46 (d, J=2.4 Hz, 1H), 7.64 (d, J=16.1 Hz, 1H), 6.46 (d, J=16.1 Hz, 1H), 4.43 and 4.28 (2q, J=7.2 Hz, 2×2H), 2.47 (d, J=7.1 Hz, 2H), 2.69 (m, 1H), 1.45 and 1.35 (2t, J=7.2 Hz, 2×3H), 1.04 (d, J=6.6 Hz, 6H). MS (ESI) m/e 349 (M+H)⁺.

### e) (E)-3-(5-hydroxy-6-isopropyl-7-oxo-7,8-dihydro-1,8-naphthyridin-3-yl)acrylic acid

Sodium bis(trimethylsilyl)amide (7.7 mL, 7.7 mmol, 1M in THF) was added to THF (10 mL) solution of (E)-ethyl 5-(3-ethoxy-3-oxoprop-1-enyl)-2-(3-methylbutanamido)nicotinate (670 mg, 19 mmol) at −78° C. The mixture was allowed to warm to ambient temperature with stirring. It was cooled to 40° C. and MeOH (10 mL) and water (10 mL) were added. After 3 hours of stirring, it was evaporated to 10 mL, washed with CH₂Cl₂ and acidified with concentrated HCl solution. The precipitate was filtered, washed with water and dried to afford 470 mg (89%) of the title compound. ¹H NMR (300 MHz, DMSO-d₆, δ) 11.76 (s, 1H), 10.36 (s, 1H), 8.73 (d, J=2.0 Hz, 1H), 8.62 (d, J=2.0 Hz, 1H), 7.66 (d, J=16.0 Hz, 1H), 6.58 (d, J=16.0 Hz, 1H), 3.44 (m, 1H), 1.27 (d, J=6.8 Hz, 6H).

### f) (E)-3-(5-hydroxy-6-isopropyl-7-oxo-7,8-dihydro-1,8-naphthyridin-3-yl)-N-methyl-N-((3-methylbenzo-furan-2-yl)methyl)acrylamide

To a solution of (E)-3-(5-hydroxy-6-isopropyl-7-oxo-7,8-dihydro-1,8-naphthyridin-3-yl)acrylic acid (137 mg, 0.75 mmol) in DMF (3 mL) was added N-methyl(3-methylbenzofuran-2-yl)methanamine (114 mg, 0.65 mmol), EDCI (125 mg, 0.65 mmol), HOBt (74 mg, 0.55 mmol) and DIPEA (0.26 mL, 1.5 mmol). The mixture was stirred at 40° C. for 60 hours. The mixture was diluted with H₂O (30 mL) and acidified with HCl and extracted with CH₂Cl₂. The crude product was purified by chromatography (silica, 0-2% MeOH in CH₂Cl₂) to give 97 mg (22%) of title compound as a mixture of amide rotamers. ¹H NMR (300 MHz, DMSO-d₆, δ) 11.71 (s, 1H), 10.27 (s, 1H), 8.79 (s, 1H), 8.57 (s, 1H), 7.65-7.45 (m, 3H), 7.26 (m, 3H), 5.00 and 4.81 (2s, 2H), 3.42 (m, 1H), 3.21 and 2.96 (2s, 3H), 2.28 (s, 3H), 1.27 (d, J=7.2 Hz, 6H). MS (ESI) m/e 432 (M+H)⁺.

### Example 35

### Preparation of (E)-N-((1,2-dihydroacenaphthylen-5-yl)methyl)-3-(5-hydroxy-6-isopropyl-7-oxo-7,8-dihydro-1,8-naphthyridin-3-yl)-N-methylacrylamide

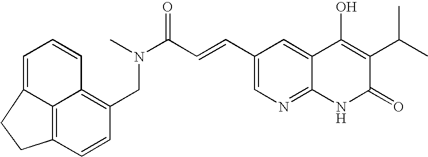

To a solution of (E)-3-(5-hydroxy-6-isopropyl-7-oxo-7,8-dihydro-1,8-naphthyridin-3-yl)acrylic acid (137 mg, 0.75 mmol) in DMF (3 mL) was added (1,2-dihydroacenaphthylen-5-yl)-N-methylmethanamine (128 mg, 0.65 mmol), EDCI (125 mg, 0.65 mmol), HOBt (74 mg, 0.55 mmol) and DIPEA (0.26 mL, 1.5 mmol). The mixture was stirred at 40° C. for 60 hours. The mixture was diluted with H₂O (30 mL) and acidified with HCl and extracted with CH₂Cl₂. The crude product was purified by chromatography (silica, 0-2% MeOH in CH₂Cl₂) to give 97 mg (28%) of title compound as a mixture of amide rotamers. ¹H NMR (300 MHz, DMSO-d₆, δ) 11.70 and 11.65 (2s, 1H), 10.25 (s, 1H), 8.80 and 8.72 (2s, 1H), 8.58 and 8.49 (2s, 1H), 7.9-7.1 (m, 7H), 5.24 and 5.04 (2s, 2H), 3.42 (m, 4H), 3.06 and 2.95 (2s, 3H), 2.28 (s, 3H), 1.27 (m, 6H). MS (ESI) mie 454 (M+H)⁺.

### Example 36

### Preparation of (E)-3-(5-hydroxy-6-ethyl-7-oxo-7,8-dihydro-1,8-naphthyridin-3-yl)-N-methyl-N-((3-methylbenzofuran-2-yl)methyl)acrylamide

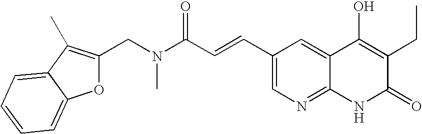

### a) (E)-ethyl 2-butyramido-5-(3-ethoxy-3-oxoprop-1-enyl)nicotinate

A mixture of (E)-ethyl 2-amino-5-(3-ethoxy-3-oxoprop-1-enyl)nicotinate (2.42 g, 9.1 mmol) in butyric anhydride (7.5 mL, 45.8 mmol) was heated in a closed tube at 145° C. for 17 hours. Upon cooling, it was diluted with CH₂Cl₂ (5 mL) and concentrated NH₄OH solution was added slowly while cooling with ice (CAUTION!, very exothermic). After an hour stirring, it was further diluted with CH₂Cl₂ and water. Crystallization from CH₂Cl₂/hexane afforded 2.24 g (73%) of the title compound. ¹H NMR (300 MHz, CDCl₃, δ) 10.92 (s, 1H), 8.72 (d, J=2.4 Hz, 1H), 8.47 (d, J=2.4 Hz, 1H), 7.64 (d, J=16.0 Hz, 1H), 6.46 (d, J=16.0 Hz, 111), 4.43 and 4.28 (2q, J=7.2 Hz, 2×2H), 2.60 (t, J=7.5 Hz, 2H), 1.81 (m, 2H), 1.45 and 1.35 (2t, J=7.2 Hz, 2×3H), 1.03 (t, J=7.4 Hz, 3H).

### b) (E)-3-(5-hydroxy-6-ethyl-7-oxo-7,8-dihydro-1,8-naphthyridin-3-yl)acrylic acid

Sodium bis(trimethylsilyl)amide (15 mL, 15 mmol, 1M in THF) was added to THF (20 mL) solution of (E)-ethyl 2-butyramido-5-(3-ethoxy-3-oxoprop-1-enyl)nicotinate (1 g, 3 mmol) at −78° C. The mixture was allowed to warn to ambient temperature with stirring. It was cooled to −40° C. and MeOH (10 mL) and water (10 mL) were added. After 3 hours of stirring, it was evaporated to 10 mL, washed with CH₂Cl₂ and acidified with concentrated HCl solution. The precipitate was filtered, washed with water and dried to afford 710 mg (98%) of the title compound. ¹H NMR (300 MHz, DMSO-d₆, δ) 11.89 (s, 1H), 10.51 (s, 1H), 8.76 (d, J=2.1 Hz, 1H), 8.54 (d, J=2.1 Hz, 1H), 7.68 (d, J=16.0 Hz, 1H), 6.62 (d, J=16.0 Hz, 1H), 2.56 (q, J=7.2 Hz, 1H), 1.02 (t, J=7.2 Hz, 3H).

### c) (E)-3-(5-hydroxy-6-isopropyl-7-oxo-7,8-dihydro-1,8-naphthyridin-3-yl)-N-methyl-N-((3-methylbenzo-furan-2-yl)methyl)acrylamide

A solution of (E)-3-(5-hydroxy-6-ethyl-7-oxo-7,8-dihydro-1,8-naphthyridin-3-yl)acrylic acid (131 mg, 0.5 mmol), N-methyl(3-methylbenzofuran-2-yl)methanamine (114 mg, 0.65 mmol), EDCI (131 mg, 0.7 mmol), HOBt (70 mg, 0.50 mmol) and DIPEA (0.36 mL, 2 mmol) in DMF (3 mL) was submitted to microwave irradiation at 135° C. for 5 minutes. The mixture was diluted with H₂O (30 mL) and acidified with HCl and extracted with EtOAc. The crude product was purified by chromatography (silica, 0-2% MeOH in CH₂Cl₂) to give 118 mg (28%) of title compound as a mixture of amide rotamers. ¹H NMR (300 MHz, DMSO-d₆, δ) 11.75 (s, 1H), 10.5 (s, br, 1H), 8.79 (s, 1H), 8.57 and 8.54 (2s, 1H), 7.65-7.45 (m, 3H), 7.26 (m, 3H), 5.01 and 4.81 (2s, 2H), 3.21 and 2.96 (2s, 3H), 2.56 (q, J=7.0 Hz, 2H), 2.29 and 2.27 (2s, 31H), 1.01 (t, J=7.0 Hz, 3H). MS (ESI) m/e 418 (M+H)⁺.

### Example 37

### Preparation of (E)-N-((1,2-dihydroacenaphthylen-5-yl)methyl)-3-(6-ethyl-5-hydroxy-7-oxo-7,8-dihydro-1,8-naphthyridin-3-yl)-N-methylacrylamide

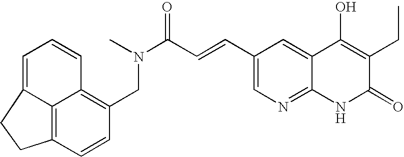

A solution of (E)-3-(5-hydroxy-6-ethyl-7-oxo-7,8-dihydro-1,8-naphthyridin-3-yl)acrylic acid (131 mg, 0.5 mmol), (1,2-dihydroacenaphthylen-5-yl)-N-methylmethanamine (130 mg, 0.65 mmol), EDCI (131 mg, 0.7 mmol), HOBt (70 mg, 0.50 mmol) and DIPEA (0.36 mL, 2 mmol) in DMF (3 mL) was submitted to microwave irradiation at 135° C. for 5 minutes. The mixture was diluted with H₂O (30 mL) and acidified with HCl and extracted with EtOAc. The crude product was purified by chromatography (silica, 0-2% MeOH in CH₂Cl₂) to give 118 mg (28%) of title compound as a mixture of amide rotamers. ¹H NMR (300 MHz, DMSO-d₆, δ) 11.77 and 11.72 (2s, 1H), 8.81 and 8.73 (2s, 1H), 8.55 and 8.44 (2s, 1H), 7.85-7.15 (m, 7H), 5.26 and 5.04 (2s, 2H), 3.4 (m, 4H), 3.06 and 2.95 (2s, 3H), 2.56 (q, J=7.0 Hz, 2H), 1.00 (t, J=7.0 Hz, 3H). MS (ESI) m/e 440 (M+H)⁺.

### Example 38

### Preparation of (E)-3-((E)-2,2-dimethyl-3-(methylimino)-3,4-dihydro-2H-pyrido[3,2-b][1,4]oxazin-7-yl)-N-methyl-N-((3-methylbenzofuran-2-yl)methyl)acrylamide hydrochloride

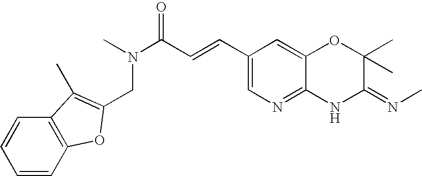

### a) (E)-N-(7-bromo-2,2-dimethyl-2H-pyrido[3,2-b][1,4]oxazin-3(4H)-ylidene)methanamine

A dichloroethane (5 mL) solution of 7-bromo-2,2-dimethyl-2H-pyrido[3,2-b][1,4]oxazin-3(4H)-one (520 mg, 2 mmol) and phosphorus pentachloride (840 mg, 4 mmol) was irradiated in a microwave oven for 10 min at 160° C. The solution was cooled to −78° C. and methylamine (2M in THF) was added it slowly until it became permanently basic. The mixture was diluted with CH₂Cl₂, washed with dilute solution of NaOH, dried and evaporated. Crystallization from CH₂Cl₂/hexane afforded 480 mg (89%) of the title compound. ¹H NMR (300 MHz, CDCl₃, δ) 8.07 (d, J=2.1 Hz, 1H), 7.21 (d, J=2.1 Hz, 1H), 4.9 (s, br, 1H), 3.06 (d, J=4.5 Hz, 3H), 1.46 (s, 6H). MS (ESI) m/e 270 (M+H)⁺.

### b) (E)-3-((E)-2,2-dimethyl-3-(methylimino)-3,4-dihydro-2H-pyrido[3,2-b][1,4]oxazin-7-yl)-N-methyl-N-((3-methylbenzofuran-2-yl)methyl)acrylamide hydrochloride

A DMF (3 mL) solution of (E)-N-(7-bromo-2,2-dimethyl-2H-pyrido[3,2-b][1,4]oxazin-3(4H)-ylidene)methanamine (271 mg, 1 mmol), N-methyl-N-((3-methylbenzofuran-2-yl)methyl)acrylamide (345 mg, 1.5 mmol) and diisopropylethylamine (0.52 mL, 3 mmol) was purged with Argon for 10 min. Pd(OAc)₂ (24 mg, 0.1 mmol) and P(o-Tol)₃ (61 mg, 0.2 mmol) was added and then the Argon purge was repeated. The mixture was irradiated in a microwave oven for 10 min at 160° C. under Argon. Upon cooling, the mixture was diluted with water and extracted with EtOAc. The crude product was purified by chromatography (silica, 0-4% MeOH in CH₂Cl₂). The free base was turned into the HCl salt by addition of HCl (1 mL, 1M in Et₂O) to its CH₂Cl₂ solution and evaporation to afford 230 mg (55%) of the title compound. as a mixture of amide rotamers. ¹H NMR (300 MHz, CDCl₃, δ, free base) 8.20 (s, 1H), 7.69 and 7.85 (2s, 1H), 7.5-6.7 (m, 6H), 5.14 (s, br, 1H), 4.83 and 4.71 (2s, 2H), 3.21 and 3.10 (2s, 3H), 3.09 (d, J=4.8 Hz, 3H), 2.31 (s, 3H), 1.49 (s, 6H). MS (ESI) m/e 419 (M+H)⁺.

### Example 39

### Preparation of (E)-3-((E)-2,2-dimethyl-3-(methylimino)-3,4-dihydro-2H-pyrido[3,2-b][1,4]oxazin-7-yl)-N-methyl-N-((3-methylbenzo[b]thiophen-2-yl)methyl)acrylamide

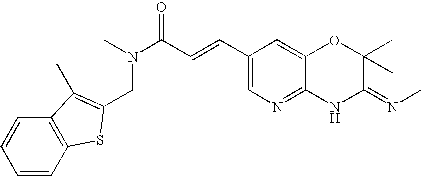

A DMF (3 mL) solution of (E)-N-(7-bromo-2,2-dimethyl-2H-pyrido[3,2-b][1,4]oxazin-3(4H)-ylidene)methanamine (271 mg, 1 mmol), N-methyl-N-((3-methylbenzo[b]thiophen-2-yl)methyl)acryl-amide (367 mg, 1.5 mmol) and diisopropylethylamine (0.52 mL, 3 mmol) was purged with Argon for 10 min. Pd(OAc)₂ (24 mg, 0.1 mmol) and P(o-Tol)₃ (61 mg, 0.2 mmol) was added and then the Argon purge was repeated. The mixture was irradiated in a microwave oven for 10 min at 160° C. under Argon. Upon cooling, the mixture was diluted with water and extracted with EtOAc. The crude product was purified by chromatography (silica, 0-4% MeOH in CH₂Cl₂). The free base was turned into the HCl salt by addition of HCl (1 mL, 1M in Et₂O) to its CH₂Cl₂ solution and evaporation to afford 287 mg (66%) of the title compound. as a mixture of amide rotamers. ¹H NMR (300 MHz, CDCl₃, 6, free base) 8.21 (s, 1H), 7.8-7.6 (m, 3H), 7.4-7.2 (m, 3H), 6.9-6.7 (m, 1H), 5.30 (s, br, 1H), 4.95 and 4.88 (2s, 2H), 3.10 (m, 6H), 2.43 (s, 3H), 1.49 (s, 6H). MS (ESI) m/e 435 (M+H)⁺.

### Example 40

### Preparation of (E)-3-(3-imino-2,2-dimethyl-3,4-dihydro-2H-pyrido[3,2-b][1,4]oxazin-7-yl)-N-methyl-N-((3-methylbenzofuran-2-yl)methyl)acrylamide

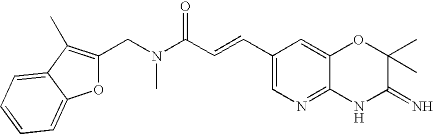

### a) (7-bromo-2,2-dimethyl-2H-pyrido[3,2-b][1,4]oxazin-3(4H)-imine

A dichloroethane (10 mL) solution of 7-bromo-2,2-dimethyl-2H-pyrido[3,2-b][1,4]oxazin-3(4H)-one (930 mg, 3.6 mmol) and phosphorus pentachloride (1518 mg, 7.8 mmol) was irradiated in a microwave oven for 10 min at 160° C. The solution was cooled to −78° C. and NH₃ gas was condensed over it. The mixture was slowly let to warm to 21° C., diluted with CH₂Cl₂, washed with dilute solution of NaOH, dried and evaporated. Crystallization from CH₂Cl₂/hexane afforded 602 mg (65%) of the title compound. ¹H NMR (300 MHz, CDCl₃, δ) 8.06 (d, J=2.0 Hz, 1H), 7.24 (d, J=2.0 Hz, 1H), 3.73 (s, 1H), 1.53 (s, 6H). MS (ESI) m/e 256 (M+H)⁺.

### b) (E)-3-(3-imino-2,2-dimethyl-3,4-dihydro-2H-pyrido[3,2-b][1,4]oxazin-7-yl)-N-methyl-N-((3-methyl-benzofuran-2-yl)methyl)acrylamide

A DMF (3 mL) solution of (E)-N-(7-bromo-2,2-dimethyl-2H-pyrido[3,2-b][1,4]oxazin-3(4H)-ylidene)methanamine (334 mg, 1.3 mmol), N-methyl-N-((3-methylbenzofuran-2-yl)methyl)acrylamide (390 mg, 1.7 mmol) and diisopropylethylamine (0.68 mL, 3.9 mmol) was purged with Argon for 10 min. Pd(OAc)₂ (30 mg, 0.13 mmol) and P(o-Tol)₃ (80 mg, 0.26 mmol) was added and then the Argon purge was repeated. The mixture was irradiated in a microwave oven for 12 min at 160° C. under Argon. Upon cooling, the mixture was diluted with water and extracted with EtOAc. The crude product was purified by chromatography (silica, 0-4% MeOH in CH₂Cl₂) to afford 80 mg (15%) of the title compound. as a mixture of amide rotamers. ¹H NMR (300 MHz, DMSO-d₆, δ) 8.08 (s, 1H), 7.7-7.0 (m, 7H), 4.99 and 4.78 (2s, 2H), 3.16 and 2.92 (2s, 3H), 2.56 (s, 3H), 1.43 (s, 6H). MS (ESI) m/e 405 (M+H)⁺.

### Example 41

### Preparation of (E)-N-methyl-N-((3-methyl-1H-indol-2-yl)methyl)-3-(2-oxo-1,2,3,5-tetrahydropyrido[2,3-e][1,4]oxazepin-7-yl)acrylamide

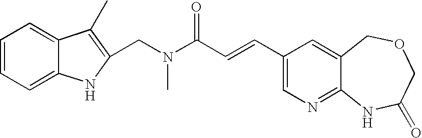

EDC (552 mg, 1.3 mmol) was added to a solution of N-methyl(3-methyl-1H-indol-2-yl)methanamine (183 mg, 1.0 mmol), (E)-3-(2-oxo-1,2,3,5-tetrahydropyrido[2,3-e][1,4]oxazepin-7-yl)acrylic acid hydrochloride (297 mg, 1.1 mmol), HOBT.H₂O (144 mg, 1.0 mmol) and DIPEA (0.76 mL, 3.0 mmol) in dry DMF (5 mL). After stirring overnight, water was added. The precipitate that formed was washed with ethyl acetate and dried to afford the title compound (20 mg, 4%). ¹H NMR (300 MHz, DMSO-d₆) δ 10.51 (s, 1H), 8.58 (s, 1H), 8.13 (s, 1H), 7.62-7.53 (m, 3H), 7.37 (d, J=6.7 Hz, 1H), 7.12-6.84 (m, 3H), 4.90-4.68 (rotamers, s, 2H), 4.31 (s, 3H), 3.40 (s, 2H), 2.34 (s, 3H); MS (ESI): m/e 391.2 (C₂₂H₂₂N₄O₃+H)⁺.

### Example 42

### (E)-N-methyl-N-((3-methylbenzofuran-2-yl)methyl)-3-(4-oxo-2,3,4,5-tetrahydro-1H-pyrido[2,3-b][1,4]diazepin-8-yl)acrylamide

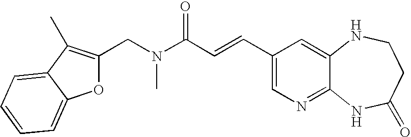

### Preparation of 1-(2-amino-5-bromopyridin-3-yl)azetidin-2-one

Step A. An argon purged round bottom flask was charged with 5-bromo-3-iodopyridin-2-amine (150 mg, 0.5 mmol), azetidin-2-one (36 mg, 0.5 mmol), tris-dibenzylideneacetone dipalladium (23 mg, 0.05 mmol), Xantphos (43 mg, 0.075 mmol) and cesium carbonate (326 mg, 1.0 mmol) followed by toluene (10 mL). The suspension was heated at 90° C. for 19 hours (overnight). After cooling, the mixture was filtered through a pad of celite and the filter cake was rinsed with 5 mL CH₂Cl₂. The filtrate was concentrated and subjected to flash chromatography on silica gel using 2.5% MeOH:97.5% CH₂Cl₂ to give a yellow solid. Yield: 100 mg (82.6%). ¹H NMR (300 MHz, DMSO-d₆) δ 7.87 (d, 1H, J=2.4 Hz), 7.45 (d, 1H, J=2.4 Hz), 6.53 (br s, 2H), 3.67 (t, 2H, J=4.4 Hz), 3.03 (t, 2H, J=4.6 Hz); ESI MS m/z 242 and 244 [C₈H₈N₃OBr+H]⁺

### Preparation of 8-bromo-2,3-dihydro-1H-pyrido[2,3-b][1,4]diazepin-4(5H)-one

Step B. A suspension of 1-(2-amino-5-bromopyridin-3-yl)azetidin-2-one (675 mg, 2.79 mmol) in 40 mL toluene was treated with titanium(IV) isopropoxide (0.4 mL, 1.39 mmol). The mixture was heated at 110° C. overnight (15 hours). The suspension was cooled to ° C., treated with hexanes (150 mL) then filtered. The solid was dissolved in 200 mL of a 15% MeOH:CH₂Cl₂ mixture then filtered through a pad of silica gel. The filtrate was concentrated to give a grayish solid. Yield: 550 mg (81.5%). ¹H NMR (300 MHz, DMSO-d₆) δ 9.76 (s, 1H), 7.71 (d, 1H, J=2.1 Hz), 7.28 (d, 1H, J=1.8 Hz), 6.34 (br s, 2H), 3.47-3.42 (m, 2H), 2.62-2.59 (m, 2H); ESI MS m/z/z 242 and 244 [C₈H₈N₃OBr+H]⁺

### Preparation of (E)-tert-butyl 3-(4-oxo-2,3,4,5-tetrahydro-1H-pyrido[2,3-b][1,4]diazepin-8-yl)acrylate

Step C. A solution of 8-bromo-2,3-dihydro-1H-pyrido[2,3-b][1,4]diazepin-4(5H)-one (1.2 g, 5.0 mmol), tert-butyl acrylate (1.4 mL, 10 mmol), (i-Pr)₂EtN (2.6 mL, 15 mmol), NBu₄CI (1.38 g, 5.0 mmol) in DMF (8 mL) and EtCN (8 mL) was de-oxygenated with Ar for 30 min. Pd₂(dba)₃ (230 mg, 0.25 mmol) and P(t-Bu)₃ (10% wt in hexanes) (1.5 mL, 0.5 mmol) was added and the solution was de-oxygenated for an additional 15 min. The reaction was heated to 100° C. for 18 hrs at which time the reaction was cooled to room temperature and then filtered through a short column of silica washing with EtOAc (50 mL). The filtrate was washed with brine (3×50 mL), dried (MgSO₄) and the solvent removed in vacuo. Purification by column chromatography (silica gel, CH₂Cl₂/MeOH, 96:4) gave the title compound (920 mg, 64%) as a yellow powder; ¹H NMR (400 MHz, acetone-d₆) δ 8.47 (s, 1H), 7.95 (s, 1H), 7.47 (d, J=16 Hz, 1H), 7.41 (s, 1H), 6.33 (d, J=16 Hz, 1H), 5.52 (s, 1H), 3.52 (m, 2H), 2.74 (m, 2H), 1.50 (s, 9H); ESI MS m/z 290 [C₁₅H₁₉N₃O₃+H]⁺

### Preparation of (E)-3-(4-oxo-2,3,4,5-tetrahydro-1H-pyrido[2,3-b][1,4]diazepin-8-yl)acrylic acid hydrochloride

Step D. A suspension of (E)-tert-butyl 3-(4-oxo-2,3,4,5-tetrahydro-1H-pyrido[2,3-b][1,4]diazepin-8-yl)acrylate (498 mg, 1.72 mmol) in 5 mL CH₂Cl₂ was treated with 5 mL of trifluoroacetic acid. The mixture became homogeneous and it was stirred at room temperature for 1 hour. The solution was concentrated to dryness and treated with 10 mL 4M HCl in dixane. The suspension was sonicated for 20 minutes, diluted with 50 mL Et₂O and sonicated for an additional 20 minutes. The solid was filtered and dried under reduced pressure overnight.

Yield: 460 mg (99%) ¹H-NMR (400 MHz, DMSO-d₆) δ 9.76 (s, 1H), 7.96 (s, 1H), 7.47 (d, 1H, J=16.0 Hz), 7.37 (s, 1H), 3.42-3.41 (m, 2H), 2.62-2.61 (m, 2H); ESI MS m/z 234 [C₁₁H₁₁N₃O₃+H]⁺

### (E)-N-methyl-N-((3-methylbenzofuran-2-yl)methyl)-3-(4-oxo-2,3,4,5-tetrahydro-1H-pyrido[2,3-b][1,4]diazepin-8-yl)acrylamide

The amide was prepared according to the general coupling procedure in a yield of 87%. ¹H NMR (400 MHz, DMSO-d₆) δ 9.69 (s, 1H), 8.00 (s, 1H), 7.57-7.04 (m, 7H), 6.02 (br s, 1H), 4.94 and 4.79 (2×s, 2H), 3.43-3.39 (m, 2H), 3.16 and 2.95 (2×s, 3H), 2.61 (t, 2H, J=5.2 Hz), 2.26 (s, 3H); ESI MS m/z 391 [C₂₂H₂₂N₄O₃+H]⁺

### Example 43

### (R,E)-N-methyl-N-(1-(3-methylbenzofuran-2-yl)ethyl)-3-(2-oxo-1,2,3,5-tetrahydropyrido[2,3-e][1,4]oxazepin-7-yl)acrylamide

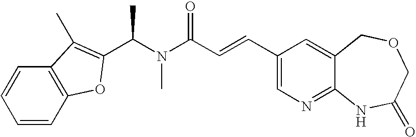

EDC (0.22 g, 1.2 mmol) was added to a suspension of (E)-3-(2-oxo-1,2,3,5-tetrahydropyrido[2,3-e][1,4]oxazepin-7-yl)acrylic acid hydrochloride (0.26 g, 0.96 mmol), HOBt (0.14 g, 1.1 mmol), (R)-N-methyl-1-(3-methylbenzofuran-2-yl)ethanamine (0.20 g, 1.1 mmol) and (i-Pr)₂EtN (1.0 mL, 5.8 mmol) in DMF (10 mL). The mixture was allowed to stir overnight at 35° C. The mixture was cooled to 0° C. and diluted with H₂O (30 mL) with rapid stirring. The resulting precipitate was filtered, washed with H₂O (20 mL) then dried under high vacuum. The solid was then triturated with Et₂O, and the resultant solid was collected, to yield 121 mg (31%); ¹H NMR (300 MHz, DMSO-d₆) δ 10.59 (s, 1H), 8.53 (s, 1H), 8.08 (s, 1H), 7.59-7.24 (m, 6H), 6.21 and 5.94 (2×q, J=7.9 Hz, 1H), 4.79 (s, 2H), 4.55 (s, 2H), 3.06 and 2.81 (2×s, 3H), 2.20 (s, 3H), 1.67 and 1.57 (2×d, J=6.7 Hz, 3H); ESI MS m/z 406 [C₂₃H₂₃N₃O₄+H]⁺

### Example 44

### (R,E)-N-methyl-N-(1-(3-methylbenzofuran-2-yl)ethyl)-3-(7-oxo-7,8-dihydro-1,8-naphthyridin-3-yl)acrylamide

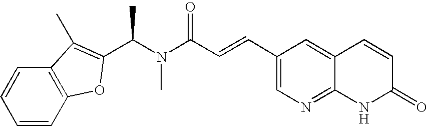

EDC (0.21 g, 1.1 mmol) was added to a suspension of (E)-3-(7-oxo-7,8-dihydro-1,8-naphthyridin-3-yl)acrylic acid acid hydrochloride (0.20 g, 0.93 mmol), HOBt (0.14 g, 1.0 mmol), (R)-N-methyl-1-(3-methylbenzofuran-2-yl)ethanamine (0.19 g, 1.0 mmol) and (i-Pr)₂EtN (0.94 mL, 5.6 mmol) in DMF (10 mL). The mixture was allowed to stir overnight at 35° C. The mixture was cooled to 0° C. and diluted with H₂O (30 mL) with rapid stirring. The resulting precipitate was filtered, washed with H₂O (20 mL) then dried under high vacuum. The solid was then triturated with Et₂O, and the resultant solid was collected, to yield 152 mg (42%); ¹H NMR (300 MHz, DMSO-d₆) δ 12.32 (s, 1H), 8.85 (s, 1H), 8.51 (s, 1H), 7.91 (d, J=9.9 Hz, 1H), 7.68-7.25 (m, 6H), 6.62 (d, J=8.5 Hz, 1H), 6.21 and 5.98 (2×q, J=6.7 Hz, 1H), 3.08 and 2.82 (2×s, 3H), 2.20 (s, 3H), 1.70 and 1.57 (2×d, J=6.4 Hz, 3H); ESI MS m/z 388 [C₂₃H₂₁N₃O₃+H]⁺

### Example 45

### (R,E)-N-methyl-N-(1-(3-methylbenzofuran-2-yl)ethyl)-3-(3-oxo-3,4-dihydro-2H-pyrido[3,2-b][1,4]oxazin-7-yl)acrylamide

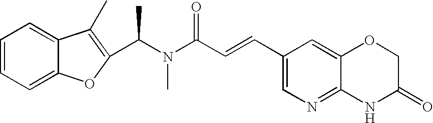

EDC (0.17 g, 0.86 mmol) was added to a suspension of (E)-3-(3-oxo-3,4-dihydro-2H-pyrido[3,2-b][1,4]oxazin-7-yl)acrylic acid trifluoroacetate (0.24 g, 0.72 mmol), HOBt (0.10 g, 0.79 mmol), (R)-N-methyl-1-(3-methylbenzofuran-2-yl)ethanamine (0.15 g, 0.79 mmol) and (i-Pr)₂EtN (0.73 mL, 4.3 mmol) in DMF (5 mL). The mixture was allowed to stir overnight at 35° C. The mixture was cooled to 0° C. and diluted with H₂O (30 mL) with rapid stirring. The resulting precipitate was filtered, washed with H₂O (20 mL) then dried under high vacuum. The solid was then triturated with Et₂O, and the resultant solid was collected, to yield 55 mg (20%); ¹H NMR (300 MHz, DMSO-d₆) δ 11.43 (s, 1H), 8.18 and 7.96 (2×s, 1H), 7.84 (s, 1H), 7.56-7.16 (m, 6H), 6.17 and 5.96 (2×m, 1H), 4.66 (s, 2H), 3.02 and 2.78 (2×s, 3H), 2.17 (s, 3H), 1.64 and 1.53 (2×d, J=6.9 Hz, 3H); ESI MS m/z 392 [C₂₂H₂₁N₃O₄+H]⁺

### Example 46

### (R,E)-N-(1-(3-methoxy-2-propoxyphenyl)ethyl)-N-methyl-3-(7-oxo-5,6,7,8-tetrahydro-1,8-naphthyridin-3-yl)acrylamide

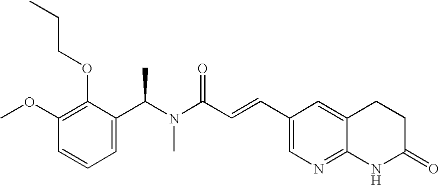

EDC (0.15 g, 0.77 mmol) was added to a suspension of (E)-3-(7-oxo-5,6,7,8-tetrahydro-1,8-naphthyridin-3-yl)acrylic acid (0.16 g, 0.64 mmol), HOBt (0.095 g, 0.70 mmol), (R)-1-(3-methoxy-2-propoxyphenyl)-N-methylethanamine (0.16 g, 0.64 mmol) and (i-Pr)₂EtN (0.65 mL, 3.8 mmol) in DMF (5 mL). The mixture was allowed to stir overnight at 35° C. The mixture was cooled to 0° C. and diluted with H₂O (30 mL) with rapid stirring. The resulting precipitate was filtered, washed with H₂O (20 mL) then dried under high vacuum. The solid was then triturated with Et₂O, and the resultant solid was collected, to yield 153 mg (57%); ¹H NMR (400 MHz, DMSO-d₆) δ 10.60 (s, 1H), 8.35 (s, 1H), 8.04 (s, 1H), 7.49-6.98 (m, 514), 6.02 and 5.75 (2×m, 1H), 3.86 and 3.70 (2×s, 2H), 3.78 (s, 3H), 2.93-2.55 (m, 7H), 1.63-1.39 (m, 5H), 0.87 and 0.80 (2×s, 3H); ESI MS m/z 424 [C₂₄H₂₉N₃O₄⁺H]⁺

### Example 47

### (E)-N-(3-methoxy-2-propoxybenzyl)-N-methyl-3-(4-oxo-2,3,4,5-tetrahydro-1H-pyrido[2,3-b][1,4]diazepin-8-yl)acrylamide

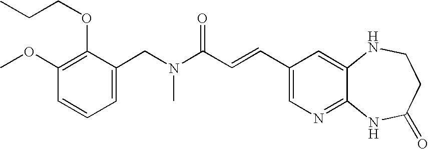

EDC (0.028 g, 0.15 mmol) was added to a suspension of (E)-3-(4-oxo-2,3,4,5-tetrahydro-1H-pyrido[2,3-b][1,4]diazepin-8-yl)acrylic acid (0.033 g, 0.12 mmol), HOBt (0.018 g, 0.13 mmol), (3-methoxy-2-propoxyphenyl)-N-methylmethanamine (0.028 g, 0.15 mmol) and (i-Pr)₂EtN (0.12 mL, 0.73 mmol) in DMF (1.5 mL). The mixture was allowed to stir overnight at 35° C. The mixture was cooled to 0° C. and diluted with H₂O (30 mL) with rapid stirring. The resulting precipitate was filtered, washed with H₂O (20 mL) then dried under high vacuum. The solid was then triturated with Et₂O, and the resultant solid was collected, to yield 16 mg (31%); ¹H NMR (400 MHz, DMSO-d₆) δ 9.64 and 9.66 (2×s, 1H), 7.97 and 7.91 (2×s, 1H), 7.40-6.92 (m, 5H), 6.64 and 6.59 (2×d, J=6.5, 1H), 5.95 (m, 1H), 4.71 and 4.60 (2×s, 2H), 3.84 (m, 2H), 3.76 (s, 3H), 3.37 (m, 2H), 3.05 and 2.85 (2×s, 3H), 2.56 (m, 2H), 1.66 (m, 2H), 0.93 (m, 3H); ESI MS nm/z 425 [C₂₃H₂₈N₄O₄+H]⁺

### Example 48

### (R,E)-N-methyl-N-(1-(3-methylbenzofuran-2-yl)ethyl)-3-(4-oxo-2,3,4,5-tetrahydro-1H-pyrido[2,3-b][1,4]diazepin-8-yl)acrylamide

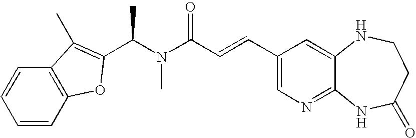

EDC (0.092 g, 0.48 mmol) was added to a suspension of (E)-3-(4-oxo-2,3,4,5-tetrahydro-1H-pyrido[2,3-b][1,4]diazepin-8-yl)acrylic acid (0.085 g, 0.32 mmol), HOBt (0.047 g, 0.35 mmol), (R)-N-methyl-1-(3-methylbenzofuran-2-yl)ethanamine (0.061 g, 0.35 mmol) and (i-Pr)₂EtN (0.32 mL, 1.89 mmol) in DMF (3.0 mL). The mixture was allowed to stir overnight at 35° C. The mixture was cooled to 0° C. and diluted with H₂O (30 mL) with rapid stirring. The resulting precipitate was filtered, washed with H₂O (20 mL) then dried under high vacuum. The solid was then triturated with Et₂O, and the resultant solid was collected, to yield 72 mg (51%); ¹H NMR (400 MHz, DMSO-d₆) δ 9.69 (s, 1H), 8.00 (s, 1H), 7.57-7.23 (m, 6H), 7.01 (d, J=16, 1H), 6.16 (m, 1H), 6.01 and 5.86 (2×s, 1H), 3.41 (s, 2H), 3.02 and 2.81 (2×s, 3H), 2.59 (s, 2H), 2.19 (s, 3H), 1.64 and 1.55 (2×s, 3H); ESI MS m/z 405 [C₂₃H₂₄N₄O₃+H]⁺

### Example 49

### (E)-N-methyl-N-((1-methyl-1H-indol-2-yl)methyl)-3-(4-oxo-2,3,4,5-tetrahydro-1H-pyrido[2,3-b][1,4]diazepin-8-yl)acrylamide

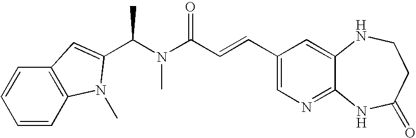

EDC (0.092 g, 0.48 mmol) was added to a suspension of (E)-3-(4-oxo-2,3,4,5-tetrahydro-1H-pyrido[2,3-b][1,4]diazepin-8-yl)acrylic acid (0.085 g, 0.32 mmol), HOBt (0.047 g, 0.35 mmol), N-methyl(1-methyl-1H-indol-2-yl)methanamine (0.060 g, 0.35 mmol) and (i-Pr)₂EtN (0.32 mL, 1.89 mmol) in DMF (3.0 mL). The mixture was allowed to stir overnight at 35° C. The mixture was cooled to 0° C. and diluted with H₂O (30 mL) with rapid stirring. The resulting precipitate was filtered, washed with H₂O (20 mL) then dried under high vacuum. The solid was then triturated with Et₂O, and the resultant solid was collected, to yield 61 mg (50%); ¹H NMR (400 MHz, DMSO-d₆) δ 9.76 and 9.72 (2×s, 1H), 8.02 and 7.99 (2×s, 1H), 7.51-7.00 (m, 7H), 6.43 and 6.17 (2×s, 1H), 6.05 and 5.97 (2×s, 1H), 5.02 and 4.85 (2×s, 2H), 3.72 and 3.68 (2×s, 3H), 3.39 (m, 2H), 3.10 and 3.00 (2×s, 3H), 2.59 (m, 2H); ESI MS m/z 390 [C₂₂H₂₃N₅O₂+H]⁺

### Example 50

### (E)-N-((5-fluoro-3-methylbenzo[b]thiophen-2-ylmethyl)-N-methyl-3-(4-oxo-2,3,4,5-tetrahydro-1H-pyrido[2,3-b][1,4]diazepin-8-yl)acrylamide

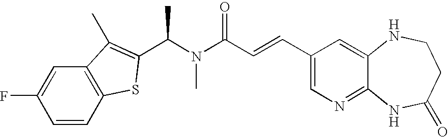

EDC (0.092 g, 0.48 mmol) was added to a suspension of (E)-3-(4-oxo-2,3,4,5-tetrahydro-1H-pyrido[2,3-b][1,4]diazepin-8-yl)acrylic acid (0.085 g, 0.32 mmol), HOBt (0.047 g, 0.35 mmol), (5-fluoro-3-methylbenzo[b]thiophen-2-yl)-N-methylmethanamine (0.068 g, 0.35 mmol) and (i-Pr)₂EtN (0.32 mL, 1.89 mmol) in DMF (3.0 mL). The mixture was allowed to stir overnight at 35° C. The mixture was cooled to 0° C. and diluted with H₂O (30 mL) with rapid stirring. The resulting precipitate was filtered, washed with H₂O (20 mL) then dried under high vacuum. The solid was then triturated with Et₂O, and the resultant solid was collected, to yield 78 mg (58%); ¹H NMR (400 MHz, DMSO-d₆) δ 9.74 (s, 1H), 8.00 (s, 1H), 7.89 (m, 1H), 7.55-7.05 (m, 5H), 6.02 (s, 1H), 5.07 and 4.86 (2×s, 2H), 3.40 (s, 2H), 3.13 and 2.94 (2×s, 3H), 2.59 (s, 2H), 2.38 (s, 3H); ESI MS m/z 425 [C₂₂H₂₁FN₄O₂S+H]⁺

### Example 51

### (R,E)-N-(1-(3-ethylbenzofuran-2-yl)ethyl)-N-methyl-3-(4-oxo-2,3,4,5-tetrahydro-3H-pyrido[2,3-b][1,4]diazepin-8-yl)acrylamide

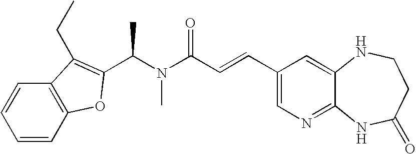

EDC (0.093 g, 0.48 mmol) was added to a suspension of (E)-3-(4-oxo-2,3,4,5-tetrahydro-1H-pyrido[2,3-b][1,4]diazepin-8-yl)acrylic acid (0.12 g, 0.45 mmol), HOBt (0.060 g, 0.45 mmol), (R)-1-(3-ethylbenzofuran-2-yl)-N-methylethanamine (0.082 g, 0.41 mmol) and (i-Pr)₂EtN (0.41 mL, 2.43 mmol) in DMF (3.0 mL). The mixture was allowed to stir overnight at 35° C. The mixture was cooled to 0° C. and diluted with H₂O (30 mL) with rapid stirring. The resulting precipitate was filtered, washed with H₂O (20 mL) then dried under high vacuum. The solid was then triturated with Et₂O, and the resultant solid was collected, to yield 37 mg (22%); ¹H NMR (400 MHz, DMSO-d₆) δ 9.69 (s, 1H), 7.99 (s, 1H), 7.62-7.22 (m, 6H), 7.00 (d, J=15 Hz, 1H), 6.18 (m, 1H), 6.00 (s, 1H), 3.41 (s, 2H), 3.03 and 2.81 (2×s, 3H), 2.67-2.59 (m, 4H), 1.66 and 1.54 (2×s, 3H), 1.13 (s, 3H); ESI MS m/z 419 [C₂₄H₂₆N₄O₃+H]⁺

### Example 52

### Preparation of (E)-3-(5-hydroxy-6,6-dimethyl-7-oxo-5,6,7,8-tetrahydro-1,8-naphthyridin-3-yl)-N-methyl-N-((3-methylbenzofuran-2-yl)methyl)acrylamide

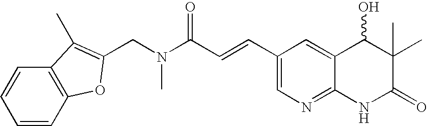

### Preparation of 6-bromo-4-hydroxy-3,3-dimethyl-3,4-dihydro-1,8-naphthyridin-2(1H)-one

Step A: A mixture of activated Zn (2.8 g, 43 mmol) and ethyl 2-bromo-2-methylpropanoate (2.6 mL, 17 mmol) in 9 mL of THF were added together at 0° C. and stirred for 6 h while allowing it to warm to R.T. To this mixture a dropwise solution of 2-amino-5-bromonicotinaldehyde in 5 mL of THF was added via canula and the reaction mixture was stirred for a further 19 h at R.T. Dilute the mixture with 25 mL of ethyl acetate and wash with water (50 mL) and brine (50 mL), dry over magnesium sulphate and concentrate in vacuo. The crude solid product is triturated with diethyl ether and filtered to obtain the yellow solid product. Yield 675 mg (72%); ¹H NMR (300 MHz, DMSO-d₆) δ 10.65 (s, 1H), 8.28 (s, 1H), 7.87 (s, 1H), 5.78 (d, J=6 Hz, 1H), 4.42 (d, J=6 Hz, 1H), 1.05 (s, 6H); ESI MS m/z 271, 273 [C₁₀H₁₁N₂O₂+H]⁺

### Preparation of (E)-tert-butyl 3-(5-hydroxy-6,6-dimethyl-7-oxo-5,6,7,8-tetrahydro-1,8-naphthyridin-3-yl)acrylate

Step B: A suspension of 6-bromo-4-hydroxy-3,3-dimethyl-3,4-dihydro-1,8-naphthyridin-2(1H)-one (945 mg, 3.5 mmol), tert-butyl acrylate (2.5 mL, 17.4 mmol) and (i-Pr)₂EtN (1.8 mL, 10.5 mmol) in 50 mL of DMF was de-oxygenated with Ar for 30 min. The mixture was treated with Pd(OAc)₂ (78 mg, 0.35 mmol) and P(o-tol)₃ (212 mg, 0.70 mmol) then heated to 110° C. for 22 h. The hot mixture was filtered through a pad of celite and wash with 50 mL ethyl acetate. The filtrate was diluted with 100 mL H₂O and the white precipitate that formed is filtered. The filtrate is extracted with 2×100 mL ethyl acetate, dried over magnesium sulphate, and concentrated in vacuo. The resulting white solids are combined and triturated with a diethyl ether followed by filtration to yield the white solid product. Yield 764 mg (69%); ¹H NMR (300 MHz, DMSO-d₆) δ 10.71 (s, 1H), 8.45 (s, 1H), 8.07 (s, 1H), 7.59 (d, J=15 Hz, 1H), 6.51 (d, J=15 Hz, 1H), 5.70 (d, J=6 Hz, 1H), 4.38 (d, J=6 Hz, 1H), 1.50 (s, 9H), 1.09 (s, 3H), 1.03 (s, 3H); ESI MS m/z 319 [C₁₇H₂₂N₂O₄+H]⁺

### Preparation of (E)-3-(5-hydroxy-6,6-dimethyl-7-oxo-5,6,7,8-tetrahydro-1,8-naphthyridin-3-yl)acrylic acid hydrochloride

Step C: A solution of (E)-tert-butyl 3-(5-hydroxy-6,6-dimethyl-7-oxo-5,6,7,8-tetrahydro-1,8-naphthyridin-3-yl)acrylate (629 mg, 2.0 mmol) in CH₂Cl₂ (20 mL) was treated with TFA (20 mL). After stirring at room temperature for 2 h, the solution was concentrated in vacuo. The resulting orange oil was treated with anhydrous HCl in dioxane (4 mL, 4.0 M) and sonicated until the oil was converted to a fine off-white solid. The mixture is diluted with diethyl ether and precipitate is formed. The solid isolated by filtration and dried under vacuum. Yield: 636 mg (quant); ¹H NMR (300 MHz, DMSO-d₆) δ 10.72 (s, 1H), 8.44 (s, 1H), 8.06 (s, 1H), 7.59 (d, J=16.2 Hz, 1H), 6.51 (d, J=16.1 Hz, 1H), 5.21 (bs, 1H), 4.39 (d, 1H) 1.08 (s, 3H), 1.03 (s, 3H); ESI MS m/z 263 [C₁₃H₁₄N₂O₄+H]⁺

### Preparation of (E)-3-(5-hydroxy-6,6-dimethyl-7-oxo-5,6,7,8-tetrahydro-1,8-naphthyridin-3-yl)-N-methyl-N-((3-methylbenzofuran-2-yl)methyl)acrylamide

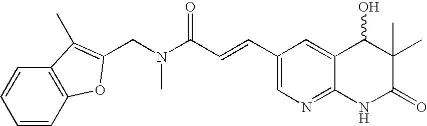

EDC (154 mg, 0.81 mmol) was added to a suspension of (E)-3-(5-hydroxy-6,6-dimethyl-7-oxo-5,6,7,8-tetrahydro-1,8-naphthyridin-3-yl)acrylic acid hydrochloride (200 mg, 0.67 mmol), HOBt (100 mg, 0.74 mmol), methyl-(3-methyl-benzofuran-2-ylmethyl)-amine (129 mg, 0.74 mmol) and (i-Pr)₂EtN (0.57 mL, 3.3 mmol) in DMF (8 mL). The mixture was allowed to stir for 17 h at 40° C. The mixture was cooled to room temperature and diluted with water (25 mL) to yield a brown precipitate, which was collected by suction filtration. The solid was then triturated with diethyl ether to obtain the product as a light brown solid. Yield: 131 mg (47%); ¹H NMR (300 MHz, DMSO-d₆) δ 10.68 (s, 1H), 8.46 (s, 1H), 8.14 (s, 1H), 7.55 (m, 3H), 7.27 (m, 3H), 5.72 (bm, 1H), 4.90 (s, 2H), 4.38 (s, 1H), 3.11 (s, 3H), 2.29 (s, 3H), 1.10 (s, 3H), 1.04 (s, 3H); ESI MS m/z 420 [C₂₄H₂₅N₃O₄+H]⁺

### Example 53

### Preparation of (E)-3-(5-hydroxy-6,6-dimethyl-7-oxo-5,6,7,8-tetrahydro-1,8-naphthyridin-3-yl)-N-methyl-N-((1-methyl-1H-indol-2-yl)methyl)acrylamide

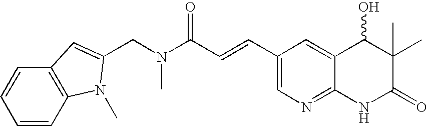

EDC (154 mg, 0.81 mmol) was added to a suspension of (E)-3-(5-hydroxy-6,6-dimethyl-7-oxo-5,6,7,8-tetrahydro-1,8-naphthyridin-3-yl)acrylic acid hydrochloride (200 mg, 0.67 mmol), HOBt (100 mg, 0.74 mmol), N-methyl(1-methyl-1H-indol-2-yl)methanamine (129 mg, 0.74 mmol) and (i-Pr)₂EtN (0.57 mL, 3.3 mmol) in DMF (8 mL). The mixture was allowed to stir for 17 h at 40° C. The mixture was cooled to room temperature and diluted with water (25 mL) to yield a brown precipitate, which was collected by suction filtration. The solid was then triturated with diethyl ether to obtain the crude product which was subjected to flash silica gel column chromatography with an 85:15 (DCM:MeOH) solvent ratio to obtain the product as a light brown solid. Yield: 130 mg (46%); ¹H NMR (300 MHz, DMSO-d₆) δ 10.68 (s, 1H), 8.44 (s, 1H), 8.12 (s, 1H), 7.66-6.96 (m, 6H), 6.35 (s, 1H), 5.65 (bs, 1H), 4.93 (s, 2H), 4.33 (s, 1H), 3.73 (s, 3H), 3.11 (s, 3H), 1.04 (m, 6H); ESI MS m/z 419 [C₂₄H₂₆N₄O₃+H]⁺

### Example 54

### Preparation of (R,E)-3-(S-hydroxy-6,6-dimethyl-7-oxo-5,6,7,8-tetrahydro-1,8-naphthyridin-3-yl)-N-methyl-N-(1-(3-methylbenzofuran-2-yl)ethyl)acrylamide

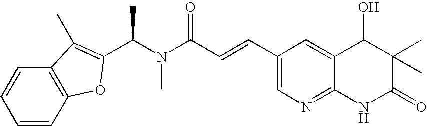

EDC (154 mg, 0.81 mmol) was added to a suspension of (E)-3-(5-hydroxy-6,6-dimethyl-7-oxo-5,6,7,8-tetrahydro-1,8-naphthyridin-3-yl)acrylic acid hydrochloride (200 mg, 0.67 mmol), HOBt (100 mg, 0.74 mmol), (R)-N-methyl-1-(3-methylbenzofuran-2-yl)ethanamine (140 mg, 0.74 mmol) and (i-Pr)₂EtN (0.57 mL, 3.3 mmol) in DMF (8 mL). The mixture was allowed to stir for 17 h at 40° C. The mixture was cooled to room temperature and diluted with water (25 mL) to yield a brown precipitate, which was collected by suction filtration. The solid was then triturated with diethyl ether to obtain the light brown solid product. Yield: 178 mg (61%); ¹H NMR (300 MHz, DMSO-d₆) δ 10.66 (s, 1H), 8.43 (s, 1H), 8.11 (s, 1H), 7.58-7.13 (m, 6H), 6.19 (m, 1H), 5.69 (s, 1H), 4.35 (d, J=6 Hz, 1H), 2.92 (s, 3H), 2.14 (s, 3H), 1.55 (s, 3H), 1.07 (s, 3H), 1.01 (s, 3H); ESI MS m/z 434 [C₂₅H₂₇N₃O₄+H]⁺

### Example 55

### Preparation of (E)-3-(3-hydroxy-2,2-dimethyl-3,4-dihydro-2H-pyrido[3,2-b][1,4]oxazin-7-yl)-N-methyl-N-((3-methylbenzofuran-2-yl)methyl)acrylamide trifluoroacetate salt

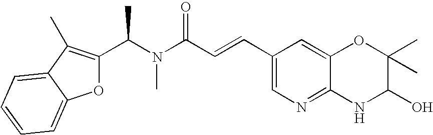

### a) (E)-tert-butyl 2,2-dimethyl-7-(3-(methyl((3-methylbenzofuran-2-yl)methyl)amino)-3-oxoprop-1-enyl)-3-oxo-2,3-dihydropyrido[3,2-b][1,4]oxazine-4-carboxylate

Di-tert-butyl dicarbonate (1.76 g, 8.0 mmol) and 4-dimethylaminopyridine (74 mg, 0.6 mmol) was added to an acetonitrile (100 mL) suspension of (E)-3-(2,2-dimethyl-3-oxo-3,4-dihydro-2H-pyrido[3,2-b][1,4]oxazin-7-yl)-N-methyl-N-((3-methylbenzofuran-2-yl)methyl)acrylamide (811 mg, 2 mmol). After stirring it for 14 hours at 40° C., the solution was evaporated to dryness. The crude product was purified by chromatography (silica, 0-1.2% MeOH in CH₂Cl₂) to give 824 mg (81%) title compound. MS (ESI) m/e 406 (M-C₅H₇O₂)⁺, 506 (M+H)⁺.

### b) (E)-3-(3-hydroxy-2,2-dimethyl-3,4-dihydro-2H-pyrido[3,2-b][1,4]oxazin-7-yl)-N-methyl-N-((3-methylbenzofuran-2-yl)methyl)acrylamide trifluoroacetate salt

LiAlH₄ (2.2 mL, 1M in THF) was added slowly to a THF (10 mL) solution of (E)-tert-butyl 2,2-dimethyl-7-(3-(methyl((3-methylbenzofuran-2-yl)methyl)amino)-3-oxoprop-1-enyl)-3-oxo-2,3-dihydropyrido[3,2-b][1,4]oxazine-4-carboxylate (547 mg, 1.08 mmol) at −78° C. After an hour stirring at this temperature, HCl (0.4 mL, 37%) and CH₂Cl₂ (50 mL) was added. After another 30 min stirring, the mixture was filtered through celite and MgSO₄ and evaporated to 20 mL. Trifluoroacetic acid (4 mL) was added and the resulting mixture was further stirred for 2 hours. Upon evaporation, the crude mixture was purified by preparative HPLC to give 120 mg (21%) title compound as rotamers. ¹H NMR (300 MHz, DMSO-d₆, δ) 8.69 (s, 1H), 7.96-7.75 (m, 2H), 7.6-7.1 (m, 6H), 4.98 (s, 1H), 4.77 and 4.73 (2s, 2H), 3.16 and 2.91 (2s, 3H), 2.26, 1.33 and 1.18 (3s, 3×3H); MS (ESI) m/e 408 (M+H)⁺.

### Example 56

### Preparation of (E)-N-methyl-N-((3-methyl-1H-indol-2-yl)methyl)-3-(2-oxo-1,2,3,5-tetrahydropyrido[2,3-e][1,4]oxazepin-7-yl)acrylamide

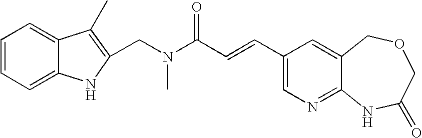

EDC (552 mg, 1.3 mmol) was added to a solution of N-methyl(3-methyl-1H-indol-2-yl)methanamine (183 mg, 1.0 mmol), (E)-3-(2-oxo-1,2,3,5-tetrahydropyrido[2,3-e][1,4]oxazepin-7-yl)acrylic acid hydrochloride (297 mg, 1.1 mmol), HOBT-H₂O (144 mg, 1.0 mmol) and DIPEA (0.76 mL, 3.0 mmol) in dry DMF (5 mL). After stirring overnight, water was added. The precipitate that formed was washed with ethyl acetate and dried to afford the title compound (20 mg, 4%). ¹H NMR (300 MHz, DMSO-d₆) δ 10.51 (s, 1H), 8.58 (s, 1H), 8.13 (s, 1H), 7.62-7.53 (m, 3H), 7.37 (d, J=6.7 Hz, 1H), 7.12-6.84 (m, 3H), 4.90-4.68 (rotamers, s, 2H), 4.31 (s, 3H), 3.40 (s, 2H), 2.34 (s, 31); MS (ESI): m/e 391.2 (C₂₂H₂₂N₄O₃+H)⁺.

### Example 57

### Preparation of (E)-N-methyl-N-((3-methyl-1H-indol-2-yl)methyl)-3-(3-oxo-3,4-dihydro-2H-pyrido[3,2-b][1,4]oxazin-7-yl)acrylamide

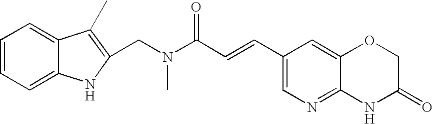

EDC (149 mg, 1.3 mmol) was added to a solution of N-methyl(3-methyl-1H-indol-2-yl)methanamine (110 mg, 1.0 mmol), (E)-3-(3-oxo-3,4-dihydro-2H-pyrido[3,2-b][1,4]oxazin-7-yl)acrylic acid hydrochloride (220 mg, 1.1 mmol), HOBT.H₂O (81 mg, 1.0 mmol) and DIPEA (0.43 mL, 3.0 mmol) in dry DMF (5 mL). After stirring overnight, water was added. The precipitate that formed was washed with ethyl acetate and dried to afford the title compound (12 mg, 4%). ¹H NMR (300 MHz, DMSO-d₆) δ 8.25 (s, 1H), 7.97 (s, 1H), 7.77 (d, J=7.1 Hz, 1H), 7.60 (s, 1H), 7.45 (d, J=7.4 Hz, 1H), 7.34 (m, 2H), 7.18 (s, 1H), 7.11 (m, 1H), 4.90-4.79 (rotamers, s, 2H), 4.72 (s, 2H), 4.60 (s, 3H), 2.31 (s, 3H); MS (ESI): m/e 377.2 (C₂₁H₂₀N₄O₃+H)⁺.

### Example 58

### Preparation of (E)-N-methyl-N-((3-methyl-1H-indol-2-yl)methyl)-3-(1,2,3,5-tetrahydropyrido[2,3-e][1,4]oxazepin-7-yl)acrylamide

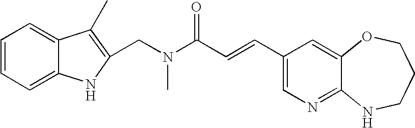

EDC (86 mg, 1.3 mmol) was added to a solution of N-methyl(3-methyl-1H-indol-2-yl)methanamine (71 mg, 1.0 mmol), (E)-3-(1,2,3,5-tetrahydropyrido[2,3-e][1,4]oxazepin-7-yl)acrylic acid hydrochloride (117 mg, 1.1 mmol), HOBT-H₂O (54 mg, 1.0 mmol) and DIPEA (0.27 mL, 3.0 mmol) in dry DMF (5 mL). After stirring overnight, water was added. The precipitate that formed was washed with ethyl acetate and dried to afford the title compound. (12 mg, 8%). ¹H NMR (300 MHz, DMSO-d₆) δ 7.85 (s, 1H), 7.70-7.55 (m, 2H), 7.52-7.37 (m, 2H), 7.10 (t, J=7.2 Hz, 1H), 6.85 (m, 2H), 6.52 (d, J=6.8 Hz, 1H), 4.89-4.79 (rotamers, s, 2H), 4.65 (s, 3H), 4.19 (m, 4H), 3.12 (m, 2H), 2.31 (s, 3H); MS (ESI): m/e 377.2 (C₂₂H₂₄N₄O₂+H)⁺.

### Preparation of (E)-3,3-dimethyl-4-oxo-2,3,4,5-tetrahydropyrido[3,2-b][1,4]oxazin-8-yl)acrylic acid

### a) methyl 2,2-dimethyl-3-(2-nitropyridin-3-yloxy)propanoate

3-Hydroxynitropyridine (10.0 g, 64 mmol), methyl 2,2-dimethyl-3-hydroxypropionate (9.29 g, 70.4.0 mmol) and PPH₃ (15.15 g, 76.8 mmol) were dissolved in dioxane (500 mL). DIAD (14.5 mL, 76.8 mmol) was added at 0° C. over 5 min and the mixture was stirred at rt for 4 h then refluxed overnight. The mixture was evaporated, dissolved in ethyl acetate, washed with water, dried over magnesium sulfate and evaporated in vacuo to afford the title compound (9.1 g, 61%). ¹H NMR (300 MHz, DMSO-d₆) δ 8.22 (d, J=1.4 Hz, 1H), 7.98 (d, J=2.0 Hz, 1H), 7.70 (m, 1H), 4.43 (s, 2H), 3.59 (s, 3H), 1.17 (s, 6H)

### b) methyl 2,2-dimethyl-3-(2-aminopyridin-3-yloxy)propanoate

A suspension of methyl 2,2-dimethyl-3-(2-nitropyridin-3-yloxy)propanoate (9.1 g, 6 mmol) and Pd/C (800 mg) in methanol (500 mL) was stirred at rt overnight under hydrogen. The cooled mixture was filtered through celite, washed with methanol and evaporated in vacuo to afford the title compound (8.23 g, 100%). ¹H NMR (300 MHz, DMSO-d₆) δ 7.51 (d, J=1.3 Hz, 1H), 7.03 (d, J=1.3 Hz, 1H), 6.47 (m, 1H), 5.47 (s, 2H), 3.94 (s, 2H), 3.62 (s, 3H), 1.17 (s, 6H)

### c) 3,3-dimethyl-2,3-dihydropyrido[3,2-b][1,4]oxazepin-4(5H)-one

NaH (60% in oil 533 mg) was added to a solution of methyl 2,2-dimethyl-3-(2-aminopyridin-3-yloxy)propanoate (8.04, 37 mmol) in DMSO (400 mL) and the mixture stirred overnight at rt. The mixture was diluted with water and separated. The aqueous layer was washed with ethyl acetate and the combined organic phases were dried over magnesium sulfate and evaporated in vacuo to afford the title compound (6.5 g, 94%). ¹H NMR (300 MHz, DMSO-d₆) δ 9.83 (s, 1H), 7.96 (d, J=4.0 Hz, 1H), 7.37 (d, J=8.0 Hz, 1H), 6.98 (m, 1H), 4.02 (s, 2H), 1.47 (s, 6H)

### d) 8-bromo-3,3-dimethyl-2,3-dihydropyrido[3,2-b][1,4]oxazepin-4(5H)-one

Bromine (13.3 mL, 83.2 mmol) was added slowly to a cooled solution of 3-dimethyl-2,3-dihydropyrido[3,2-b][1,4]oxazepin-4(5H)-one (4 g, 20.8 mmol) in CH₂Cl₂ (400 mL) with Na₂CO₃ (1 g). The mixture was stirred at rt overnight and poured into saturated NaHSO₃ (200 mL). The mixture was separated and the aqueous layer washed with CH₂Cl₂. The combined organic phases were dried and evaporated in vacuo to afford the title compound (4.21 g, 78%). ¹H NMR (300 MHz, DMSO-d₆) δ 10.13 (s, 1H), 8.16 (s, 1H), 7.77 (s, 1H), 4.05 (s, 2H), 1.47 (s, 6H)

### e) (E)-tert-butyl 3-(3,3-dimethyl-4-oxo-2,3,4,5-tetrahydropyrido[3,2-b][1,4]oxazepin-8-yl)acrylate

A solution of 8-bromo-3,3-dimethyl-2,3-dihydropyrido[3,2-b][1,4]oxazepin-4(5H)-one (1 g, 3.6 mmol), t-butyl acrylate (1.38 g, 10.8 mmol) and DIPEA (1.86 mL, 10.1 mmol) in DMF (10 mL) was purged with Ar for 10 min. Pd(OAc)₂ (81 mg, 0.36 mmol) and P(o-tol)₃ (218 mg, 0.72 mmol) were added and the mixture purged again then refluxed overnight. The crude mixture was evaporated in vacuo and chromatographed over silica eluting with methanol/dichloromethane to afford the title compound (1.0 g, 87%). ¹H NMR (300 MHz, DMSO-d₆) δ 10.13 (s, 1H), 8.26 (s, 1H), 7.77 (s, 1H), 7.47 (d, J=16.1 Hz, 1H), 6.53 (d, J=16.1 Hz, 1H), 4.05 (s, 2H), 1.53 (s, 9H), 1.47 (s, 6H)

### f) (E)-3-(3,3-dimethyl-4-oxo-2,3,4,5-tetrahydropyrido[3,2-b][1,4]oxazepin-8-yl)acrylic acid

TFA (3 mL) was added to a cooled solution of (E)-tert-butyl 3-(3,3-dimethyl-4-oxo-2,3,4,5-tetrahydropyrido[3,2-b][1,4]oxazepin-8-yl)acrylate (1 g, 3 mmol) in CH₂Cl₂ (5 mL) and stirred for 30 min at rt. The mixture was evaporated and HCl/dioxane (4M, 5 mL) was added. The precipitate that formed was washed with ether and dried to afford the title compound (530 mg, 66%). ¹H NMR (300 MHz, DMSO-d₆) δ 8.45 (s, 1H), 8.28 (s, 1H), 7.82 (d, J=15.5 Hz, 1H), 6.84 (d, J=20 Hz, 1H), 4.05 (s, 2H), 1.47 (s, 6H).

### Example 59

### Preparation of (E)-N-((3-ethyl-1H-indol-2-yl)methyl)-N-methyl-3-(3-oxo-3,4-dihydro-2H-pyrido[3,2-b][1,4]oxazin-7-yl)acrylamide

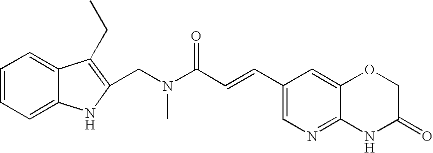

EDC (116 mg, 0.6 mmol) was added to a solution of (3-ethyl-1H-indol-2-yl)-N-methylmethanamine (87 mg, 0.46 mmol), (E)-3-(3-oxo-3,4-dihydro-2H-pyrido[3,2-b][1,4]oxazin-7-yl)acrylic acid hydrochloride (123.65 mg, 1.05 mmol), HOBT.H₂O (62 mg, 0.46 mmol) and DIPEA (0.33 mL, 1.8 mmol) in dry DMF (5 mL). After stirring overnight, water was added. The precipitate that formed was washed with ethyl acetate and dried to afford the title compound (76.2 mg, 42%). ¹H NMR (400 MHz, DMSO-d₆) δ 11.41 (s, 1H), 8.18 (s, 1H), 7.88 (s, 1H), 7.46 (t, J=7.9 Hz, 2H), 7.25 (d, J=7.9 Hz, 1H), 7.03 (t, J=7.6 Hz, 1H), 6.95 (t, J=7.1 Hz, 2H), 4.92 (s, 2H), 4.90-4.74 (rotamers, s, 2H), 4.68 (s, 3H), 3.08 (m, 2H), 1.13 (t, J=7.4 Hz, 3H); MS (ESI): m/e 391.1 (C₂₂H₂₂N₄O₃+H)⁺.

### Example 60

### Preparation of (E)-N-methyl-3-(3-oxo-3,4-dihydro-2H-pyrido[3,2-b][1,4]oxazin-7-yl)-N-((3-vinyl-1H-indol-2-yl)methyl)acrylamide

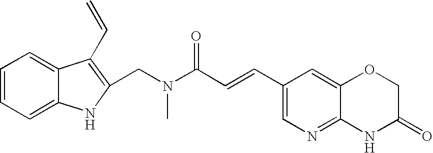

EDC (73 mg, 0.3 mmol) was added to a solution of N-methyl(3-vinyl-1H-indol-2-yl)methanamine (54.7 mg, 0.29 mmol), (E)-3-(3-oxo-3,4-dihydro-2H-pyrido[3,2-b][1,4]oxazin-7-yl)acrylic acid hydrochloride (79 mg, 0.3 mmol), HOBT.H₂O (39 mg, 0.29 mmol) and DIPEA (0.21 mL, 1.1 mmol) in dry DMF (5 mL). After stirring overnight, water was added. The precipitate that formed was washed with ethyl acetate and dried to afford the title compound (26 mg, 23%). ¹H NMR (400 MHz, DMSO-d₆) δ 11.41 (s, 1H), 11.20-11.05 (rotamers, s, 1H), 8.18 (s, 1H), 7.87 (s, 1H), 7.80 (d, J=7.8 Hz, 1H), 7.51 (d, J=15.6 Hz, 1H), 7.38 (d, J=7.7 Hz, 1H), 7.22 (d, J=15.6 Hz, 1H), 7.10 (t, J=7.1 Hz, 1H), 7.07-6.98 (m, 2H), 5.62 (d, J=17.9 Hz, 1H), 5.12 (d, J=11.0 Hz, 1H), 5.03 (s, 2H), 4.68 (s, 3H), 3.09 (s, 2H); MS (ESI): m/e 389.1 (C₂₂H₂₀N₄O₃+H)⁺.

### Preparation of (1,3-dimethyl-1H-indol-2-yl)-N-methylmethanamine

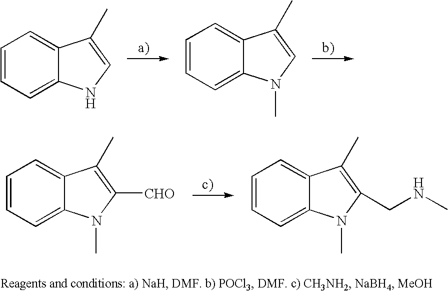

### a) 1,3-dimethyl-1H indole

Sodium hydride (600 mg, 16.6 mmol) was added to a solution of 3-methylindole (2 g, 15.2 mmol) in DMF (10 mL). The mixture was stirred for 30 min and iodomethane was added in one portion. The mixture was cooled in an icebath and left to warm to rt overnight. The mixture was evaporated and the residue dissolved in ethyl acetate. The solution was washed with water and brine, dried over magnesium sulfate and evaporated. The crude reaction was chromatographed over silica gel eluting with hexane and ethyl acetate/hexane (20 and 50%) to afford the title compound (1.3 g, 59%). ¹H NMR (400 MHz, CDCl₃) δ 7.46 (d, J=8 Hz, 1H), 7.34 (d, J=8 Hz, 1H), 7.12 (t, J=7.8 Hz, 1H), 7.06 (s, 1H), 7.00 (t, J=7.6 Hz, 1H), 3.70 (s, 3H), 2.23 (s, 3H)

### b) 1,3-dimethyl-1H indole-2-carbaldehyde

Phosphorous oxychloride (0.0.93 mL, 9.7 mmol) was added dropwise with stirring to DMF 5 mL) at 10° C. over 20 min. 1,3-dimethyl-1H indole (1.3 g mg, 8.9 mmol) in DMF (5 mL) was added slowly with stirring and the mixture was heated for 3 h at 98-100° C. Excess concentrated aqueous solution of sodium acetate was added. The mixture was stirred for 30 min at 28° C. and extracted with ethyl acetate, dried and evaporated. The crude mixture was chromatographed over silica gel eluting with hexane/ether to afford the title compound (1.5 g, 97%). ¹H NMR (400 MHz, DMSO-d₆) δ 10.15 (s, 1H), 7.74 (d, J=7.6 Hz, 1H), 7.53 (d, J=7.8 Hz, 1H), 7.42 (t, J=8.0 Hz, 1H), 7.15 (t, J=7.6 Hz, 1H), 3.99 (s, 3H), 2.60 (s, 3H)

### c) (1,3-dimethyl-1H-indol-2-yl)-N-methylmethanamine

Methylamine (0.53 mL, 13.1 mmol) was added to a solution of 1,3-dimethyl-1H indole-2-carbaldehyde (760 mg, 4.3 mmol) in methanol (15 mL) and stirred for 5 h. The mixture was cooled to 0° C. and sodium borohydride (159 mg, 4.3 mmol) added slowly. The mixture was warmed to rt and stirred overnight. Water (3 mL) was added slowly at 0° C. and evaporated to a paste. Water was added and the mixture extracted with dichloromethane. The organic phase was washed with water, dried and evaporated to afford the title compound (690 mg, 85%). ¹H NMR (400 MHz, DMSO-d₆) δ 7.44 (d, J=7.8 Hz, 1H), 7.34 (d, J=8.1 Hz, 1H), 7.10 (t, J=7.6 Hz, 1H), 6.98 (t, J=8.0 Hz, 1H), 3.77 (s, 2H), 3.71 (s, 3H), 2.27 (s, 3H), 2.23 (s, 3H)

### Example 61

(E)-N-((1,3-dimethyl-1H-indol-2-yl)methyl)-N-methyl-3-(3-oxo-3,4-dihydro-2H-pyrido[3,2-b][1,4]oxazin-7-yl)acrylamide

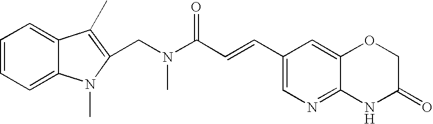

EDC (132.5 mg, 0.6 mmol) was added to a solution of (1,3-dimethyl-1H-indol-2-yl)-N-methylmethanamine (100 mg, 0.5 mmol), (E)-3-(3-oxo-3,4,dihydro-2H-pyrido[3,2-b][1,4]oxazin-7-yl)acrylic acid hydrochloride (143 mg, 0.55 mmol), HOBT-H₂O (72 mg, 0.5 mmol)) and DIPEA (0.38 mL, 2.1 mmol) in dry DMF (5 mL). After stirring overnight, water was added. The precipitate that formed was washed with ethyl acetate and dried to afford the title compound (144 mg, 74%). ¹H NMR (400 MHz, DMSO-d₆) δ 11.51 (s, 1H), 8.24 (s, 1H), 7.94 (d, J=7.6 Hz, 1H), 7.60-7.48 (m, 2H), 7.38 (m, 10H), 7.20-7.28 (m, 1H), 7.14 (t, J=7.8 Hz, 1H), 7.07 (d, J=7.5 Hz, 1H), 5.11 (s, 2H), 5.01-4.98 (rotamers, s, 2H), 4.77 (s, 3H), 3.80 (s, 3H), 2.31 (s, 3H); MS (EST): m/e 391.2 (C₂₂H₂₂N₄O₃+H)⁺.

### Example 62

(E)-N-((1,3-dimethyl-1H-indol-2-yl)methyl)-N-methyl-3-3-(7-oxo-5,6,7,8-tetrahydro-1,8-naphthyridin-3-yl)acrylamide

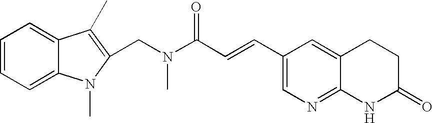

EDC (132.5 mg, 0.6 mmol) was added to a solution of (1,3-dimethyl-1H-indol-2-yl)-N-methylmethanamine (100 mg, 0.5 mmol), (E)-3-(2-methylene-1,2,3,4-tetrahydroquinolin-6-yl)acrylic acid hydrochloride (149 mg, 0.55 mmol), HOBT.H₂O (72 mg, 0.5 mmol)) and DIPEA (0.38 mL, 2.1 mmol) in dry DMF (5 mL). After stirring overnight, water was added. The precipitate that formed was washed with ethyl acetate and dried to afford the title compound (144 mg, 74%). ¹H NMR (400 MHz, DMSO-d₆) δ 10.66 (s, 1H), 8.40 (s, 1H), 8.04 (s, 1H), 7.59-7.48 (m, 2H), 7.36 (d, J=7.8 Hz, 1H), 7.24-7.11 (m, 2H), 7.02 (t, J=7.7 Hz, 1H), 5.10-4.90 (rotamers, s, 2H), 4.85 (s, 3H), 3.77 (s, 3H), 2.99-2.81 (m, 4H), 2.32 (s, 3H); MS (ESI): m/e 389.2 (C₂₃H₂₄N₄O₂+H)⁺.

### Example 63

### (E)-N-((1,3-dimethyl-1H-indol-2-yl)methyl)-N-methyl-3-(2-oxo-2,3,4,5-tetrahydro-1H-pyrido[2,3-b][1,4]diazepin-8-yl)acrylamide

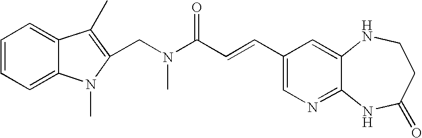

EDC (86 mg, 0.4 mmol) was added to a solution of (1,3-dimethyl-1H-indol-2-yl)-N-methylmethanamine (72 mg, 0.38 mmol), (E)-3-(2-oxo-2,3,4,5-tetrahydro-1H-benzo[b][1,4]diazepin-7-yl)acrylic acid hydrochloride (94 mg, 0.3 mmol), HOBT.H₂O (47 mg, 0.3 mmol)) and DIPEA (0.25 mL, 1.3 mmol) in dry DMF (5 mL). After stirring overnight, water was added. The precipitate that formed was washed with ethyl acetate and dried to afford the title compound (144 mg, 74%). ¹H NMR (400 MHz, DMSO-d₆) δ 9.69 (s, 1H), 7.95 (d, J=8.0 Hz, 1H), 7.48 (d, J=7.7 Hz, 1H), 7.41 (s, 1H), 7.35 (m, 2H), 7.16 (m, 1H), 7.08 (m, 2H), 6.09 (s, 1H), 4.98-4.87 (rotamers, s, 2H), 3.62 (s, 3H), 3.42 (m, 2H), 2.98 (s, 3H), 2.60 (m, 2H), 2.31 (s, 3H); MS (ESI): m/e 404.2 (C₂₃H₂₅N₅O₂+H)⁺.

### Preparation of (3,7-Dimethyl-1H-indol-2-yl)-N-methanamine

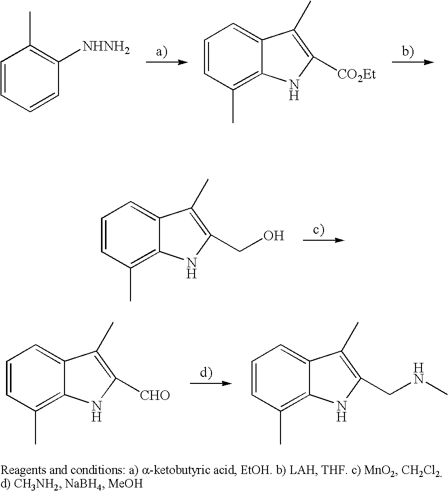

### a) ethyl 3,7-dimethyl-1H-indole-2-carboxylate

A suspension of 1-o-tolylhydrazine (3.8 g, 30.9 mmol) in ethanol was warmed to 50° C. A solution of α-ketobutyric acid (3.16 g, 30.9 mmol) in ethanol was added and the mixture stirred at rt overnight. Hydrogen chloride was bubbled through the solution for 30 min and the mixture heated at reflux for 2 h then evaporated in vacuo. The crude reaction was chromatographed over silica gel eluting with ethyl acetate/hexane (5%) to afford the title compound (1.84 g, 27%). ¹H NMR (400 MHz, CDCl₃) δ 8.55 (s, 1H), 7.50 (d, J=7.8 Hz, 1H), 7.09 (d, J=6.9 Hz, 1H), 7.03 (t, J=7.6 Hz, 1H), 4.41 (q, J=7.1 Hz, 2H), 2.59 (s, 3H), 2.48 (s, 3H), 1.44 (t, J=7.2 Hz, 3H)

### b) (3,7-Dimethyl-1H-indol-2-yl)methanol

A solution of ethyl 3,7-dimethyl-1H-indole-2-carboxylate (1.84 g, 8.4 mmol) in THF (20 mL) was added to an ice-cooled solution of 1.0 M LAH in THF (17.8 mL 17.8 mmol) and stirred overnight. The reaction was quenched with ethyl acetate (5 mL) and 15% aqueous sodium hydroxide (5 mL), filtered through celite and evaporated in vacuo. The crude reaction was chromatographed over silica gel eluting with methanol/dichloromethane (1%) to afford the title compound (440 mg, 30%). ¹H NMR (400 MHz, CDCl₃) δ 8.39 (s, 1H), 7.39 (d, J=7.7 Hz, 1H), 7.06 (t, J=7.2 Hz, 1H), 6.99 (d, J=7.1 Hz, 1H), 4.82 (s, 2H), 4.11 (q, J=7.1 Hz, 2H), 2.45 (s, 3H), 2.28 (s, 3H), 1.26 (t, J=7.1 Hz, 3H)

### c) 3,7-Dimethyl-1H-indole-2-carbaldehyde

A mixture of (3,7-dimethyl-1H-indol-2-yl)methanol (440 mg, 2.5 mmol) and manganese dioxide (1.09 g, 12.5 mmol) in dichloromethane (15 mL) was stirred overnight at rt. The mixture was filtered and evaporated. The crude was chromatographed over silica gel eluting with ethyl acetate/hexane (5% and 7.5%) to afford the title compound (200 mg, 46%). ¹H NMR (400 MHz, CDCl₃) δ 10.00 (s, 1H), 8.75 (s, 1H), 7.51 (d, J=7.2 Hz, 1H), 7.15 (d, J=7.4 Hz, 1H), 7.05 (t, J=7.3 Hz, 1H), 2.61 (s, 3H), 2.45 (s, 3H)

### d) (3,7-Dimethyl-1H-indol-2-yl)-N-methanamine

Methylamine (0.43 mL, 3.4 mmol) was added to a solution of 3,7-dimethyl-1H-indole-2-carbaldehyde (200 mg, 1.1 mmol) in methanol (5 mL) and stirred for 5 h. The mixture was cooled to 0° C. and sodium borohydride (40.7 mg, 1.1 mmol) added slowly. The mixture was warmed to rt and stirred overnight. Water (3 mL) was added slowly at 0° C. and evaporated to a paste. Water was added and the mixture extracted with dichloromethane. The organic phase was washed with water, dried and evaporated to afford the title compound (120 mg, 57%). ¹H NMR (400 MHz, CDCl₃) δ 8.40 (s, 1H), 7.36 (d, J=7.7 Hz, 1H), 7.00 (t, J=7.6 Hz, 1H), 6.94 (d, J=7.0 Hz, 1H), 3.89 (s, 2H), 2.48 (s, 3H), 2.45 (s, 3H), 2.27 (s, 3H)

### Example 64

(E)-N-((3,7-dimethyl-1H-indol-2-yl)methyl)-N-methyl-3-(3-oxo-3,4-dihydro-2H-pyrido[3,2-b][1,4]oxazin-7-yl)acrylamide

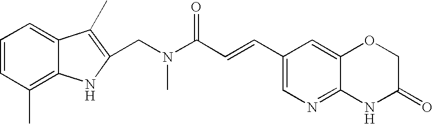

EDC (157 mg, 0.8 mmol) was added to a solution of (3,7-dimethyl-1H-indol-2-yl)-N-methanamine (120 mg, 0.6 mmol), (E)-3-(3-oxo-3,4,dihydro-2H-pyrido[3,2-b][1,4]oxazin-7-yl)acrylic acid hydrochloride (177 mg, 0.7 mmol), HOBT.H₂O (85 mg, 0.6 mmol) and DIPEA (0.45 mL, 2.5 mmol) in dry DMF (5 mL). After stirring overnight, water was added. The precipitate that formed was washed with ethyl acetate and dried (14 mg, 6%). ¹H NMR (400 MHz, DMSO-d₆) δ 11.41 (s, 1H), 10.60-10.52 (rotamers, s, 1H), 8.19 (s, 1H), 7.87 (s, 1H), 7.50 (d, J=7.6 Hz, 1H), 7.25 (d, J=6.8 Hz, 1H), 6.88 (m, 2H), 4.90-4.77 (rotamers, s, 2H), 4.68 (s, 3H), 3.05 (s, 2H), 2.84 (s, 1H), 2.44 (s, 3H), 2.21 (s, 3H); MS (ESI): m/e 391.1 (C₂₂H₂₂N₄O₃+H)⁺.

### Example 65

### (E)-N-methyl-N-((3,7-methyl-1H-indol-2-yl)methyl-3-(7-oxo-5,6,7,8-tetrahydro-1,8-naphthyridin-3-yl)acrylamide

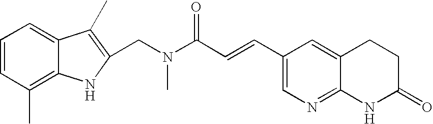

EDC (250 mg, 1.3 mmol) was added to a solution of (3,7-dimethyl-1H-indol-2-yl)-N-methanamine (174 mg, 1.0 mmol), (E)-3-(2-methylene-1,2,3,4-tetrahydroquinolin-6-yl)acrylic acid hydrochloride (369 mg, 1.1 mmol), HOBT.H₂O (136 mg, 1.0 mmol) and DIPEA (0.72 mL, 4.0 mmol) in dry DMF (5 mL). After stirring overnight, water was added. The precipitate that formed was washed with ethyl acetate and dried to afford the title compound (3 mg, 0.7%). ¹H NMR (400 MHz, DMSO-d₆) δ 9.56 (s, 1H), 8.76 (s, 1H), 8.30 (s, 1H), 7.77 (s, 1H), 7.65 (d, J=7.8 Hz, 1H), 7.38 (d, J=7.2 Hz, 1H), 7.0 (m, 2H), 6.84 (d, J=7.2 Hz, 1H), 4.72 (s, 2H), 3.15 (s, 3H), 3.01 (t, J=6.8 Hz, 2H), 2.71 (t, J=6.9 Hz, 2H), 2.44 (s, 3H), 2.38 (s, 3H); MS (ESI): m/e 389.2 (C₂₃H₂₄N₄O₂+H)⁺.

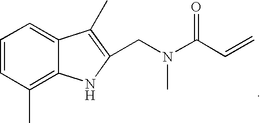

### N-((3,7-dimethyl-1H-indol-2-yl)methyl)-N-methylacrylamide

Triethylamine (0.17 mL, 1.2 mmol) and acryloyl chloride (0.10 mL, 1.2 mmol) were added one at a time to a solution of (3,7-dimethyl-1H-indol-2-yl)-N-methanamine (161 mg, 0.8 mmol) in dichloromethane (10 mL). The reaction was stirred for 2 h at rt then water (2 mL) was added. The mixture was separated and the aqueous phase extracted with ethyl acetate. The combined organics was washed with sodium bicarbonate and brine, dried over magnesium sulfate and evaporated to give the title product (117 mg, 64%). ¹H NMR (400 MHz, CDCl₃) δ 8.00 (s, 1H), 7.34 (d, J=7.5 Hz, 1H), 6.94 (m, 3H), 6.51 (d, J=6.3 Hz, 1H), 6.34 (d, J=6.8 Hz, 2H), 4.63 (s, 2H), 3.05 (s, 3), 2.46 (s, 3H), 2.30 (s, 3H)

### Example 66

(E)-N-((3,7-dimethyl-1H-indol-2-yl)methyl)-N-methyl-3-(8-oxo-6,7,8,9-tetrahydro-5H-pyrido[2,3-b]azepin-3-yl)acrylamide

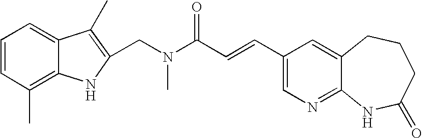

A mixture of N-((3,7-dimethyl-1H-indol-2-yl)methyl)-N-methylacrylamide (116 mg, 0.5 mmol), 3-bromo-6,7-dihydro-5H-pyrido[2,3-b]azepin-8(9H)-one (40 mg, 0.16 mmol), DIPEA (0.10 mL, 0.5 mmol), Pd(OAc)₂ (11 mg, 0.05 mmol) and P(o-tol)₃ (31 mg, 0.1 mmol) in DMF (2 mL) were stirred under Ar for 10 min then microwaved at 160° C. for 5 min. The cooled mixture was filtered through celite and water added. The precipitate that formed was washed with water and diethylether and dried to afford the title compound (15 mg, 7.4%). ¹H NMR (400 MHz, DMSO-d₆) δ 10.80-10.62 (rotamers, s, 1H), 10.07 (s, 1H), 8.53 (s, 1H), 8.17 (s, 1H), 7.55 (d, J=6.9 Hz, 1H), 7.27 (m, 2H), 6.89 (m, 2H), 4.90-4.80 (rotamers, s, 2H), 3.14 (s, 3H), 2.80 (m, 2H), 2.44 (s, 3H), 2.30 (m, 6H). MS (ESI): m/e 403.2 (C₂₄H₂₆N₄O₂+H)⁺.

### Preparation of N-methyl(3-methyl-7-trifluoromethyl)-1H-indol-2-yl)methanamine

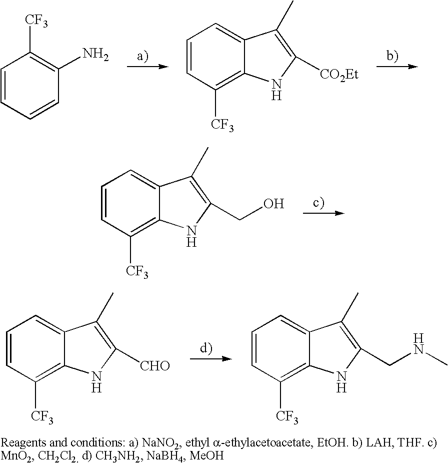

### a) Ethyl 3-methyl-7-(trifluoromethyl)-1H-indole-2-carboxylate

A solution of sodium nitrite (2.3 g, 34 mmol) was added dropwise to a mixture of trifluoromethyl aniline (3.85 mL, 31 mmol), HCl (7.5 mL) and water (15 mL) at −5° C. After the addition, the mixture was stirred at 0° C. for 15 min and brought to pH 3-4 by addition of sodium acetate. In a separate flask, a solution of ethyl α-ethylacetoacetate (5 mL, 31 mmol) in ethanol (25 mL) at 0° C. was treated with a solution of potassium hydroxide (1.74 g, 31 mmol) in water (10 mL) followed by addition of ice. The diazonium salt was immediately added to this alkaline solution. The mixture was adjusted to pH 5-6 by adding sodium acetate and stirred at 0° C. for 3 h. The solution was kept overnight at 4° C. and extracted with ethyl acetate, washed with brine, dried over magnesium sulfate and most of the solvent removed. The crude mixture was added dropwise to a solution of ethanolic HCl (25 mL) at 78° C. and stirred for 2 h at 78° C. The mixture was evaporated and chromatographed over silica gel eluting with ethyl acetate/hexane (3%) to afford the title compound (2.26 g, 26%). ¹H NMR (400 MHz, CDCl₃) δ 8.92 (s, 1H), 7.79 (d, J=8 Hz, 1H), 7.55 (d, J=7.2 Hz, 1H), 7.15 (t, J=7.6 Hz, 1H), 4.41 (q, J=6.9 Hz, 2H), 2.60 (s, 3H), 1.42 (t, J=6.9 Hz, 3H)

### b) (3-Methyl-7-(trifluoromethyl)-1H-indol-2-yl)methanol

A solution of ethyl 3-methyl-7-(trifluoromethyl 1H-indole-2-carboxylate (2.2 g, 8.1 mmol) in THF (50 mL) was added to an ice cooled solution of 1M LAH in THF (16.2 mL, 16.2 mmol) and stirred overnight. The reaction was quenched with ethyl acetate and sodium hydroxide, filtered through celite and evaporated to afford the title compound (1.03 g, 57%). ¹H NMR (400 MHz, CDCl₃) δ 8.74 (s, 1H), 7.66 (d, J=7.9 Hz, 1H), 7.40 (d, J=7.3 Hz, 1H), 7.12 (t, J=4.7 Hz, 1H), 4.81 (s, 2H), 2.26 (s, 3H)

### c) 3-Methyl-7-(trifluoromethyl)-1H-indole-2-carbaldehyde

A mixture of (3-methyl-7-(trifluoromethyl)-1H-indol-2-yl)methanol (1.03 g, 4.4 mmol) and manganese (IV) oxide (1.95 g, 22.4 mmol) in dichloromethane (15 mL) was stirred overnight. The mixture was filtered through celite and evaporated to afford the title compound (510 mg, 51%). ¹H NMR (400 MHz, CDCl₃) δ 10.08 (s, 1H), 8.95 (s, 1H), 7.87 (d, J=7.9 Hz, 1H), 7.62 (d, J=7.1 Hz, 1H), 7.20 (t, J=7.7 Hz, 1H), 2.65 (s, 3H)

### d) N-methyl(3-methyl-7-trifluoromethyl)-1H-indol-2-yl)methanamine

Methylamine (0.28 mL, 6.7 mmol) was added to a solution of 3-methyl-7-(trifluoromethyl)-1H-indole-2-carbaldehyde (510 mg, 2.2 mmol) in methanol (5 mL) and stirred for 5 h. The mixture was cooled to 0° C. and sodium borohydride (83 mg, 2.2 mmol) added slowly. The mixture was warmed to rt and stirred overnight. Water (3 mL) was added slowly at 0° C. and evaporated to a paste. Water was added and the mixture extracted with dichloromethane. The organic phase was washed with water, dried and evaporated to afford the title compound (310 mg, 58%). ¹H NMR (400 MHz, CDCl₃) δ 8.93 (s, 1H), 7.66 (d, J=7.8 Hz, 1H), 7.38 (d, J=7.4 Hz, 1H), 7.11 (t, J=7.5 Hz, 1H), 3.90 (s, 2H), 2.48 (s, 3H), 2.28 (s, 3H)

### Example 67

### (E)-N-methyl-N-((3-methyl-7-(trifluoromethyl)-1H-indol-2-yl)methyl)-3-(3-oxo-3,4-dihydro-2H-pyrido[3,2-b][1,4]oxazin-7-yl)acrylamide

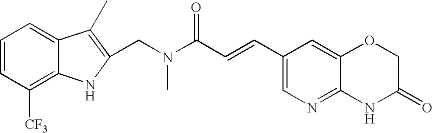

EDC (160 mg, 0.78 mmol) was added to a solution of N-methyl(3-methyl-7-trifluoromethyl)-1H-indol-2-yl)methanamine (155 mg, 0.6 mmol), (E)-3-(3-oxo-3,4,dihydro-2H-pyrido[3,2-b][1,4]oxazin-7-yl)acrylic acid hydrochloride (180 mg, 0.7 mmol), HOBT.H₂O (86 mg, 0.6 mmol) and DIPEA (0.46 mL, 2.5 mmol) in dry DMF (5 mL). After stirring overnight, water was added. The precipitate that formed was washed with ethyl acetate and dried to afford the title compound (72 mg, 27%). ¹H NMR (400 MHz, DMSO-d₆) δ 11.41 (s, 1H), 11.10-10.89 (rotamers, s, 1H), 8.20 (s, 1H), 7.80 (d, J=8.0 Hz, 1H), 7.76 (d, J=7.5 Hz, 1H), 7.54 (s, 1H), 7.41 (d, J=7.5 Hz, 1H), 7.31-7.20 (m, 2H), 5.05-4.81 (rotamers, s, 2H), 4.68 (s, 2H), 3.08 (s, 3H), 2.21 (s, 3H); MS (ESI): m/e 445.1 (C₂₂H₁₉F₃N₄O₃+H)⁺.

### Example 68

### (E)-N-((7-ethyl-3-methyl-1H-indol-2-yl)methyl)-N-methyl-3-(3-oxo-3,4-dihydro-2H-pyrido[3,2-b][1,4]oxazin-7-yl)acrylamide

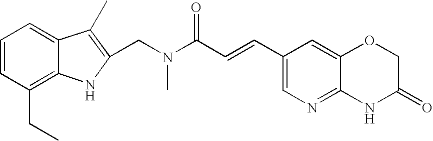

EDC (147 mg, 0.7 mmol) was added to a solution of (7-ethyl-3-methyl-1H-indol-2-yl)-N-methanamine (108.7 mg, 0.5 mmol), (E)-3-(3-oxo-3,4,dihydro-2H-pyrido[3,2-b][1,4]oxazin-7-yl)acrylic acid hydrochloride (151 mg, 0.6 mmol), HOBT.H₂O (73 mg, 0.5 mmol) and DIPEA (0.39 mL, 2.1 mmol) in dry DMF (5 mL). After stirring overnight, water was added. The precipitate that formed was washed with ethyl acetate and dried to afford the title compound (25 mg, 0.001%). ¹H NMR (400 MHz, DMSO-d₆) δ 9.34 (s, 1H), 8.92 (s, 1H), 8.15 (s, 1H), 7.65 (d, J=7.6 Hz, 1H), 7.41 (s, 1H), 7.38 (d, J=7.2 Hz, 1H), 7.05 (m, 2H), 6.80 (d, J=7.5 Hz, 1H), 4.70 (s, 2H), 3.15 (s, 3H), 2.88 (q, J=7.0 Hz, 2H), 2.40 (s, 3H), 1.30 (t, J=7.2 Hz, 3H); MS (ESI): m/e 405.2 (C₂₃H₂₄N₄O₃+H)⁺.

### Example 69

### Preparation of (E)-N-((3,6-dimethyl-1H-indol-5-yl)methyl)-N-methyl-3-(3-oxo-3,4-dihydro-2H-pyrido[3,2-b][1,4]oxazin-yl)acrylamide)

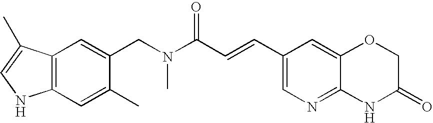

EDC (198 mg, 1.0 mmol) was added to a solution of (7-ethyl-3-methyl-1H-indol-2-yl)-N-methanamine (150 mg, 0.8 mmol), (E)-3-(3-oxo-3,4,dihydro-2H-pyrido[3,2-b][1,4]oxazin-7-yl)acrylic acid hydrochloride (224 mg, 0.87 mmol) HOBT.H₂O (107 mg, 0.8 mmol) and DIPEA (0.57 mL, 3.1 mmol) in dry DMF (5 mL). After heating overnight, water was added. The precipitate that formed was washed with ethyl acetate and dried (184 mg, 59%). ¹H NMR (300 MHz, DMSO-d₆, δ, 11.42 (s, 1H), 10, 45 (s, 1H), 8.24-8.05 (rotamers, s, 1H), 7.98-7.80 (rotamers, s, 2H), 7.54 (d, J=7.4 Hz, 1H), 7.24 (m, 1H), 7.14 (m, 1H), 6.98 (s, 1H), 4.90-4.78 (rotamers, s, 2H), 3.05 (s, 2H), 2.84 (s, 3H), 2.45 (s, 3H), 2.31 (s, 3H) n/e 391.1 (C₂₂H₂₂N₄O₃+H)⁺.

### Example 70

### (E)-3-(2,2-dimethyl-3,4-dihydro-2H-pyrido[3,2-b][1,4]oxazin-7-yl)-N-methyl-N-((3-methylbenzofuran-2-yl)methyl)acrylamide hydrochloride

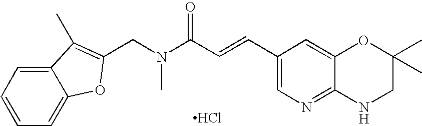

To a solution of methyl-(3-methyl-benzofuran-2-ylmethyl)-amine (88 mg, 0.5 mmol) in DMF (5 mL) were added in sequential order 3-(2,2-dimethyl-3,4-dihydro-2H-pyrido[3,2-b][1,4]oxazin-7-yl)acrylic acid hydrochloride (107 mg, 0.46 mmol), 1-hydroxybenzotriazole (68 mg, 0.5 mmol), diisopropylethylamine (240 uL, 1.38 mmol), and N-(3-dimethylaminopropyl)-N′-ethylcarbodiimide (100 mg, 0.5 mmol). The mixture was stirred at room temperature overnight, cooled in an ice bath and water added with rapid stirring. The product was extracted with ethyl acetate (3×10 mL), dried with sodium sulfate, filtered and concentrated. The free base was re-solvated in methylene chloride (5 mL) and a solution of 4M HCl in dioxane (1 mL) was added to precipitate the hydrogen chloride salt as a pale yellow solid (84 mg, 43%): ¹H NMR (400 MHz, DMSO-d₆) δ 8.94 (Is, 1H), 8.01-8.06 (m, 2H), 7.47-7.58 (m, 3H), 7.16-7.25 (m, 3H), 4.73-4.94 (rotamers, 2s, 2H), 3.42 (s, 2H), 3.04 (s, 3H), 2.42 (s, 3H), 1.34 (s, 6H); MS (ESI) m/e 392 (C₂₃H₂₅N₃O₃+H)⁺.

### Example 71

### (E)-N-((3-chlorobenzofuran-2-yl)methyl)-N-methyl-3-(3-oxo-3,4-dihydro-2H-pyrido[3,2-b][1,4]oxazin-7-yl)acrylamide

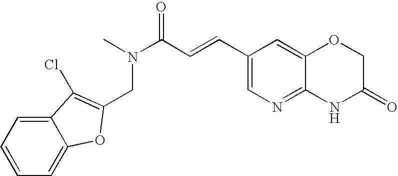

To a solution of (3-chlorobenzofuran-2-yl)-N-methylmethanamine (100 mg, 0.51 mmol) in DMF (5 mL) were added in sequential order (E)-3-(3-oxo-3,4-dihydro-2H-pyrido[3,2-b][1,4]oxazin-7-yl)acrylic acid (107 mg, 0.46 mmol), 1-hydroxybenzotriazole (71 mg, 0.51 mmol), diisopropylethylamine (243 uL, 1.39 mmol), and N-(3-dimethylaminopropyl)-N′-ethylcarbodiimide (102 mg, 0.51 mmol). The mixture was stirred at room temperature overnight, cooled in an ice bath and water was added with rapid stirring. The product precipitated and was filtered, triturated with ether and dried to yield title compound as a white solid (55 mg, 30%): ¹H NMR (400 MHz, CDCl₃) δ 9.21 (s, 1H), 8.12-8.19 (m, 1H), 7.28-7.6 (m, 7H), 4.81-4.95 (rotamers, 2s, 2H), 4.714 (s, 2H); 3.21 (s, 3H); MS (ESI) m/e 398 (C₂₀H₁₆ClN₃O₄+H)⁺.

### Example 72

### (E)-N-(3-methoxy-2-propoxybenzyl)-N-methyl-3-(3-oxo-3,4-dihydro-2H-pyrido[3,2-b][1,4]oxazin-7-yl)acrylamide

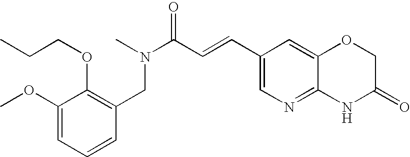

To a solution of (3-methoxy-2-propoxyphenyl)-N-methylmethanamine (75 mg, 0.36 mmol) in DMF (5 mL) were added in sequential order (E)-3-(3-oxo-3,4-dihydro-2H-pyrido[3,2-b][1,4]oxazin-7-yl)acrylic acid (75 mg, 0.26 mmol), 1-hydroxybenzotriazole (50 mg, 0.36 mmol), diisopropylethylamine (150 uL, 0.67 mmol), and N-(3-dimethylaminopropyl)-N′-ethylcarbodiimide (72 mg, 0.36 mmol). The mixture was placed in a microwave at a temperature of 140° C. for 8 minutes. The product precipitated with the addition of water and was filtered, triturated with ether and dried to yield title compound as a white solid (38 mg, 36%): ¹H NMR (400 MHz, CDCl₃) δ 8.03-8.12 (rotamers, 2s, 1H), 7.63-7.69 (dd, J=15.2 Hz, J=11.2 Hz, 1H), 7.44-7.35 (rotamers, 2s, 1H), 7.02-7.04 (m, 1H), 6.80-6.91 (m, 2H), 6.72 (d, J=7.2 Hz, 1H), 4.67-4.81 (rotamers, 4s, 4H), 3.91-4.01 (m, 2H), 3.90 (2s, rotomers, 3H), 3.10 (s, 3H), 1.76-1.89 (m, 2H), 1.05 (t, J=7.2 Hz, 3H); MS (ESI) m/e 412 (C₂₂H₂₅N₃O₅+H)⁺.

### Example 73

### (E)-N-((3-isopropylbenzofuran-2-yl)methyl)-N-methyl-3-(3-oxo-3,4-dihydro-2H-pyrido[3,2-b][1,4]oxazin-7-yl)acrylamide

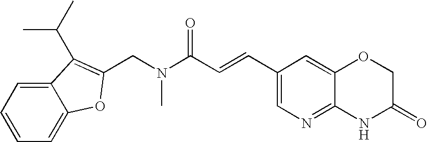

### 1-iodo-2-(3-methylbut-2-enyloxy)benzene

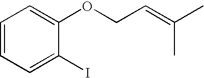

To a solution of 2-iodophenol (3.69 g, 16.8 mmol) in THF (50 mL) is added NaH (804 mg, 33.5 mmol) portion wise and stirred for 30 min at room temperature. 3,3-Dimethylallylbromide (3.9 mL, 33.5 mmol) was added and the reaction was stirred over night at room temperature. The reaction is quenched with water (20 mL) and extracted with diethyl ether (3×25 mL), the organic layers are dried over magnesium sulfate, filtered and concentrated. The compound was purified on silica gel using 100% hexanes as the eluent to yield 4.78 g (98%) of the title compound as a pale yellow oil: ¹H NMR (300 MHz, CDCl₃) δ 7.77 (d, J=6.0 Hz, 1H), 7.35 (t, J=9.0 Hz, 1H), 7.02 (d, J=9.0 Hz, 1H), 6.74 (t, J=9.0 Hz, 1H), 5.45 (m, 1H), 4.60 (d, J=6.0 Hz, 2H), 1.75 (2s, 6H)

### 3-isopropylbenzofuran

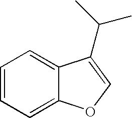

A solution of 1-iodo-2-(3-methylbut-2-enyloxy)benzene (5 g, 17.3 mmol) in propionitrile (10 mL) and diisopropylethylamine (9 mL, 52 mmol) is degassed with argon for 15 min. To this solution is added palladium acetate (423 mg, 1.73 mmol) and the reaction is heated to 100° C. overnight. The reaction is then cooled to room temperature and passed through a pad of celite, washing the filter cake with ethyl acetate (50 mL). The ethyl acetate and amine base are then removed under vacuum. The crude reaction mixture is then chromatographed using 100% hexanes to yield title compound as a colorless oil in 52% yield (1.3 g): ¹H NMR (300 MHz, CDCl₃) δ 7.7 (s, 1H), 7.68 (d, J=9.0 Hz, 1H), 7.55 (d, J=9.0 Hz, 1H), 7.32-7.24 (m, 2H), 3.11-3.07 (m, 1H), 1.32 (2s, 6H).

### 3-isopropylbenzofuran-2-carbaldehyde

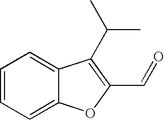

To a cooled (0° C.) solution of 3-isopropylbenzofuran (250 mg, 1.56 mmol) in THF (1 mL) is added nBuLi (2 mL, 2 mmol) drop wise and the reaction is stirred for 30 minutes. DMF (1 mL) was added to the reaction and stirred at room temperature overnight. The solution is placed in an ice bath and carefully quenched with 5% aqueous HCl solution (2 mL), and extracted with ethyl acetate (3×5 mL), dried over sodium sulfate and concentrated. The product was purified on silica with 100% hexanes to yield title compound in as a colorless oil (250 mg, 85%): ¹H NMR (400 MHz, CDCl₃) δ 10.10 (s, 1H), 7.78 (d, 1H, J=9.0 Hz), 7.55 (d, 1H, J=9.0 Hz), 7.32-7.24 (m, 2H), 3.11-3.07 (m, 1H), 1.33-1.31 (2s, 6H).

### (3-isopropylbenzofuran-2-yl)-N-methylmethanamine

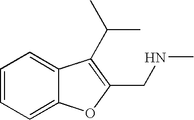

To a solution of 3-isopropylbenzofuran-2-carbaldehyde (250 mg, 1.33 mmol) in anhydrous methanol (8 mL) is added a solution of n-methylamine in ethanol (0.281 mL, 5.32 mmol) and the reaction is stirred at room temperature overnight under an atmosphere of argon. The solution is then concentrated, and re-solvated in methanol (8 mL) and cooled in an ice bath. Sodium borohydride (0.152 g, 4 mmol) was added portion wise and the reaction was stirred at room temperature under argon for 6 h. The solution is concentrated, and re-solvated in 1.3N NaOH (5 mL) and ether (5 mL) and stirred for 1 h. The ether layer was collected. The aqueous layer was washed with ether (2×10 mL), and the combined organic layers were dried over magnesium sulfate, filtered and concentrated in vacuo. Purification by chromatography (silica gel, 9:1 DCM:MeOH) yielded the title compound as a yellow oil (228 mg, 85%): ¹H NMR (400 MHz, DMSO-d₆) δ 7.70 (d, J=4.0 Hz, 1H), 7.46 (d, J=8.0 Hz, 1H), 7.25-7.16 (m, 2H), 3.75 (s, 2H), 3.15-3.22 (m, 1H), 2.25 (s, 3H), 1.36-1.34 (2s, 6H).

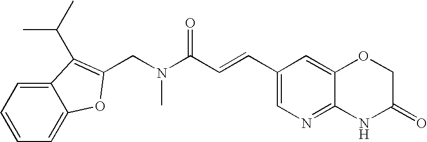

To a solution of (3-isopropylbenzofuran-2-yl)-N-methylmethanamine (115 mg, 0.57 mmol) in DMF (5 mL) were added in sequential order 3-(2,2-dimethyl-3,4-dihydro-2H-pyrido[3,2-b][1,4]oxazin-7-yl)acrylic acid hydrochloride (118 mg, 0.51 mmol), 1-hydroxybenzotriazole (77 mg, 0.57 mmol), diisopropylethylamine (289 uL, 1.54 mmol), and N-(3-dimethylaminopropyl)-N′-ethylcarbodiimide (109 mg, 0.57 mmol). The mixture was stirred at room temperature overnight, cooled in an ice bath and water was added with rapid stirring. The product was extracted with ethyl acetate (3×10 mL), dried over sodium sulfate, filtered and concentrated to give a light brown solid (14 mg, 7%): ¹H NMR (400 MHz, CD₃OD) δ 11.42 (s, 1H), 8.17-8.16 (m, 1H), 7.89-7.86 (m, 1H), 7.74-7.72 (m, 2H), 7.24-7.30 (m, 2H), 7.22-7.17 (m, 2H), 4.97 (s, 2H), 4.78 (s, 2H), 3.15 (s, 3H), 3.10-3.14 (m, 1H), 1.34 (s, 6H); MS (ESI) m/e 406 (C₂₃H₂₃N₃O₄+H)⁺.

### Example 74

### (E)-N-((3-ethylbenzofuran-2-yl)methyl)-N-methyl-3-(3-oxo-3,4-dihydro-2H-pyrido[3,2-b][1,4]oxazin-7-yl)acrylamide

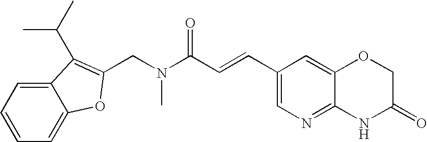

### a) (3-ethylbenzofuran-2-yl)-N-methylmethanamine, EDC, HOBt, DIPEA, DMF

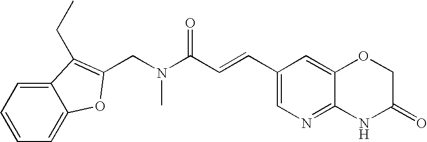

To a solution of (3-ethylbenzofuran-2-yl)-N-methylmethanamine (115 mg, 0.6 mmol) in DMF (5 mL) were added in sequential order (E)-3-(3-oxo-3,4-dihydro-2H-pyrido[3,2-b][1,4]oxazin-7-yl)acrylic acid (140 mg, 0.54 mmol), 1-hydroxybenzotriazole (84 mg, 0.6 mmol), diisopropylethylamine (282 uL, 1.62 mmol), and N-(3-dimethylaminopropyl)-N′-ethylcarbodiimide (120 mg, 0.6 mmol). The mixture was stirred at room temperature overnight, cooled in an ice bath and water added with rapid stirring. The product precipitated and was filtered, triturated with ether and dried to yield the title compound as a white solid (113 mg, 52%): ¹H NMR (400 MHz, DMSO-d₆) δ 11.41 (s, 1H), 8.19 (s, 1H), 7.88 (d, J=6.4 Hz, 1H), 7.66 (d, J=7.6 Hz, 1H), 7.51-7.49 (m, 2H), 7.29-7.25 (m, 3H), 4.99-4.79 (rotamers, 2s, 2H), 4.68 (s, 2H), 3.39 (s, 3H), 2.79-2.73 (m, 2H), 1.23-1.20 (m, 3H); MS (ESI) m/e 392 (C₂₂H₂₁N₃O₄+H)⁺.

### Example 75

### (E)-N-((5-fluoro-3-methylbenzo[b]thiophen-2-yl)methyl)-N-methyl-3-(3-oxo-3,4-dihydro-2H-pyrido[3,2-b][1,4]oxazin-7-yl)acrylamide

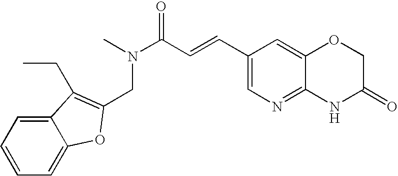

### a) ((5-fluoro-3-methylbenzo[b]thiophen-2-yl)-N-methyl methanamine, EDC, HOBt, DIPEA,

To a solution of (5-fluoro-3-methylbenzo[b]thiophen-2-yl)-N-methyl methanamine (200 mg, 0.96 mmol) in DMF (5 mL) were added in sequential order (E)-3-(3-oxo-3,4-dihydro-2H-pyrido[3,2-b][1,4]oxazin-7-yl)acrylic acid (185 mg, 0.87 mmol), 1-hydroxybenzotriazole (133 mg, 0.96 mmol), diisopropylethylamine (454 uL, 2.61 mmol), and N-(3-dimethylaminopropyl)-N′-ethylcarbodiimide (192 mg, 0.96 mmol). The mixture was stirred at room temperature overnight, cooled in an ice bath and water added with rapid stirring. The product was extracted with ethyl acetate (3×10 mL), dried with sodium sulfate, filtered and concentrated. The crude product was purified using preparative HPLC to give the title compound (45 mg, 13%): ¹H NMR (400 MHz, DMSO-d₆) δ 11.42 (bs, 1H), 8.19 (s, 1H), 7.89-7.87 (m, 2H), 7.55-7.51 (m, 2H), 7.22-7.18 (m, 2H), 5.12-4.87 (2s, 2H, rotamers), 4.68 (s, 2H), 3.54 (s, 3H), 2.39 (s, 3H). MS (ESI) m/e 412 (C₂₁H₁₈FN₃O₃S+H)⁺.

### Example 76

### Preparation of (E)-N-methyl-N-((3-methylbenzofuran-2-yl)methyl)-3-(8-oxo-6,7,8,9-tetrahydro-5H-pyrido[2,3-b]azepin-3-yl)acrylamide

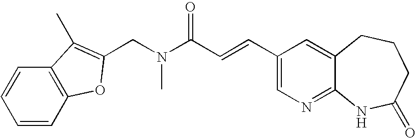

### (6,7-dihydroquinolin-8(5H)-one oxime)

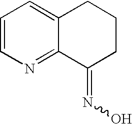

The procedure was performed as described in the literature (E. J. McEachem, W. Yang, G. Chen, R. T. Skerlj, G. J. Bridger; Syn Comm, 33, 20, 3497 (2003)). The title compound (6,7-dihydroquinolin-8(5H)-one oxime) (7.06 g, 58%) was obtained as a brown solid: ¹H NMR (400 MHz, DMSO-d₆) δ 11.38 (s, 1H), 8.42 (s, 1H), 7.59 (d, J=7.6 Hz, 1H), 7.25 (bs, 1H), 2.74-2.72 (m, 4H), 1.76 (m, 2H).

### (O-tosyl-6,7-dihydroquinolin-8(5H)-one oxime

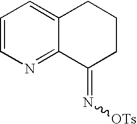

To a stirring solution of 6,7-dihydroquinolin-8(5H)-one oxime (100 mg, 0.617 mmol) and TEA (0.25 mL, 1.80 mmol) in anhydrous CH₂Cl₂ (1.5 mL) was added p-toluenesulfonyl chloride (352 mg, 1.85 mmol). After stirring for 12 h at room temperature the reaction was diluted with water (10 mL) and extracted with CH₂Cl₂ (3×10 mL). Combined organic layers were washed with brine (20 mL), dried over Na₂SO₄, filtered and concentrated to give a brown solid. After purification by column chromatography (gradient elution of CH₂Cl₂ to 5% MeOH/CH₂Cl₂) the title compound (O-tosyl-6,7-dihydroquinolin-8(5H)-one oxime) (153 mg, 78%) was obtained as a brown solid: ¹H NMR (400 MHz, DMSO-d₆) δ 8.51-8.49 (m, 1H), 7.87 (d, J=8.4 Hz, 2H), 7.69 (d, J=8.4 Hz, 1H), 7.48 (d, J=8.4 Hz, 2H), 7.42-7.40 (m, 1H), 2.91-2.89 (m, 2H), 2.80-2.77 (m, 2H), 2.41 (s, 3H), 1.80-1.77 (m, 2H).

### 6,7-dihydro-5H-pyrido[2,3-b]azepin-8(9H)-one

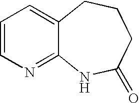

Prepared according to the standard procedure. Purification by preparative HPLC (water/acetonitrile/0.05% TFA mixture) gave the title compound (6,7-dihydro-5H-pyrido[2,3-b]azepin-8(9H)-one) (3.97 g, 84%) as an off-white solid: ¹H NMR (400 MHz, DMSO-d₆) δ 9.88 (bs, 1H), 8.25-8.23 (m, 1H), 7.71-7.68 (m, 1H), 7.13-7.10 (m, 1H), 2.71-2.67 (m, 2H), 2.23-2.19 (m, 2H), 2.16-2.10 (m, 2H).

### 3-bromo-6,7-dihydro-5H-pyrido[2,3-b]azepin-8(9H)-one

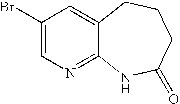

A solution of 10% Bromine (0.15 mL, 2.92 mmol) in CH₂Cl₂ was slowly added dropwise to a solution of 6,7-dihydro-5H-pyrido[2,3-b]azepin-8(9H)-one (325 mg, 2.01 mmol) and K₂CO₃ (832 mg, 6.02 mmol) in CH₂Cl₂ (3.5 mL). After stirring for 12 h at room temperature the reaction was concentrated and the resulting orange solid was dissolved in EtOAc (20 mL). Water (20 mL) was added and the aqueous layer was extracted with EtOAC (3×20 mL). The combined organic layers were washed with 10% NaHSO₃ (2×20 mL), brine (30 mL), dried over Na₂SO₄, filtered and concentrated to give the title compound (3-bromo-6,7-dihydro-5H-pyrido[2,3-b]azepin-8(9H)-one) (228 mg, 47%) as an off-white solid: ¹H NMR (400 MHz, DMSO-d₆) δ 10.0 (s, 1H), 8.35 (s, 1H), 7.97 (s, 1H), 2.71-2.67 (m, 2H), 2.25-2.21 (m, 2H), 2.16-2.11 (m, 2H).

### (E)-N-methyl-N-((3-methylbenzofuran-2-yl)methyl)-3-(8-oxo-6,7,8,9-tetrahydro-5H-pyrido[2,3-b]azepin-3-yl)acrylamide

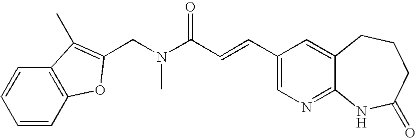

Prepared according to the standard procedure. Purification by preparative HPLC (water/acetonitrile/0.05% TFA mixture) gave the title compound ((E)-N-methyl-N-((3-methylbenzofuran-2-yl)methyl)-3-(8-oxo-6,7,8,9-tetrahydro-5H-pyrido[2,3-b]azepin-3-yl)acrylamide) (275 mg, 76%) as a white solid and a mixture of amide rotomers: ¹H NMR (400 MHz, DMSO-d₆) δ 10.0 (s, 1H), 8.53-8.51 (m, 1H), 8.14 (bs, 1H), 7.58-7.48 (m, 4H), 7.30-7.22 (m, 2H), 5.00-4.80 (m, 2H), 3.19-2.93 (m, 3H), 2.74-2.70 (m, 2H), 2.27-2.24 (m, 5H), 2.16-2.13 (m, 2H); ESI MS m/z 390 [C₂₃H₂₃N₃O₃+H]⁺.

### Example 77

### Preparation of (E)-N-methyl-N-((3-methylbenzo[b]thiophen-2-yl)methyl)-3-(8-oxo-6,7,8,9-tetrahydro-5H-pyrido[2,3-b]azepin-3-yl)acrylamide

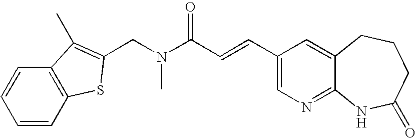

Prepared according to the standard procedure. Purification by preparative HPLC (water/acetonitrile/0.05% TFA mixture) gave the title compound ((E)-N-methyl-N-((3-methylbenzo[b]thiophen-2-yl)methyl)-3-(8-oxo-6,7,8,9-tetrahydro-5H-pyrido[2,3-b]azepin-3-yl)acrylamide) (14.8 mg, 8.8%) as a yellow solid and a mixture of amide rotomers: ¹H NMR (400 MHz, DMSO-d₆) δ 10.07 (s, 1H), 8.53 (s, 1H), 8.17-8.15 (m, 1H), 7.88-7.86 (m, 1H), 7.74-7.73 (m, 1H), 7.59-7.55 (m, 1H), 7.49-7.27 (m, 3H), 5.13-4.89 (m, 2H), 3.16-2.94 (m, 3H), 2.74-2.68 (m, 2H), 2.42 (s, 3H), 2.25-2.23 (m, 2H), 2.16-2.07 (m, 2H); ESI MS m/z 406 [C₂₃H₂₃N₃O₂S+H]⁺.

### Example 78

### Preparation of (E)-N-(3-methoxy-2-propoxybenzyl)-N-methyl-3-(8-oxo-6,7,8,9-tetrahydro-5H-pyrido[2,3-b]azepin-3-yl)acrylamide

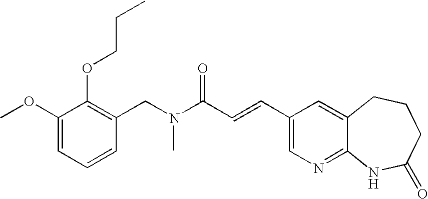

### (a) N-(3-methoxy-2-propoxybenzyl)-N-methylacrylamide, DIPEA, Pd(OAc)₂, P(o-tol)₃, DMF

Step A: Prepared according to the standard procedure. Purification by preparative HPLC (water/acetonitrile/0.05% TFA mixture) gave the title compound ((E)-N-(3-methoxy-2-propoxybenzyl)-N-methyl-3-(8-oxo-6,7,8,9-tetrahydro-5H-pyrido[2,3-b]azepin-3-yl)acrylamide) (41.0 mg, 23%) as a yellow solid and a mixture of amide rotomers: ¹H NMR (400 MHz, DMSO-d₆) δ 10.07-10.05 (m, 1H), 8.52-8.46 (m, 1H), 8.16-8.09 (m, 1H), 7.56-7.52 (m, 1H), 7.34-7.30 (m, 1H), 7.07-6.94 (m, 2H), 6.68-6.64 (m, 1H), 4.80-4.64 (m, 2H), 3.91-3.82 (m, 2H), 3.74 (s, 3H), 3.11-2.87 (m, 3H), 2.74-2.67 (m, 2H), 2.25-2.21 (m, 2H), 2.17-2.11 (m, 2H), 1.73-1.69 (m, 2H), 1.00-0.87 (m, 3H); ESI MS m/z 424 [C₂₄H₂₉N₃O₄+H]⁺.

### Example 79

### Preparation of (E)-N-((3,6-dimethyl-1H-indol-5-yl)methyl)-N-methyl-3-(8-oxo-6,7,8,9-tetrahydro-5H-pyrido[2,3-b]azepin-3-yl)acrylamide

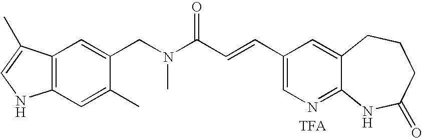

### (E)-benzyl 3-(8-oxo-6,7,8,9-tetrahydro-5H-pyrido[2,3-b]azepin-3-yl)acrylate

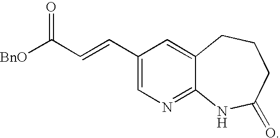

Prepared according to the standard procedure. Purification by preparative HPLC (water/acetonitrile/0.05% TFA mixture) gave the title compound ((E)-benzyl 3-(8-oxo-6,7,8,9-tetrahydro-5H-pyrido[2,3-b]azepin-3-yl)acrylate) (425 mg, 64%) as a yellow solid: ¹H NMR (400 MHz, DMSO-d₆) δ 10.12 (s, 1H), 8.55 (s, 1H), 8.15 (s, 1H), 7.70 (d, J=16.0 Hz, 1H), 7.44-7.33 (m, 5H), 6.76 (d, J=16.0 Hz, 1H), 5.23 (s, 2H), 2.72-2.69 (m, 2H), 2.27-2.24 (m, 2H), 2.17-2.11 (m, 2H).

### (E)-3-(8-oxo-6,7,8,9-tetrahydro-5H-pyrido[2,3-b]azepin-3-yl)acrylic acid hydrochloride

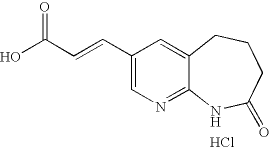

To a stirring solution of (E)-benzyl 3-(8-oxo-6,7,8,9-tetrahydro-5H-pyrido[2,3-b]azepin-3-yl)acrylate (425 mg, 1.32 mmol) in THF (15 mL) and MeOH (2.0 mL) was added 2N NaOH (3.3 mL). After stirring for 12 h at room temperature the reaction mixture was diluted with water (40 mL) and was acidified to a pH of 2 with 2N HCl. The aqueous layer was extracted with EtOAc (3×20 mL) and the combined organic layers were washed with brine (40 mL), dried over Na₂SO₄, filtered and concentrated to give a yellow oil. The oil was stirred in water (10 mL) and the solid precipitate was filtered and washed with ether (20 mL) to give the title compound ((E)-3-(8-oxo-6,7,8,9-tetrahydro-5H-pyrido[2,3-b]azepin-3-yl)acrylic acid hydrochloride) (268 mg, 76%) as a white solid: ¹H NMR (400 MHz, DMSO-d₆) δ 8.08 (s, 1H), 7.77 (s, 1H), 7.44 (d, J=16.0 Hz, 1H), 6.99 (bs, 1H), 6.33 (d, J=15.6 Hz, 1H), 2.49-2.45 (m, 2H) 2.30-2.27 (m, 2H), 1.76-1.72 (m, 2H).

### (E)-N-((3,6-dimethyl-1H-indol-5-yl)methyl)-N-methyl-3-(8-oxo-6,7,8,9-tetrahydro-5H-pyrido[2,3-b]azepin-3-yl)acrylamide

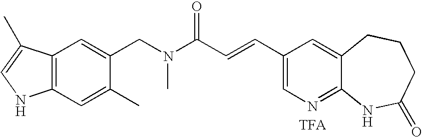

Prepared according to the standard procedure. Purification by preparative HPLC (water/acetonitrile/0.05% TFA mixture) gave the title compound ((E)-N-((3,6-dimethyl-1H-indol-5-yl)methyl)-N-methyl-3-(8-oxo-6,7,8,9-tetrahydro-5H-pyrido[2,3-b]azepin-3-yl)acrylamide)(trifluoroacetic acid salt) (33.0 mg, 15%) as a brown solid and a mixture of amide rotomers: ¹H NMR (400 MHz, DMSO-d₆) δ 10.58-10.56 (m, 1H), 10.07-10.03 (m, 1H), 8.53-8.52 (m, 1H), 8.18-8.08 (m, 1H), 7.60-7.54 (m, 1H), 7.35-7.29 (m, 1H), 7.24-7.10 (m, 2H), 7.00 (s, 1H), 4.86-4.71 (m, 2H), 3.02-2.90 (m, 3H), 2.74-2.65 (m, 2H), 2.35-2.07 (m, 10H); ESI MS m/z 403 [C₂₄H₂₆N₄O₂+H]⁺.

### Example 80

### (E)-N-methyl-N-((3-methyl-1H-indol-2-yl)methyl)-3-(4-oxo-2,3,4,5-tetrahydro-1H-pyrido[2,3-b][1,4]diazepin-8-yl)acrylamide

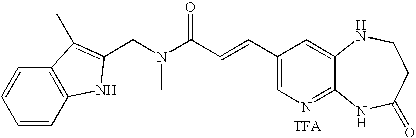

The amide was prepared according to the general amide coupling procedure in a yield of 36%. ¹H NMR (400 MHz, DMSO-d₆) δ 10.79 and 10.55 (2×s, 1H), 9.69 (s, 1H), 8.00 (s, 1H), 7.42-6.96 (m, 7H), 6.00 (br s, 1H), 4.85 and 4.75 (2×s, 2H), 3.41-3.39 (m, 2H), 3.07 and 2.85 (2×s, 3H), 2.60-2.58 (m, 2H), 2.25 (s, 3H); ESI MS m/z 390 [C₂₂H₂₃N₅O₂+H]⁺.

### REFERENCES

All publications and patents mentioned herein, including those items listed below, are hereby incorporated by reference in their entirety as if each individual publication or patent was specifically and individually incorporated by reference. In case of conflict, the present application, including any definitions herein, will control.

Heath, et al. Nature 406: 145 2000; Bergler, et al, 1994, J. Biol. Chem. 269, 5493-5496; Heath, et al, 1996, J. Biol. Chem. 271, 1833-1836; Grassberger, et al, 1984 J. Med Chem 27 947-953; Turnowsky, et al, 1989, J. Bacteriol., 171, 6555-6565; McMurry, et al, 1998 Nature 394, 531-532; Levy, et al, 1999 Nature 398, 383-384; Ward, et al, 1999 Biochem. 38, 12514-12525; Heck, Org. Reactions 1982, 27, 345; J. Het. Chem. 1978, 15, 249-251; Morb. Mortal Wkly Rep. 1998; 46:71-80; Standards, N.C.f.C.L., Methods for Dilution Antimicrobial Susceptibility Tests for Bacteria that Grow Aerobically; Approved Standard—Fifth Edition. 2000; Baxter, D. F., et al., A novel membrane potential-sensitive fluorescent dye improves cell-based assays for ion channels. J Biomol Screen, 2002 7(1): p. 79-85; Ahmed, S. A., R. M. Gogal, Jr., and J. E. Walsh, A new rapid and simple non-radioactive assay to monitor and determine the proliferation of lymphocytes: an alternative to [3H]thymidine incorporation assay. J Immunol Methods, 1994 170(2): p. 211-24; http://bbrp.llnl.gov/bbrp/html/microbe.html; http://artedi.ebc.uu.se/Projects/Francisella/; U.S. patent application Ser. Nos. 08/790,043; 10/009,219, 10/089,019; 09/968,129; 09/968,123; 09/968,236; 09/959,172; 09/979,560; 09/980,369; 10/089,755; 10/089,739; 10/089,740; PCT Application Nos. PCT/US03/38706; WO 0027628; WO 0210332; U.S. Provisional Application Nos. 60/431,406; 60/465,583; U.S. Pat. Nos. 6,531,126; 6,527,759; 6,518,270; 6,518,239; 6,517,827; 6,461,829; 6,448,054; 6,423,341; 6,495,551; 6,486,149; 6,441,162; 6,436,980; 6,399,629; 6,518,263; 6,503,881; 6,503,881; 6,486,148; 6,465,429; 6,388,070; 6,531,649; 6,531,465; 6,528,089; 6,521,408; 6,518,487; 6,531,508; 6,514,962; 6,503,953; 6,492,351; 6,486,148; 6,461,607; 6,448,054; 6,495,161; 6,495,158; 6,492,351; 6,486,165; 6,531,465; 6,514,535; 6,489,318; 6,497,886; 6,503,953; 6,503,539, 6,500,459; 6,492,351; 6,500,463; 6,461,829; 6,448,238; 6,432,444; 6,333,045; 6,291,462; 6,221,859; 6,514,986; 6,340,689; 6,309,663; 6,303,572; 6,277,836; 6,367,985; 6,468,964; 6,461,607; 6,448,449; 6,436,980; 6,423,741; 6,406,880; 6,395,746; 6,346,391; 6,294,192; 6,267,985; 6,235,908; 6,515,113; 6,509,327; 6,503,955; 6,525,066; 6,531,291; 6,517,827; 6,514,953; 6,514,541; 6,428,579; 6,451,339; 6,461,607; 6,461,829; 6,503,906; 6,518,239; 6,133,260; 6,174,878; 6,184,380; 6,187,341; 6,194,429; 6,194,441; 6,198,000; 6,221,859; 6,221,864; 6,239,113; 6,239,141; and 6,248,363.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

What is claimed is:

1. A compound of formula I: 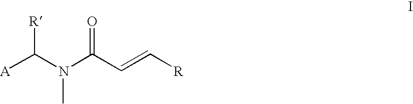
wherein, independently for each occurrence,
A is a monocyclic ring of 4-7 atoms containing 0-2 heteroatoms, a bicyclic ring of 8-12 atoms containing 0-4 heteroatoms or a tricyclic ring of 8-12 atoms containing 0-6 heteroatoms wherein the rings are independently aliphatic, aromatic, heteroaryl or heterocyclic in nature, the heteroatoms are selected from N, S or O and the rings are optionally substituted with one or more groups selected from C₁₋₄ alkyl, OR″, CN, OCF₃, F, Cl, Br, I; wherein R″ is H, alkyl, aralkyl, or heteroaralkyl;
R is 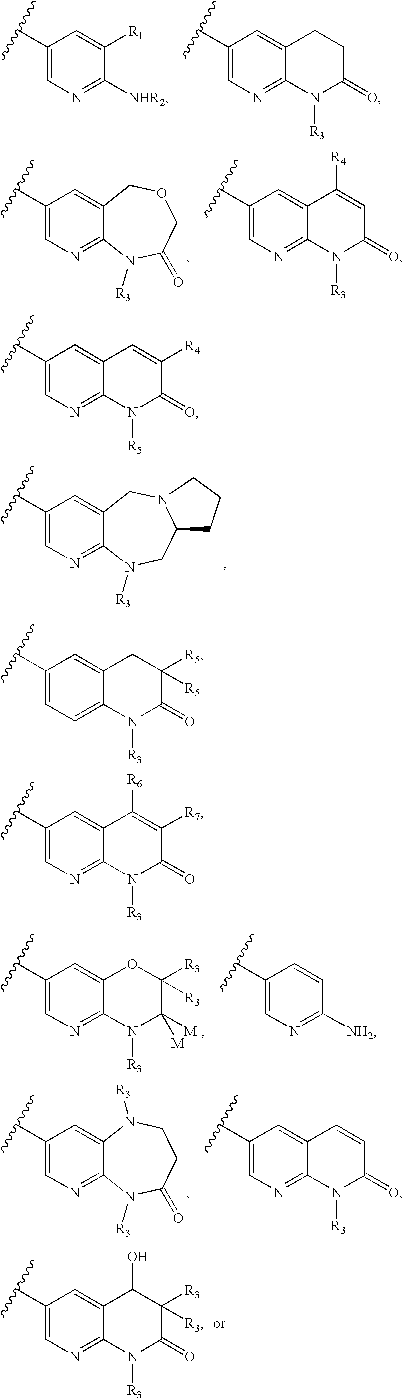 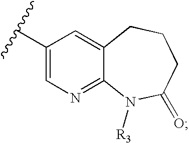
wherein, independently for each occurrence,
R₁ is OH or —O(CH₂)ₙ—Ar;
wherein,
n is an integer from 1 to 6 inclusive, and
Ar is aryl or heteroaryl;
R₂ is H or —C(O)R₃;
R₃ is H, alkyl, or aryl;
R₄ is OH or N(R₃)₂;
the two R₅ taken together form a spirocyloalkane, a spiroaryl, or a spiroheterocycloalkane;
R₆ is H, OH, alkyl, or aryl;
R₇ is alkyl, aryl, cycloalkane, or heterocycloalkane;
M is H or OH, or two M taken together form O or N(R₃); provided that when R is 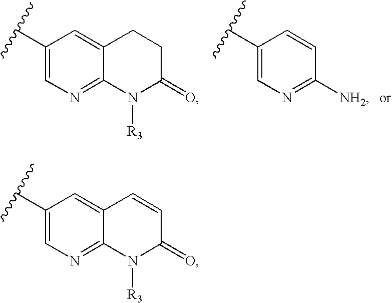
R′ is (R)-Me; and
pharmaceutically acceptable salts thereof.

2. The compound of claim 1, wherein A is selected from the following: 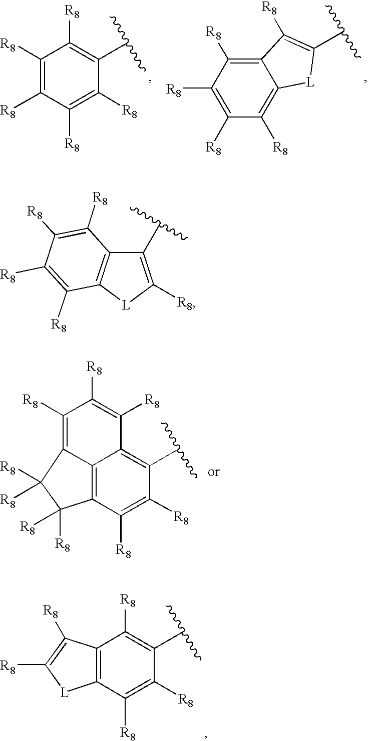
wherein, independently for each occurrence,
R₈ is H, C₁₋₄ alkyl, C₁₋₄ haloalkyl, C₁₋₄ alkenyl, OR″, CN, OCF₃, F, Cl, Br, I; wherein R″ is H, alkyl, aralkyl, or heteroaralkyl; and
L is O, S, or NR₃.

3. The compound of claim 1, wherein A is selected from the following:

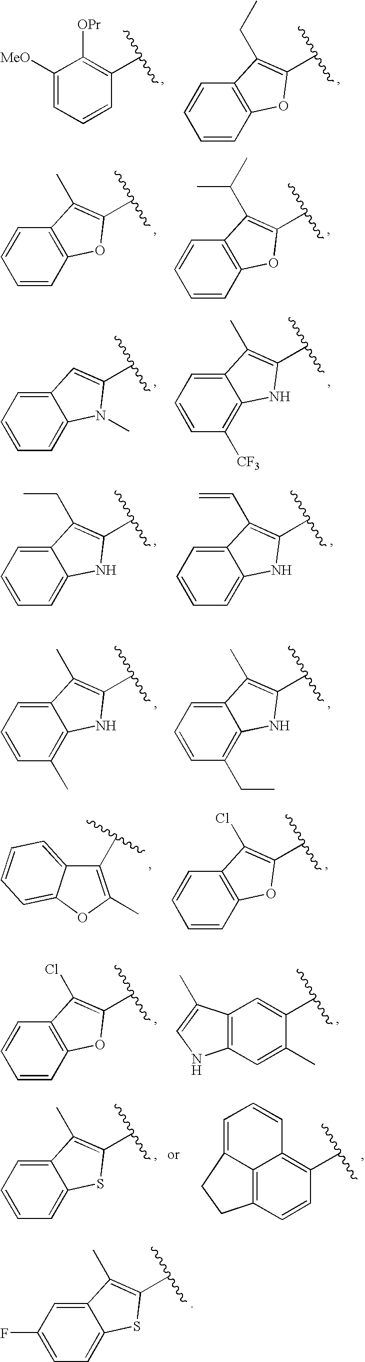

4. The compound of claim 2, wherein the compound has formula Ia: 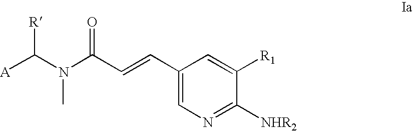
wherein,
A is selected from the following:

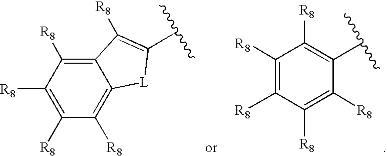

5. The compound of claim 2, wherein the compound has formula Ib: 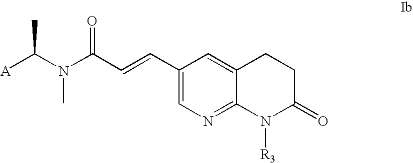
wherein,
A is selected from the following:

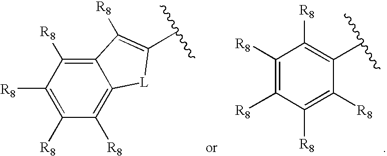

6. The compound of claim 2, wherein the compound has formula Ic: 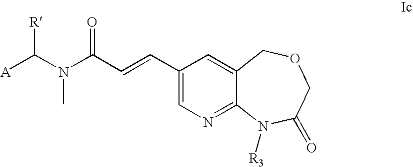
wherein,
A is selected from the following:

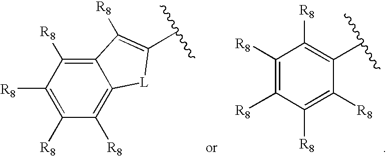

7. The compound of claim 2, wherein the compound has formula Id: 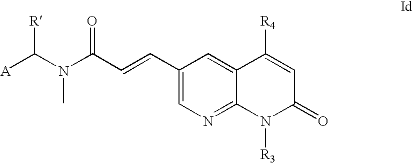
wherein,
A is selected from the following:

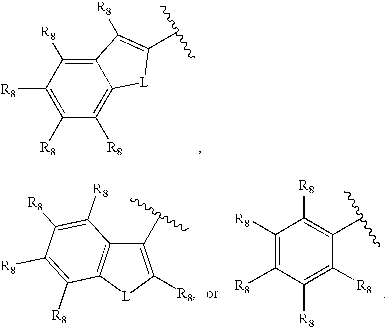

8. The compound of claim 2, wherein the compound has formula Ie:

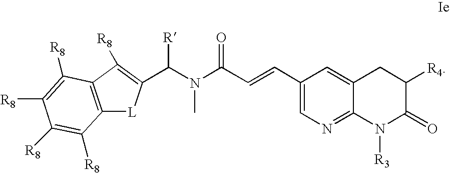

9. The compound of claim 2, wherein the compound has formula If:

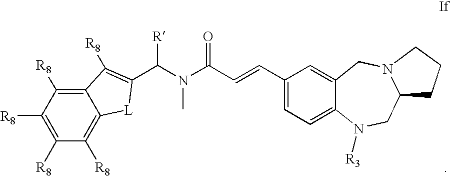

10. The compound of claim 2, wherein the compound has formula Ig: 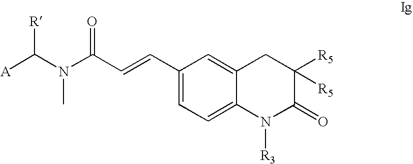
wherein,
A is selected from the following:

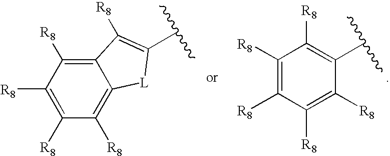

11. The compound of claim 2, wherein the compound has formula Ih: 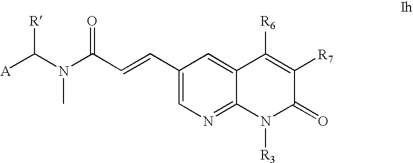
wherein,
A is selected from the following:

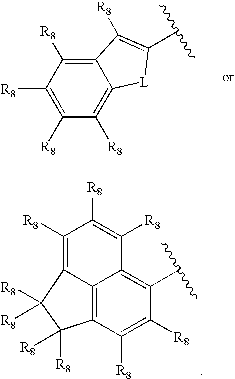

12. The compound of claim 2, wherein the compound has formula Ii: 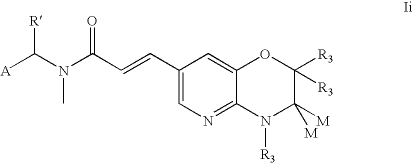
wherein,
A is selected from the following:

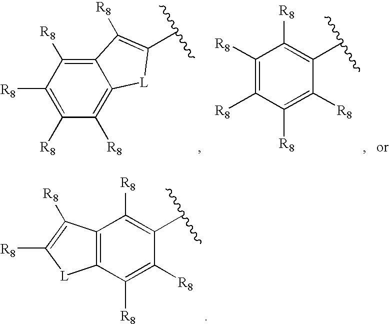

13. The compound of claim 2, wherein the compound has formula Ij:

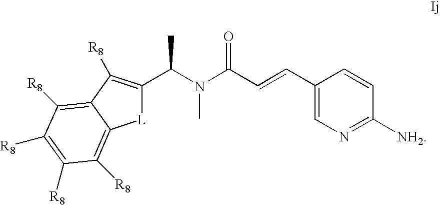

14. The compound of claim 2, wherein the compound has formula Ik: 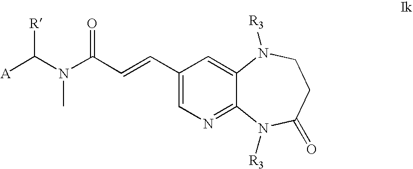
wherein,
A is selected from the following: 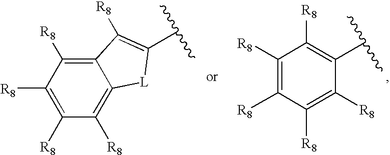
wherein R₈ and L are as defined previously.

15. The compound of claim 2, wherein the compound has formula Il:

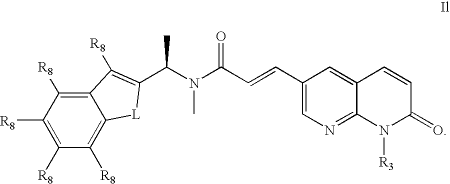

16. The compound of claim 2, wherein the compound has formula Im:

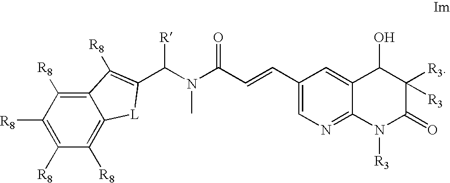

17. The compound of claim 2, wherein the compound has formula In: 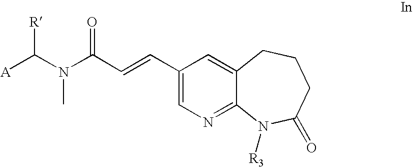
wherein,
A is selected from the following:

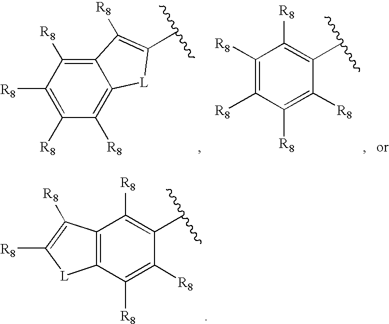

18. The compound of claim 1, wherein the compound is selected from the following:
(E)-3-[6-Amino-5-(pyridin-2-ylmethoxy)-pyridin-3-yl]-N-methyl-N-(3-methyl-benzofuran-2-ylmethyl)acrylamide;
(E)-3-[6-Amino-5-(pyridin-3-ylmethoxy)-pyridin-3-yl]-N-methyl-N-(3-methyl-benzofuran-2-ylmethyl)acrylamide;
(E)-3-(6-Acetylamino-5-hydroxy-pyridin-3-yl)-N-(3-methoxy-2-propoxy-benzyl)-N-methylacrylamide hydrochloride;
(E)-3-(6-Acetylamino-5-hydroxy-pyridin-3-yl)-N-methyl-N-(3-methyl-benzofuran-2-ylmethyl)acrylamide hydrochloride;
(E)-3-(6-Amino-5-benzyloxy-pyridin-3-yl)-N-methyl-N-(3-methyl-benzofuran-2-ylmethyl)acrylamide;
(E)-N-methyl-N-[1-(R)-(3-methyl-benzofuran-2-yl)-ethyl]-3-(7-oxo-5,6,7,8-tetrahydro-[1,8]naphthyridin-3-yl)acrylamide;
(E)-N-Methyl-N-(1-methyl-1H-indol-2-ylmethyl)-3-(8-oxo-5,7,8,9-tetrahydro-6-oxa-1,9-diazabenzocyclohepten-3-yl)-acrylamide hydrochloride;
(E)-N-Methyl-N-(3-methyl-benzofuran-2-ylmethyl)-3-(8-oxo-5,7,8,9-tetrahydro-6-oxa-1,9-diazabenzocyclohepten-3-yl)-acrylamide;
(E)-N-(3-Methoxy-2-propoxy-benzyl)-N-3-(8-oxo-5,7,8,9-tetrahydro-6-oxa-1,9-diazabenzocyclohepten-3-yl)-acrylamide;
(E)-N-Methyl-N-[1-(R)-(3-methyl-benzo[b]thiophen-2-yl)-ethyl]-3-(7-oxo-5,6,7,8-tetrahydro-[1,8]naphthyridin-3-yl)-acrylamide;
(E)-N-Methyl-N-[1-(3-methyl-benzo[b]thiophen-2-yl)-ethyl]-3-(8-oxo-5,7,8,9-tetrahydro-6-oxa-1,9-diaza-benzocyclohepten-3-yl)-acrylamide;
(E)-3-(5-Amino-7-oxo-7,8-dihydro-[1,8]naphthyridin-3-yl)-N-methyl-N-(1-methyl-1H-indol-2-ylmethyl)-acrylamide hydrochloride;
(E)-3-(5-hydroxy-7-oxo-7,8-dihydro-[1,8]naphthyridin-3-yl)-N-methyl-N-(1-methyl-1H-indol-2-ylmethyl)-acrylamide;
(E)-3-(5-hydroxy-7-oxo-7,8-dihydro-[1,8]naphthyridin-3-yl)-N-methyl-N-(3-methyl-benzofuran-2-ylmethyl)-acrylamide;
(E)-N-(2-ethoxy-3-methoxy-benzyl)-3-(5-hydroxy-7-oxo-7,8-dihydro-[1,8]naphthyridin-3-yl)-N-methyl-acrylamide;
(E)-3-(5-hydroxy-7-oxo-7,8-dihydro-[1,8]naphthyridin-3-yl)-N-(3-methoxy-2-propoxy-benzyl)-N-methyl-acrylamide;
(E)-N-(3-Chloro-benzofuran-2-ylmethyl)-N-methyl-3-(2-oxo-1,2,3,5-tetrahydro-benzo[e][1,4]oxazepin-7-yl)-acrylamide;
(S,E)-3-(3,4-cyclopentyl-2,3,4,5-tetrahydro-1H-pyrido[2,3-e][1,4]diazepin-7-yl)-N-methyl-(E)-N-((3-methylbenzofuran-2-yl)methyl)acrylamide trifluoroacetic acid salt;
(E)-N-methyl-N-((3-methylbenzofuran-2-yl)methyl)-3-[7-oxo-(4′-N-Boc-6-spiropiperidinyl)-1,8-naphthyridin-3-yl]acrylamide;
(E)-N-methyl-N-((3-methylbenzofuran-2-yl)methyl)-3-[7-oxo-(6-spiropiperidinyl)-1,8-naphthyridin-3-yl]acrylamide hydrochloride;
(E)-N-methyl-N-((3-methylbenzothiophene-2-yl)methyl)-3-[7-oxo-(4′-N-Boc-6-spiropiperidinyl)-1,8-naphthyridin-3-yl]acrylamide;
(E)-N-methyl-N-((3-methylbenzothiophen-2-yl)methyl)-3-[7-oxo-(6-spiropiperidinyl)-1,8-naphthyridin-3-yl]acrylamide trifluoroacetate salt;
(E)-N-methyl-N-((1-methyl-1H-indol-2-yl)methyl)-3-[7-oxo-(4′-N-Boc-6-spiropiperidinyl)-1,8-naphthyridin-3-yl]acrylamide;
(E)-N-methyl-N-((1-methyl-1H-indol-2-yl)methyl)-3-[7-oxo-(4′-6-spiropiperidinyl)-1,8-naphthyridin-3-yl]acrylamide trifluoroacetate salt;
(E)-N-(3-methoxy-2-propoxybenzyl)-N-methyl-3-[7-oxo-(4′-N-Boc-6-spiropiperidinyl)-1,8-naphthyridin-3-yl]acrylamide;
(E)-N-(3-methoxy-2-propoxybenzyl)-N-methyl-3-[7-oxo-(6-spiropiperidinyl)-1,8-naphthyridin-3-yl]acrylamide hydrochloride;
(E)-N-methyl-N-((3-methylbenzofuran-2-yl)methyl)-3-[7-oxo-(6-4′-N-methyl-spiropiperidinyl)-1,8-naphthyridin-3-yl]acrylamide hydrochloride;
(E)-N-(3-methoxy-2-propoxybenzyl)-N-methyl-3-[7-oxo-(4′-N-methyl-6-spiropiperidinyl)-1,8-naphthyridin-3-yl]acrylamide hydrochloride;
(E)-N-methyl-N-((3-methylbenzothiophen-2-yl)methyl)-3-[7-oxo-(4′-N-methyl-6-spiropiperidinyl)-1,8-naphthyridin-3-yl]acrylamide trifluoroacetate salt;
(E)-3-(6,6-(4-N-methylpiperidine)-7-oxo-5,6,7,8-tetrahydro-1,8-naphthyridin-3-yl)-N-((5-fluoro-3-methylbenzo[b]thiophen-2-yl)methyl)-N-methylacrylamide;
(E)-N-methyl-N-((3-methylbenzofuran-2-yl)methyl)-3-(6-morpholino-7-oxo-7,8-dihydro-1,8-naphthyridin-3-yl)acrylamide;
(E)-N-methyl-N-((3-methylbenzofuran-2-yl)methyl)-3-(6-morpholino-7-oxo-7,8-dihydro-1,8-naphthyridin-3-yl)acrylamide;
(E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride;
(E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride;
(R,E)-N-(1-(3-ethylbenzofuran-2-yl)ethyl)-N-methyl-3-(7-oxo-5,6,7,8-tetrahydro-1,8-naphthyridin-3-yl)acrylamide;
(R,E)-3-(2,2-dimethyl-3-oxo-3,4-dihydro-2H-pyrido[3,2-b][1,4]oxazin-7-yl)-N-(1-(3-ethylbenzofuran-2-yl)ethyl)-N-methylacrylamide;
(R,E)-3-(6-aminopyridin-3-yl)-N-(1-(3-ethylbenzofuran-2-yl)ethyl)-N-methylacrylamide;
(E)-3-(3-hydroxy-2,2-dimethyl-3,4-dihydro-2H-pyrido[3,2-b][1,4]oxazin-7-yl)-N-methyl-N-((3-methylbenzofuran-2-yl)methyl)acrylamide trifluoroacetate salt;
(E)-3-(5-hydroxy-6-isopropyl-7-oxo-7,8-dihydro-1,8-naphthyridin-3-yl)-N-methyl-N-((3-methylbenzofuran-2-yl)methyl)acrylamide;
(E)-N-((1,2-dihydroacenaphthylen-5-yl)methyl)-3-(5-hydroxy-6-isopropyl-7-oxo-7,8-dihydro-1,8-naphthyridin-3-yl)-N-methylacrylamide;
(E)-3-(5-hydroxy-6-ethyl-7-oxo-7,8-dihydro-1,8-naphthyridin-3-yl)-N-methyl-N-((3-methylbenzofuran-2-yl)methyl)acrylamide;
(E)-N-((1,2-dihydroacenaphthylen-5-yl)methyl)-3-(6-ethyl-5-hydroxy-7-oxo-7,8-dihydro-1,8-naphthyridin-3-yl)-N-methylacrylamide;
(E)-3-((E)-2,2-dimethyl-3-(methylimino)-3,4-dihydro-2H-pyrido[3,2-b][1,4]oxazin-7-yl)-N-methyl-N-((3-methylbenzofuran-2-yl)methyl)acrylamide hydrochloride;
(E)-3-((E)-2,2-dimethyl-3-(methylimino)-3,4-dihydro-2H-pyrido[3,2-b][1,4]oxazin-7-yl)-N-methyl-N-((3-methylbenzo[b]thiophen-2-yl)methyl)acrylamide;
(E)-3-(3-imino-2,2-dimethyl-3,4-dihydro-2H-pyrido[3,2-b][1,4]oxazin-7-yl)-N-methyl-N-((3-methylbenzofuran-2-yl)methyl)acrylamide;
(E)-N-methyl-N-((3-methyl-1H-indol-2-yl)methyl)-3-(2-oxo-1,2,3,5-tetrahydropyrido[2,3-e][1,4]oxazepin-7-yl)acrylamide;
(E)-N-((1,3-dimethyl-1H-indol-2-yl)methyl)-N-methyl-3-(2-oxo-2,3,4,5-tetrahydro-1H-pyrido[2,3-b][1,4]diazepin-8-yl)acrylamide;
(E)-N-methyl-N-((3-methylbenzofuran-2-yl)methyl)-3-(4-oxo-2,3,4,5-tetrahydro-1H-pyrido[2,3-b][1,4]diazepin-8-yl)acrylamide;
(E)-N-methyl-N-((3-methylbenzofuran-2-yl)methyl)-3-(4-oxo-2,3,4,5-tetrahydro-1H-pyrido[2,3-b][1,4]diazepin-8-yl)acrylamide;
(E)-N-methyl-N-((3-methyl-1H-indol-2-yl)methyl) 3-(4-oxo-2,3,4,5-tetrahydro-1H-pyrido[2,3-b][1,4]diazepin-8-yl)acrylamide;
(R,E)-N-methyl-N-(1-(3-methylbenzofuran-2-yl)ethyl)-3-(2-oxo-1,2,3,5-tetrahydropyrido[2,3-e][1,4]oxazepin-7-yl)acrylamide;
(R,E)-N-methyl-N-(1-(3-methylbenzofuran-2-yl)ethyl)-3-(7-oxo-7,8-dihydro-1,8-naphthyridin-3-yl)acrylamide;
(R,E)-N-methyl-N-(1-(3-methylbenzofuran-2-yl)ethyl)-3-(3-oxo-3,4-dihydro-2H-pyrido[3,2-b][1,4]oxazin-7-yl)acrylamide;
(R,E)-N-(1-(3-methoxy-2-propoxyphenyl)ethyl)-N-methyl-3-(7-oxo-5,6,7,8-tetrahydro-1,8-naphthyridin-3-yl)acrylamide;
(E)-N-(3-methoxy-2-propoxybenzyl)-N-methyl-3-(4-oxo-2,3,4,5-tetrahydro-1H-pyrido[2,3-b][1,4]diazepin-8-yl)acrylamide;
(R,E)-N-methyl-N-(1-(3-methylbenzofuran-2-yl)ethyl)-3-(4-oxo-2,3,4,5-tetrahydro-1H-pyrido[2,3-b]diazepin-8-yl)acrylamide;
(E)-N-methyl-N-((1-methyl-1H-indol-2-yl)methyl)-3-(4-oxo-2,3,4,5-tetrahydro-1H-pyrido[2,3-b][1,4]diazepin-8-yl)acrylamide;
(E)-N-((5-fluoro-3-methylbenzo[b]thiophen-2-yl)methyl)-N-methyl-3-(4-oxo-2,3,4,5-tetrahydro-1H-pyrido[2,3-b][1,4]diazepin-8-yl)acrylamide;
(R,E)-N-(1-(3-ethylbenzofuran-2-yl)ethyl)-N-methyl-3-(4-oxo-2,3,4,5-tetrahydro-1H-pyrido[2,3-b][1,4]diazepin-8-yl)acrylamide;
(E)-3-(5-hydroxy-6,6-dimethyl-7-oxo-5,6,7,8-tetrahydro-1,8-naphthyridin-3-yl)-N-methyl-N-((3-methylbenzofuran-2-yl)methyl)acrylamide;
(E)-3-(5-hydroxy-6,6-dimethyl-7-oxo-5,6,7,8-tetrahydro-1,8-naphthyridin-3-yl)-N-methyl-N-((1-methyl-1H-indol-2-yl)methyl)acrylamide;
(R,E)-3-(5-hydroxy-6,6-dimethyl-7-oxo-5,6,7,8-tetrahydro-1,8-naphthyridin-3-yl)-N-methyl-N-(1-(3-methylbenzofuran-2-yl)ethyl)acrylamide;
(R,E)-N-(1-(3-ethylbenzofuran-2-yl)ethyl)-N-methyl-3-(7-oxo-5,6,7,8-tetrahydro-1,8-naphthyridin-3-yl)acrylamide;
N-methyl-N-[1-(R)-(3-ethyl-benzofuran-2-yl)-ethyl]-3-(7-oxo-5,6,7,8-tetrahydro-[1,8]naphthyridin-3-yl)acrylamide;
(R,E)-3-(2,2-dimethyl-3-oxo-3,4-dihydro-2H-pyrido[3,2-b][1,4]oxazin-7-yl)-N-(1-(3-ethylbenzofuran-2-yl)ethyl)-N-methylacrylamide;
(R,E)-3-(6-aminopyridin-3-yl)-N-(1-(3-ethylbenzofuran-2-yl)ethyl)-N-methylacrylamide;
(E)-3-(3-hydroxy-2,2-dimethyl-3,4-dihydro-2H-pyrido[3,2-b][1,4]oxazin-7-yl)-N-methyl-N-((3-methylbenzofuran-2-yl)methyl)acrylamide trifluoroacetate salt;
(E)-3-(5-hydroxy-6-isopropyl-7-oxo-7,8-dihydro-1,8-naphthyridin-3-yl)-N-methyl-N-((3-methylbenzofuran-2-yl)methyl)acrylamide;
(E)-N-((1,2-dihydroacenaphthylen-5-yl)methyl)-3-(5-hydroxy-6-isopropyl-7-oxo-7,8-dihydro-1,8-naphthyridin-3-yl)-N-methylacrylamide;
(E)-3-(5-hydroxy-6-ethyl-7-oxo-7,8-dihydro-1,8-naphthyridin-3-yl)-N-methyl-N-((3-methylbenzofuran-2-yl)methyl)acrylamide;
(E)-N-((1,2-dihydroacenaphthylen-5-yl)methyl)-3-(6-ethyl-5-hydroxy-7-oxo-7,8-dihydro-1,8-naphthyridin-3-yl)-N-methylacrylamide;
(E)-3-((E)-2,2-dimethyl-3-(methylimino)-3,4-dihydro-2H-pyrido[3,2-b][1,4]oxazin-7-yl)-N-methyl-N-((3-methylbenzofuran-2-yl)methyl)acrylamide hydrochloride;
(E)-3-((E)-2,2-dimethyl-3-(methylimino)-3,4-dihydro-2H-pyrido[3,2-b][1,4]oxazin-7-yl)-N-methyl-N-((3-methylbenzo[b]thiophen-2-yl)methyl)acrylamide;
(E)-3-(3-imino-2,2-dimethyl-3,4-dihydro-2H-pyrido[3,2-b][1,4]oxazin-7-yl)-N-methyl-N-((3-methylbenzofuran-2-yl)methyl)acrylamide;
(E)-N-methyl-N-((3-methyl-1H-indol-2-yl)methyl)-3-(2-oxo-1,2,3,5-tetrahydropyrido[2,3-e][1,4]oxazepin-7-yl)acrylamide;
(E)-N-methyl-N-((3-methyl-1H-indol-2-yl)methyl)-3-(3-oxo-3,4-dihydro-2H-pyrido[3,2-b][1,4]oxazin-7-yl)acrylamide;
(E)-N-methyl-N-((3-methyl-1H-indol-2-yl)methyl)-3-(1,2,3,5-tetrahydropyrido[2,3-e][1,4]oxazepin-7-yl)acrylamide;
(E)-N-((3-ethyl-1H-indol-2-yl)methyl)-N-methyl-3-(3-oxo-3,4-dihydro-2H-pyrido[3,2-b][1,4]oxazin-7-yl)acrylamide;
(E)-N-methyl-3-(3-oxo-3,4-dihydro-2H-pyrido[3,2-b][1,4]oxazin-7-yl)-N-((3-vinyl-1H-indol-2-yl)methyl)acrylamide;
(E)-N-((1,3-dimethyl-1H-indol-2-yl)methyl)-N-methyl-3-(3-oxo-3,4-dihydro-2H-pyrido[3,2-b][1,4]oxazin-7-yl)acrylamide;
(E)-N-((1,3-dimethyl-1H-indol-2-yl)methyl)-N-methyl-3-3-(7-oxo-5,6,7,8-tetrahydro-1,8-naphthyridin-3-yl)acrylamide;
(E)-N-((1,3-dimethyl-1H-indol-2-yl)methyl)-N-methyl-3-(2-oxo-2,3,4,5-tetrahydro-1H-pyrido[2,3-b][1,4]diazepin-8-yl)acrylamide;
(E)-N-((3,7-dimethyl-1H-indol-2-yl)methyl)-N-methyl-3-(3-oxo-3,4-dihydro-2H-pyrido[3,2-b][1,4]oxazin-7-yl)acrylamide;
(E)-N-methyl-N-((3,7-methyl-1H-indol-2-yl)methyl-3-(7-oxo-5,6,7,8-tetrahydro-1,8-naphthyridin-3-yl)acrylamide;
(E)-N-((3,7-dimethyl-1H-indol-2-yl)methyl)-N-methyl-3-(8-oxo-6,7,8,9-tetrahydro-5H-pyrido[2,3-b]azepin-3-yl)acrylamide;
(E)-N-methyl-N-((3-methyl-7-(trifluoromethyl)-1H-indol-2-yl)methyl)-3-(3-oxo-3,4-dihydro-2H-pyrido[3,2-b][1,4]oxazin-7-yl)acrylamide;
(E)-N-((7-ethyl-3-methyl-1H-indol-2-yl)methyl)-N-methyl-3-(3-oxo-3,4-dihydro-2H-pyrido[3,2-b][1,4]oxazin-7-yl)acrylamide;
(E)-N-((3,6-dimethyl-1H-indol-5-yl)methyl)-N-methyl-3-(3-oxo-3,4-dihydro-2H-pyrido[3,2-b][1,4]oxazin-yl)acrylamide);
(E)-3-(2,2-dimethyl-3,4-dihydro-2H-pyrido[3,2-b][1,4]oxazin-7-yl)-N-methyl-N-((3-methylbenzofuran-2-yl)methyl)acrylamide hydrochloride;
(E)-N-((3-chlorobenzofuran-2-yl)methyl)-N-methyl-3-(3-oxo-3,4-dihydro-2H-pyrido[3,2-b][1,4]oxazin-7-yl)acrylamide;
(E)-N-(3-methoxy-2-propoxybenzyl)-N-methyl-3-(3-oxo-3,4-dihydro-2H-pyrido[3,2-b][1,4]oxazin-7-yl)acrylamide;
(E)-N-((3-isopropylbenzofuran-2-yl)methyl)-N-methyl-3-(3-oxo-3,4-dihydro-2H-pyrido[3,2-b][1,4]oxazin-7-yl)acrylamide;
(E)-N-((3-ethylbenzofuran-2-yl)methyl)-N-methyl-3-(3-oxo-3,4-dihydro-2H-pyrido[3,2-b][1,4]oxazin-7-yl)acrylamide;
(E)-N-((5-fluoro-3-methylbenzo[b]thiophen-2-yl)methyl)-N-methyl-3-(3-oxo-3,4-dihydro-2H-pyrido[3,2-b][1,4]oxazin-7-yl)acrylamide;
(E)-N-methyl-N-((3-methylbenzofuran-2-yl)methyl)-3-(8-oxo-6,7,8,9-tetrahydro-5H-pyrido[2,3-b]azepin-3-yl)acrylamide;
(E)-N-methyl-N-((3-methylbenzofuran-2-yl)methyl)-3-(8-oxo-6,7,8,9-tetrahydro-5H-pyrido[2,3-b]azepin-3-yl)acrylamide;
(E)-N-methyl-N-((3-methylbenzo[b]thiophen-2-yl)methyl)-3-(8-oxo-6,7,8,9-tetrahydro-5H-pyrido[2,3-b]azepin-3-yl)acrylamide;
(E)-N-(3-methoxy-2-propoxybenzyl)-N-methyl-3-(8-oxo-6,7,8,9-tetrahydro-5H-pyrido[2,3-b]azepin-3-yl)acrylamide;
(E)-N-((3,6-dimethyl-1H-indol-5-yl)methyl)-N-methyl-3-(8-oxo-6,7,8,9-tetrahydro-5H-pyrido[2,3-b]azepin-3-yl)acrylamide;
(E)-N-((3,6-dimethyl-1H-indol-5-yl)methyl)-N-methyl-3-(8-oxo-6,7,8,9-tetrahydro-5H-pyrido[2,3-b]azepin-3-yl)acrylamide;
(S,E)-3-(3,4-cyclopentyl-2,3,4,5-tetrahydro-1H-pyrido[2,3-e][1,4]diazepin-7-yl)-N-methyl-N-((3-methylbenzofuran-2-yl)methyl)acrylamide trifluoroacetic acid salt;
(E)-N-methyl-N-((3-methylbenzofuran-2-yl)methyl)-3-[7-oxo-(4′-N-Boc-6-spiropiperidinyl)-1,8-naphthyridin-3-yl]acrylamide;
(E)-N-methyl-N-((3-methylbenzofuran-2-yl)methyl)-3-[7-oxo-(6-spiropiperidinyl)-1,8-naphthyridin-3-yl]acrylamide hydrochloride;
(E)-N-methyl-N-((3-methylbenzothiophen-2-yl)methyl)-3-[7-oxo-(6-spiropiperidinyl)-1,8-naphthyridin-3-yl]acrylamide trifluoroacetate salt;
(E)-N-methyl-N-((3-methylbenzothiophene-2-yl)methyl)-3-[7-oxo-(4′-N-Boc-6-spiropiperidinyl)-1,8-naphthyridin-3-yl]acrylamide;
(E)-N-methyl-N-((1-methyl-1H-indol-2-yl)methyl)-3-[7-oxo-(4′-6-spiropiperidinyl)-1,8-naphthyridin-3-yl]acrylamide trifluoroacetate salt;
(E)-N-methyl-N-((1-methyl-1H-indol-2-yl)methyl)-3-[7-oxo-(4′-N-Boc-6-spiropiperidinyl)-1,8-naphthyridin-3-yl]acrylamide;
(E)-N-(3-methoxy-2-propoxybenzyl)-N-methyl-3-[7-oxo-(4′-N-Boc-6-spiropiperidinyl)-1,8-naphthyridin-3-yl]acrylamide;
(E)-N-(3-methoxy-2-propoxybenzyl)-N-methyl-3-[7-oxo-(6-spiropiperidinyl)-1,8-naphthyridin-3-yl]acrylamide hydrochloride;
(E)-N-methyl-N-((3-methylbenzofuran-2-yl)methyl)-3-[7-oxo-(6-4′-N-methyl-spiropiperidinyl)-1,8-naphthyridin-3-yl]acrylamide hydrochloride;
(E)-N-(3-methoxy-2-propoxybenzyl)-N-methyl-3-[7-oxo-(4′-N-methyl-6-spiropiperidinyl)-1,8-naphthyridin-3-yl]acrylamide hydrochloride;
(E)-N-methyl-N-((3-methylbenzothiophen-2-yl)methyl)-3-[7-oxo-(4′-N-methyl-6-spiropiperidinyl)-1,8-naphthyridin-3-yl]acrylamide trifluoroacetate salt;
(E)-3-(6,6-(4-N-methylpiperidine)-7-oxo-5,6,7,8-tetrahydro-1,8-naphthyridin-3-yl)-N-((5-fluoro-3-methylbenzo[b]thiophen-2-yl)methyl)-N-methylacrylamide;
(E)-N-methyl-N-((3-methylbenzofuran-2-yl)methyl)-3-(6-morpholino-7-oxo-7,8-dihydro-1,8-naphthyridin-3-yl)acrylamide;
(E)-N-methyl-N-((3-methylbenzofuran-2-yl)methyl)-3-(6-morpholino-7-oxo-7,8-dihydro-1,8-naphthyridin-3-yl)acrylamide;
(E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride;
(E)-N-methyl-N-((3-methylbenzofuran-2-yl)methyl)-3-(7-oxo-6-(piperazin-1-yl)-7,8-dihydro-1,8-naphthyridin-3-yl)acrylamide hydrochloride;
(E)-N-methyl-N-((3-methylbenzofuran-2-yl)methyl)-3-(4-oxo-2,3,4,5-tetrahydro-1H-pyrido[2,3-b][1,4]diazepin-8-yl)acrylamide;
(E)-3-(4-oxo-2,3,4,5-tetrahydro-1H-pyrido[2,3-b][1,4]diazepin-8-yl)acrylic acid hydrochloride;
(E)-N-methyl-N-((3-methyl-1H-indol-2-yl)methyl)-3-(4-oxo-2,3,4,5-tetrahydro-1H-pyrido[2,3-b][1,4]diazepin-8-yl)acrylamide;
(R,E)-3-(2,2-dimethyl-3-oxo-3,4-dihydro-2H-pyrido[3,2-b]oxazin-7-yl)-N-methyl-N-(1-(3-methylbenzofuran-2-yl)ethyl)acrylamide;
(R,E)-N-methyl-N-(1-(3-methylbenzofuran-2-yl)ethyl)-3-(2-oxo-1,2,3,5-tetrahydropyrido[2,3-e][1,4]oxazepin-7-yl)acrylamide;
(R,E)-N-methyl-N-(1-(3-methylbenzofuran-2-yl)ethyl)-3-(7-oxo-7,8-dihydro-1,8-naphthyridin-3-yl)acrylamide;
(R,E)-N-methyl-N-(1-(3-methylbenzofuran-2-yl)ethyl)-3-(3-oxo-3,4-dihydro-2H-pyrido[3,2-b][1,4]oxazin-7-yl)acrylamide;
(R,E)-N-(1-(3-methoxy-2-propoxyphenyl)ethyl)-N-methyl-3-(7-oxo-5,6,7,8-tetrahydro-1,8-naphthyridin-3-yl)acrylamide;
(E)-N-(3-methoxy-2-propoxybenzyl)-N-methyl-3-(4-oxo-2,3,4,5-tetrahydro-1H-pyrido[2,3-b][1,4]diazepin-8-yl)acrylamide;
(R,E)-N-methyl-N-(1-(3-methylbenzofuran-2-yl)ethyl)-3-(4-oxo-2,3,4,5-tetrahydro-1H-pyrido[2,3-b][1,4]diazepin-8-yl)acrylamide;
(E)-N-methyl-N-((1-methyl-1H)-indol-2-yl)methyl)-3-(4-oxo-2,3,4,5-tetrahydro-1H-pyrido[2,3-b][1,4]diazepin-8-yl)acrylamide;
(E)-N-((5-fluoro-3-methylbenzo[b]thiophen-2-yl)methyl)-N-methyl-3-(4-oxo-2,3,4,5-tetrahydro-1H-pyrido[2,3-b][1,4]diazepin-8-yl)acrylamide;
(R,E)-N-(1-(3-ethylbenzofuran-2-yl)ethyl)-N-methyl-3-(4-oxo-2,3,4,5-tetrahydro-1H-pyrido[2,3-b][1,4]diazepin-8-yl)acrylamide;
(E)-3-(5-hydroxy-6,6-dimethyl-7-oxo-5,6,7,8-tetrahydro-1,8-naphthyridin-3-yl)-N-methyl-N-((3-methylbenzofuran-2-yl)methyl)acrylamide;
(E)-3-(5-hydroxy-6,6-dimethyl-7-oxo-5,6,7,8-tetrahydro-1,8-naphthyridin-3-yl)-N-methyl-N-((1-methyl-1H-indol-2-yl)methyl)acrylamide; or
(R,E)-3-(5-hydroxy-6,6-dimethyl-7-oxo-5,6,7,8-tetrahydro-1,8-naphthyridin-3-yl)-N-methyl-N-(1-(3-methylbenzofuran-2-yl)ethyl)acrylamide.

19. The compound of claim 1, wherein the compound inhibits FabI with a Kᵢ of about 5 μM or less, about 1 μM or less, about 100 nM or less, about 10 nM or less, or about 1 nM or less.

20. The compound of claim 1, wherein the compound inhibits FabI with an IC₅₀ of about 30 μM or less, about 1 μM or less, about 100 nM or less, or about 10 nM or less.

21. The compound of claim 1, wherein the compound inhibits FabI with an MIC of about 32 μg/mL or less, about 16 μg/mL or less, or about 8 μg/mL or less, about 4 μg/mL or less, about 2 μg/mL or less, about 1 μg/mL or less, about 0.5 μg/mL or less, about 0.25 μl/mL or less, or about 0.125 μg/mL or less.

22. A pharmaceutical composition comprising a compound of claim 1 and a pharmaceutically acceptable carrier or excipient.

23. The composition of claim 22, wherein the composition is formulated for intravenous administration.

24. The composition of claim 22, wherein the composition is formulated for injectable administration.

25. The composition of claim 22, wherein the composition is formulated for topical application.

26. The composition of claim 22, wherein the composition is formulated as a suppository.

27. The composition of claim 22, wherein the composition is formulated for systemic administration.

28. The composition of claim 22, wherein the composition is formulated for oral administration.

29. The composition of claim 28, wherein the composition is formulated in tablets such that the amount of compound provided in 20 tablets, if taken together, provides a dose of at least the ED₅₀ but no more than ten times the ED₅₀.

30. The composition of claim 22, wherein the composition is formulated for parenteral administration such that the amount of compound provided in 20 cc bolus injection provides a dose of at least the ED₅₀ but no more that ten times the ED₅₀.

31. The composition of claim 22, wherein the composition is formulated for intravenous infusion such that the amount of compound provided in one liter of intravenous injectable solution provides a dose of at least the ED₅₀ but no more that ten times the ED₅₀.

32. A pill for reducing bacterial levels in a subject with a bacteria related illness, comprising a compound of claim 1.

33. The pill of claim 32, wherein the pill provides effective bacterial treatment for at least about 8 hours.

34. The pill of claim 32, wherein the pill provides effective bacterial treatment for at least about 12 hours.

35. The pill of claim 32, wherein the pill provides effective bacterial treatment for at least about 24 hours.

36. The pill of claim 32, wherein the pill provides effective bacterial treatment for at least about one week.

37. A pack of pills in number sufficient for treatment of a bacterial illness, comprising a plurality of pills wherein each pill comprises a compound of claim 1.

38. The pack of pills of claim 37, wherein the pack contains at least 5 pills.

39. The pack of pills of claim 37, wherein the pack contains at least 10 pills.

40. The pack of pills of claim 37, wherein the pack contains at least 20 pills.

41. A method of treating a subject with a bacterial illness comprising administering to the subject the pharmaceutical composition of claim 22.

42. The method of claim 41, wherein the compound inhibits the Fab I activity of a microbe with an IC₅₀ at least 1 order of magnitude lower than the IC₅₀ for inhibiting enoyl CoA hydratase of a mammal.

43. The method of claim 42, wherein the mammal is a human.

44. The method of claim 41, wherein the compound inhibits the Fab I activity of a microbe with a Kᵢ at least 1 order of magnitude lower than the Kᵢ for inhibiting enoyl CoA hydratase of a mammal.

45. The method of claim 44, wherein the mammal is a human.

46. A method of disinfecting an inanimate surface comprising contacting the inanimate surface with a compound of claim 1.

47. A kit comprising the pharmaceutical composition of claim 22 and instructions for use thereof.
